SEQUENCE LISTING (146 sequences)

SEQ ID NO: 1 - protein, 5 aa, Artificial Sequence
  Description of Artificial Sequence Synthetic polypeptide
  FAP(4B9)  CDR-H1
SYAMS

SEQ ID NO: 2 - protein, 17 aa, Artificial Sequence
  Description of Artificial Sequence Synthetic polypeptide
  FAP(4B9)  CDR-H2
AIIGSGASTYYADSVKG

SEQ ID NO: 3 - protein, 8 aa, Artificial Sequence
  Description of Artificial Sequence Synthetic polypeptide
  FAP(4B9)  CDR-H3
GWFGGFNY

SEQ ID NO: 4 - protein, 12 aa, Artificial Sequence
  Description of Artificial Sequence Synthetic polypeptide
  FAP(4B9)  CDR-L1
RASQSVSRSYLA

SEQ ID NO: 5 - protein, 7 aa, Artificial Sequence
  Description of Artificial Sequence Synthetic polypeptide
  FAP(4B9)  CDR-L2
VGSRRAT

SEQ ID NO: 6 - protein, 9 aa, Artificial Sequence
  Description of Artificial Sequence Synthetic polypeptide
  FAP(4B9)  CDR-L3
QQGIMLPPT

SEQ ID NO: 7 - protein, 117 aa, Artificial Sequence
  Description of Artificial Sequence Synthetic polypeptide
  FAP(4B9)  VH
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 8 - protein, 108 aa, Artificial Sequence
  Description of Artificial Sequence Synthetic polypeptide
  FAP(4B9)  VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 9 - protein, 5 aa, Artificial Sequence
  Description of Artificial Sequence Synthetic polypeptide
  FAP(28H1)  CDR-H1
SHAMS

SEQ ID NO: 10 - protein, 16 aa, Artificial Sequence
  Description of Artificial Sequence Synthetic polypeptide
  FAP(28H1)  CDR-H2
AIWASGEQYYADSVKG

SEQ ID NO: 11 - protein, 8 aa, Artificial Sequence
  Description of Artificial Sequence Synthetic polypeptide
  FAP(28H1)  CDR-H3
GWLGNFDY

SEQ ID NO: 12 - protein, 12 aa, Artificial Sequence
  Description of Artificial Sequence Synthetic polypeptide
  FAP(28H1)  CDR-L1
RASQSVSRSYLA

SEQ ID NO: 13 - protein, 7 aa, Artificial Sequence
  Description of Artificial Sequence Synthetic polypeptide
  FAP(28H1)  CDR-L2
GASTRAT

SEQ ID NO: 14 - protein, 9 aa, Artificial Sequence
  Description of Artificial Sequence Synthetic polypeptide
  FAP(28H1)  CDR-L3
QQGQVIPPT

SEQ ID NO: 15 - protein, 116 aa, Artificial Sequence
  Description of Artificial Sequence Synthetic polypeptide
  FAP(28H1) VH
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 16 - protein, 108 aa, Artificial Sequence
  Description of Artificial Sequence Synthetic polypeptide
  FAP(28H1) VL
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 17 - protein, 5 aa, Artificial Sequence
  Description of Artificial Sequence Synthetic polypeptide
  anti-OX40 CDR-H1
SYAIS

SEQ ID NO: 18 - protein, 5 aa, Artificial Sequence
  Description of Artificial Sequence Synthetic polypeptide
  anti-OX40 CDR-H1
SYAMS

SEQ ID NO: 19 - protein, 17 aa, Artificial Sequence
  Description of Artificial Sequence Synthetic polypeptide
  anti-OX40 CDR-H2
GIIPIFGTANYAQKFQG

SEQ ID NO: 20 - protein, 17 aa, Artificial Sequence
  Description of Artificial Sequence Synthetic polypeptide
  anti-OX40 CDR-H2
AISGSGGSTYYADSVKG

SEQ ID NO: 21 - protein, 7 aa, Artificial Sequence
  Description of Artificial Sequence Synthetic polypeptide
  anti-OX40 CDR-H3
EYGWMDY

SEQ ID NO: 22 - protein, 9 aa, Artificial Sequence
  Description of Artificial Sequence Synthetic polypeptide
  anti-OX40 CDR-H3
EYYRGPYDY

SEQ ID NO: 23 - protein, 7 aa, Artificial Sequence
  Description of Artificial Sequence Synthetic polypeptide
  anti-OX40 CDR-H3
EYGSMDY

SEQ ID NO: 24 - protein, 13 aa, Artificial Sequence
  Description of Artificial Sequence Synthetic polypeptide
  anti-OX40 CDR-H3
VNYPYSYWGDFDY

SEQ ID NO: 25 - protein, 7 aa, Artificial Sequence
  Description of Artificial Sequence Synthetic polypeptide
  anti-OX40 CDR-H3
DVGAFDY

SEQ ID NO: 26 - protein, 7 aa, Artificial Sequence
  Description of Artificial Sequence Synthetic polypeptide
  anti-OX40 CDR-H3
DVGPFDY

SEQ ID NO: 27 - protein, 11 aa, Artificial Sequence
  Description of Artificial Sequence Synthetic polypeptide
  anti-OX40 CDR-H3
VFYRGGVSMDY

SEQ ID NO: 28 - protein, 11 aa, Artificial Sequence
  Description of Artificial Sequence Synthetic polypeptide
  anti-OX40 CDR-L1
RASQSISSWLA

SEQ ID NO: 29 - protein, 12 aa, Artificial Sequence
  Description of Artificial Sequence Synthetic polypeptide
  anti-OX40 CDR-L1
RASQSVSSSYLA

SEQ ID NO: 30 - protein, 11 aa, Artificial Sequence
  Description of Artificial Sequence Synthetic polypeptide
  anti-OX40 CDR-L1
QGDSLRSYYAS

SEQ ID NO: 31 - protein, 7 aa, Artificial Sequence
  Description of Artificial Sequence Synthetic polypeptide
  anti-OX40 CDR-L2
DASSLES

SEQ ID NO: 32 - protein, 7 aa, Artificial Sequence
  Description of Artificial Sequence Synthetic polypeptide
  anti-OX40 CDR-L2
GASSRAT

SEQ ID NO: 33 - protein, 7 aa, Artificial Sequence
  Description of Artificial Sequence Synthetic polypeptide
  anti-OX40 CDR-L2
GKNNRPS

SEQ ID NO: 34 - protein, 10 aa, Artificial Sequence
  Description of Artificial Sequence Synthetic polypeptide
  anti-OX40 CDR-L3
QQYLTYSRFT

SEQ ID NO: 35 - protein, 9 aa, Artificial Sequence
  Description of Artificial Sequence Synthetic polypeptide
  anti-OX40 CDR-L3
QQYSSQPYT

SEQ ID NO: 36 - protein, 10 aa, Artificial Sequence
  Description of Artificial Sequence Synthetic polypeptide
  anti-OX40 CDR-L3
QQYISYSMLT

SEQ ID NO: 37 - protein, 9 aa, Artificial Sequence
  Description of Artificial Sequence Synthetic polypeptide
  anti-OX40 CDR-L3
QQYQAFSLT

SEQ ID NO: 38 - protein, 9 aa, Artificial Sequence
  Description of Artificial Sequence Synthetic polypeptide
  anti-OX40 CDR-L3
QQYGSSPLT

SEQ ID NO: 39 - protein, 10 aa, Artificial Sequence
  Description of Artificial Sequence Synthetic polypeptide
  anti-OX40 CDR-L3
NSRVMPHNRV

SEQ ID NO: 40 - protein, 118 aa, Artificial Sequence
  Description of Artificial Sequence Synthetic polypeptide
  49B4 VH
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSS

SEQ ID NO: 41 - protein, 107 aa, Artificial Sequence
  Description of Artificial Sequence Synthetic polypeptide
  49B4 VL
DIQMTQSPSTLSASVGDRVTITCRASQSISSWLAWYQQKPGKAPKLLIYDASSLESGVPSRFSGSGSGTEFTLTISSLQPDDFATYYCQQYSSQPYTFGQGTKVEIK

SEQ ID NO: 42 - protein, 116 aa, Artificial Sequence
  Description of Artificial Sequence Synthetic polypeptide
  8H9 VH
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYGWMDYWGQGTTVTVSS

SEQ ID NO: 43 - protein, 108 aa, Artificial Sequence
  Description of Artificial Sequence Synthetic polypeptide
  8H9 VL
DIQMTQSPSTLSASVGDRVTITCRASQSISSWLAWYQQKPGKAPKLLIYDASSLESGVPSRFSGSGSGTEFTLTISSLQPDDFATYYCQQYLTYSRFTFGQGTKVEIK

SEQ ID NO: 44 - protein, 116 aa, Artificial Sequence
  Description of Artificial Sequence Synthetic polypeptide
  1G4 VH
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYGSMDYWGQGTTVTVSS

SEQ ID NO: 45 - protein, 108 aa, Artificial Sequence
  Description of Artificial Sequence Synthetic polypeptide
  1G4 VL
DIQMTQSPSTLSASVGDRVTITCRASQSISSWLAWYQQKPGKAPKLLIYDASSLESGVPSRFSGSGSGTEFTLTISSLQPDDFATYYCQQYISYSMLTFGQGTKVEIK

SEQ ID NO: 46 - protein, 122 aa, Artificial Sequence
  Description of Artificial Sequence Synthetic polypeptide
  20B7 VH
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARVNYPYSYWGDFDYWGQGTTVTVSS

SEQ ID NO: 47 - protein, 107 aa, Artificial Sequence
  Description of Artificial Sequence Synthetic polypeptide
  20B7 VL
DIQMTQSPSTLSASVGDRVTITCRASQSISSWLAWYQQKPGKAPKLLIYDASSLESGVPSRFSGSGSGTEFTLTISSLQPDDFATYYCQQYQAFSLTFGQGTKVEIK

SEQ ID NO: 48 - protein, 116 aa, Artificial Sequence
  Description of Artificial Sequence Synthetic polypeptide
  CLC-563 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCALDVGAFDYWGQGALVTVSS

SEQ ID NO: 49 - protein, 108 aa, Artificial Sequence
  Description of Artificial Sequence Synthetic polypeptide
  CLC-563 VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 50 - protein, 116 aa, Artificial Sequence
  Description of Artificial Sequence Synthetic polypeptide
  CLC-564  VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAFDVGPFDYWGQGTLVTVSS

SEQ ID NO: 51 - protein, 108 aa, Artificial Sequence; identical to SEQ ID NO: 49
  Description of Artificial Sequence Synthetic polypeptide
  CLC-564 VL

SEQ ID NO: 52 - protein, 120 aa, Artificial Sequence
  Description of Artificial Sequence Synthetic polypeptide
  17A9 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARVFYRGGVSMDYWGQGTLVTVSS

SEQ ID NO: 53 - protein, 106 aa, Artificial Sequence
  Description of Artificial Sequence Synthetic polypeptide
  17A9 VL
SSELTQDPAVSVALGQTVRITCQGDSLRSYYASWYQQKPGQAPVLVIYGKNNRPSGIPDRFSGSSSGNTASLTITGAQAEDEADYYCNSRVMPHNRVFGGGTKLTV

SEQ ID NO: 54 - protein, 816 aa, Artificial Sequence
  Description of Artificial Sequence Synthetic polypeptide
  HC 1 (49B4) VHCH1_VHCH1 Fc knob VH (4B9)
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 55 - protein, 807 aa, Artificial Sequence
  Description of Artificial Sequence Synthetic polypeptide
  HC 2 (49B4) VHCH1_VHCH1 Fc hole VL (4B9)
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 56 - protein, 214 aa, Artificial Sequence
  Description of Artificial Sequence Synthetic polypeptide
  LC (49B4)
DIQMTQSPSTLSASVGDRVTITCRASQSISSWLAWYQQKPGKAPKLLIYDASSLESGVPSRFSGSGSGTEFTLTISSLQPDDFATYYCQQYSSQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 815 aa, Artificial Sequence
  Description of Artificial Sequence Synthetic polypeptide
  HC 1 (49B4) VHCH1_VHCH1 Fc knob VH (28H1)
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 58 - protein, 807 aa, Artificial Sequence
  Description of Artificial Sequence Synthetic polypeptide
  HC 2 (49B4) VHCH1_VHCH1 Fc hole VL (28H1)
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 59 - protein, 807 aa, Artificial Sequence
  Description of Artificial Sequence Synthetic polypeptide
  HC 1 (49B4) VHCH1_VHCH1 Fc knob VL (4B9)
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 60 - protein, 816 aa, Artificial Sequence
  Description of Artificial Sequence Synthetic polypeptide
  HC 2 (49B4) VHCH1_VHCH1 Fc hole VH (4B9)
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 61 - protein, 816 aa, Artificial Sequence
  Description of Artificial Sequence Synthetic polypeptide
  HC 1 (49B4) VHCH1_VHCH1 Fc wt knob VH (4B9)
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 62 - protein, 807 aa, Artificial Sequence
  Description of Artificial Sequence Synthetic polypeptide
  HC 2 (49B4) VHCH1_VHCH1 Fc wt hole VL (4B9)
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAREYYRGPYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 63 - protein, 5 aa, Artificial Sequence
  Description of Artificial Sequence Synthetic polypeptide
  CD3-HCDR1
TYAMN

SEQ ID NO: 64 - protein, 19 aa, Artificial Sequence
  Description of Artificial Sequence Synthetic polypeptide
  CD3-HCDR2
RIRSKYNNYATYYADSVKG

SEQ ID NO: 65 - protein, 14 aa, Artificial Sequence
  Description of Artificial Sequence Synthetic polypeptide
  CD3-HCDR3
HGNFGNSYVSWFAY

SEQ ID NO: 66 - protein, 14 aa, Artificial Sequence
  Description of Artificial Sequence Synthetic polypeptide
  CD3-LCDR1
GSSTGAVTTSNYAN

SEQ ID NO: 67 - protein, 7 aa, Artificial Sequence
  Description of Artificial Sequence Synthetic polypeptide
  CD3-LCDR2
GTNKRAP

SEQ ID NO: 68 - protein, 9 aa, Artificial Sequence
  Description of Artificial Sequence Synthetic polypeptide
  CD3-LCDR3
ALWYSNLWV

SEQ ID NO: 69 - protein, 125 aa, Artificial Sequence
  Description of Artificial Sequence Synthetic polypeptide
  CD3 VH
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 70 - protein, 109 aa, Artificial Sequence
  Description of Artificial Sequence Synthetic polypeptide
  CD3 VL
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 71 - protein, 5 aa, Artificial Sequence
  Description of Artificial Sequence Synthetic polypeptide
  CEA-HCDR1
EFGMN

SEQ ID NO: 72 - protein, 17 aa, Artificial Sequence
  Description of Artificial Sequence Synthetic polypeptide
  CEA-HCDR2
WINTKTGEATYVEEFKG

SEQ ID NO: 73 - protein, 12 aa, Artificial Sequence
  Description of Artificial Sequence Synthetic polypeptide
  CEA-HCDR3
WDFAYYVEAMDY

SEQ ID NO: 74 - protein, 11 aa, Artificial Sequence
  Description of Artificial Sequence Synthetic polypeptide
  CEA-LCDR1
KASAAVGTYVA

SEQ ID NO: 75 - protein, 7 aa, Artificial Sequence
  Description of Artificial Sequence Synthetic polypeptide
  CEA-LCDR2
SASYRKR

SEQ ID NO: 76 - protein, 10 aa, Artificial Sequence
  Description of Artificial Sequence Synthetic polypeptide
  CEA-LCDR3
HQYYTYPLFT

SEQ ID NO: 77 - protein, 121 aa, Artificial Sequence
  Description of Artificial Sequence Synthetic polypeptide
  CEA VH
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 78 - protein, 108 aa, Artificial Sequence
  Description of Artificial Sequence Synthetic polypeptide
  CEA VL
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 79 - protein, 5 aa, Artificial Sequence
  Description of Artificial Sequence Synthetic polypeptide
  CEA-HCDR1 (CEACAM5)
DTYMH

SEQ ID NO: 80 - protein, 17 aa, Artificial Sequence
  Description of Artificial Sequence Synthetic polypeptide
  CEA-HCDR2 (CEACAM5)
RIDPANGNSKYVPKFQG

SEQ ID NO: 81 - protein, 12 aa, Artificial Sequence
  Description of Artificial Sequence Synthetic polypeptide
  CEA-HCDR3 (CEACAM5)
FGYYVSDYAMAY

SEQ ID NO: 82 - protein, 15 aa, Artificial Sequence
  Description of Artificial Sequence Synthetic polypeptide
  CEA-LCDR1 (CEACAM5)
RAGESVDIFGVGFLH

SEQ ID NO: 83 - protein, 7 aa, Artificial Sequence
  Description of Artificial Sequence Synthetic polypeptide
  CEA-LCDR2 (CEACAM5)
RASNRAT

SEQ ID NO: 84 - protein, 9 aa, Artificial Sequence
  Description of Artificial Sequence Synthetic polypeptide
  CEA-LCDR3 (CEACAM5)
QQTNEDPYT

SEQ ID NO: 85 - protein, 121 aa, Artificial Sequence
  Description of Artificial Sequence Synthetic polypeptide
  CEA VH (CEACAM5)
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSS

SEQ ID NO: 86 - protein, 111 aa, Artificial Sequence
  Description of Artificial Sequence Synthetic polypeptide
  CEA VL (CEACAM5)
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIK

SEQ ID NO: 87 - protein, 215 aa, Artificial Sequence
  Description of Artificial Sequence Synthetic polypeptide
  Light chain CEA (CEA TCB)
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 214 aa, Artificial Sequence
  Description of Artificial Sequence Synthetic polypeptide
  Light chain CD3 (CEA TCB)
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 89 - protein, 694 aa, Artificial Sequence
  Description of Artificial Sequence Synthetic polypeptide
  CEA CD3 crossfab VHck fc knob P329GLALA (CEA TCB)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 451 aa, Artificial Sequence
  Description of Artificial Sequence Synthetic polypeptide
  CEA VHCH1 Fc hole P329GLALA (CEA TCB)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 91 - protein, 232 aa, Artificial Sequence
  Description of Artificial Sequence Synthetic polypeptide
  CD3 VH-CL (CEACAM5 TCB)
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 449 aa, Artificial Sequence
  Description of Artificial Sequence Synthetic polypeptide
  CEACAM5 VH-CH1(EE)-Fc (hole, P329G LALA)
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 93 - protein, 674 aa, Artificial Sequence
  Description of Artificial Sequence Synthetic polypeptide
  CEACAM5 VH-CH1(EE)-CD3 VL-CH1-Fc (knob, P329G LALA)
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 94 - protein, 218 aa, Artificial Sequence
  Description of Artificial Sequence Synthetic polypeptide
  CEACAM5  VL-CL(RK)
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 5 aa, Artificial Sequence
  Description of Artificial Sequence Synthetic polypeptide
  (CH2527) CD3-HCDR1
TYAMN

SEQ ID NO: 96 - protein, 19 aa, Artificial Sequence
  Description of Artificial Sequence Synthetic polypeptide
  (CH2527) CD3-HCDR2
RIRSKYNNYATYYADSVKG

SEQ ID NO: 97 - protein, 14 aa, Artificial Sequence
  Description of Artificial Sequence Synthetic polypeptide
  (CH2527) CD3-HCDR3
HGNFGNSYVSWFAY

SEQ ID NO: 98 - protein, 5 aa, Artificial Sequence
  Description of Artificial Sequence Synthetic polypeptide
  (16D5) FolR1-HCDR1
NAWMS

SEQ ID NO: 99 - protein, 19 aa, Artificial Sequence
  Description of Artificial Sequence Synthetic polypeptide
  (16D5) FolR1-HCDR2
RIKSKTDGGTTDYAAPVKG

SEQ ID NO: 100 - protein, 9 aa, Artificial Sequence
  Description of Artificial Sequence Synthetic polypeptide
  (16D5) FolR1-HCDR3
PWEWSWYDY

SEQ ID NO: 101 - protein, 14 aa, Artificial Sequence
  Description of Artificial Sequence Synthetic polypeptide
  (CH2527-VL7-46-13)-LCDR1
GSSTGAVTTSNYAN

SEQ ID NO: 102 - protein, 7 aa, Artificial Sequence
  Description of Artificial Sequence Synthetic polypeptide
  (CH2527-VL7-46-13)-LCDR2
GTNKRAP

SEQ ID NO: 103 - protein, 9 aa, Artificial Sequence
  Description of Artificial Sequence Synthetic polypeptide
  (CH2527-VL7-46-13)-LCDR3
ALWYSNLWV

SEQ ID NO: 104 - protein, 125 aa, Artificial Sequence; identical to SEQ ID NO: 69
  Description of Artificial Sequence Synthetic polypeptide
  (CH2527) CD3 VH

SEQ ID NO: 105 - protein, 120 aa, Artificial Sequence
  Description of Artificial Sequence Synthetic polypeptide
  (16D5) FolR1 VH
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSS

SEQ ID NO: 106 - protein, 109 aa, Artificial Sequence; identical to SEQ ID NO: 70
  Description of Artificial Sequence Synthetic polypeptide
  (CH2527-VL7-46-13)VL

SEQ ID NO: 107 - protein, 689 aa, Artificial Sequence
  Description of Artificial Sequence Synthetic polypeptide
  (16D5)VH-CH1-(CH2527)VH-CH1 Fc knob PGLALA
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 450 aa, Artificial Sequence
  Description of Artificial Sequence Synthetic polypeptide
  (16D5)VH-CH1-Fc hole PGLALA H435R-Y436F
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 109 - protein, 215 aa, Artificial Sequence
  Description of Artificial Sequence Synthetic polypeptide
  (CH2527-VL7-46-13)VL-CL
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 110 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 111 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 112 - protein, 118 aa, Artificial Sequence [sequence omitted]

SEQ ID NO: 113 - protein, 107 aa, Artificial Sequence [sequence omitted]

SEQ ID NO: 114 - protein, 121 aa, Artificial Sequence
  Description of Artificial Sequence Synthetic polypeptide
  VH (PD-L1) 2
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 115 - protein, 108 aa, Artificial Sequence
  Description of Artificial Sequence Synthetic polypeptide
  VL (PD-L1) 2
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 116 - protein, 120 aa, Artificial Sequence [sequence omitted]

SEQ ID NO: 117 - protein, 111 aa, Artificial Sequence [sequence omitted]

SEQ ID NO: 118 - protein, 113 aa, Artificial Sequence [sequence omitted]

SEQ ID NO: 119 - protein, 107 aa, Artificial Sequence [sequence omitted]

SEQ ID NO: 120 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 121 - protein, 748 aa, Artificial Sequence
  Description of Artificial Sequence Synthetic polypeptide
  hu FAP ectodomain+poly-lys-tag+his6-tag
RPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 122 - protein, 761 aa, Mus musculus
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 123 - protein, 749 aa, Artificial Sequence
  Description of Artificial Sequence Synthetic polypeptide
  Murine FAP ectodomain+poly-lys-tag+his6-tag
RPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGILSGRSQNHLYTHMTHFLKQCFSLSDGKKKKKKGHHHHHH

SEQ ID NO: 124 - protein, 748 aa, Artificial Sequence
  Description of Artificial Sequence Synthetic polypeptide
  Cynomolgus FAP ectodomain+poly-lys-tag+his6-tag
RPPRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPFVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEDYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 125 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 126 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 127 - protein, 255 aa, Mus musculus
MAHLMTVQLLLLVMWMAECAQSRATRARTELLNVCMDAKHHKEKPGPEDNLHDQCSPWKTNSCCSTNTSQEAHKDISYLYRFNWNHCGTMTSECKRHFIQDTCLYECSPNLGPWIQQVDQSWRKERILDVPLCKEDCQQWWEDCQSSFTCKSNWHKGWNWSSGHNECPVGASCHPFTFYFPTSAALCEEIWSHSYKLSNYSRGSGRCIQMWFDPAQGNPNEEVARFYAEAMSGAGFHGTWPLLCSLSLVLLWVIS

SEQ ID NO: 128 - protein, 257 aa, Macaca fascicularis
MAQRMTTQLLLLLVWVAVVGEAQTRTARARTELLNVCMNAKHHKEKPGPEDKLHEQCRPWKKNACCSTNTSQEAHKDVSYLYRFNWNHCGEMAPACKRHFIQDTCLYECSPNLGPWIQQVDQSWRKERVLNVPLCKEDCERWWEDCRTSYTCKSNWHKGWNWTSGFNKCPVGAACQPFHFYFPTPTVLCNEIWTYSYKVSNYSRGSGRCIQMWFDPAQGNPNEEVARFYAAAMSGAGPWAAWPLLLSLALTLLWLLS

SEQ ID NO: 129 - protein, 2322 aa, Homo sapiens
MQSGPRPPLPAPGLALALTLTMLARLASAASFFGENHLEVPVATALTDIDLQLQFSTSQPEALLLLAAGPADHLLLQLYSGRLQVRLVLGQEELRLQTPAETLLSDSIPHTVVLTVVEGWATLSVDGFLNASSAVPGAPLEVPYGLFVGGTGTLGLPYLRGTSRPLRGCLHAATLNGRSLLRPLTPDVHEGCAEEFSASDDVALGFSGPHSLAAFPAWGTQDEGTLEFTLTTQSRQAPLAFQAGGRRGDFIYVDIFEGHLRAVVEKGQGTVLLHNSVPVADGQPHEVSVHINAHRLEISVDQYPTHTSNRGVLSYLEPRGSLLLGGLDAEASRHLQEHRLGLTPEATNASLLGCMEDLSVNGQRRGLREALLTRNMAAGCRLEEEEYEDDAYGHYEAFSTLAPEAWPAMELPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVNDPPHIIFPHGSLMVILEHTQKPLGPEVFQAYDPDSACEGLTFQVLGTSSGLPVERRDQPGEPATEFSCRELEAGSLVYVHRGGPAQDLTFRVSDGLQASPPATLKVVAIRPAIQIHRSTGLRLAQGSAMPILPANLSVETNAVGQDVSVLFRVTGALQFGELQKQGAGGVEGAEWWATQAFHQRDVEQGRVRYLSTDPQHHAYDTVENLALEVQVGQEILSNLSFPVTIQRATVWMLRLEPLHTQNTQQETLTTAHLEATLEEAGPSPPTFHYEVVQAPRKGNLQLQGTRLSDGQGFTQDDIQAGRVTYGATARASEAVEDTFRFRVTAPPYFSPLYTFPIHIGGDPDAPVLTNVLLVVPEGGEGVLSADHLFVKSLNSASYLYEVMERPRHGRLAWRGTQDKTTMVTSFTNEDLLRGRLVYQHDDSETTEDDIPFVATRQGESSGDMAWEEVRGVFRVAIQPVNDHAPVQTISRIFHVARGGRRLLTTDDVAFSDADSGFADAQLVLTRKDLLFGSIVAVDEPTRPIYRFTQEDLRKRRVLFVHSGADRGWIQLQVSDGQHQATALLEVQASEPYLRVANGSSLVVPQGGQGTIDTAVLHLDTNLDIRSGDEVHYHVTAGPRWGQLVRAGQPATAFSQQDLLDGAVLYSHNGSLSPRDTMAFSVEAGPVHTDATLQVTIALEGPLAPLKLVRHKKIYVFQGEAAEIRRDQLEAAQEAVPPADIVFSVKSPPSAGYLVMVSRGALADEPPSLDPVQSFSQEAVDTGRVLYLHSRPEAWSDAFSLDVASGLGAPLEGVLVELEVLPAAIPLEAQNFSVPEGGSLTLAPPLLRVSGPYFPTLLGLSLQVLEPPQHGALQKEDGPQARTLSAFSWRMVEEQLIRYVHDGSETLTDSFVLMANASEMDRQSHPVAFTVTVLPVNDQPPILTTNTGLQMWEGATAPIPAEALRSTDGDSGSEDLVYTIEQPSNGRVVLRGAPGTEVRSFTQAQLDGGLVLFSHRGTLDGGFRFRLSDGEHTSPGHFFRVTAQKQVLLSLKGSQTLTVCPGSVQPLSSQTLRASSSAGTDPQLLLYRVVRGPQLGRLFHAQQDSTGEALVNFTQAEVYAGNILYEHEMPPEPFWEAHDTLELQLSSPPARDVAATLAVAVSFEAACPQRPSHLWKNKGLWVPEGQRARITVAALDASNLLASVPSPQRSEHDVLFQVTQFPSRGQLLVSEEPLHAGQPHFLQSQLAAGQLVYAHGGGGTQQDGFHFRAHLQGPAGASVAGPQTSEAFAITVRDVNERPPQPQASVPLRLTRGSRAPISRAQLSVVDPDSAPGEIEYEVQRAPHNGFLSLVGGGLGPVTRFTQADVDSGRLAFVANGSSVAGIFQLSMSDGASPPLPMSLAVDILPSAIEVQLRAPLEVPQALGRSSLSQQQLRVVSDREEPEAAYRLIQGPQYGHLLVGGRPTSAFSQFQIDQGEVVFAFTNFSSSHDHFRVLALARGVNASAVVNVTVRALLHVWAGGPWPQGATLRLDPTVLDAGELANRTGSVPRFRLLEGPRHGRVVRVPRARTEPGGSQLVEQFTQQDLEDGRLGLEVGRPEGRAPGPAGDSLTLELWAQGVPPAVASLDFATEPYNAARPYSVALLSVPEAARTEAGKPESSTPTGEPGPMASSPEPAVAKGGFLSFLEANMFSVIIPMCLVLLLLALILPLLFYLRKRNKTGKHDVQVLTAKPRNGLAGDTETFRKVEPGQAIPLTAVPGQGPPPGGQPDPELLQFCRTPNPALKNGQYWV

SEQ ID NO: 130 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 131 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 132 - protein, 5 aa, Artificial Sequence
  Description of Artificial Sequence Synthetic polypeptide
  G4S peptide linker
GGGGS

SEQ ID NO: 133 - protein, 10 aa, Artificial Sequence
  Description of Artificial Sequence Synthetic polypeptide
  (G4S)2
GGGGSGGGGS

SEQ ID NO: 134 - protein, 10 aa, Artificial Sequence
  Description of Artificial Sequence Synthetic polypeptide
  (SG4)2
SGGGGSGGGG

SEQ ID NO: 135 - protein, 14 aa, Artificial Sequence
  Description of Artificial Sequence Synthetic polypeptide
  peptide linker
GGGGSGGGGSGGGG

SEQ ID NO: 136 - protein, 10 aa, Artificial Sequence
  Description of Artificial Sequence Synthetic polypeptide
  peptide linker
GSPGSSSSGS

SEQ ID NO: 137 - protein, 15 aa, Artificial Sequence
  Description of Artificial Sequence Synthetic polypeptide
  peptide linker 2
GGGGSGGGGSGGGGS

SEQ ID NO: 138 - protein, 20 aa, Artificial Sequence
  Description of Artificial Sequence Synthetic polypeptide
  peptide linker 3
GGGGSGGGGSGGGGSGGGGS

SEQ ID NO: 139 - protein, 8 aa, Artificial Sequence
  Description of Artificial Sequence Synthetic polypeptide
  peptide linker 4
GSGSGSGS

SEQ ID NO: 140 - protein, 8 aa, Artificial Sequence
  Description of Artificial Sequence Synthetic polypeptide
  peptide linker 5
GSGSGNGS

SEQ ID NO: 141 - protein, 8 aa, Artificial Sequence
  Description of Artificial Sequence Synthetic polypeptide
  peptide linker 6
GGSGSGSG

SEQ ID NO: 142 - protein, 6 aa, Artificial Sequence
  Description of Artificial Sequence Synthetic polypeptide
  peptide linker 7
GGSGSG

SEQ ID NO: 143 - protein, 4 aa, Artificial Sequence
  Description of Artificial Sequence Synthetic polypeptide
  peptide linker 8
GGSG

SEQ ID NO: 144 - protein, 8 aa, Artificial Sequence
  Description of Artificial Sequence Synthetic polypeptide
  peptide linker 9
GGSGNGSG

SEQ ID NO: 145 - protein, 8 aa, Artificial Sequence
  Description of Artificial Sequence Synthetic polypeptide
  peptide linker 10
GGNGSGSG

SEQ ID NO: 146 - protein, 6 aa, Artificial Sequence
  Description of Artificial Sequence Synthetic polypeptide
  peptide linker 11
GGNGSG

(19) United States
(12) Patent Application Publication
(10) Pub. No.: US 2020/0392237 A1
(43) Pub. Date: Dec. 17, 2020

(54) Combination therapy with targeted OX40 agonists

(71) Applicant: Hoffmann-La Roche Inc., Little Falls, NJ (US)

(72) Inventors: Marina BACAC, Schlieren (CH); Sandra GRAU-RICHARDS, Schlieren (CH); Christian KLEIN, Schlieren (CH); Johannes SAM, Schlieren (CH); Pablo UMANA, Schlieren (CH); Sabine LANG, Schlieren (CH); Maria AMANN, Schlieren (CH); Mudita PINCHA, Schlieren (CH)

(73) Assignee: Hoffmann-La Roche Inc., Little Falls, NJ (US)

(21) Appl. No.: 16/860,552

(22) Filed: Apr. 28, 2020

Related U.S. Application Data
(63) Continuation of application No. PCT/EP2018/079781, filed on Oct. 31, 2018.

(30) Foreign Application Priority Data
Nov. 1, 2017 (EP) 17199542.6

(51) Int. Cl. C07K 16/28; C07K 16/30; C07K 16/40

(52) U.S. Cl. CPC C07K 16/2878; C07K 16/2809; C07K 16/2827; C07K 16/2818; C07K 16/3007; C07K 2317/522; C07K 2317/31; C07K 2317/565; C07K 2317/76; C07K 2317/71; C07K 2317/55; C07K 16/40

(57) ABSTRACT

The present invention relates to combination therapies employing tumor targeted bispecific OX40 antibodies, in particular anti-FAP/anti-OX40 antibodies in combination with T-cell activating anti-CD3 bispecific antibodies specific for a tumor-associated antigen, the use of these combination therapies for the treatment of cancer and methods of using the combination therapies.

### CROSS-REFERENCE TO RELATED APPLICATIONS

This application is a Continuation of International Application No. PCT/EP2018/079781, filed Oct. 31, 2018, which claims benefit of priority to EP Application No. 17199542.6 filed Nov. 1, 2017, each of which is incorporated herein by reference in its entirety.

### SEQUENCE LISTING

This application contains a Sequence Listing which has been submitted electronically in ASCII format and is hereby incorporated by reference in its entirety. Said ASCII copy, created on Aug. 3, 2020, is named P34512-US_Seq_Listing.txt and is 235,740 bytes in size.

### FIELD OF THE INVENTION

The present invention relates to combination therapies employing tumor targeted anti-CD3 bispecific antibodies and targeted OX40 agonists, in particular bispecific OX40 antibodies comprising at least one antigen binding domain capable of specific binding to a tumor-associated antigen, the use of these combination therapies for the treatment of cancer and methods of using the combination therapies. Included are also combination therapies employing OX40 agonists comprising at least one antigen binding domain capable of specific binding to a tumor-associated antigen with a tumor targeted anti-CD3 bispecific antibody and with an agent blocking PD-L1/PD-1 interaction, in particular a PD-L1 antibody.

### BACKGROUND

Cancer is one of the leading causes of death worldwide. Despite advances in treatment options, prognosis of patients with advanced cancer remains poor. Consequently, there is a persisting and urgent medical need for optimal therapies to increase survival of cancer patients without causing unacceptable toxicity. Recent results from clinical trials have shown that immune therapies, particularly immune checkpoint inhibitors, can extend the overall survival of cancer patients and lead to durable responses. Despite these promising results, current immune-based therapies are only effective in a proportion of patients and combination strategies are needed to improve therapeutic benefit.

One way to recruit the patient's own immune system to fight cancer is the use of T cell bispecific antibodies (TCBs). These molecules are comprised of an agonistic anti-CD3 unit, specific for the T cell receptor (TCR) on T cells, and a targeting moiety specific for a unique cancer antigen. For example, an anti-CEA/anti-CD3 bispecific antibody is a molecule that targets CEA expressed on tumor cells and CD3 epsilon chain (CD3ε) present on T cells. TCBs redirect polyclonal T cells to lyse cancer cells expressing the respective target antigen on their cell surface. No T cell activation occurs in the absence of such target antigen. In the presence of CEA positive cancer cells, whether circulating or tissue resident, pharmacologically active doses will trigger T-cell activation and associated cytokine release. Parallel to tumor cell depletion anti-CEA/anti-CD3 bispecific antibody leads to a transient decrease of T cells in the peripheral blood within 24 hours after the first administration and to a peak in cytokine release, followed by rapid T-cell recovery and return of cytokine levels to baseline within 72 hours. Thus, in order to achieve complete elimination of tumor cells, there is a need of an additional agent that conserves T-cell activation and immune response to cancer cells.

Triggering of the TCR increases, depending on the strength and duration of this primary stimulus, the expression of costimulatory molecules, e.g. OX40, which is a member of the Tumor necrosis factor receptor (TNFR) superfamily. Concomitant agonistic ligation of this receptor by its respective ligand promotes in turn hallmark T cell effector functions like proliferation, survival and secretion of certain proinflammatory cytokines (IFN-γ, IL-2, TNF-α) while it inhibits suppressive mechanisms, e.g. expression of FoxP3 and secretion of IL-10 (M. Croft et al., Immunol. Rev. 2009, 229(1), 173-191, I. Gramaglia et al., J. Immunol. 1998, 161(12), 6510-6517; S. M. Jensen et al., Seminars in Oncology 2010, 37(5), 524-532). This co-stimulation is needed to raise the full potential of T cells against tumor cells, especially in the context of weak tumor antigen priming, and to sustain the anti-tumor response beyond the first attack allowing for protective memory formation.

However, the immune suppressive microenvironment in certain tumors is high in coinhibitory signals, e.g. PD-L1, but lacks sufficient expression of OX40 ligand. Persistent priming of T cells in this context can result in attenuation of T cell activation, exhaustion and evasion of immune surveillance (Sharpe and Freeman, Nature Rev. Immunol. 2002, 2, 116-126.) (Keir M E et al., 2008 Annu. Rev. Immunol. 26:677).

One means to restore OX40 costimulation specifically in the tumor microenviroment, are bispecific antibodies comprised of at least one antigen binding domain for a tumor associated antigen, for example fibroblast activating protein (FAP) in the tumor stroma, and at least one antigen binding domain for OX40. For example, such bispecific antibodies have been described in WO 2017/055398 A2 and WO 2017/060144 A1. Crosslinking and surface immobilization of such bispecific molecules by cell surface FAP creates a highly agonistic matrix for OX40 positive T cells, where it supports NFκB mediated effector functions and can replace ligation by OX40 Ligand. High FAP expression is reported for a plethora of human tumor indications, either on tumor cells themselves or on immune suppressive cancer associated fibroblasts (CAFs).

In certain patients with a strong immunesupressed or exhausted phenotype, only the combination of polyclonal, yet tumor specific T cell recruitment (signal 1) and the restoration of tumor-restricted positive co-stimulation (signal 2) might facilitate sufficient anti-tumor efficacy and prolonged adaptive immune protection. This can persistently drive the tumor microenvironment towards a more immune-activating and less immune-supressive state. FAP dependent costimulation of OX40 may also facilitate TCB mediated killing of tumor cells at lower intratumoral concentrations which would allow reduction of systemic exposure and correlated side effects. Additionally, the treatment intervals might be prolonged as lower TCB concentration could still be active.

In the present patent application in vitro and in vivo data for the combination of TCBs (anti-CEA/anti-CD3 bispecific antibodies and anti-FolR/anti-CD3 bispecific antibodies) with bispecific anti-FAP/anti-OX40 antibodies are provided which support the rationale of combining T cell recruiters with a tumor targeted OX40 agonist to improve the quantity and quality of an anti-tumor response.

### SUMMARY OF THE INVENTION

The present invention relates to bispecific OX40 antibodies comprising at least one antigen binding domain capable of specific binding to a tumor-associated antigen, in particular anti-Fibroblast activation protein (FAP)/anti-OX40 bispecific antibodies and their use in combination with T-cell activating anti-CD3 bispecific antibodies specific for a tumor-associated antigen, in particular to their use in a method for treating or delaying progression of cancer, more particularly for treating or delaying progression of solid tumors. It has been found that the combination therapy described herein is more effective in inhibiting tumor growth and eliminating tumor cells than treatment with the anti-CD3 bispecific antibodies alone.

In one aspect, the invention provides a bispecific OX40 antibody comprising at least one antigen binding domain capable of specific binding to a tumor-associated antigen for use in a method for treating or delaying progression of cancer, wherein the bispecific OX40 antibody comprising at least one antigen binding domain capable of specific binding to a tumor-associated antigen is used in combination with a T-cell activating anti-CD3 bispecific antibody specific for a tumor-associated antigen. In one aspect, provided is a bispecific OX40 antibody comprising at least one antigen binding domain capable of specific binding to a tumor-associated antigen for use in a method for treating or delaying progression of cancer, wherein the bispecific OX40 antibody comprising at least one antigen binding domain capable of specific binding to a tumor-associated antigen is used in combination with a T-cell activating anti-CD3 bispecific antibody specific for another tumor-associated antigen. In one aspect, the T-cell activating anti-CD3 bispecific antibody specific for a tumor-associated antigen is the T-cell activating anti-CD3 bispecific antibody specific for a tumor-associated antigen is an anti-CEA/anti-CD3 bispecific antibody or an anti-FolR1/anti-CD3 bispecific antibody. Particularly, the T-cell activating anti-CD3 bispecific antibody specific for a tumor-associated antigen is an anti-CEA/anti-CD3 bispecific antibody.

In a further aspect, the bispecific OX40 antibody comprising at least one antigen binding domain capable of specific binding to a tumor-associated antigen is for use in a method as described herein before, wherein the bispecific OX40 antibody comprising at least one antigen binding domain capable of specific binding to a tumor-associated antigen and the T-cell activating anti-CD3 bispecific antibody specific for a tumor-associated antigen are administered together in a single composition or administered separately in two or more different compositions.

In another aspect, the bispecific OX40 antibody comprising at least one antigen binding domain capable of specific binding to a tumor-associated antigen is for use in a method as described herein before, wherein the bispecific OX40 antibody comprising at least one antigen binding domain capable of specific binding to a tumor-associated antigen acts synergistically with the T-cell activating anti-CD3 bispecific antibody specific for a tumor-associated antigen.

In another aspect, provided is a bispecific OX40 antibody comprising at least one antigen binding domain capable of specific binding to a tumor-associated antigen for use in a method for treating or delaying progression of cancer, wherein the bispecific OX40 antibody comprising at least one antigen binding domain capable of specific binding to a tumor-associated antigen is administered concurrently with, prior to, or subsequently to the T-cell activating anti-CD3 bispecific antibody specific for a tumor-associated antigen.

In particular, the bispecific OX40 antibody comprising at least one antigen binding domain capable of specific binding to a tumor-associated antigen is an anti-Fibroblast activation protein (FAP)/anti-OX40 bispecific antibody. In one aspect, the anti-FAP/anti-OX40 antibody is an OX40 agonist. In one aspect, the anti-FAP/anti-OX40 antibody is an antigen binding molecule comprising a Fc domain. In a particular aspect, the anti-FAP/anti-OX40 antibody is an antigen binding molecule comprising a Fc domain with modifications reducing Fcγ receptor binding and/or effector function. The crosslinking by a tumor associated antigen makes it possible to avoid unspecific FcγR-mediated crosslinking and thus higher and more efficacious doses of the anti-FAP/anti-OX40 antibody may be administered in comparison to common OX40 antibodies.

In one aspect, the invention provides a bispecific OX40 antibody comprising at least one antigen binding domain capable of specific binding to a tumor-associated antigen, in particular an anti-FAP/anti-OX40 bispecific antibody, for use in a method for treating or delaying progression of cancer, wherein the bispecific OX40 antibody is used in combination with a T-cell activating anti-CD3 bispecific antibody specific for a tumor-associated antigen and wherein the bispecific OX40 antibody comprises at least one antigen binding domain capable of specific binding to FAP comprising

(a) a heavy chain variable region (V_{H}FAP) comprising (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3, and a light chain variable region (V_{L}FAP) comprising (iv) CDR-L1 comprising the amino acid sequence of SEQ ID NO:4, (v) CDR-L2 comprising the amino acid sequence of SEQ ID NO:5, and (vi) CDR-L3 comprising the amino acid sequence of SEQ ID NO:6, or
(b) a heavy chain variable region (V_{H}FAP) comprising (i) CDR-H1 comprising the amino acid sequence of SEQ ID NO:9, (ii) CDR-H2 comprising the amino acid sequence of SEQ ID NO:10, and (iii) CDR-H3 comprising the amino acid sequence of SEQ ID NO:11, and a light chain variable region (V_{L}FAP) comprising (iv) CDR-L1 comprising the amino acid sequence of SEQ ID NO:12, (v) CDR-L2 comprising the amino acid sequence of SEQ ID NO:13, and (vi) CDR-L3 comprising the amino acid sequence of SEQ ID NO:14.

In a further aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as defined herein before, wherein the bispecific OX40 antibody comprises at least one antigen binding domain capable of specific binding to FAP comprising a heavy chain variable region (V_{H}FAP) comprising an amino acid sequence of SEQ ID NO:7 and a light chain variable region (V_{L}FAP) comprising an amino acid sequence of SEQ ID NO:8 or an antigen binding domain capable of specific binding to FAP comprising a heavy chain variable region (V_{H}FAP) comprising an amino acid sequence of SEQ ID NO:15 and a light chain variable region (V_{L}FAP) comprising an amino acid sequence of SEQ ID NO:16. In a particular aspect, the bispecific OX40 antibody comprises at least one antigen binding domain capable of specific binding to FAP comprising a heavy chain variable region (V_{H}FAP) comprising an amino acid sequence of SEQ ID NO:7 and a light chain variable region (V_{L}FAP) comprising an amino acid sequence of SEQ ID NO:8. In another aspect, the bispecific OX40 antibody comprises at least one an antigen binding domain capable of specific binding to FAP comprising a heavy chain variable region (V_{H}FAP) comprising an amino acid sequence of SEQ ID NO:15 and a light chain variable region (V_{L}FAP) comprising an amino acid sequence of SEQ ID NO:16.

In a further aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as defined herein before, wherein the bispecific OX40 antibody comprises at least one antigen binding domain capable of specific binding to OX40 comprising

(a) a heavy chain variable region (V_{H}OX40) comprising (i) CDR-H1 comprising the amino acid sequence of SEQ ID NO:17, (ii) CDR-H2 comprising the amino acid sequence of SEQ ID NO:19, and (iii) CDR-H3 comprising the amino acid sequence of SEQ ID NO:22, and a light chain variable region (V_{L}OX40) comprising (iv) CDR-L1 comprising the amino acid sequence of SEQ ID NO:28, (v) CDR-L2 comprising the amino acid sequence of SEQ ID NO:31, and (vi) CDR-L3 comprising the amino acid sequence of SEQ ID NO:35, or
(b) a heavy chain variable region (V_{H}OX40) comprising (i) CDR-H1 comprising the amino acid sequence of SEQ ID NO:17, (ii) CDR-H2 comprising the amino acid sequence of SEQ ID NO:19, and (iii) CDR-H3 comprising the amino acid sequence of SEQ ID NO:21, and a light chain variable region (V_{L}OX40) comprising (iv) CDR-L1 comprising the amino acid sequence of SEQ ID NO:28, (v) CDR-L2 comprising the amino acid sequence of SEQ ID NO:31, and (vi) CDR-L3 comprising the amino acid sequence of SEQ ID NO:34, or
(c) a heavy chain variable region (V_{H}OX40) comprising (i) CDR-H1 comprising the amino acid sequence of SEQ ID NO:17, (ii) CDR-H2 comprising the amino acid sequence of SEQ ID NO:19, and (iii) CDR-H3 comprising the amino acid sequence of SEQ ID NO:23, and a light chain variable region (V_{L}OX40) comprising (iv) CDR-L1 comprising the amino acid sequence of SEQ ID NO:28, (v) CDR-L2 comprising the amino acid sequence of SEQ ID NO:31, and (vi) CDR-L3 comprising the amino acid sequence of SEQ ID NO:36, or
(d) a heavy chain variable region (V_{H}OX40) comprising (i) CDR-H1 comprising the amino acid sequence of SEQ ID NO:17, (ii) CDR-H2 comprising the amino acid sequence of SEQ ID NO:19, and (iii) CDR-H3 comprising the amino acid sequence of SEQ ID NO:24, and a light chain variable region (V_{L}OX40) comprising (iv) CDR-L1 comprising the amino acid sequence of SEQ ID NO:28, (v) CDR-L2 comprising the amino acid sequence of SEQ ID NO:31, and (vi) CDR-L3 comprising the amino acid sequence of SEQ ID NO:37, or
(e) a heavy chain variable region (V_{H}OX40) comprising (i) CDR-H1 comprising the amino acid sequence of SEQ ID NO:18, (ii) CDR-H2 comprising the amino acid sequence of SEQ ID NO:20, and (iii) CDR-H3 comprising the amino acid sequence of SEQ ID NO:25, and a light chain variable region (V_{L}OX40) comprising (iv) CDR-L1 comprising the amino acid sequence of SEQ ID NO:29, (v) CDR-L2 comprising the amino acid sequence of SEQ ID NO:32, and (vi) CDR-L3 comprising the amino acid sequence of SEQ ID NO:38, or
(f) a heavy chain variable region (V_{H}OX40) comprising (i) CDR-H1 comprising the amino acid sequence of SEQ ID NO:18, (ii) CDR-H2 comprising the amino acid sequence of SEQ ID NO:20, and (iii) CDR-H3 comprising the amino acid sequence of SEQ ID NO:26, and a light chain variable region (V_{L}OX40) comprising (iv) CDR-L1 comprising the amino acid sequence of SEQ ID NO:29, (v) CDR-L2 comprising the amino acid sequence of SEQ ID NO:32, and (vi) CDR-L3 comprising the amino acid sequence of SEQ ID NO:38, or
(g) a heavy chain variable region (V_{H}OX40) comprising (i) CDR-H1 comprising the amino acid sequence of SEQ ID NO:18, (ii) CDR-H2 comprising the amino acid sequence of SEQ ID NO:20, and (iii) CDR-H3 comprising the amino acid sequence of SEQ ID NO:27, and a light chain variable region (V_{L}OX40) comprising (iv) CDR-L1 comprising the amino acid sequence of SEQ ID NO:30, (v) CDR-L2 comprising the amino acid sequence of SEQ ID NO:33, and (vi) CDR-L3 comprising the amino acid sequence of SEQ ID NO:39.

More particularly, the the bispecific OX40 antibody comprises at least one antigen binding domain capable of specific binding to OX40 comprising

(a) a heavy chain variable region (V_{H}OX40) comprising (i) CDR-H1 comprising the amino acid sequence of SEQ ID NO:17, (ii) CDR-H2 comprising the amino acid sequence of SEQ ID NO:19, and (iii) CDR-H3 comprising the amino acid sequence of SEQ ID NO:22, and a light chain variable region (V_{L}OX40) comprising (iv) CDR-L1 comprising the amino acid sequence of SEQ ID NO:28, (v) CDR-L2 comprising the amino acid sequence of SEQ ID NO:31, and (vi) CDR-L3 comprising the amino acid sequence of SEQ ID NO:35.

In a further aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer, wherein the bispecific OX40 antibody comprises at least one antigen binding domain capable of specific binding to OX40 comprising

(a) a heavy chain variable region (V_{H}OX40) comprising an amino acid sequence of SEQ ID NO:40 and a light chain variable region (V_{L}OX40) comprising an amino acid sequence of SEQ ID NO:41, or
(b) a heavy chain variable region (V_{H}OX40) comprising an amino acid sequence of SEQ ID NO:42 and a light chain variable region (V_{L}OX40) comprising an amino acid sequence of SEQ ID NO:43, or
(c) a heavy chain variable region (V_{H}OX40) comprising an amino acid sequence of SEQ ID NO:44 and a light chain variable region (V_{L}OX40) comprising an amino acid sequence of SEQ ID NO:45, or
(d) a heavy chain variable region (V_{H}OX40) comprising an amino acid sequence of SEQ ID NO:46 and a light chain variable region (V_{L}OX40) comprising an amino acid sequence of SEQ ID NO:47, or
(a) a heavy chain variable region (V_{H}OX40) comprising an amino acid sequence of SEQ ID NO:48 and a light chain variable region (V_{L}OX40) comprising an amino acid sequence of SEQ ID NO:49, or
(a) a heavy chain variable region (V_{H}OX40) comprising an amino acid sequence of SEQ ID NO:50 and a light chain variable region (V_{L}OX40) comprising an amino acid sequence of SEQ ID NO:51, or
(a) a heavy chain variable region (V_{H}OX40) comprising an amino acid sequence of SEQ ID NO:52 and a light chain variable region (V_{L}OX40) comprising an amino acid sequence of SEQ ID NO:53.

In a particular aspect, the bispecific OX40 antibody comprises at least one antigen binding domain capable of specific binding to OX40 comprising

(a) a heavy chain variable region (V_{H}OX40) comprising an amino acid sequence of SEQ ID NO:40 and a light chain variable region (V_{L}OX40) comprising an amino acid sequence of SEQ ID NO:41.

In one aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer, wherein the bispecific OX40 antibody comprising at least one antigen binding domain capable of specific binding to a tumor-associated antigen is an antigen binding molecule further comprising a Fc domain composed of a first and a second subunit capable of stable association. In particular, the bispecific OX40 antibody is an antigen binding molecule comprising an IgG Fc domain, specifically an IgG1 Fc domain or an IgG4 Fc domain. More particularly, the bispecific OX40 antibody is an antigen binding molecule comprising a Fc domain that comprises one or more amino acid substitution that reduces binding to an Fc receptor and/or effector function. In a particular aspect, the bispecific OX40 antibody comprises an IgG1 Fc domain comprising the amino acid substitutions L234A, L235A and P329G.

In another aspect of the invention, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as described herein before, wherein the bispecific OX40 antibody comprises monovalent binding to a tumor associated target and and at least bivalent binding to OX40. In one aspect, the anti-FAP/anti-OX40 bispecific antibody comprises monovalent binding to a tumor associated target and and bivalent binding to OX40. In a particular aspect, the anti-FAP/anti-OX40 bispecific antibody comprises monovalent binding to a tumor associated target and and tetravalent binding to OX40.

In another aspect, the invention provides a bispecific OX40 antibody comprising at least one antigen binding domain capable of specific binding to a tumor-associated antigen, in particular an anti-FAP/anti-OX40 bispecific antibody, for use in a method for treating or delaying progression of cancer as described herein before, wherein the bispecific OX40 antibody comprises a first Fab fragment capable of specific binding to OX40 fused at the C-terminus of the CH1 domain to the VH domain of a second Fab fragment capable of specific binding to OX40 and a third Fab fragment capable of specific binding to OX40 fused at the C-terminus of the CH1 domain to the VH domain of a fourth Fab fragment capable of specific binding to OX40.

In one aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as described herein before, wherein the bispecific OX40 antibody comprises

(i) a first heavy chain comprising an amino acid sequence of SEQ ID NO:54, a second heavy chain comprising an amino acid sequence of SEQ ID NO:55, and four light chains comprising an amino acid sequence of SEQ ID NO:56, or
(ii) a first heavy chain comprising an amino acid sequence of SEQ ID NO:57, a second heavy chain comprising an amino acid sequence of SEQ ID NO:58, and four light chains comprising an amino acid sequence of SEQ ID NO:56, or
(i) a first heavy chain comprising an amino acid sequence of SEQ ID NO:59, a second heavy chain comprising an amino acid sequence of SEQ ID NO:60, and four light chains comprising an amino acid sequence of SEQ ID NO:56, or
(ii) a first heavy chain comprising an amino acid sequence of SEQ ID NO:61, a second heavy chain comprising an amino acid sequence of SEQ ID NO:62, and four light chains comprising an amino acid sequence of SEQ ID NO:56.

In another aspect, the invention provides a bispecific OX40 antibody comprising at least one antigen binding domain capable of specific binding to a tumor-associated antigen, in particular an anti-FAP/anti-OX40 bispecific antibody, for use in a method for treating or delaying progression of cancer, wherein the bispecific OX40 antibody is used in combination with a T-cell activating anti-CD3 bispecific antibody specific for a tumor-associated antigen and wherein the T-cell activating anti-CD3 bispecific antibody is an anti-CEA/anti-CD3 bispecific antibody.

In one aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as decribed herein before, wherein the T-cell activating anti-CD3 bispecific antibody comprises a first antigen binding domain comprising a heavy chain variable region (V_{H}CD3) and a light chain variable region (V_{L}CD3), and a second antigen binding domain comprising a heavy chain variable region (V_{H}CEA) and a light chain variable region (V_{L}CEA).

In another aspect, the invention provides a bispecific OX40 antibody comprising at least one antigen binding domain capable of specific binding to a tumor-associated antigen, in particular an anti-FAP/anti-OX40 bispecific antibody, for use in a method for treating or delaying progression of cancer as decribed herein before, wherein the T-cell activating anti-CD3 bispecific antibody comprises a first antigen binding domain comprising a heavy chain variable region (V_{H}CD3) comprising CDR-H1 sequence of SEQ ID NO:63, CDR-H2 sequence of SEQ ID NO:64, and CDR-H3 sequence of SEQ ID NO:65; and/or a light chain variable region (V_{L}CD3) comprising CDR-L1 sequence of SEQ ID NO:66, CDR-L2 sequence of SEQ ID NO:67, and CDR-L3 sequence of SEQ ID NO:68.

In a further aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as decribed herein before, wherein the T-cell activating anti-CD3 bispecific antibody comprises a first antigen binding domain comprising a heavy chain variable region (V_{H}CD3) comprising the amino acid sequence of SEQ ID NO:69 and/or a light chain variable region (V_{L}CD3) comprising the amino acid sequence of SEQ ID NO:70. In one aspect, provided is a bispecific OX40 antibody comprising at least one antigen binding domain capable of specific binding to a tumor-associated antigen for use in a method for treating or delaying progression of cancer, wherein the T-cell activating anti-CD3 bispecific antibody comprises a second antigen binding domain comprising

(a) a heavy chain variable region (V_{H}CEA) comprising CDR-H1 sequence of SEQ ID NO:71, CDR-H2 sequence of SEQ ID NO:72, and CDR-H3 sequence of SEQ ID NO:73, and/or a light chain variable region (V_{L}CEA) comprising CDR-L1 sequence of SEQ ID NO:74, CDR-L2 sequence of SEQ ID NO:75, and CDR-L3 sequence of SEQ ID NO:76, or
(b) a heavy chain variable region (V_{H}CEA) comprising CDR-H1 sequence of SEQ ID NO:79, CDR-H2 sequence of SEQ ID NO:80, and CDR-H3 sequence of SEQ ID NO:81, and/or a light chain variable region (V_{L}CEA) comprising CDR-L1 sequence of SEQ ID NO:82, CDR-L2 sequence of SEQ ID NO:83, and CDR-L3 sequence of SEQ ID NO:84.

In a particular aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as decribed herein before, wherein the T-cell activating anti-CD3 bispecific antibody comprises a second antigen binding domain comprising a heavy chain variable region (V_{H}CEA) comprising the amino acid sequence of SEQ ID NO:77 and/or a light chain variable region (V_{L}CEA) comprising the amino acid sequence of SEQ ID NO:78 or a second antigen binding domain comprising a heavy chain variable region (V_{H}CEA) comprising the amino acid sequence of SEQ ID NO:85 and/or a light chain variable region (V_{L}CEA) comprising the amino acid sequence of SEQ ID NO:86.

In another aspect, the invention further provides a bispecific OX40 antibody comprising at least one antigen binding domain capable of specific binding to a tumor-associated antigen, in particular an anti-FAP/anti-OX40 bispecific antibody, for use in a method for treating or delaying progression of cancer as decribed herein before, wherein the anti-CEA/anti-CD3 bispecific antibody further comprises a third antigen binding domain that binds to CEA. In particular, the third antigen binding domain comprises (a) a heavy chain variable region (V_{H}CEA) comprising CDR-H1 sequence of SEQ ID NO:71, CDR-H2 sequence of SEQ ID NO:72, and CDR-H3 sequence of SEQ ID NO:73, and/or a light chain variable region (V_{L}CEA) comprising CDR-L1 sequence of SEQ ID NO:74, CDR-L2 sequence of SEQ ID NO:75, and CDR-L3 sequence of SEQ ID NO:76, or (b) a heavy chain variable region (V_{H}CEA) comprising CDR-H1 sequence of SEQ ID NO:79, CDR-H2 sequence of SEQ ID NO:80, and CDR-H3 sequence of SEQ ID NO:81, and/or a light chain variable region (V_{L}CEA) comprising CDR-L1 sequence of SEQ ID NO:82, CDR-L2 sequence of SEQ ID NO:83, and CDR-L3 sequence of SEQ ID NO:84. More particularly, the third antigen binding domain comprises a heavy chain variable region (V_{H}CEA) comprising the amino acid sequence of SEQ ID NO:77 and/or a light chain variable region (V_{L}CEA) comprising the amino acid sequence of SEQ ID NO:78 or wherein the second antigen binding domain comprises a heavy chain variable region (V_{H}CEA) comprising the amino acid sequence of SEQ ID NO:85 and/or a light chain variable region (V_{L}CEA) comprising the amino acid sequence of SEQ ID NO:86

In a further aspect, the T-cell activating anti-CD3 bispecific antibody is an anti-CEA/anti-CD3 bispecific antibody, wherein the first antigen binding domain is a cross-Fab molecule wherein the variable domains or the constant domains of the Fab heavy and light chain are exchanged, and the second and third, if present, antigen binding domain is a conventional Fab molecule.

In a further aspect, the T-cell activating anti-CD3 bispecific antibody is an anti-CEA/anti-CD3 bispecific antibody, wherein (i) the second antigen binding domain is fused at the C-terminus of the Fab heavy chain to the N-terminus of the Fab heavy chain of the first antigen binding domain, the first antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain, or (ii) the first antigen binding domain is fused at the C-terminus of the Fab heavy chain to the N-terminus of the Fab heavy chain of the second antigen binding domain, the second antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain.

In one aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as decribed herein before, wherein the anti-CEA/anti-CD3 bispecific antibody comprises a third antigen binding domain that binds to CEA. In a further aspect, the anti-CEA/anti-CD3 bispecific antibody comprises a Fc domain composed of a first and a second subunit capable of stable association. In particular, the anti-CEA/anti-CD3 bispecific antibody comprises an IgG Fc domain, specifically an IgG1 Fc domain or an IgG4 Fc domain. More particularly, the anti-CEA/anti-CD3 bispecific antibody comprises a Fc domain that comprises one or more amino acid substitutions that reduce binding to an Fc receptor and/or effector function. In a particular aspect, the anti-CEA/anti-CD3 bispecific antibody comprises an IgG1 Fc domain comprising the amino acid substitutions L234A, L235A and P329G.

In another aspect, the invention provides a bispecific OX40 antibody comprising at least one antigen binding domain capable of specific binding to a tumor-associated antigen, in particular an anti-FAP/anti-OX40 bispecific antibody, for use in a method for treating or delaying progression of cancer, wherein the bispecific OX40 antibody is used in combination with a T-cell activating anti-CD3 bispecific antibody specific for a tumor-associated antigen and wherein the T-cell activating anti-CD3 bispecific antibody is an anti-FolR1/anti-CD3 bispecific antibody.

In one aspect, the invention provides a bispecific OX40 antibody comprising at least one antigen binding domain capable of specific binding to a tumor-associated antigen, in particular an anti-FAP/anti-OX40 bispecific antibody, for use in a method for treating or delaying progression of cancer as described herein before, wherein the T-cell activating anti-CD3 bispecific antibody comprises a first antigen binding domain comprising a heavy chain variable region (V_{H}CD3), a second antigen binding domain comprising a heavy chain variable region (V_{H}FolR1) and a common light chain variable region.

In another aspect of the invention, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as described herein before, wherein the T-cell activating anti-CD3 bispecific antibody comprises a first antigen binding domain comprising a heavy chain variable region (V_{H}CD3) comprising CDR-H1 sequence of SEQ ID NO:95, CDR-H2 sequence of SEQ ID NO:96, and CDR-H3 sequence of SEQ ID NO:97; a second antigen binding domain comprising a heavy chain variable region (V_{H}FolR1) comprising CDR-H1 sequence of SEQ ID NO:98, CDR-H2 sequence of SEQ ID NO:99, and CDR-H3 sequence of SEQ ID NO:100; and a common light chain comprising a CDR-L1 sequence of SEQ ID NO:101, CDR-L2 sequence of SEQ ID NO:102, and CDR-L3 sequence of SEQ ID NO:103.

In a further aspect, the invention provides a bispecific OX40 antibody comprising at least one antigen binding domain capable of specific binding to a tumor-associated antigen, in particular an anti-FAP/anti-OX40 bispecific antibody, for use in a method for treating or delaying progression of cancer as described herein before, wherein the T-cell activating anti-CD3 bispecific antibody comprises a first antigen binding domain comprising a heavy chain variable region (V_{H}CD3) comprising the sequence of SEQ ID NO:104 and a second antigen binding domain comprising a heavy chain variable region (V_{H}FolR1) comprising the sequence of SEQ ID NO:105; and wherein the common light chain comprises the sequence of SEQ ID NO:106.

In one aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as described herein before, wherein the anti-FolR1/anti-CD3 bispecific antibody comprises a third antigen binding domain that binds to FolR1.

In a further aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as described herein before, wherein the anti-FolR1/anti-CD3 bispecific antibody comprises a first heavy chain comprising the amino acid sequence of SEQ ID NO:107, a second heavy chain comprising the amino acid sequence of SEQ ID NO:108 and a common light chain of SEQ ID NO: 109.

In another aspect, the invention provides a bispecific OX40 antibody comprising at least one antigen binding domain capable of specific binding to a tumor-associated antigen, in particular an anti-FAP/anti-OX40 bispecific antibody, for use in a method for treating or delaying progression of cancer as described herein before, wherein the bispecific OX40 antibody is used in combination with a T-cell activating anti-CD3 bispecific antibody specific for a tumor-associated antigen and wherein the combination is administered at intervals from about about one week to three weeks.

In yet another aspect, the invention provides a bispecific OX40 antibody comprising at least one antigen binding domain capable of specific binding to a tumor-associated antigen, in particular an anti-FAP/anti-OX40 bispecific antibody, for use in a method for treating or delaying progression of cancer, wherein the bispecific OX40 antibody is used in combination with a T-cell activating anti-CD3 bispecific antibody specific for a tumor-associated antigen and in combination with an agent blocking PD-L1/PD-1 interaction. In particular, the agent blocking PD-L1/PD-1 interaction is an anti-PD-L1 antibody or an anti-PD1 antibody. More particularly, the agent blocking PD-L1/PD-1 interaction is selected from the group consisting of atezolizumab, durvalumab, pembrolizumab and nivolumab. In a specific aspect, the agent blocking PD-L1/PD-1 interaction is atezolizumab.

In a further aspect, the invention provides a pharmaceutical product comprising (A) a first composition comprising as active ingredient a bispecific OX40 antibody comprising at least one antigen binding domain capable of specific binding to a tumor-associated antigen, in particular an anti-FAP/anti-OX40 bispecific antibody, and a pharmaceutically acceptable excipient; and (B) a second composition comprising as active ingredient a T-cell activating anti-CD3 bispecific antibody specific for a tumor-associated antigen, in particular an anti-CEA/anti-CD3 bispecific antibody or anti-FolR1/anti-CD3 bispecific antibody, and a pharmaceutically acceptable excipient, for use in the combined, sequential or simultaneous treatment of a disease, in particular for the treatment of cancer.

In another aspect, provided is a pharmaceutical composition comprising a bispecific OX40 antibody comprising at least one antigen binding domain capable of specific binding to a tumor-associated antigen, in particular an anti-FAP/anti-OX40 bispecific antibody, and a T-cell activating anti-CD3 bispecific antibody specific for a tumor-associated antigen, in particular an anti-CEA/anti-CD3 bispecific antibody or anti-FolR1/anti-CD3 bispecific antibody. In one aspect, the pharmaceutical composition further comprises blocking PD-L1/PD-1 interaction. In particular, the agent blocking PD-L1/PD-1 interaction is an anti-PD-L1 antibody or an anti-PD1 antibody. More particularly, the agent blocking PD-L1/PD-1 interaction is selected from the group consisting of atezolizumab, durvalumab, pembrolizumab and nivolumab. In a specific aspect, the agent blocking PD-L1/PD-1 interaction is atezolizumab. In one particular aspect, the pharmaceutical composition is for use in the treatment of solid tumors.

In an additional aspect, the invention provides a kit for treating or delaying progression of cancer in a subject, comprising a package comprising (A) a first composition comprising as active ingredient a bispecific OX40 antibody comprising at least one antigen binding domain capable of specific binding to a tumor-associated antigen, in particular an anti-FAP/anti-OX40 bispecific antibody, and a pharmaceutically acceptable excipient; (B) a second composition comprising as active ingredient a T-cell activating anti-CD3 bispecific antibody specific for a tumor-associated antigen, in particular an anti-CEA/anti-CD3 bispecific antibody or anti-FolR1/anti-CD3 bispecific antibody, and a pharmaceutically acceptable excipient, and (C) instructions for using the compositions in a combination therapy. In one aspect, provided is a kit for treating or delaying progression of cancer in a subject, comprising a package comprising (A) a first composition comprising as active ingredient a bispecific OX40 antibody comprising at least one antigen binding domain capable of specific binding to a tumor-associated antigen, in particular an anti-FAP/anti-OX40 bispecific antibody, and a pharmaceutically acceptable excipient; (B) a second composition comprising as active ingredient a T-cell activating anti-CD3 bispecific antibody specific for a tumor-associated antigen, in particular an anti-CEA/anti-CD3 bispecific antibody or anti-FolR1/anti-CD3 bispecific antibody, and a pharmaceutically acceptable excipient, (c) a third composition comprising as active ingredient an agent blocking PD-L1/PD-1 interaction, in particular atezolizumab, and a pharmaceutically acceptable excipient, and (C) instructions for using the compositions in a combination therapy.

In a further aspect, the invention relates to the use of a combination of a bispecific OX40 antibody comprising at least one antigen binding domain capable of specific binding to a tumor-associated antigen, in particular an anti-FAP/anti-OX40 bispecific antibody, and a T-cell activating anti-CD3 bispecific antibody specific for a tumor-associated antigen, in particular an anti-CEA/anti-CD3 bispecific antibody or anti-FolR1/anti-CD3 bispecific antibody, in the manufacture of a medicament for treating or delaying progression of a proliferative disease, in particular cancer.

In particular, provided is the use of a combination of a T bispecific OX40 antibody comprising at least one antigen binding domain capable of specific binding to a tumor-associated antigen, in particular an anti-FAP/anti-OX40 bispecific antibody, and a T-cell activating anti-CD3 bispecific antibody specific for a tumor-associated antigen, in particular an anti-CEA/anti-CD3 bispecific antibody or anti-FolR1/anti-CD3 bispecific antibody in the manufacture of a medicament for treating a disease selected from the group consisting of colon cancer, lung cancer, ovarian cancer, gastric cancer, bladder cancer, pancreatic cancer, endometrial cancer, breast cancer, kidney cancer, esophageal cancer, or prostate cancer.

In another aspect, the invention provides a method for treating or delaying progression of cancer in a subject comprising administering to the subject an effective amount of a T bispecific OX40 antibody comprising at least one antigen binding domain capable of specific binding to a tumor-associated antigen, in particular an anti-FAP/anti-OX40 bispecific antibody, and a T-cell activating anti-CD3 bispecific antibody specific for a tumor-associated antigen, in particular an anti-CEA/anti-CD3 bispecific antibody or anti-FolR1/anti-CD3 bispecific antibody. In another aspect, provided is a method for treating or delaying progression of cancer in a subject comprising administering to the subject an effective amount of a T bispecific OX40 antibody comprising at least one antigen binding domain capable of specific binding to a tumor-associated antigen, in particular an anti-FAP/anti-OX40 bispecific antibody, a T-cell activating anti-CD3 bispecific antibody specific for a tumor-associated antigen, in particular an anti-CEA/anti-CD3 bispecific antibody or anti-FolR1/anti-CD3 bispecific antibody, and an agent blocking PD-L1/PD-1 interaction, in particular an anti-PD-L1 antibody or an anti-PD1 antibody.

In a further aspect, provided is an anti-FAP/anti-OX40 bispecific antibody for use in a method for treating or delaying progression of cancer, wherein the anti-FAP/anti-OX40 bispecific antibody is used in combination with an agent blocking PD-L1/PD-1 interaction. In particular, the agent blocking PD-L1/PD-1 interaction is an anti-PD-L1 antibody or an anti-PD1 antibody. More particularly, the agent blocking PD-L1/PD-1 interaction is selected from the group consisting of atezolizumab, durvalumab, pembrolizumab and nivolumab. In a specific aspect, the agent blocking PD-L1/PD-1 interaction is atezolizumab.

### BRIEF DESCRIPTION OF THE DRAWINGS

FIGS. 1A-1C show a particular anti-FAP/anti-OX40 bispecific antibody and a particular anti-CEA/anti-CD3 bispecific antibody, respectively, as used in the Examples These molecules are described in more detail in Examples 1 and 2, respectively. The thick black point stands for the knob-into-hole modification. * symbolizes amino acid modifications in the CH1 and CL domain (so-called charged residues). FIG. 1A shows a particular anti-FAP/anti-OX40 bispecific antibody with tetravalent binding to OX40 and monovalent binding to FAP (4+1 format, FAP VH and VL fused to the C-termini of the Fc domain). The molecule is called herein FAP OX40 iMab. In FIG. 1B an exemplary bispecific anti-CEA/anti-CD3 antibody in 2+1 format is shown (named CEACAM5 CD3 TCB). Another anti-CEA/anti-CD3 antibody in 2+1 format (called CEA CD3 TCB) is shown in FIG. 1C.

FIG. 2 shows TCB mediated lysis of MKN45 NucLight red tumor cells by various human immune cell preparations (Example 3). Different human immune effector cell preparations (resting PBMC, CD4 or CD8 T cells) were cocultured with MKN-45 NucLight Red cells and irradiated NIH/3T3 huFAP in the presence of a serial dilution row of CEACAM5 CD3 TCB (CEA CD3 TCB (2)) for 48 hours. The amount of living tumor cells was quantified by fluorescence microscopy high content life imaging using the Incucyte Zoom System (Essenbioscience, HD phase-contrast, green fluorescence and red fluorescence, 10× objective) in a 3 hours interval for 48 hours at 37° C. and 5% CO₂. The integrated red fluorescence of healthy tumor cells (RCU×μm²/image) of triplicates (median) was used to calculate the specific lysis which was plotted against the used TCB concentration to show the cytolytic potential of T cells.

FIGS. 3A-3D show the expression of OX40 on T cells upon TCB stimulation. Different human immune effector cell preparations (resting PBMC, CD4 or CD8 T cells) were cocultured with MKN-45 NucLight Red cells and irradiated NIH/3T3 huFAP in the presence of a serial dilution row of CEACAM5 CD3 TCB (CEA CD3 TCB (2)) for 48 hours. The expression of OX40 was determined on CD4⁺ and CD8⁺ T cells by flow cytometry. The percentage of positive cells (FIGS. 3A and 3C) and MFI (FIGS. 3B and 3D) of triplicates (median) was plotted against the used TCB concentration for CD4 positive T cells (FIGS. 3A and 3B) and CD8 positive (FIGS. 3C and 3D) T cells. Error bars indicate the SEM. TCB mediated a dose dependent cell surface expression of OX40 on CD4⁺ T cells and on CD8⁺ T cell, albeit to a higher extent on CD4⁺ T cells.

FIGS. 4A-4C show that OX40 costimulation did not influence the cytolytic potential of FolR1 CD3 TCB. Resting CD4 T cells were cocultured for 48 hrs with HeLa NucLight Red cells and irradiated NIH/3T3 huFAP in the presence of a serial dilution row of FolR1 CD3 TCB with (FIG. 4B) or without a fixed concentration of FAP OX40 iMAB (FIG. 4A). The amount of living tumor cells was quantified by fluorescence microscopy high content life imaging using the Incucyte Zoom System (Essenbioscience, HD phase-contrast, green fluorescence and red fluorescence, 10× objective) in a 3 hours interval for 42 hrs at 37° C. and 5% CO₂. The integrated red fluorescence of healthy tumor cells (RCU×μm²/image) of triplicates (median) was plotted against the used TCB concentration for various time points to show the cytolytic potential of T cells. Error bars indicate the SEM. The Area under the curve for each timepoint was calculated as measure for cytotoxicity and plotted against the time point. For comparison of the AUC values for both FolR1 CD3 TCB alone and in combination with FAP OX40 iMAB were plotted against the time in FIG. 4C showing that the addition of FAP OX40 iMab had no influence on the the cytolytic potential of FolR1 CD3 TCB.

FIGS. 5A-5C show that OX40 costimulation did not influence the cytolytic potential of CEACAM5 CD3 TCB (CEA CD3 TCB (2)). Different human immune effector cell preparations (resting PBMC in FIG. 5C, CD4 T cells in FIG. 5A and CD8 T cells in FIG. 5B) were cocultured for 48 hours with MKN-45 NucLight Red cells and irradiated NIH/3T3 huFAP in the presence of a serial dilution row of CEACAM5 CD3 TCB with or without a fixed concentration of FAP OX40 iMab. The amount of living tumor cells was quantified by fluorescence microscopy high content life imaging using the Incucyte Zoom System (Essenbioscience, HD phase-contrast, green fluorescence and red fluorescence, 10× objective) in a 3 hours interval for 48 hours at 37° C. and 5% CO₂. The integrated red fluorescence of healthy tumor cells (RCU×μm²/image) of triplicates (median) was used to calculate the specific lysis which was plotted against the used TCB concentration to show the cytolytic potential of T cells. Here, the 42 hours timepoint is shown exemplary. Error bars indicate the SEM.

FIGS. 6A-6D show that FAP OX40 iMAB co-stimulation did increase FolR1 CD3 TCB mediated TNF-α secretion and was depending on agonistic TCR stimulation. Resting CD4 T cells were cocultured for 48 hrs with irradiated TNF-α sensor cells, NIH/3T3 huFAP and HeLa NucLight Red cells in the presence of a serial dilution row of FolR1 CD3 TCB with or without a fixed concentration of FAP OX40 iMAB. The amount of TNF-α was quantified as GFP induction in TNF-α sensor cells by fluorescence microscopy high content life imaging using the Incucyte Zoom System (Essenbioscience, HD phase-contrast, green fluorescence and red fluorescence, 10× objective) in a 3 hours interval for 42 hrs at 37° C. and 5% CO₂. The integrated green fluorescence of TNF-α sensor cells (GCU×μm²/image) of triplicates (median) was plotted against the used TCB concentration to quantify TNFα secretion of T cells. Error bars indicate the SEM. The results for FolR1 CD3 TCB are shown in FIG. 6A (without costimulation) and FIG. 6C (with FAP OX40 iMAB co-stimulation) whereas FIGS. 6B and 6D show the results with a negative control CD3 TCB.

FIGS. 7A-7D show that FAPOx40iMAB costimulation did increase CEA CD3 TCB or CEACAM5 CD3 TCB mediated TNF-α secretion. Resting CD4 T cells were cocultured for 48 hrs with irradiated TNF-α sensor cells, NIH/3T3 huFAP and MKN-45 NLR cells in the presence of a serial dilution row of CEA CD3 TCB and CEACAM5 CD3 TCB, respectively, with or without a fixed concentration of FAP OX40 iMAB. The amount of TNF-α was quantified as GFP induction in TNF-α sensor cells by fluorescence microscopy high content life imaging as described above. The integrated green fluorescence of TNF-α sensor cells (GCUxum2/image) of triplicates (median) was plotted against the used TCB concentration to quantify TNF-α secretion of T cells. Error bars indicate the SEM. The results for CEACAM5 CD3 TCB (CEA CD3 TCB (2)) are shown in FIG. 7A (without costimulation) and in FIG. 7C (with FAP OX40 iMAB costimulation). The results for CEA CD3 TCB are shown in FIG. 7B (without costimulation) and in FIG. 7D (with FAP OX40 iMAB costimulation).

FIGS. 8A-8D summarize the effects seen with the different TCBs or different cell lines, respectively. Resting CD4 T cells were cocultured for 48 hrs with TNF-α sensor cell, irradiated NIH/3T3 huFAP and different target cell lines HeLa NucLight Red cells (FIG. 8B), MKN-45 NucLight Red cells (FIGS. 8A and 8C) or Skov-3 cells (FIG. 8D) with or without a fixed concentration of FAP Ox40 iMAB in the presence of a serial dilution row of FolR CD3 TCB (FIGS. 8B and 8D), CEA CD3 TCB (FIG. 8C) or CEACAM5 CD3 TCB (FIG. 8A). The amount of TNF-α was quantified as GFP induction in TNF-α sensor cells by fluorescence microscopy high content life imaging 2. The AUC of GFP was calculated for each condition and time point and was plotted against each timepoint to quantify TNF-α secretion of T cells. OX40 costimulation did increase CEA CD3 TCB, CEACAM5 CD3 TCB and FolR CD3 TCB mediated TNF-α release.

FIGS. 9A-9D show that OX40 costimulation did modulate CEACAM5 CD3 TCB mediated cytokine secretion. Resting CD4 T cells were cocultured for 48 hrs with MKN-45 NucLight Red cells and irradiated NIH/3T3 huFAP in the presence of a serial dilution row of CEACAM5 CD3 TCB with or without a fixed concentration of FAP Ox40 iMAB. The secreted amount of TNF-α, IFN-γ, IL-2, IL-10, IL-9 and IL-17A was quantified at the 48h end point using cytometric bead array technology. The respective cytokine concentrations were plotted against the TCB concentration. Off note—secretion of proinflammatory cytokine TNF-α (FIG. 9A), IFN-γ (FIG. 9C), and IL-2 (FIG. 9B) was enhanced by OX40 costimulation, whereas that of immunesupressive IL-10 (FIG. 9D) was decreased.

FIGS. 10A-10D show that OX40 costimulation did modulate CEA CD3 TCB mediated cytokine secretion. Resting CD4 T cells were cocultured for 48 hrs with MKN-45 NucLight Red cells and irradiated NIH/3T3 huFAP in the presence of a serial dilution row of CEA CD3 TCB with or without a fixed concentration of FAP OX40 iMAB. The secreted amount of TNF-α, IFN-γ, IL-2, IL-10 (FIG. 10D), IL-9 and IL-17A was quantified at the 48h end point using cytometric bead array technology. The respective cytokine concentrations were plotted against the TCB concentration. Off note—secretion of proinflammatory cytokine TNF-α (FIG. 10A), IFN-γ (FIG. 10C), and IL-2 (FIG. 10B) was enhanced by OX40 costimulation.

FIGS. 11A-11D show that OX40 costimulation did modulate FolR1 CD3 TCB mediated cytokine secretion. Resting CD4 T cells were cocultured for 48 hrs with HeLa NucLight Red cells and irradiated NIH/3T3 huFAP in the presence of a serial dilution row of FolR1 CD3 TCB with or without a fixed concentration of FAP OX40 iMAB. The secreted amount of TNF-α, IFN-γ, IL-2, and IL-10 was quantified at the 48h end point using cytometric bead array technology. The respective cytokine concentrations were plotted against the TCB concentration. Off note—secretion of proinflammatory cytokine TNF-α (FIG. 11A), IFN-γ (FIG. 11C), and IL-2 (FIG. 11B) was enhanced by OX40 costimulation whereas that of immunesupressive IL-10 (FIG. 11D) was strongly decreased.

FIGS. 12A-12D show the results of the same experiment as shown in FIGS. 11A-11D, however here the HeLa NucLight Red cells were replaced with Skov-3 cells. The secretion of proinflammatory cytokine TNF-α (FIG. 12A), IFN-γ (FIG. 12C), and IL-2 (FIG. 12B) and IL-10 (FIG. 12D) was not much changed by OX40 costimulation in this experiment.

FIG. 13 is a summary of the results shown in FIGS. 9A-9D, FIGS. 10A-10D, FIGS. 11A-11D and FIGS. 12A-12D. The changes of cytokine concentration were calculated in percent, whereby the respective sample w/o FAP OX40 iMab costimulation was considered 100%. The extent of changes depended on the tumor cell line and the respective TCB used.

The ability of FAP OX40 iMab costimulation to modulate the CEACAM5 CD3 TCB mediated cytokine secretion in resting CD4 T cells (FIGS. 14A-14H), in resting CD8 T cells (FIGS. 15A-15H) and in resting human PMBCs (FIGS. 16A-16H) was compared. The graphs show the secreted amount of the cytokines IL-2 (FIGS. 14A, 15A and 16A), IFN-γ (FIGS. 14B, 15B and 16B), TNF-α (FIGS. 14C, 15C and 16C), IL-4 (FIGS. 14D, 15D and 16D), IL-9 (FIGS. 14E, 15E and 16E), MIP-1α (FIGS. 14F, 15F and 16F), IL-17a (FIGS. 14G, 15G and 16G) and IL-10 (FIGS. 14H, 15H and 16H). Resting CD4 or CD8 T cells or PBMC were cocultured for 72 hrs with MKN-45 NucLight Red cells and irradiated NIH/3T3 huFAP in the presence of a serial dilution row of CEACAM5 CD3 TCB (CEA CD3 TCB (2)) with or without a fixed concentration of FAP Ox40 iMAB. The secreted amount of TNF-α, IFN-γ, IL-2, IL-10, IL-9, IL-4, Mip-1α and IL-17A was quantified at the 48h end point using cytometric bead array technology. The respective cytokine concentrations were plotted against the TCB concentration.

FIG. 17 shows a comparison of the increases in cytokine concentration caused by FAP Ox40 iMAB costimulation for the TCB top concentration.

FIGS. 18A and 18B show the pharmacokinetic profile of injected compounds during the first week of treatment in the in vivo experiment 1 as described in Example 4.4. 2 mice per group were bled 10 min, 6h, 24h, 96h and 7d after the first therapy and the exposure of injected compounds was analysed. Blood was processed to serum and sandwich ELISAs were performed to determine the exposure of FAP OX40 iMab (FIG. 18A) and CEACAM5 CD3 TCB (FIG. 18B) during the first week. The systemic exposure was comparable for mice receiving monotherapy or for mice receiving combination therapy.

FIGS. 19A-19B show that only the combination of CEACAM5 CD3 TCB with FAP(4B9) OX40 iMab mediated regression of subcutaneous tumors compared to all other groups. This can be clearly seen from the waterfall plot as shown in FIG. 19B. Stem cell humanized NOG mice were s.c. injected with a mixture of MKN45 gastric tumor cells and 3T3huFAP fibroblasts in matrigel. Mice were randomized on day 10 for tumor size and human T-cell count with an average T-cell count/μl blood of 140 and an average tumor size of 170 mm³. On the day of randomization mice were injected i.v. with Vehicle, CEACAM5 CD3 TCB (CEA CD3 TCB (2)), FAP OX40 iMAB or the combination thereof once per week for 5 consecutive weeks. The tumor volume was measured three times a week and plotted against the study time. Error bars show standard error for 6 to 8 animals per group (FIG. 19A). Percent change of tumor volume at day 41 of experiment compared to tumor volume at treatment start was calculated for each animal and plotted as waterfall plot (FIG. 19B).

FIGS. 20A and 20B show the pharmacokinetic profile of injected compounds during the first week of treatment in the in vivo experiment 2 as described in Example 4.5. 2 mice per group were bled 10 min, 6h, 24h, 96h and 7d after the first therapy and the exposure of injected compounds was analysed. Blood was processed to serum and sandwich ELISAs were performed to determine the exposure of the different doses of FAP OX40 iMab and its combinations with CEACAM5 CD3 TCB (FIG. 20A) and of CEACAM5 CD3 TCB and its combination with different doses of FAP OX40 iMab (FIG. 20B) during the first week. In FIG. 20A a clear dose dependency of the different dosages of FAP OX40 iMab can be seen. The exposure of CEACAM CD3 TCB was comparable for mice receiving monotherapy or for mice receiving combination therapy.

FIGS. 21A-21C show that only the combination of CEACAM5 CD3 TCB with the highest dose of FAP(4B9) OX40 iMab (12.5 mg/kg, FIG. 21C) showed improved efficacy in terms of tumor growth inhibition compared to all other groups. Stem cell humanized NOG mice were s.c. injected with a mixture of MKN45 gastric tumor cells and 3T3huFAP fibroblasts in matrigel. Mice were randomized day 26 for tumor size and human T-cell count with an average T-cell count/μl blood of 115 and an average tumor size of 490 mm3. One day after randomization mice were injected i.v. with Vehicle, CEACAM5 CD3 TCB (CEA CD3 TCB (2)), and different doses of FAP OX40 iMab (12.5 mg/kg, 4.2 mg/kg and 1.4 mg/kg, respectively) or the combinations of the OX40 targeted molecule with CEACAM5 CD3 TCB for 4 weeks. The tumor volume was measured three times a week and plotted against the study time. Error bars show standard error for 8 to 10 animals per group. FIG. 21A shows the tumor regression obtained with FAP OX40 iMab 1.4 mg/kg, the tumor regression observed with FAP OX40 iMab 4.2 mg/kg or with FAP OX40 iMab 12.5 mg/kg are shown in FIGS. 21B and 21C, respectively.

FIG. 22 summarizes the dose dependency of the anti-tumor efficacy of the combination of CEACAM5 CD3 TCB with different amounts of FAP(4B9) OX40 iMab. Percent change of tumor volume at treatment day 35 of experiment 2 compared to tumor volume at treatment start was calculated for each animal and plotted as waterfall plot.

FIGS. 23A-23D show that the combination of CEACAM5 CD3 TCB and FAP(4B9) OX40 iMab significantly increases the number of intratumoral leukocytes compared to all monotherapies. On day 50 of experiment 2 described in Example 4.5, tumor infiltrating lymphocytes were isolated and evaluated for the presence of human leukocytes and T cells by flow cytometry. Living human leukocytes (DAPI−, CD45+), NON-CD3 leukocytes (DAPI−, CD45+, CD3−), CD4 and CD8 T cells (DAPI−, CD45+, CD3+, CD4 or CD8+) were gated, normalized counts (per or μg tumor) calculated and values plotted for the respective treatment groups: FIG. 23A for living human leukocytes, FIG. 23B for NON-CD3 leukocytes, FIG. 23C for CD4 T cells and FIG. 23D for CD8 T cells. Error bars show standard error for 5 to 8 animals per group.

FIGS. 24A and 24B show that FAP OX40 iMAB costimulation and CEA CD3 TCB (2) act tumor specific and do not change systemic leuokocyte counts in spleen (FIG. 24A) and in blood (FIG. 24B).

FIGS. 25A and 25B show that the combination of CEACAM5 CD3 TCB and FAP(4B9) OX40 iMab significantly increased the number of intratumoral T cells and CD8 T cells compared to all monotherapies. The number of CD3 positive T cells as detected by huCD3 immunohistochemistry is shown in FIG. 25A and the number of CD8 positive T cells as detected by huCD8 immunohistochemistry is shown in FIG. 25B. HuCD8 and HuCD3 immunohistochemistry was performed on 4 μm paraffin sections.

FIGS. 26A-26C show that the combination of CEACAM5 CD3 TCB and FAP(4B9) OX40 iMab significantly increased the concentration of intratumoral cytokines compared to all monotherapies. No significant changes were detected in the periphery. On day 50 of experiment 2, tumor, spleen and blood were sampled and snap frozen. Cytokine concentrations were determined in the homogenates using the Bio-Plex Pro™ Human Cytokine 17-plex Assay. The whole protein content was analysis by the BCA protein assay kit and concentrations were normalized to the protein content of the samples. The median cytokine concentration of 4 animals per treatment group is depicted in FIG. 26A for the tumor, in FIG. 26B for spleen and in FIG. 26C for blood.

FIGS. 27A-27F show that intratumoral cytokine concentrations, but not the intratumoral leukocyte count, correlate inversely with the progression of tumor growth in the animals treated with the combination of FAP OX40 iMab and CEACAM5 CD3 TCB. This was not observed in animals treated with CEACAM5 CD3 TCB monotherapy. Each open symbol stands for an individual animal treated with CEACAM5 CD3 TCB monotherapy and each filled symbol stands for an individual animal treated with the combination. In FIG. 27A the count of T cells is plotted against the change in tumor volume [%], the concentration of TNF-a (FIG. 27B), IFN-g (FIG. 27C), MCP-1 (FIG. 27D), IL-8 (FIG. 27E) and IL-6 (FIG. 27F) is also plotted against the change in tumor volume [%].

FIGS. 28A and 28B show that the combination of CEA CD3 TCB with anti-PD-L1 and with FAP OX40 iMab mediated improved efficacy in terms of tumor growth inhibition compared to all other therapies (Example 5). FIGS. 28A and 28B show the tumor growth over time either as average of tumor volume or as average fold change of tumor volume, respectively.

FIGS. 29A-29C show the pharmacokinetic profile of injected compounds during the first week of treatment in the in vivo experiment as described in Example 5. 2 mice per group were bled 1 h and 72h after 1″ and 3^{rd} therapy and the exposure of injected compounds was analysed. Blood was processed to serum and sandwich ELISAs were performed to determine the exposure of FAP OX40 iMab in combination with CEACAM5 CD3 TCB or the triple combination (FIG. 29A), of CEA CD3 TCB and its different combinations (FIG. 29B) and of CEA CD3 TCB in combination with anti-PD-L1 or the triple combination (FIG. 29C). The exposure of all three compounds was comparable for mice receiving monotherapy or for mice receiving combination therapy.

FIGS. 30A and 30B show that the combination of CEACAM5 CD3 TCB with anti-PD-L1 and FAP(4B9) OX40 iMab significantly increased the number of intratumoral T cells and CD8 T cells compared to all mono- or doublet therapies. The number of CD3 positive T cells as detected by huCD3 immunohistochemistry is shown in FIG. 30A and the number of CD8 positive T cells as detected by huCD8 immunohistochemistry is shown in FIG. 30B. HuCD8 and HuCD3 immunohistochemistry was performed on 4 μm paraffin sections.

FIGS. 31A and 31B show that combination treatment with 100 nM CEA CD3 TCB and 2 nM FAP OX40 iMAB or triple combination treatment with anti-PD-L1 antibody increases the percentage of CD25 expressing CD4 (FIG. 31A) and CD8 (FIG. 31B) T cells.

FIGS. 32A and 32B show that combination treatment with 100 nM CEA CD3 TCB and 2 nM FAP OX40 iMAB or triple combination treatment with 80 nM anti-PD-L1 antibody increases the percentage of proliferating CD4 T cells (FIG. 32A) and CD8 T cells (FIG. 32B). PBMCs were labelled with proliferation dye CFSE prior to the start of the experiment and proliferation was measured by dilution of the CFSE dye using FACS.

FIGS. 33A and 33B show that combination treatment with 100 nM CEA CD3 TCB and 2 nM FAP OX40 iMAB or triple combination treatment with 80 nM anti-PD-L1 antibody increases the percentage of T-bet expressing CD4 T cells (FIG. 33A) and MFI (mean fluorescent intensity) of T-bet on CD8 T cells (FIG. 33B). FIGS. 33C and 33D show that combination treatment with 100 nM CEA CD3 TCB and 2 nM FAP OX40 iMAB increases the percentage of Granzyme B expressing CD4 T cells (FIG. 33C) and of Granzyme B expressing CD8 T cells (FIG. 33D). Triple combination with anti-PD-L1 antibody further increases the percentages of Granzyme B expressing CD4 and CD8 T cells as compared to CEA CD3 TCB and FAP OX40 iMAb combination treatment with statistical significance. Secreted cytokines IFNγ, GM-CSF, TNFα, IL-2, IL-8, Granzyme B and IL-10 were analyzed in the supernatant after 4 days of incubation using cytometric bead array according to manufacturer's instructions. Each symbol indicates one donor (pooled experimental triplicates per group), each color/pattern indicates a specific treatment combination, the bar indicates the mean with SEM.

FIGS. 34A-34C show that combination treatment with 100 nM CEA CD3 TCB and 2 nM FAP OX40 iMAB increases the secretion of IFNγ (FIG. 34A), Granzyme B (FIG. 34B) and IL-8 (FIG. 34C). Triple combination with aPD-L1 significantly increases the secretion of all three cytokines stated above.

FIGS. 35A-35C show the fold increase of cytokines in 6 donors after treatment with the triple combination of CEA CD3 TCB, FAP OX40 iMAb and a-PD-L1 as compared to cytokines after treatment with CEA CD3 TCB and aPD-L1 combination treatment, taken as baseline. The solid black line indicates 2 fold changes. Shown is fold increase of IFNγ (FIG. 35A), Granzyme B (FIG. 35B) and IL-8 (FIG. 35C).

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that specifically binds an antigenic determinant. Examples of antigen binding molecules are antibodies, antibody fragments and scaffold antigen binding proteins.

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” as used within the current application denotes the presence of a specified number of binding sites in an antigen binding molecule. As such, the terms “bivalent”, “tetravalent”, and “hexavalent” denote the presence of two binding sites, four binding sites, and six binding sites, respectively, in an antigen binding molecule.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ, (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′-SH are Fab′ fragments in which the cysteine residue(s) of the constant domains bear a free thiol group. Pepsin treatment yields an F(ab′)₂ fragment that has two antigen-combining sites (two Fab fragments) and a part of the Fc region.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab_{(VLVH)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab_{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a V_{H} domain, namely being able to assemble together with a V_{L} domain, or of a V_{L} domain, namely being able to assemble together with a V_{H} domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a kunitz type domain of human protease inhibitors, microbodies such as the proteins from the knottin family, peptide aptamers and fibronectin (adnectin).

Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633.

Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1.

A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks.

An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more.

The term “antigen binding domain” refers to the part of an antigen binding molecule that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions).

Preferably, an antigen binding domain comprises an antibody light chain variable region (V_{L}) and an antibody heavy chain variable region (V_{H}).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, a molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g. from 10⁻⁹ M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

The term “tumor-associated antigen (TAA)” means any antigen that is highly expressed by tumor cells or in the tumor stroma. The term tumor-associated indicates that TAA are not completely specific for the tumor, but are rather over-expressed on the tumor or its stroma. Particular tumor-associated antigens are CEA or FAP, but also other targets such as Folate Receptor (FolR1), MCSP, the EGFR family (HER2, HER3 and EGFR/HER1), VEGFR, CD20, CD19, CD22, CD33, PD1, PD-L1, TenC, EpCAM, PSA, PSMA, STEAP1, MUC1 (CA15-3) MUC16 (CA125) and 5T4 (trophoblast glycoprotein). Particular TAA include FAP, CEA and FolR1.

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP which results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149, SEQ ID NO:120), or NCBI (www.ncbi.nlm.nih.gov/) RefSeq NP_004451.2. The extracellular domain (ECD) of human FAP extends from amino acid position 26 to 760. The amino acid sequence of a His-tagged human FAP ECD is shown in SEQ ID NO: 121. The amino acid sequence of mouse FAP is shown in UniProt accession no. P97321 (version 126, SEQ ID NO:122), or NCBI RefSeq NP_032012.1. The extracellular domain (ECD) of mouse FAP extends from amino acid position 26 to 761. SEQ ID NO: 123 shows the amino acid sequence of a His-tagged mouse FAP ECD. SEQ ID NO: 124 shows the amino acid sequence of a His-tagged cynomolgus FAP ECD. Preferably, an anti-FAP binding molecule of the invention binds to the extracellular domain of FAP. Exemplary anti-FAP binding molecules are described in International Patent Application No. WO 2012/020006 A2.

The term “Carcinoembroynic antigen (CEA)”, also known as Carcinoembryonic antigen-related cell adhesion molecule 5 (CEACAM5), refers to any native CEA from any vertebrate source, including mammals such as primates (e.g. humans) non-human primates (e.g. cynomolgus monkeys) and rodents (e.g. mice and rats), unless otherwise indicated. The amino acid sequence of human CEA is shown in UniProt accession no. P06731 (version 151, SEQ ID NO:125). CEA has long been identified as a tumor-associated antigen (Gold and Freedman, J Exp Med., 121:439-462, 1965; Berinstein N. L., J Clin Oncol., 20:2197-2207, 2002). Originally classified as a protein expressed only in fetal tissue, CEA has now been identified in several normal adult tissues. These tissues are primarily epithelial in origin, including cells of the gastrointestinal, respiratory, and urogential tracts, and cells of colon, cervix, sweat glands, and prostate (Nap et al., Tumour Biol., 9(2-3):145-53, 1988; Nap et al., Cancer Res., 52(8):2329-23339, 1992). Tumors of epithelial origin, as well as their metastases, contain CEA as a tumor associated antigen. While the presence of CEA itself does not indicate transformation to a cancerous cell, the distribution of CEA is indicative. In normal tissue, CEA is generally expressed on the apical surface of the cell (Hammarstrom S., Semin Cancer Biol. 9(2):67-81 (1999)), making it inaccessible to antibody in the blood stream. In contrast to normal tissue, CEA tends to be expressed over the entire surface of cancerous cells (Hammarstrom S., Semin Cancer Biol. 9(2):67-81 (1999)). This change of expression pattern makes CEA accessible to antibody binding in cancerous cells. In addition, CEA expression increases in cancerous cells. Furthermore, increased CEA expression promotes increased intercellular adhesions, which may lead to metastasis (Marshall J., Semin Oncol., 30(a Suppl. 8):30-6, 2003). The prevalence of CEA expression in various tumor entities is generally very high. In concordance with published data, own analyses performed in tissue samples confirmed its high prevalence, with approximately 95% in colorectal carcinoma (CRC), 90% in pancreatic cancer, 80% in gastric cancer, 60% in non-small cell lung cancer (NSCLC, where it is co-expressed with HER3), and 40% in breast cancer; low expression was found in small cell lung cancer and glioblastoma.

CEA is readily cleaved from the cell surface and shed into the blood stream from tumors, either directly or via the lymphatics. Because of this property, the level of serum CEA has been used as a clinical marker for diagnosis of cancers and screening for recurrence of cancers, particularly colorectal cancer (Goldenberg D M., The International Journal of Biological Markers, 7:183-188, 1992; Chau I., et al., J Clin Oncol., 22:1420-1429, 2004; Flamini et al., Clin Cancer Res; 12(23):6985-6988, 2006).

The term “FolR1” refers to Folate receptor alpha and has been identified as a potential prognostic and therapeutic target in a number of cancers. It refers to any native FolR1 from any vertebrate source, including mammals such as primates (e.g. humans) non-human primates (e.g. cynomolgus monkeys) and rodents (e.g. mice and rats), unless otherwise indicated. The amino acid sequence of human FolR1 is shown in UniProt accession no. P15328 (SEQ ID NO:126), murine FolR1 has the amino acid sequence of UniProt accession no. P35846 (SEQ ID NO:127) and cynomolgus FolR1 has the amino acid sequence as shown in UniProt accession no. G7PR14 (SEQ ID NO:128). FolR1 is an N-glycosylated protein expressed on plasma membrane of cells. FolR1 has a high affinity for folic acid and for several reduced folic acid derivatives and mediates delivery of the physiological folate, 5-methyltetrahydrofolate, to the interior of cells. FOLR1 is a desirable target for FOLR1-directed cancer therapy as it is overexpressed in vast majority of ovarian cancers, as well as in many uterine, endometrial, pancreatic, renal, lung, and breast cancers, while the expression of FOLR1 on normal tissues is restricted to the apical membrane of epithelial cells in the kidney proximal tubules, alveolar pneumocytes of the lung, bladder, testes, choroid plexus, and thyroid. Recent studies have identified that FolR1 expression is particularly high in triple negative breast cancers (Necela et al. PloS One 2015, 10(3), e0127133).

The term “MCSP” refers to Melanoma-associated Chondroitin Sulfate Proteoglycan, also known as Chondroitin Sulfate Proteoglycan 4 (CSPG4). It refers to any native FolR1 from any vertebrate source, including mammals such as primates (e.g. humans) non-human primates (e.g. cynomolgus monkeys) and rodents (e.g. mice and rats), unless otherwise indicated. The amino acid sequence of human MCSP is shown in UniProt accession no. Q6UVK1 (SEQ ID NO:129). MCSP is a highly glycosylated integral membrane chondroitin sulfate proteoglycan consisting of an N-linked 280 kDa glycoprotein component and a 450-kDa chondroitin sulfate proteoglycan component expressed on the cell membrane (Ross et al., Arch. Biochem. Biophys. 1983, 225:370-38). MCSP is more broadly distributed in a number of normal and transformed cells. in particular, MCSP is found in almost all basal cells of the epidermis. MCSP is differentially expressed in melanoma cells, and was found to be expressed in more than 90% of benign nevi and melanoma lesions analyzed. MCSP has also been found to be expressed in tumors of nonmelanocytic origin, including basal cell carcinoma, various tumors of neural crest origin, and in breast carcinomas.

A “T-cell antigen” as used herein refers to an antigenic determinant presented on the surface of a T lymphocyte, particularly a cytotoxic T lymphocyte.

A “T cell activating therapeutic agent” as used herein refers to a therapeutic agent capable of inducing T cell activation in a subject, particularly a therapeutic agent designed for inducing T-cell activation in a subject. Examples of T cell activating therapeutic agents include bispecific antibodies that specifically bind an activating T cell antigen, such as CD3, and a target cell antigen, such as CEA or Folate Receptor.

An “activating T cell antigen” as used herein refers to an antigenic determinant expressed by a T lymphocyte, particularly a cytotoxic T lymphocyte, which is capable of inducing or enhancing T cell activation upon interaction with an antigen binding molecule. Specifically, interaction of an antigen binding molecule with an activating T cell antigen may induce T cell activation by triggering the signaling cascade of the T cell receptor complex. An exemplary activating T cell antigen is CD3.

The term “CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one embodiment, CD3 is human CD3, particularly the epsilon subunit of human CD3 (CD3ε). The amino acid sequence of human CD3ε is shown in UniProt (www.uniprot.org) accession no. P07766 (version 144), or NCBI (www.ncbi.nlm.nih.gov/) RefSeq NP_000724.1. See also SEQ ID NO: 130. The amino acid sequence of cynomolgus [Macaca fascicularis] CD3ε is shown in NCBI GenBank no. BAB71849.1. See also SEQ ID NO: 131.

The term “variable region” or “variable domain” refers to the domain of an antibody heavy or light chain that is involved in binding the antigen binding molecule to antigen. The variable domains of the heavy chain and light chain (V_{H} and V_{L}, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single V_{H} or V_{L} domain may be sufficient to confer antigen-binding specificity.

The term “hypervariable region” or “HVR,” as used herein refers to each of the regions of an antibody variable domain which are hypervariable in sequence and/or form structurally defined loops (“hypervariable loops”). Generally, native four-chain antibodies comprise six HVRs; three in the V_{H} (H1, H2, H3), and three in the V_{L} (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) Hypervariable regions (HVRs) are also referred to as complementarity determining regions (CDRs), and these terms are used herein interchangeably in reference to portions of the variable region that form the antigen binding regions. This particular region has been described by Kabat et al., U.S. Dept. of Health and Human Services, “Sequences of Proteins of Immunological Interest” (1983) and by Chothia et al., J. Mol. Biol. 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table B as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE A
CDR Definitions¹

| CDR | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table A is according to the numbering conventions set forth by Kabat et al. (see below).
²“AbM” with a lowercase “b” as used in Table A refers to the CDRs as defined by Oxford Molecular's “AbM” antibody modeling software.

Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

With the exception of CDR1 in V_{H}, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

As used herein, the term “affinity matured” in the context of antigen binding molecules (e.g., antibodies) refers to an antigen binding molecule that is derived from a reference antigen binding molecule, e.g., by mutation, binds to the same antigen, preferably binds to the same epitope, as the reference antibody; and has a higher affinity for the antigen than that of the reference antigen binding molecule. Affinity maturation generally involves modification of one or more amino acid residues in one or more CDRs of the antigen binding molecule. Typically, the affinity matured antigen binding molecule binds to the same epitope as the initial reference antigen binding molecule.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in V_{H} (or V_{L}): FR1-H1(L1)-FR2-H2(L2)-FR3-H3(L3)-FR4.

An “acceptor human framework” for the purposes herein is a framework comprising the amino acid sequence of a light chain variable domain (V_{L}) framework or a heavy chain variable domain (V_{H}) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the V_{L} acceptor human framework is identical in sequence to the V_{L} human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

A “human” antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “Fc domain” or “Fe region” herein is used to define a C-terminal region of an antibody heavy chain that contains at least a portion of the constant region. The term includes native sequence Fc regions and variant Fc regions. An IgG Fc region comprises an IgG CH2 and an IgG CH3 domain. The “CH2 domain” of a human IgG Fc region usually extends from an amino acid residue at about position 231 to an amino acid residue at about position 340. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 2 and 4, in particular 2, i.e. the peptides selected from the group consisting of GGGGS (SEQ ID NO:132), GGGGSGGGGS (SEQ ID NO:133), SGGGGSGGGG (SEQ ID NO:134) and GGGGSGGGGSGGGG (SEQ ID NO:135), but also include the sequences GSPGSSSSGS (SEQ ID NO:136), (G4S)₃ (SEQ ID NO:137), (G4S)₄ (SEQ ID NO:138), GSGSGSGS (SEQ ID NO:139), GSGSGNGS (SEQ ID NO:140), GGSGSGSG (SEQ ID NO:141), GGSGSG (SEQ ID NO:142), GGSG (SEQ ID NO:143), GGSGNGSG (SEQ ID NO:144), GGNGSGSG (SEQ ID NO:145) and GGNGSG (SEQ ID NO:146). Peptide linkers of particular interest are (G4S) (SEQ ID NO:132), (G₄S)₂ (SEQ ID NO:133), (G4S)₃ (SEQ ID NO:137) and (G4S)₄ (SEQ ID NO:138.

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

By “fused” or “connected” is meant that the components (e.g. a polypeptide and an ectodomain of 4-1BBL) are linked by peptide bonds, either directly or via one or more peptide linkers.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN. SAWI or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In certain embodiments, amino acid sequence variants of the antigen binding molecules provided herein are contemplated. For example, it may be desirable to improve the binding affinity and/or other biological properties of the antigen binding molecules. Amino acid sequence variants of the antigen binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and Framework (FRs). Conservative substitutions are provided in Table C under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

TABLE B

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp: Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more CDR residues are mutated and the variant antigen binding molecules displayed on phage and screened for a particular biological activity (e.g. binding affinity). In certain embodiments, substitutions, insertions, or deletions may occur within one or more CDRs so long as such alterations do not substantially reduce the ability of the antigen binding molecule to bind antigen. For example, conservative alterations (e.g., conservative substitutions as provided herein) that do not substantially reduce binding affinity may be made in CDRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include antigen binding molecules with an N-terminal methionyl residue. Other insertional variants of the molecule include the fusion to the N- or C-terminus to a polypeptide which increases the serum half-life of the antigen binding molecules.

In certain embodiments, the antigen binding molecules provided herein are altered to increase or decrease the extent to which the antibody is glycosylated. Glycosylation variants of the molecules may be conveniently obtained by altering the amino acid sequence such that one or more glycosylation sites is created or removed. Where the antigen binding molecule comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the antigen binding molecules may be made in order to create variants with certain improved properties. In one aspect, variants of antigen binding molecules are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). Further variants of the antigen binding molecules of the invention include those with bisected oligosaccharides, e.g., in which a biantennary oligosaccharide attached to the Fc region is bisected by GlcNAc. Such variants may have reduced fucosylation and/or improved ADCC function., see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain embodiments, it may be desirable to create cysteine engineered variants of the antigen binding molecules of the invention, e.g., “thioMAbs,” in which one or more residues of the molecule are substituted with cysteine residues. In particular embodiments, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antigen binding molecules may be generated as described, e.g., in U.S. Pat. No. 7,521,541.

In certain aspects, the antigen binding molecules provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the bispecific antibody derivative will be used in a therapy under defined conditions, etc. In another aspect, conjugates of an antibody and non-proteinaceous moiety that may be selectively heated by exposure to radiation are provided. In one embodiment, the non-proteinaceous moiety is a carbon nanotube (Kam, N. W. et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed. In another aspect, immunoconjugates of the 4-1BBL-containing antigen binding molecules provided herein maybe obtained. An “immunoconjugate” is an antibody conjugated to one or more heterologous molecule(s), including but not limited to a cytotoxic agent.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NSO cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

The combination therapies in accordance with the invention have a synergistic effect. A “synergistic effect” of two compounds is one in which the effect of the combination of the two agents is greater than the sum of their individual effects and is statistically different from the controls and the single drugs. In another embodiment, the combination therapies disclosed herein have an additive effect. An “additive effect” of two compounds is one in which the effect of the combination of the two agents is the sum of their individual effects and is statistically different from either the controls and/or the single drugs.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical composition, other than an active ingredient, which is nontoxic to a subject. A pharmaceutically acceptable excipient includes, but is not limited to, a buffer, a stabilizer, or a preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “cancer” as used herein refers to proliferative diseases, such as solid tumors, or melanoma.

Exemplary Targeted OX40 Agonists for Use in the Invention

In particular, the targeted OX40 agonists as used in combination with the T-cell activating anti-CD3 bispecific antibodies specific for a tumor-associated antigen are bispecific OX40 antibodies comprising at least one antigen binding domain capable of specific binding to a tumor-associated antigen.

In particular, the bispecific OX40 antibody comprising at least one antigen binding domain capable of specific binding to a tumor-associated antigen is an anti-Fibroblast activation protein (FAP)/anti-OX40 bispecific antibody. In one aspect, the anti-FAP/anti-OX40 antibody is an OX40 agonist. In one aspect, the anti-FAP/anti-OX40 antibody is an antigen binding molecule comprising a Fc domain. In a particular aspect, the anti-FAP/anti-OX40 antibody is an antigen binding molecule comprising a Fc domain with modifications reducing Fcγ receptor binding and/or effector function. The crosslinking by a tumor associated antigen makes it possible to avoid unspecific FcγR-mediated crosslinking and thus higher and more efficacious doses of the anti-FAP/anti-OX40 antibody may be administered in comparison to common OX40 antibodies.

In one aspect, the invention provides a bispecific OX40 antibody comprising at least one antigen binding domain capable of specific binding to a tumor-associated antigen, in particular an anti-FAP/anti-OX40 bispecific antibody, for use in a method for treating or delaying progression of cancer, wherein the bispecific OX40 antibody is used in combination with a T-cell activating anti-CD3 bispecific antibody specific for a tumor-associated antigen and wherein the bispecific OX40 antibody comprises at least one antigen binding domain capable of specific binding to FAP comprising

(a) a heavy chain variable region (V_{H}FAP) comprising (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3, and a light chain variable region (V_{L}FAP) comprising (iv) CDR-L1 comprising the amino acid sequence of SEQ ID NO:4, (v) CDR-L2 comprising the amino acid sequence of SEQ ID NO:5, and (vi) CDR-L3 comprising the amino acid sequence of SEQ ID NO:6, or
(b) a heavy chain variable region (V_{H}FAP) comprising (i) CDR-H1 comprising the amino acid sequence of SEQ ID NO:9, (ii) CDR-H2 comprising the amino acid sequence of SEQ ID NO:10, and (iii) CDR-H3 comprising the amino acid sequence of SEQ ID NO:11, and a light chain variable region (V_{L}FAP) comprising (iv) CDR-L1 comprising the amino acid sequence of SEQ ID NO:12, (v) CDR-L2 comprising the amino acid sequence of SEQ ID NO:13, and (vi) CDR-L3 comprising the amino acid sequence of SEQ ID NO:14.

In a further aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as defined herein before, wherein the bispecific OX40 antibody comprises at least one antigen binding domain capable of specific binding to FAP comprising a heavy chain variable region (V_{H}FAP) that is at least 90%, 95%, 96%, 97%, 98%, or 99% identical to an amino acid sequence of SEQ ID NO:7 and a light chain variable region (V_{L}FAP) that is at least 90%, 95%, 96%, 97%, 98%, or 99% identical to an amino acid sequence of SEQ ID NO:8 or an antigen binding domain capable of specific binding to FAP comprising a heavy chain variable region (V_{H}FAP) that is at least 90%, 95%, 96%, 97%, 98%, or 99% identical to an amino acid sequence of SEQ ID NO:15 and a light chain variable region (V_{L}FAP) that is at least 90%, 95%, 96%, 97%, 98%, or 99% identical to an amino acid sequence of SEQ ID NO:16.

In a particular aspect, the bispecific OX40 antibody comprises at least one antigen binding domain capable of specific binding to FAP comprising a heavy chain variable region (V_{H}FAP) comprising an amino acid sequence of SEQ ID NO:7 and a light chain variable region (V_{L}FAP) comprising an amino acid sequence of SEQ ID NO:8. In another aspect, the bispecific OX40 antibody comprises at least one an antigen binding domain capable of specific binding to FAP comprising a heavy chain variable region (V_{H}FAP) comprising an amino acid sequence of SEQ ID NO:15 and a light chain variable region (V_{L}FAP) comprising an amino acid sequence of SEQ ID NO:16.

In a further aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as defined herein before, wherein the bispecific OX40 antibody comprises at least one antigen binding domain capable of specific binding to OX40 comprising

(a) a heavy chain variable region (V_{H}OX40) comprising (i) CDR-H1 comprising the amino acid sequence of SEQ ID NO:17, (ii) CDR-H2 comprising the amino acid sequence of SEQ ID NO:19, and (iii) CDR-H3 comprising the amino acid sequence of SEQ ID NO:22, and a light chain variable region (V_{L}OX40) comprising (iv) CDR-L1 comprising the amino acid sequence of SEQ ID NO:28, (v) CDR-L2 comprising the amino acid sequence of SEQ ID NO:31, and (vi) CDR-L3 comprising the amino acid sequence of SEQ ID NO:35, or
(b) a heavy chain variable region (V_{H}OX40) comprising (i) CDR-H1 comprising the amino acid sequence of SEQ ID NO:17, (ii) CDR-H2 comprising the amino acid sequence of SEQ ID NO:19, and (iii) CDR-H3 comprising the amino acid sequence of SEQ ID NO:21, and a light chain variable region (V_{L}OX40) comprising (iv) CDR-L1 comprising the amino acid sequence of SEQ ID NO:28, (v) CDR-L2 comprising the amino acid sequence of SEQ ID NO:31, and (vi) CDR-L3 comprising the amino acid sequence of SEQ ID NO:34, or
(c) a heavy chain variable region (V_{H}OX40) comprising (i) CDR-H1 comprising the amino acid sequence of SEQ ID NO:17, (ii) CDR-H2 comprising the amino acid sequence of SEQ ID NO:19, and (iii) CDR-H3 comprising the amino acid sequence of SEQ ID NO:23, and a light chain variable region (V_{L}OX40) comprising (iv) CDR-L1 comprising the amino acid sequence of SEQ ID NO:28, (v) CDR-L2 comprising the amino acid sequence of SEQ ID NO:31, and (vi) CDR-L3 comprising the amino acid sequence of SEQ ID NO:36, or
(d) a heavy chain variable region (V_{H}OX40) comprising (i) CDR-H1 comprising the amino acid sequence of SEQ ID NO:17, (ii) CDR-H2 comprising the amino acid sequence of SEQ ID NO:19, and (iii) CDR-H3 comprising the amino acid sequence of SEQ ID NO:24, and a light chain variable region (V_{L}OX40) comprising (iv) CDR-L1 comprising the amino acid sequence of SEQ ID NO:28, (v) CDR-L2 comprising the amino acid sequence of SEQ ID NO:31, and (vi) CDR-L3 comprising the amino acid sequence of SEQ ID NO:37, or
(e) a heavy chain variable region (V_{H}OX40) comprising (i) CDR-H1 comprising the amino acid sequence of SEQ ID NO:18, (ii) CDR-H2 comprising the amino acid sequence of SEQ ID NO:20, and (iii) CDR-H3 comprising the amino acid sequence of SEQ ID NO:25, and a light chain variable region (V_{L}OX40) comprising (iv) CDR-L1 comprising the amino acid sequence of SEQ ID NO:29, (v) CDR-L2 comprising the amino acid sequence of SEQ ID NO:32, and (vi) CDR-L3 comprising the amino acid sequence of SEQ ID NO:38, or
(f) a heavy chain variable region (V_{H}OX40) comprising (i) CDR-H1 comprising the amino acid sequence of SEQ ID NO:18, (ii) CDR-H2 comprising the amino acid sequence of SEQ ID NO:20, and (iii) CDR-H3 comprising the amino acid sequence of SEQ ID NO:26, and a light chain variable region (V_{L}OX40) comprising (iv) CDR-L1 comprising the amino acid sequence of SEQ ID NO:29, (v) CDR-L2 comprising the amino acid sequence of SEQ ID NO:32, and (vi) CDR-L3 comprising the amino acid sequence of SEQ ID NO:38, or
(g) a heavy chain variable region (V_{H}OX40) comprising (i) CDR-H1 comprising the amino acid sequence of SEQ ID NO:18, (ii) CDR-H2 comprising the amino acid sequence of SEQ ID NO:20, and (iii) CDR-H3 comprising the amino acid sequence of SEQ ID NO:27, and a light chain variable region (V_{L}OX40) comprising (iv) CDR-L1 comprising the amino acid sequence of SEQ ID NO:30, (v) CDR-L2 comprising the amino acid sequence of SEQ ID NO:33, and (vi) CDR-L3 comprising the amino acid sequence of SEQ ID NO:39.

More particularly, the the bispecific OX40 antibody comprises at least one antigen binding domain capable of specific binding to OX40 comprising

(a) a heavy chain variable region (V_{H}OX40) comprising (i) CDR-H1 comprising the amino acid sequence of SEQ ID NO:17, (ii) CDR-H2 comprising the amino acid sequence of SEQ ID NO:19, and (iii) CDR-H3 comprising the amino acid sequence of SEQ ID NO:22, and a light chain variable region (V_{L}OX40) comprising (iv) CDR-L1 comprising the amino acid sequence of SEQ ID NO:28, (v) CDR-L2 comprising the amino acid sequence of SEQ ID NO:31, and (vi) CDR-L3 comprising the amino acid sequence of SEQ ID NO:35.

In a further aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer, wherein the bispecific OX40 antibody comprises at least one antigen binding domain capable of specific binding to OX40 comprising

(a) a heavy chain variable region (V_{H}OX40) comprising an amino acid sequence of SEQ ID NO:40 and a light chain variable region (V_{L}OX40) comprising an amino acid sequence of SEQ ID NO:41, or
(b) a heavy chain variable region (V_{H}OX40) comprising an amino acid sequence of SEQ ID NO:42 and a light chain variable region (V_{L}OX40) comprising an amino acid sequence of SEQ ID NO:43, or
(c) a heavy chain variable region (V_{H}OX40) comprising an amino acid sequence of SEQ ID NO:44 and a light chain variable region (V_{L}OX40) comprising an amino acid sequence of SEQ ID NO:45, or
(d) a heavy chain variable region (V_{H}OX40) comprising an amino acid sequence of SEQ ID NO:46 and a light chain variable region (V_{L}OX40) comprising an amino acid sequence of SEQ ID NO:47, or
(a) a heavy chain variable region (V_{H}OX40) comprising an amino acid sequence of SEQ ID NO:48 and a light chain variable region (V_{L}OX40) comprising an amino acid sequence of SEQ ID NO:49, or
(a) a heavy chain variable region (V_{H}OX40) comprising an amino acid sequence of SEQ ID NO:50 and a light chain variable region (V_{L}OX40) comprising an amino acid sequence of SEQ ID NO:51, or
(a) a heavy chain variable region (V_{H}OX40) comprising an amino acid sequence of SEQ ID NO:52 and a light chain variable region (V_{L}OX40) comprising an amino acid sequence of SEQ ID NO:53.

In a particular aspect, the bispecific OX40 antibody comprises at least one antigen binding domain capable of specific binding to OX40 comprising

(a) a heavy chain variable region (V_{H}OX40) that is at least 90%, 95%, 96%, 97%, 98%, or 99% identical to an amino acid sequence of SEQ ID NO:40 and a light chain variable region (V_{L}OX40) that is at least 90%, 95%, 96%, 97%, 98%, or 99% identical to an amino acid sequence of SEQ ID NO:41.

More particularly, bispecific OX40 antibody comprises at least one antigen binding domain capable of specific binding to OX40 comprising a heavy chain variable region (V_{H}OX40) comprising an amino acid sequence of SEQ ID NO:40 and a light chain variable region (V_{L}OX40) comprising an amino acid sequence of SEQ ID NO:41.

In one aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer, wherein the bispecific OX40 antibody comprising at least one antigen binding domain capable of specific binding to a tumor-associated antigen is an antigen binding molecule further comprising a Fc domain composed of a first and a second subunit capable of stable association. In particular, the bispecific OX40 antibody is an antigen binding molecule comprising an IgG Fc domain, specifically an IgG1 Fc domain or an IgG4 Fc domain. More particularly, the bispecific OX40 antibody is an antigen binding molecule comprising a Fc domain that comprises one or more amino acid substitution that reduces binding to an Fc receptor and/or effector function. In a particular aspect, the bispecific OX40 antibody comprises an IgG1 Fc domain comprising the amino acid substitutions L234A, L235A and P329G.

In another aspect of the invention, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as described herein before, wherein the bispecific OX40 antibody comprises monovalent binding to a tumor associated target and and at least bivalent binding to OX40. In one aspect, the anti-FAP/anti-OX40 bispecific antibody comprises monovalent binding to a tumor associated target and and bivalent binding to OX40. In a particular aspect, the anti-FAP/anti-OX40 bispecific antibody comprises monovalent binding to a tumor associated target and and tetravalent binding to OX40.

In another aspect, the invention provides a bispecific OX40 antibody comprising at least one antigen binding domain capable of specific binding to a tumor-associated antigen, in particular an anti-FAP/anti-OX40 bispecific antibody, for use in a method for treating or delaying progression of cancer as described herein before, wherein the bispecific OX40 antibody comprises a first Fab fragment capable of specific binding to OX40 fused at the C-terminus of the CH1 domain to the VH domain of a second Fab fragment capable of specific binding to OX40 and a third Fab fragment capable of specific binding to OX40 fused at the C-terminus of the CH1 domain to the VH domain of a fourth Fab fragment capable of specific binding to OX40.

In one aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as described herein before, wherein the bispecific OX40 antibody comprises

(i) a first heavy chain comprising an amino acid sequence of SEQ ID NO:54, a second heavy chain comprising an amino acid sequence of SEQ ID NO:55, and four light chains comprising an amino acid sequence of SEQ ID NO:56, or
(ii) a first heavy chain comprising an amino acid sequence of SEQ ID NO:57, a second heavy chain comprising an amino acid sequence of SEQ ID NO:58, and four light chains comprising an amino acid sequence of SEQ ID NO:56, or
(i) a first heavy chain comprising an amino acid sequence of SEQ ID NO:59, a second heavy chain comprising an amino acid sequence of SEQ ID NO:60, and four light chains comprising an amino acid sequence of SEQ ID NO:56, or
(ii) a first heavy chain comprising an amino acid sequence of SEQ ID NO:61, a second heavy chain comprising an amino acid sequence of SEQ ID NO:62, and four light chains comprising an amino acid sequence of SEQ ID NO:56.

In one particular aspect, provided is a bispecific OX40 antibody comprising at least one antigen binding domain capable of specific binding to a tumor-associated antigen, in particular an anti-FAP/anti-OX40 bispecific antibody, for use in a method for treating or delaying progression of cancer as described herein, wherein the bispecific OX40 antibody comprises a first heavy chain comprising an amino acid sequence of SEQ ID NO:54, a second heavy chain comprising an amino acid sequence of SEQ ID NO:55, and four light chains comprising an amino acid sequence of SEQ ID NO:56.

Exemplary Anti-CEA/Anti-CD3 Bispecific Antibodies for Use in the Invention

The present invention relates to targeted OX40 agonists and their use in combination with T-cell activating anti-CD3 bispecific antibodies specific for a tumor-associated antigen, in particular to their use in a method for treating or delaying progression of cancer, more particularly for treating or delaying progression of solid tumors. In particular, tumor-associated antigen is CEA. The anti-CEA/anti-CD3 bispecific antibodies as used herein are bispecific antibodies comprising a first antigen binding domain that binds to CD3, and a second antigen binding domain that binds to CEA.

Thus, the anti-CEA/anti-CD3 bispecific antibody as used herein comprises a first antigen binding domain comprising a heavy chain variable region (V_{H}CD3) and a light chain variable region (V_{L}CD3), and a second antigen binding domain comprising a heavy chain variable region (V_{H}CEA) and a light chain variable region (V_{L}CEA).

In a particular aspect, the anti-CEA/anti-CD3 bispecific antibody for use in the combination comprises a first antigen binding domain comprising a heavy chain variable region (V_{H}CD3) comprising CDR-H1 sequence of SEQ ID NO:63, CDR-H2 sequence of SEQ ID NO:64, and CDR-H3 sequence of SEQ ID NO:65; and/or a light chain variable region (V_{L}CD3) comprising CDR-L1 sequence of SEQ ID NO:66, CDR-L2 sequence of SEQ ID NO:67, and CDR-L3 sequence of SEQ ID NO:68. More particularly, the anti-CEA/anti-CD3 bispecific antibody comprises a first antigen binding domain comprising a heavy chain variable region (V_{H}CD3) that is at least 90%, 95%, 96%, 97%, 98%, or 99% identical to the amino acid sequence of SEQ ID NO:69 and/or a light chain variable region (V_{L}CD3) that is at least 90%, 95%, 96%, 97%, 98%, or 99% identical to the amino acid sequence of SEQ ID NO:70. In a further aspect, the anti-CEA/anti-CD3 bispecific antibody comprises a heavy chain variable region (V_{H}CD3) comprising the amino acid sequence of SEQ ID NO:69 and/or a light chain variable region (V_{L}CD3) comprising the amino acid sequence of SEQ ID NO:70.

In one aspect, the antibody that specifically binds to CD3 is a full-length antibody. In one aspect, the antibody that specifically binds to CD3 is an antibody of the human IgG class, particularly an antibody of the human IgG₁ class. In one aspect, the antibody that specifically binds to CD3 is an antibody fragment, particularly a Fab molecule or a scFv molecule, more particularly a Fab molecule. In a particular aspect, the antibody that specifically binds to CD3 is a crossover Fab molecule wherein the variable domains or the constant domains of the Fab heavy and light chain are exchanged (i.e. replaced by each other). In one aspect, the antibody that specifically binds to CD3 is a humanized antibody.

In another aspect, the anti-CEA/anti-CD3 bispecific antibody comprises a second antigen binding domain comprising

(a) a heavy chain variable region (V_{H}CEA) comprising CDR-H1 sequence of SEQ ID NO:71, CDR-H2 sequence of SEQ ID NO:72, and CDR-H3 sequence of SEQ ID NO:73, and/or a light chain variable region (V_{L}CEA) comprising CDR-L1 sequence of SEQ ID NO:74, CDR-L2 sequence of SEQ ID NO:75, and CDR-L3 sequence of SEQ ID NO:76, or
(b) a heavy chain variable region (V_{H}CEA) comprising CDR-H1 sequence of SEQ ID NO:79, CDR-H2 sequence of SEQ ID NO:80, and CDR-H3 sequence of SEQ ID NO:81, and/or a light chain variable region (V_{L}CEA) comprising CDR-L1 sequence of SEQ ID NO:82, CDR-L2 sequence of SEQ ID NO:83, and CDR-L3 sequence of SEQ ID NO:84.

More particularly, the anti-CEA/anti-CD3 bispecific comprises a second antigen binding domain comprising a heavy chain variable region (V_{H}CEA) that is at least 90%, 95%, 96%, 97%, 98%, or 99% identical to the amino acid sequence of SEQ ID NO:77 and/or a light chain variable region (V_{L}CEA) that is at least 90%, 95%, 96%, 97%, 98%, or 99% identical to the amino acid sequence of SEQ ID NO:78. In a further aspect, the anti-CEA/anti-CD3 bispecific comprises a second antigen binding domain comprising a heavy chain variable region (V_{H}CEA) comprising the amino acid sequence of SEQ ID NO:77 and/or a light chain variable region (V_{L}CEA) comprising the amino acid sequence of SEQ ID NO:78. In another aspect, the anti-CEA/anti-CD3 bispecific comprises a second antigen binding domain comprising a heavy chain variable region (V_{H}CEA) that is at least 90%, 95%, 96%, 97%, 98%, or 99% identical to the amino acid sequence of SEQ ID NO:85 and/or a light chain variable region (V_{L}CEA) that is at least 90%, 95%, 96%, 97%, 98%, or 99% identical to the amino acid sequence of SEQ ID NO:86. In a further aspect, the anti-CEA/anti-CD3 bispecific comprises a second antigen binding domain comprising a heavy chain variable region (V_{H}CEA) comprising the amino acid sequence of SEQ ID NO:85 and/or a light chain variable region (V_{L}CEA) comprising the amino acid sequence of SEQ ID NO:86.

In another particular aspect, the anti-CEA/anti-CD3 bispecific antibody comprises a third antigen binding domain that binds to CEA. In particular, the anti-CEA/anti-CD3 bispecific antibody comprises a third antigen binding domain comprising

(a) a heavy chain variable region (V_{H}CEA) comprising CDR-H1 sequence of SEQ ID NO:71, CDR-H2 sequence of SEQ ID NO:72, and CDR-H3 sequence of SEQ ID NO:73, and/or a light chain variable region (V_{L}CEA) comprising CDR-L1 sequence of SEQ ID NO:74, CDR-L2 sequence of SEQ ID NO:75, and CDR-L3 sequence of SEQ ID NO:76, or
(b) a heavy chain variable region (V_{H}CEA) comprising CDR-H1 sequence of SEQ ID NO:79, CDR-H2 sequence of SEQ ID NO:80, and CDR-H3 sequence of SEQ ID NO:81, and/or a light chain variable region (V_{L}CEA) comprising CDR-L1 sequence of SEQ ID NO:82, CDR-L2 sequence of SEQ ID NO:83, and CDR-L3 sequence of SEQ ID NO:84.

More particularly, the anti-CEA/anti-CD3 bispecific comprises a third antigen binding domain comprising a heavy chain variable region (V_{H}CEA) that is at least 90%, 95%, 96%, 97%, 98%, or 99% identical to the amino acid sequence of SEQ ID NO:77 and/or a light chain variable region (V_{L}CEA) that is at least 90%, 95%, 96%, 97%, 98%, or 99% identical to the amino acid sequence of SEQ ID NO:78. In a further aspect, the anti-CEA/anti-CD3 bispecific comprises a third antigen binding domain comprising a heavy chain variable region (V_{H}CEA) comprising the amino acid sequence of SEQ ID NO:77 and/or a light chain variable region (V_{L}CEA) comprising the amino acid sequence of SEQ ID NO:78. In another particular aspect, the anti-CEA/anti-CD3 bispecific comprises a third antigen binding domain comprising a heavy chain variable region (V_{H}CEA) that is at least 90%, 95%, 96%, 97%, 98%, or 99% identical to the amino acid sequence of SEQ ID NO:85 and/or a light chain variable region (V_{L}CEA) that is at least 90%, 95%, 96%, 97%, 98%, or 99% identical to the amino acid sequence of SEQ ID NO:86. In a further aspect, the anti-CEA/anti-CD3 bispecific comprises a third antigen binding domain comprising a heavy chain variable region (V_{H}CEA) comprising the amino acid sequence of SEQ ID NO:85 and/or a light chain variable region (V_{L}CEA) comprising the amino acid sequence of SEQ ID NO:86.

In a further aspect, the anti-CEA/anti-CD3 bispecific antibody is bispecific antibody, wherein the first antigen binding domain is a cross-Fab molecule wherein the variable domains or the constant domains of the Fab heavy and light chain are exchanged, and the second and third, if present, antigen binding domain is a conventional Fab molecule.

In another aspect, the anti-CEA/anti-CD3 bispecific antibody is bispecific antibody, wherein (i) the second antigen binding domain is fused at the C-terminus of the Fab heavy chain to the N-terminus of the Fab heavy chain of the first antigen binding domain, the first antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain, or (ii) the first antigen binding domain is fused at the C-terminus of the Fab heavy chain to the N-terminus of the Fab heavy chain of the second antigen binding domain, the second antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain.

The Fab molecules may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G4S)ₙ, (SG₄)ₙ, (G₄S)ₙ or G₄(SG₄)ₙ peptide linkers. “n” is generally an integer from 1 to 10, typically from 2 to 4. In one embodiment said peptide linker has a length of at least 5 amino acids, in one embodiment a length of 5 to 100, in a further embodiment of 10 to 50 amino acids. In one embodiment said peptide linker is (GxS)ₙ or (GxS)ₙGₘ with G=glycine, S=serine, and (x=3, n=3, 4, 5 or 6, and m=0, 1, 2 or 3) or (x=4, n=2, 3, 4 or 5 and m=0, 1, 2 or 3), in one embodiment x=4 and n=2 or 3, in a further embodiment x=4 and n=2. In one embodiment said peptide linker is (G₄S)₂. A particularly suitable peptide linker for fusing the Fab light chains of the first and the second Fab molecule to each other is (G₄S)₂. An exemplary peptide linker suitable for connecting the Fab heavy chains of the first and the second Fab fragments comprises the sequence (D)-(G₄S)₂. Another suitable such linker comprises the sequence (G₄S)₄. Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In a further aspect, the anti-CEA/anti-CD3 bispecific antibody comprises an Fc domain comprising one or more amino acid substitutions that reduce binding to an Fc receptor and/or effector function. In particular, the anti-CEA/anti-CD3 bispecific antibody comprises an IgG1 Fc domain comprising the amino acid substitutions L234A, L235A and P329G.

In a particular aspect, the anti-CEA/anti-CD3 bispecific antibody comprises two polypeptides that are at least 95%, 96%, 97%, 98%, or 99% identical to the sequence of SEQ ID NO: 87, a polypeptide that is at least 95%, 96%, 97%, 98%, or 99% identical to the sequence of SEQ ID NO: 88, a polypeptide that is at least 95%, 96%, 97%, 98%, or 99% identical to the sequence of SEQ ID NO: 89, and a polypeptide that is at least 95%, 96%, 97%, 98%, or 99% identical to the sequence of SEQ ID NO: 90. In a further particular embodiment, the bispecific antibody comprises two polypeptides of SEQ ID NO: 87, a polypeptide of SEQ ID NO: 88, a polypeptide of SEQ ID NO: 89 and a polypeptide of SEQ ID NO: 90 (CEA CD3 TCB).

In a further particular aspect, the anti-CEA/anti-CD3 bispecific antibody comprises two polypeptides that are at least 95%, 96%, 97%, 98%, or 99% identical to the sequence of SEQ ID NO:91, a polypeptide that is at least 95%, 96%, 97%, 98%, or 99% identical to the sequence of SEQ ID NO:92, a polypeptide that is at least 95%, 96%, 97%, 98%, or 99% identical to the sequence of SEQ ID NO:93, and a polypeptide that is at least 95%, 96%, 97%, 98%, or 99% identical to the sequence of SEQ ID NO:94. In a further particular embodiment, the bispecific antibody comprises two polypeptides of SEQ ID NO:91, a polypeptide of SEQ ID NO:92, a polypeptide of SEQ ID NO:93 and a polypeptide of SEQ ID NO:94 (CEACAM5 CD3 TCB).

Particular bispecific antibodies are described in PCT publication no. WO 2014/131712 A1.

In a further aspect, the anti-CEA/anti-CD3 bispecific antibody may also comprise a bispecific T cell engager (BiTE®). In a further aspect, the anti-CEA/anti-CD3 bispecific antibody is a bispecific antibody as described in WO 2007/071426 or WO 2014/131712. In another aspect, the bispecific antibody is MEDI565 (AMG211).

Exemplary Anti-FolR1/Anti-CD3 Bispecific Antibodies for Use in the Invention

The present invention also relates to anti-FolR1/anti-CD3 bispecific antibodies and their use in combination with targeted OX40 agonists, in particular to their use in a method for treating or delaying progression of cancer, more particularly for treating or delaying progression of solid tumors. The anti-FolR1/anti-CD3 bispecific antibodies as used herein are bispecific antibodies comprising a first antigen binding domain that binds to CD3, and a second antigen binding domain that binds to FolR1. In a particular, the anti-FolR1/anti-CD3 bispecific antibodies as used herein comprise a third antigen binding domain that binds to FolR1.

In one aspect, the T-cell activating anti-CD3 bispecific antibody comprises a first antigen binding domain comprising a heavy chain variable region (V_{H}CD3), a second antigen binding domain comprising a heavy chain variable region (V_{H}FolR1), a third antigen binding domain comprising a heavy chain variable region (V_{H}FolR1) and three times a common light chain variable region.

In another aspect, the first antigen binding domain comprises a heavy chain variable region (V_{H}CD3) comprising CDR-H1 sequence of SEQ ID NO:95, CDR-H2 sequence of SEQ ID NO:96, and CDR-H3 sequence of SEQ ID NO:97; the second antigen binding domain comprises a heavy chain variable region (V_{H}FolR1) comprising CDR-H1 sequence of SEQ ID NO:98, CDR-H2 sequence of SEQ ID NO:99, and CDR-H3 sequence of SEQ ID NO:100; the third antigen binding domain comprises a heavy chain variable region (V_{H}FolR1) comprising CDR-H1 sequence of SEQ ID NO:98, CDR-H2 sequence of SEQ ID NO:99, and CDR-H3 sequence of SEQ ID NO:100; and the common light chains comprise a CDR-L1 sequence of SEQ ID NO:101, CDR-L2 sequence of SEQ ID NO:102, and CDR-L3 sequence of SEQ ID NO:103. In another aspect, the first antigen binding domain comprises a heavy chain variable region (V_{H}CD3) comprising the sequence of SEQ ID NO:104; the second antigen binding domain comprises a heavy chain variable region (V_{H}FolR1) comprising the sequence of SEQ ID NO:105; the third antigen binding domain comprises a heavy chain variable region (V_{H}FolR1) comprising the sequence of SEQ ID NO:105; and the common light chains comprise the sequence of SEQ ID NO:106.

In a particular aspect, the anti-FolR1/anti-CD3 bispecific antibody comprises a first heavy chain comprising the amino acid sequence of SEQ ID NO:107, a second heavy chain comprising the amino acid sequence of SEQ ID NO:108 and three times a common light chain of SEQ ID NO: 109.

Agents Blocking PD-L1/PD-1 Interaction for Use in the Invention

In one aspect of the invention, the targeted OX40 agonists, in particular bispecific OX40 antibodies comprising at least one antigen binding domain capable of specific binding to a tumor-associated antigen are for use in a method for treating or delaying progression of cancer, wherein the targeted OX40 agonists are used in combination with T-cell activating anti-CD3 bispecific antibodies specific for a tumor-associated antigen, in particular anti-CEA/anti-CD3 bispecific antibodies or anti-FolR1/anti-CD3 bispecific antibodies, and additionally they are combined with an agent blocking PD-L1/PD-1 interaction. In one aspect, an agent blocking PD-L1/PD-1 interaction is a PD-L1 binding antagonist or a PD-1 binding antagonist. In particular, the agent blocking PD-L1/PD-1 interaction is an anti-PD-L1 antibody or an anti-PD-1 antibody.

The term “PD-L1”, also known as CD274 or B7-H1, refers to any native PD-L1 from any vertebrate source, including mammals such as primates (e.g. humans) non-human primates (e.g. cynomolgus monkeys) and rodents (e.g. mice and rats), in particular to “human PD-L1”. The amino acid sequence of complete human PD-L1 is shown in UniProt (www.uniprot.org) accession no. Q9NZQ7 (SEQ ID NO:110). The term “PD-L1 binding antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In particular, a PD-L1 binding antagonist is an anti-PD-L1 antibody. The term “anti-PD-L1 antibody” or “antibody binding to human PD-L1” or “antibody that specifically binds to human PD-L1” or “antagonistic anti-PD-L1” refers to an antibody specifically binding to the human PD-L1 antigen with a binding affinity of KD-value of 1.0×10⁻⁸ mol/l or lower, in one aspect of a KD-value of 1.0×10⁻⁹ mol/l or lower. The binding affinity is determined with a standard binding assay, such as surface plasmon resonance technique (BIAcore®, GE-Healthcare Uppsala, Sweden).

In a particular aspect, the agent blocking PD-L1/PD-1 interaction is an anti-PD-L1 antibody. In a specific aspect, the anti-PD-L1 antibody is selected from the group consisting of atezolizumab (MPDL3280A, RG7446), durvalumab (MEDI4736), avelumab (MSB0010718C) and MDX-1105. In a specific aspect, an anti-PD-L1 antibody is YW243.55 S70 described herein. In another specific aspect, an anti-PD-L1 antibody is MDX-1105 described herein. In still another specific aspect, an anti-PD-L1 antibody is MEDI4736 (durvalumab). In yet a further aspect, an anti-PD-L1 antibody is MSB0010718C (avelumab). More particularly, the agent blocking PD-L1/PD-1 interaction is atezolizumab (MPDL3280A). In another aspect, the agent blocking PD-L1/PD-1 interaction is an anti-PD-L1 antibody comprising a heavy chain variable domain VH(PDL-1) of SEQ ID NO:112 and a light chain variable domain V_{L}(PDL-1) of SEQ ID NO:113. In another aspect, the agent blocking PD-L1/PD-1 interaction is an anti-PD-L1 antibody comprising a heavy chain variable domain VH(PDL-1) of SEQ ID NO:114 and a light chain variable domain VL(PDL-1) of SEQ ID NO:115.

The term “PD-1”, also known as CD279, PD1 or programmed cell death protein 1, refers to any native PD-L1 from any vertebrate source, including mammals such as primates (e.g. humans) non-human primates (e.g. cynomolgus monkeys) and rodents (e.g. mice and rats), in particular to the human protein PD-1 with the amino acid sequence as shown in UniProt (www.uniprot.org) accession no. Q15116 (SEQ ID NO:111). The term “PD-1 binding antagonist” refers to a molecule that inhibits the binding of PD-1 to its ligand binding partners. In some embodiments, the PD-1 binding antagonist inhibits the binding of PD-1 to PD-L1. In some embodiments, the PD-1 binding antagonist inhibits the binding of PD-1 to PD-L2. In some embodiments, the PD-1 binding antagonist inhibits the binding of PD-1 to both PD-L1 and PD-L2. In particular, a PD-L1 binding antagonist is an anti-PD-L1 antibody. The term “anti-PD-1 antibody” or “antibody binding to human PD-1” or “antibody that specifically binds to human PD-1” or “antagonistic anti-PD-1” refers to an antibody specifically binding to the human PD1 antigen with a binding affinity of KD-value of 1.0×10⁻⁸ mol/l or lower, in one aspect of a KD-value of 1.0×10⁻⁹ mol/l or lower. The binding affinity is determined with a standard binding assay, such as surface plasmon resonance technique (BIAcore®, GE-Healthcare Uppsala, Sweden).

In one aspect, the agent blocking PD-L1/PD-1 interaction is an anti-PD-1 antibody. In a specific aspect, the anti-PD-1 antibody is selected from the group consisting of MDX 1106 (nivolumab), MK-3475 (pembrolizumab), CT-011 (pidilizumab), MEDI-0680 (AMP-514), PDR001, REGN2810, and BGB-108, in particular from pembrolizumab and nivolumab. In another aspect, the agent blocking PD-L1/PD-1 interaction is an anti-PD-1 antibody comprising a heavy chain variable domain VH(PD-1) of SEQ ID NO:116 and a light chain variable domain VL(PD-1) of SEQ ID NO:117. In another aspect, the agent blocking PD-L1/PD-1 interaction is an anti-PD-1 antibody comprising a heavy chain variable domain VH(PD-1) of SEQ ID NO:118 and a light chain variable domain VL(PD-1) of SEQ ID NO:119.

Preparation of Bispecific Antibodies for Use in the Invention

In certain aspects, the therapeutic agents used in the combination comprise multispecific antibodies, e.g. bispecific antibodies. Multispecific antibodies are monoclonal antibodies that have binding specificities for at least two different sites. In certain aspects, the binding specificities are for different antigens. In certain aspects, the binding specificities are for different epitopes on the same antigen. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking of two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibodies or fragmentsa herein also include a “Dual Acting FAb” or “DAF” comprising an antigen binding site that binds to two different antigens (see, US 2008/0069820, for example). “Crossmab” antibodies are also included herein (see e.g. WO 2009/080251, WO 2009/080252, WO2009/080253, or WO2009/080254).

Another technique for making bispecific antibody fragments is the “bispecific T cell engager” or BiTE® approach (see, e.g., WO2004/106381, WO2005/061547, WO2007/042261, and WO2008/119567). This approach utilizes two antibody variable domains arranged on a single polypeptide. For example, a single polypeptide chain includes two single chain Fv (scFv) fragments, each having a variable heavy chain (VH) and a variable light chain (VL) domain separated by a polypeptide linker of a length sufficient to allow intramolecular association between the two domains. This single polypeptide further includes a polypeptide spacer sequence between the two scFv fragments. Each scFv recognizes a different epitope, and these epitopes may be specific for different cell types, such that cells of two different cell types are brought into close proximity or tethered when each scFv is engaged with its cognate epitope. One particular embodiment of this approach includes a scFv recognizing a cell-surface antigen expressed by an immune cell, e.g., a CD3 polypeptide on a T cell, linked to another scFv that recognizes a cell-surface antigen expressed by a target cell, such as a malignant or tumor cell.

As it is a single polypeptide, the bispecific T cell engager may be expressed using any prokaryotic or eukaryotic cell expression system known in the art, e.g., a CHO cell line. However, specific purification techniques (see, e.g., EP1691833) may be necessary to separate monomeric bispecific T cell engagers from other multimeric species, which may have biological activities other than the intended activity of the monomer. In one exemplary purification scheme, a solution containing secreted polypeptides is first subjected to a metal affinity chromatography, and polypeptides are eluted with a gradient of imidazole concentrations. This eluate is further purified using anion exchange chromatography, and polypeptides are eluted using with a gradient of sodium chloride concentrations. Finally, this eluate is subjected to size exclusion chromatography to separate monomers from multimeric species. In one aspect, the bispecific bispecific antibodies used in the invention are composed of a single polypeptide chain comprising two single chain FV fragments (scFV) fused to each other by a peptide linker.

Fc Domain Modifications Reducing Fc Receptor Binding and/or Effector Function

The Fc domain of the antigen binding molecules of the invention consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

The Fc domain confers favorable pharmacokinetic properties to the antigen binding molecules of the invention, including a long serum half-life which contributes to good accumulation in the target tissue and a favorable tissue-blood distribution ratio. At the same time it may, however, lead to undesirable targeting of the bispecific antibodies of the invention to cells expressing Fc receptors rather than to the preferred antigen-bearing cells. Accordingly, in particular aspects, the Fc domain of the antigen binding molecules of the invention exhibits reduced binding affinity to an Fc receptor and/or reduced effector function, as compared to a native IgG1 Fc domain. In one aspect, the Fc does not substantially bind to an Fc receptor and/or does not induce effector function. In a particular aspect the Fc receptor is an Fcγ receptor. In one aspect, the Fc receptor is a human Fc receptor. In a specific aspect, the Fc receptor is an activating human Fcγ receptor, more specifically human FcγRIIIa, FcγRI or FcγRIIa, most specifically human FcγRIIIa. In one aspect, the Fc domain does not induce effector function. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

In certain aspects, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In a particular aspect, the invention provides an antibody, wherein the Fc domain comprises one or more amino acid substitution that reduces binding to an Fc receptor, in particular towards Fcγ receptor.

In one aspect, the Fc domain of the antibody of the invention comprises one or more amino acid mutation that reduces the binding affinity of the Fc domain to an Fc receptor and/or effector function. Typically, the same one or more amino acid mutation is present in each of the two subunits of the Fc domain. In particular, the Fc domain comprises an amino acid substitution at a position of E233, L234, L235, N297, P331 and P329 (EU numbering). In particular, the Fc domain comprises amino acid substitutions at positions 234 and 235 (EU numbering) and/or 329 (EU numbering) of the IgG heavy chains. More particularly, provided is an antibody according to the invention which comprises an Fc domain with the amino acid substitutions L234A, L235A and P329G (“P329G LALA”, EU numbering) in the IgG heavy chains. The amino acid substitutions L234A and L235A refer to the so-called LALA mutation. The “P329G LALA” combination of amino acid substitutions almost completely abolishes Fcγ receptor binding of a human IgG1 Fc domain and is described in International Patent Appl. Publ. No. WO 2012/130831 A1 which also describes methods of preparing such mutant Fc domains and methods for determining its properties such as Fc receptor binding or effector functions.

Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In another aspect, the Fc domain is an IgG4 Fc domain. IgG4 antibodies exhibit reduced binding affinity to Fc receptors and reduced effector functions as compared to IgG1 antibodies. In a more specific aspect, the Fc domain is an IgG4 Fc domain comprising an amino acid substitution at position 5228 (Kabat numbering), particularly the amino acid substitution S228P. In a more specific aspect, the Fc domain is an IgG4 Fc domain comprising amino acid substitutions L235E and S228P and P329G (EU numbering). Such IgG4 Fc domain mutants and their Fcγ receptor binding properties are also described in WO 2012/130831.

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. Alternatively, binding affinity of Fc domains or cell activating antibodies comprising an Fc domain for Fc receptors may be evaluated using cell lines known to express particular Fc receptors, such as human NK cells expressing FcγIIIa receptor.

Effector function of an Fc domain, or antibodies of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some aspects, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the bispecific antibodies of the invention are able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

Fc Domain Modifications Promoting Heterodimerization

The bispecific antigen binding molecules of the invention comprise different antigen-binding sites, fused to one or the other of the two subunits of the Fc domain, thus the two subunits of the Fc domain may be comprised in two non-identical polypeptide chains. Recombinant co-expression of these polypeptides and subsequent dimerization leads to several possible combinations of the two polypeptides. To improve the yield and purity of the bispecific antibodies of the invention in recombinant production, it will thus be advantageous to introduce in the Fc domain of the bispecific antigen binding molecules of the invention a modification promoting the association of the desired polypeptides.

Accordingly, in particular aspects the invention relates to the bispecific antigen binding molecule comprising (a) at least one antigen binding domain capable of specific binding to a tumor-associated antigen, (b) at least one antigen binding domain capable of specific binding to OX40, and (c) a Fc domain composed of a first and a second subunit capable of stable association, wherein the Fc domain comprises a modification promoting the association of the first and second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

In a specific aspect said modification is a so-called “knob-into-hole” modification, comprising a “knob” modification in one of the two subunits of the Fc domain and a “hole” modification in the other one of the two subunits of the Fc domain. Thus, the invention relates to an antigen binding molecule comprising (a) at least one antigen binding domain capable of specific binding to a tumor-associated antigen, (b) at least one antigen binding domain capable of specific binding to OX40, and (c) a Fc domain composed of a first and a second subunit capable of stable association, wherein the first subunit of the Fc domain comprises knobs and the second subunit of the Fc domain comprises holes according to the knobs into holes method. In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in one aspect, in the CH3 domain of the first subunit of the Fc domain of the bispecific antigen binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. The protuberance and cavity can be made by altering the nucleic acid encoding the polypeptides, e.g. by site-specific mutagenesis, or by peptide synthesis. In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A).

In yet a further aspect, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter (2001), J Immunol Methods 248, 7-15). In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

The C-terminus of the heavy chain of the bispecific antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, a bispecific antibody comprising a heavy chain including a C-terminal CH3 domain as specified herein, comprises the C-terminal glycine-lysine dipeptide (G446 and K447, numbering according to Kabat EU index). In one embodiment of all aspects as reported herein, a bispecific antibody comprising a heavy chain including a C-terminal CH3 domain, as specified herein, comprises a C-terminal glycine residue (G446, numbering according to Kabat EU index).

Modifications in the Fab Domains

In one aspect, the invention relates to bispecific antibodies comprising at least one Fab fragment, wherein either the variable domains VH and VL or the constant domains CH1 and CL are exchanged. The bispecific antibodies are prepared according to the Crossmab technology.

Multispecific antibodies with a domain replacement/exchange in one binding arm (CrossMabVH-VL or CrossMabCH-CL) are described in detail in WO2009/080252 and Schaefer, W. et al, PNAS, 108 (2011) 11187-1191. They clearly reduce the byproducts caused by the mismatch of a light chain against a first antigen with the wrong heavy chain against the second antigen (compared to approaches without such domain exchange).

In one aspect, the invention relates to a bispecific antigen binding molecule comprising a Fab fragment, wherein the constant domains CL and CH1 are replaced by each other so that the CH1 domain is part of the light chain and the CL domain is part of the heavy chain.

In another aspect, the invention relates to a bispecific antigen binding molecule comprising a Fab fragment, wherein the variable domains VL and VH are replaced by each other so that the VH domain is part of the light chain and the VL domain is part of the heavy chain.

In another aspect, and to further improve correct pairing, the bispecific antigen binding can contain different charged amino acid substitutions (so-called “charged residues”). These modifications are introduced in the crossed or non-crossed CH1 and CL domains. In a particular aspect, the invention relates to a bispecific antigen binding molecule, wherein in one of CL domains the amino acid at position 123 (EU numbering) has been replaced by arginine (R) and the amino acid at position 124 (EU numbering) has been substituted by lysine (K) and wherein in one of the CH1 domains the amino acids at position 147 (EU numbering) and at position 213 (EU numbering) have been substituted by glutamic acid (E).

Polynucleotides

The invention further provides isolated polynucleotides encoding an antibody as described herein or a fragment thereof.

The isolated polynucleotides encoding the antibodies of the invention may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin.

In some aspects, the isolated polynucleotide encodes the entire antibody according to the invention as described herein. In other embodiments, the isolated polynucleotide encodes a polypeptide comprised in the antibody according to the invention as described herein.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

Recombinant Methods

Bispecific antibodies as used in the invention may be obtained, for example, by solid-state peptide synthesis (e.g. Merrifield solid phase synthesis) or recombinant production. For recombinant production one or more polynucleotide encoding the antibody or polypeptide fragments thereof, e.g., as described above, is isolated and inserted into one or more vectors for further cloning and/or expression in a host cell. Such polynucleotide may be readily isolated and sequenced using conventional procedures. In one aspect of the invention, a vector, preferably an expression vector, comprising one or more of the polynucleotides of the invention is provided. Methods which are well known to those skilled in the art can be used to construct expression vectors containing the coding sequence of the antibody (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). The expression vector can be part of a plasmid, virus, or may be a nucleic acid fragment. The expression vector includes an expression cassette into which the polynucleotide encoding the antibody or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the antibody of the invention or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the antibody or polypeptide fragments thereof is desired, DNA encoding a signal sequence may be placed upstream of the nucleic acid an antibody of the invention or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the fusion protein may be included within or at the ends of the polynucleotide encoding an antibody of the invention or polypeptide fragments thereof

In a further aspect of the invention, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one aspect, a host cell comprises (e.g. has been transformed or transfected with) a vector comprising a polynucleotide that encodes (part of) an antibody of the invention of the invention. As used herein, the term “host cell” refers to any kind of cellular system which can be engineered to generate the fusion proteins of the invention or fragments thereof. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr-CHO cells (Urlaub et al., Proc Natl Acad Sci USA 77, 4216 (1980)); and myeloma cell lines such as YO, NSO, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., YO, NSO, Sp20 cell). Standard technologies are known in the art to express foreign genes in these systems. Cells expressing a polypeptide comprising either the heavy or the light chain of an immunoglobulin, may be engineered so as to also express the other of the immunoglobulin chains such that the expressed product is an immunoglobulin that has both a heavy and a light chain.

In one aspect, a method of producing an antibody of the invention or polypeptide fragments thereof is provided, wherein the method comprises culturing a host cell comprising polynucleotides encoding the antibody of the invention or polypeptide fragments thereof, as provided herein, under conditions suitable for expression of the antibody of the invention or polypeptide fragments thereof, and recovering the antibody of the invention or polypeptide fragments thereof from the host cell (or host cell culture medium).

In certain embodiments the moieties capable of specific binding to a target cell antigen (e.g. Fab fragments) forming part of the antigen binding molecule comprise at least an immunoglobulin variable region capable of binding to an antigen. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty).

Any animal species of immunoglobulin can be used in the invention. Non-limiting immunoglobulins useful in the present invention can be of murine, primate, or human origin. If the fusion protein is intended for human use, a chimeric form of immunoglobulin may be used wherein the constant regions of the immunoglobulin are from a human. A humanized or fully human form of the immunoglobulin can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Particular immunoglobulins according to the invention are human immunoglobulins. Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N.J., 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain aspects, the antibodies are engineered to have enhanced binding affinity according to, for example, the methods disclosed in PCT publication WO 2012/020006 (see Examples relating to affinity maturation) or U.S. Pat. Appl. Publ. No. 2004/0132066. The ability of the antigen binding molecules of the invention to bind to a specific antigenic determinant can be measured either through an enzyme-linked immunosorbent assay (ELISA) or other techniques familiar to one of skill in the art, e.g. surface plasmon resonance technique (Liljeblad, et al., Glyco J 17, 323-329 (2000)), and traditional binding assays (Heeley, Endocr Res 28, 217-229 (2002)). Competition assays may be used to identify an antigen binding molecule that competes with a reference antibody for binding to a particular antigen. In certain embodiments, such a competing antigen binding molecule binds to the same epitope (e.g. a linear or a conformational epitope) that is bound by the reference antigen binding molecule. Detailed exemplary methods for mapping an epitope to which an antigen binding molecule binds are provided in Morris (1996) “Epitope Mapping Protocols”, in Methods in Molecular Biology vol. 66 (Humana Press, Totowa, N.J.). In an exemplary competition assay, immobilized antigen is incubated in a solution comprising a first labeled antigen binding molecule that binds to the antigen and a second unlabeled antigen binding molecule that is being tested for its ability to compete with the first antigen binding molecule for binding to the antigen. The second antigen binding molecule may be present in a hybridoma supernatant. As a control, immobilized antigen is incubated in a solution comprising the first labeled antigen binding molecule but not the second unlabeled antigen binding molecule. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antigen binding molecule is competing with the first antigen binding molecule for binding to the antigen. See Harlow and Lane (1988) Antibodies: A Laboratory Manual ch. 14 (Cold Spring Harbor Laboratory, Cold Spring Harbor, N.Y.).

Antibodies of the invention prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the antigen binding molecule binds. For example, for affinity chromatography purification of bispecific antibodies of the invention, a matrix with protein A or protein G may be used. Sequential Protein A or G affinity chromatography and size exclusion chromatography can be used to isolate an antigen binding molecule essentially as described in the Examples. The purity of the antigen binding molecule or fragments thereof can be determined by any of a variety of well-known analytical methods including gel electrophoresis, high pressure liquid chromatography, and the like. For example, the bispecific antibodies as described in the Examples were shown to be intact and properly assembled as demonstrated by reducing and non-reducing SDS-PAGE.

Assays

The antigen binding molecules provided herein may be identified, screened for, or characterized for their physical/chemical properties and/or biological activities by various assays known in the art.

1. Affinity Assays

The affinity of the bispecific antigen binding molecules provided herein for the corresponding receptor can be determined in accordance with the methods set forth in the Examples by surface plasmon resonance (SPR), using standard instrumentation such as a BIAcore instrument (GE Healthcare), and receptors or target proteins such as may be obtained by recombinant expression. The affinity of the bispecific antigen binding molecule for the target cell antigen can also be determined by surface plasmon resonance (SPR), using standard instrumentation such as a BIAcore instrument (GE Healthcare), and receptors or target proteins such as may be obtained by recombinant expression. For the FAP-OX40 bispecific antibodies the methods have been described in more detail in International Patent Appl. Publ. No. WO 2017/055398 A2 or WO 2017/060144 A1. According to one aspect, K_{D} is measured by surface plasmon resonance using a BIACORE® T100 machine (GE Healthcare) at 25° C.

2. Binding Assays and Other Assays

In one aspect, the FAP-OX40 bispecific antibody as reported herein is tested for its antigen binding activity as described in more detail in International Patent Appl. Publ. No. WO 2017/055398 A2 or WO 2017/060144 A1.

3. Activity Assays

In one aspect, assays are provided for identifying the biological activity of targeted OX40 bispecific antigen binding molecules.

In certain embodiments, an antibody as reported herein is tested for such biological activity.

Pharmaceutical Compositions, Formulations and Routes of Administration

In a further aspect, the invention provides pharmaceutical compositions comprising the bispecific OX40 antibody comprising at least one antigen binding domain capable of specific binding to a tumor-associated antigen and the T-cell activating anti-CD3 bispecific antibody specific for a tumor-associated antigen, in particular an anti-CEA/anti-CD3 bispecific antibody or anti-FolR1/anti-CD3 bispecific antibody, provided herein, e.g., for use in any of the below therapeutic methods. In one embodiment, a pharmaceutical composition comprises an antibody provided herein and at least one pharmaceutically acceptable excipient. In another embodiment, a pharmaceutical composition comprises the antibody provided herein and at least one additional therapeutic agent, e.g., as described below.

In one aspect, the invention provides pharmaceutical compositions comprising an anti-FAP/anti-OX40 bispecific antibody and the T-cell activating anti-CD3 bispecific antibody specific for a tumor-associated antigen, in particular an anti-CEA/anti-CD3 bispecific antibody or anti-FolR1/anti-CD3 bispecific antibody.

In another aspect, the invention provides pharmaceutical compositions comprising the bispecific OX40 antibody comprising at least one antigen binding domain capable of specific binding to a tumor-associated antigen, a T-cell activating anti-CD3 bispecific antibody specific for a tumor-associated antigen and an agent blocking PD-L1/PD-1 interaction. In particular, the agent blocking PD-L1/PD-1 interaction is an antagonistic anti-PD-L1 antibody or an antagonistic anti-PD1 antibody. More particularly, the agent blocking PD-L1/PD-1 interaction is selected from the group consisting of atezolizumab, durvalumab, pembrolizumab and nivolumab. In a specific aspect, the agent blocking PD-L1/PD-1 interaction is atezolizumab.

Pharmaceutical compositions of the present invention comprise a therapeutically effective amount of one or more antibodies dissolved or dispersed in a pharmaceutically acceptable excipient. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition comprising the active ingredients (e.g. an bispecific OX40 antibody comprising at least one antigen binding domain capable of specific binding to a tumor-associated antigen, a T-cell activating anti-CD3 bispecific antibody specific for a tumor-associated antigen and/or an agent blocking PD-L1/PD-1 interaction) will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intralesional, intravenous, intraarterial intramuscular, intrathecal or intraperitoneal injection. For injection, the antigen binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the fusion proteins may be in powder form for constitution with a suitable vehicle, e.g., sterile pyrogen-free water, before use. Sterile injectable solutions are prepared by incorporating the fusion proteins of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

Exemplary pharmaceutically acceptable excipients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

In addition to the compositions described previously, the active ingredients may also be formulated as a depot preparation. Such long acting formulations may be administered by implantation (for example subcutaneously or intramuscularly) or by intramuscular injection. Thus, for example, the fusion proteins may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the active ingredients of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The antibody of the invention may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The composition herein may also contain more than one active ingredients as necessary for the particular indication being treated, preferably those with complementary activities that do not adversely affect each other. Such active ingredients are suitably present in combination in amounts that are effective for the purpose intended.

The formulations to be used for in vivo administration are generally sterile. Sterility may be readily accomplished, e.g., by filtration through sterile filtration membranes.

Administration of the Anti-FAP/Anti-OX40 Bispecific Antibody and the T-Cell Activating Anti-CD3 Bispecific Antibody Specific for a Tumor-Associated Antigen, in Particular an Anti-CEA/Anti-CD3 Bispecific Antibody

Both the anti-FAP/anti-OX40 bispecific antibody and the T-cell activating anti-CD3 bispecific antibody specific for a tumor-associated antigen, in particular an anti-CEA/anti-CD3 bispecific antibody (both called substance herein) can be administered by any suitable means, including parenteral, intrapulmonary, and intranasal, and, if desired for local treatment, intralesional administration. The methods of the present invention are particularly useful, however, in relation to therapeutic agents administered by parenteral, particularly intravenous, infusion.

Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein. In one embodiment, the therapeutic agent is administered parenterally, particularly intravenously. In a particular embodiment, the therapeutic agent is administered by intravenous infusion.

Both the anti-FAP/anti-OX40 bispecific antibody and the T-cell activating anti-CD3 bispecific antibody specific for a tumor-associated antigen, in particular an anti-CEA/anti-CD3 bispecific antibody, would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. Both the anti-FAP/anti-OX40 bispecific antibody and the T-cell activating anti-CD3 bispecific antibody specific for a tumor-associated antigen, in particular an anti-CEA/anti-CD3 bispecific antibody, need not be, but are optionally formulated with one or more agents currently used to prevent or treat the disorder in question. The effective amount of such other agents depends on the amount of therapeutic agent present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of the anti-FAP/anti-OX40 bispecific antibody and the T-cell activating anti-CD3 bispecific antibody specific for a tumor-associated antigen, in particular an anti-CEA/anti-CD3 bispecific antibody (when used in their combination or with one or more other additional therapeutic agents) will depend on the type of disease to be treated, the type of the anti-FAP/anti-OX40 bispecific antibody, the severity and course of the disease, whether both agents are administered for preventive or therapeutic purposes, previous therapy, the patient's clinical history and response to the therapeutic agent, and the discretion of the attending physician. Each substance is suitably administered to the patient at one time or over a series of treatments. Depending on the type and severity of the disease, about 1 μg/kg to 15 mg/kg (e.g. 0.1 mg/kg-10 mg/kg) of the substance can be an initial candidate dosage for administration to the subject, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of each substance would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the subject. Such doses may be administered intermittently, e.g. every week, every two weeks, or every three weeks (e.g. such that the subject receives from about two to about twenty, or e.g. about six doses of the therapeutic agent). An initial higher loading dose, followed by one or more lower doses, or an initial lower dose, followed by one or more higher doses may be administered. An exemplary dosing regimen comprises administering an initial dose of about 10 mg, followed by a bi-weekly dose of about 20 mg of the therapeutic agent. However, other dosage regimens may be useful. The progress of this therapy is easily monitored by conventional techniques and assays.

In one aspect, the administration of both the anti-FAP/anti-OX40 bispecific antibody and the T-cell activating anti-CD3 bispecific antibody specific for a tumor-associated antigen, in particular an anti-CEA/anti-CD3 bispecific antibody, is a single administration. In certain aspects, the administration of the therapeutic agent is two or more administrations. In one such aspect, the substances are administered every week, every two weeks, or every three weeks, particularly every two weeks. In one aspect, the substance is administered in a therapeutically effective amount. In one aspect, the substance is administered at a dose of about 50 μg/kg, about 100 μg/kg, about 200 μg/kg, about 300 μg/kg, about 400 μg/kg, about 500 μg/kg, about 600 μg/kg, about 700 μg/kg, about 800 μg/kg, about 900 μg/kg or about 1000 μg/kg. In one embodiment, the anti-CEA/anti-CD3 bispecific antibody is administered at a dose which is higher than the dose of the anti-CEA/anti-CD3 bispecific antibody in a corresponding treatment regimen without the administration of the anti-FAP/anti-OX40 bispecific antibody. In one aspect the administration of the anti-CEA/anti-CD3 bispecific antibody comprises an initial administration of a first dose of the the anti-CEA/anti-CD3 bispecific antibody, and one or more subsequent administrations of a second dose of the anti-CEA/anti-CD3 bispecific antibody, wherein the second dose is higher than the first dose. In one aspect, the administration of the anti-CEA/anti-CD3 bispecific antibody comprises an initial administration of a first dose of the anti-CEA/anti-CD3 bispecific antibody, and one or more subsequent administrations of a second dose of the anti-CEA/anti-CD3 bispecific antibody, wherein the first dose is not lower than the second dose.

In one aspect, the administration of the anti-CEA/anti-CD3 bispecific antibody in the treatment regimen according to the invention is the first administration of that the anti-CEA/anti-CD3 bispecific antibody to the subject (at least within the same course of treatment). In one aspect, no administration of the anti-FAP/anti-OX40 bispecific antibody is made to the subject prior to the administration of the anti-CEA/anti-CD3 bispecific antibody.

In the present invention, the combination of the anti-CEA/anti-CD3 bispecific antibody and the anti-FAP/anti-OX40 bispecific antibody can be used in combination with further agents in a therapy. For instance, at least one additional therapeutic agent may be co-administered. In certain aspects, an additional therapeutic agent is an immunotherapeutic agent.

In one aspect, the combination of the anti-FAP/anti-OX40 bispecific antibody and the anti-CEA/anti-CD3 bispecific antibody can be used in combination with a PD-1 axis binding antagonist. In one aspect, the PD-1 axis binding antagonist is selected from the group consisting of a PD-1 binding antagonist, a PD-L1 binding antagonist and a PD-L2 binding antagonist. In a particular aspect, PD-1 axis binding antagonist is a PD-1 binding antagonist, in particular an antagonistic PD-1 antibody. In one aspect, the PD-1 axis binding antagonist is selected MDX 1106 (nivolumab, CAS Reg. No. 946414-94-4), MK-3475 (pembrolizumab), CT-011 (pidilizumab), MEDI-0680 (AMP-514), PDR001, REGN2810, and BGB-108. In another particular aspect, the PD-1 axis binding antagonist is a PD-L1 binding antagonist, in particular an antagonistic PD-L1 antibody. In one aspect, the PD-1 axis binding antagonist is selected from MPDL3280A (atezolizumab), YW243.55.570, MDX-1105, MEDI4736 (durvalumab), and MSB0010718C (avelumab). In one aspect, the PD-L1 antagonistic antibody is selected from the group consisting of atezolizumab, durvalumab and avelumab. More particularly, the combination of the anti-FAP/anti-OX40 bispecific antibody and the anti-CEA/anti-CD3 bispecific antibody can be used in combination with MPDL3280A (atezolizumab). In some aspects, atezolizumab may be administered at a dose of about 800 mg to about 1500 mg every three weeks (e.g., about 1000 mg to about 1300 mg every three weeks, e.g., about 1100 mg to about 1200 mg every three weeks). In one particular aspect, atezolizumab is administered at a dose of about 1200 mg every three weeks.

The period of time between the administration of the PD-1 axis binding antagonist and the administration of the combination therapy comprising the anti-CEA/anti-CD3 bispecific antibody and the anti-FAP/anti-OX40 bispecific antibody and the doses are chosen such as to effectively shrink the tumor in the subject prior to administration of the combination therapy.

Such combination therapies noted above encompass combined administration (where two or more therapeutic agents are included in the same or separate formulations), and separate administration, in which case, administration of the therapeutic agent can occur prior to, simultaneously, and/or following, administration of an additional therapeutic agent or agents. In one embodiment, administration of the therapeutic agent and administration of an additional therapeutic agent occur within about one month, or within about one, two or three weeks, or within about one, two, three, four, five, or six days, of each other.

Therapeutic Methods and Compositions

Bispecific antibodies recognizing two cell surface proteins on different cell populations hold the promise to redirect cytotoxic immune cells for destruction of pathogenic target cells.

In one aspect, provided is a method for treating or delaying progression of cancer in a subject comprising administering to the subject an effective amount of an anti-FAP/anti-OX40 bispecific antibody and and an anti-CEA/anti-CD3 antibody.

In one such aspect, the method further comprises administering to the subject an effective amount of at least one additional therapeutic agent. In further embodiments, herein is provided a method for tumor shrinkage comprising administering to the subject an effective amount of an anti-FAP/anti-OX40 bispecific antibody and an anti-CEA/anti-CD3 antibody. An “individual” or a “subject” according to any of the above aspects is preferably a human.

In further aspects, a composition for use in cancer immunotherapy is provided comprising an anti-FAP/anti-OX40 bispecific antibody and an anti-CEA/anti-CD3 antibody. In certain embodiments, a composition comprising an anti-FAP/anti-OX40 bispecific antibody and an anti-CEA/anti-CD3 antibody for use in a method of cancer immunotherapy is provided.

In a further aspect, herein is provided the use of a composition comprising an anti-FAP/anti-OX40 bispecific antibody and an anti-CEA/anti-CD3 antibody in the manufacture or preparation of a medicament. In one embodiment, the medicament is for treatment of solid tumors. In a further embodiment, the medicament is for use in a method of tumor shrinkage comprising administering to an individual having a solid tumor an effective amount of the medicament. In one such embodiment, the method further comprises administering to the individual an effective amount of at least one additional therapeutic agent. In a further embodiment, the medicament is for treating solid tumors. In some aspects, the individual has CEA positive cancer. In some aspects, CEA positive cancer is colon cancer, lung cancer, ovarian cancer, gastric cancer, bladder cancer, pancreatic cancer, endometrial cancer, breast cancer, kidney cancer, esophageal cancer, or prostate cancer. In some aspects, the breast cancer is a breast carcinoma or a breast adenocarcinoma. In some aspects, the breast carcinoma is an invasive ductal carcinoma. In some aspects, the lung cancer is a lung adenocarcinoma. In some embodiments, the colon cancer is a colorectal adenocarcinoma. An “individual” according to any of the above embodiments may be a human.

The combination therapies noted above encompass combined administration (where two or more therapeutic agents are included in the same or separate formulations), and separate administration, in which case, administration of the antibody as reported herein can occur prior to, simultaneously, and/or following, administration of the additional therapeutic agent or agents. In one aspect, administration of an anti-FAP/anti-OX40 bispecific antibody and an anti-CEA/anti-CD3 antibody and optionally the administration of an additional therapeutic agent occur within about one month, or within about one, two or three weeks, or within about one, two, three, four, five, or six days, of each other.

Both the anti-FAP/anti-OX40 bispecific antibody and the anti-CEA/anti-CD3 bispecific antibody as reported herein (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Both the anti-FAP/anti-OX40 bispecific antibody and the anti-CEA/anti-CD3 bispecific antibody as reported herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibodies need not be, but are optionally formulated with one or more agents currently used to prevent or treat the disorder in question. The effective amount of such other agents depends on the amount of antibodies present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

Articles of Manufacture (Kits)

In another aspect of the invention, a kit containing materials useful for the treatment, prevention and/or diagnosis of the disorders described above is provided. The kit comprises at least one container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). In one aspect, at least two active agents in the kit are an anti-CEA/anti-CD3 bispecific antibody and an anti-FAP/anti-OX40 bispecific antibody of the invention.

In a particular aspect, provided is a kit for treating or delaying progression of cancer in a subject, comprising a package comprising (A) a first composition comprising as active ingredient an anti-FAP/anti-OX40 bispecific antibody and a pharmaceutically acceptable excipient, (B) a second composition comprising as active ingredient the anti-CEA/anti-CD3 bispecific antibody and a pharmaceutically acceptable excipient, and (C) instructions for using the compositions in a combination therapy.

In one further aspect, provided is a kit for treating or delaying progression of cancer in a subject, comprising a package comprising (A) a first composition comprising as active ingredient an anti-FAP/anti-OX40 bispecific antibody and a pharmaceutically acceptable excipient, (B) a second composition comprising as active ingredient the anti-CEA/anti-CD3 bispecific antibody and a pharmaceutically acceptable excipient, (C) a third composition comprising as active ingredient the agent blocking PD-L1/PD-1 interaction and a pharmaceutically acceptable excipient, and (C) instructions for using the compositions in a combination therapy.

The label or package insert indicates how the composition is used for treating the condition of choice and provides the instructions for using the compositions in a combination therapy. Moreover, the kit may comprise (a) a first container with a composition contained therein, wherein the composition comprises an anti-FAP/anti-OX40 bispecific antibody of the invention; and (b) a second container with a composition contained therein, wherein the composition comprises an anti-CEA/anti-CD3 bispecific antibody of the invention. In addition, the kit may comprise one or more further containers comprising further active ingredients that can be used in combination. The article of manufacture in this embodiment of the invention may further comprise a package insert indicating that the compositions can be used to treat a particular condition.

Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE C
(Sequences):

| SEQ ID | | |
|---|---|---|
| NO: | Name | Sequence |
| 1 | FAP(4B9) CDR-H1 | SYAMS |
| 2 | FAP(4B9) CDR-H2 | AIIGSGASTYYADSVKG |
| 3 | FAP(4B9) CDR-H3 | GWFGGFNY |
| 4 | FAP(4B9) CDR-L1 | RASQSVTSSYLA |
| 5 | FAP(4B9) CDR-L2 | VGSRRAT |
| 6 | FAP(4B9) CDR-L3 | QQGIMLPPT |
| 7 | FAP(4B9) VH | EVQLLESGGGLVQPGGSLRLSCAASGFTFSSYA |
| | | MSWVRQAPGKGLEWVSAIIGSGASTYYADSVKG |
| | | RFTISRDNSKNTLYLQMNSLRAEDTAVYYCAKG |
| | | WFGGFNYWGQGTLVTVSS |
| 8 | FAP(4B9) VL | EIVLTQSPGTLSLSPGERATLSCRASQSVTSSY |
| | | LAWYQQKPGQAPRLLINVGSRRATGIPDRFSGS |
| | | GSGTDFTLTISRLEPEDFAVYYCQQGIMLPPTF |
| | | GQGTKVEIK |
| 9 | FAP (28H1) CDR-H1 | SHAMS |
| 10 | FAP (28H1) CDR-H2 | AIWASGEQYYADSVKG |
| 11 | FAP (28H1) CDR-H3 | GWLGNFDY |
| 12 | FAP (28H1) CDR-L1 | RASQSVSRSYLA |
| 13 | FAP (28H1) CDR-L2 | GASTRAT |
| 14 | FAP (28H1) CDR-L3 | QQGQVIPPT |
| 15 | FAP(28H1) VH | EVQLLESGGGLVQPGGSLRLSCAASGFTFSSHA |
| | | MSWVRQAPGKGLEWVSAIWASGEQYYADSVKGR |
| | | FTISRDNSKNTLYLQMNSLRAEDTAVYYCAKGW |
| | | LGNFDYWGQGTLVTVSS |
| 16 | FAP(28H1) VL | EIVLTQSPGTLSLSPGERATLSCRASQSVSRSY |
| | | LAWYQQKPGQAPRLLIIGASTRATGIPDRFSGS |
| | | GSGTDFTLTISRLEPEDFAVYYCQQGQVIPPTF |
| | | GQGTKVEIK |
| 17 | OX40(8H9,49B4,1G4, | SYAIS |
| | 20B7) CDR-H1 | |
| 18 | OX40(CLC-563, CLC- | SYAMS |
| | 564, 17A9) CDR-H1 | |
| 19 | OX40(8H9,49B4,1G4, | GIIPIFGTANYAQKFQG |
| | 20B7) CDR-H2 | |
| 20 | OX40(CLC-563, CLC- | AISGSGGSTYYADSVKG |
| | 564, 17A9) CDR-H2 | |
| 21 | OX40(8H9) CDR-H3 | EYGWMDY |
| 22 | OX40(49B4) CDR-H3 | EYYRGPYDY |
| 23 | OX40(1G4) CDR-H3 | EYGSMDY |
| 24 | OX40(20B7) CDR-H3 | VNYPYSYWGDFDY |
| 25 | OX40(CLC-563) CDR-H3 | DVGAFDY |
| 26 | OX40(CLC-564) CDR-H3 | DVGPFDY |
| 27 | OX40(17A9)-CDR-H3 | VFYRGGVSMDY |
| 28 | OX40(8H9,49B4,1G4, | RASQSISSWLA |
| | 20B7) CDR-L1 | |
| 29 | OX40(CLC-563, CLC564) | RASQSVSSSYLA |
| | CDR-L1 | |
| 30 | OX40(17A9) CDR-L1 | QGDSLRSYYAS |
| 31 | OX40(8H9,49B4,1G4, | DASSLES |
| | 20B7) CDR-L2 | |
| 32 | OX40(CLC-563, CLC564) | GASSRAT |
| | CDR-L2 | |
| 33 | OX40(17A9) CDR-L2 | GKNNRPS |
| 34 | OX40(8H9) CDR-L3 | QQYLTYSRFT |
| 35 | OX40(49B4) CDR-L3 | QQYSSQPYT |
| 36 | OX40(1G4) CDR-L3 | QQYISYSMLT |
| 37 | OX40(20B7) CDR-L3 | QQYQAFSLT |
| 38 | OX40(CLC-563, CLC- | QQYGSSPLT |
| | 564) CDR-L3 | |
| 39 | OX40(17A9) CDR-L3 | NSRVMPHNRV |
| 40 | OX40(49B4) VH | QVQLVQSGAEVKKPGSSVKVSCKASGGTFSSYA |
| | | ISWVRQAPGQGLEWMGGIIPIFGTANYAQKFQG |
| | | RVTITADKSTSTAYMELSSLRSEDTAVYYCARE |
| | | YYRGPYDYWGQGTTVTVSS |
| 41 | OX40(49B4) VL | DIQMTQSPSTLSASVGDRVTITCRASQSISSWL |
| | | AWYQQKPGKAPKLLIYDASSLESGVPSRFSGSG |
| | | SGTEFTLTISSLQPDDFATYYCQQYSSQPYTFG |
| | | QGTKVEIK |
| 42 | OX40(8H9) VH | QVQLVQSGAEVKKPGSSVKVSCKASGGTFSSYA |
| | | ISWVRQAPGQGLEWMGGIIPIFGTANYAQKFQG |
| | | RVTITADKSTSTAYMELSSLRSEDTAVYYCARE |
| | | YGWMDYWGQGTTVTVSS |
| 43 | OX40(8H9) VL | DIQMTQSPSTLSASVGDRVTITCRASQSISSWL |
| | | AWYQQKPGKAPKLLIYDASSLESGVPSRFSGSG |
| | | SGTEFTLTISSLQPDDFATYYCQQYLTYSRFTF |
| | | GQGTKVEIK |
| 44 | OX40(1G4) VH | QVQLVQSGAEVKKPGSSVKVSCKASGGTFSSYA |
| | | ISWVRQAPGQGLEWMGGIIPIFGTANYAQKFQG |
| | | RVTITADKSTSTAYMELSSLRSEDTAVYYCARE |
| | | YGSMDYWGQGTTVTVSS |
| 45 | OX40(1G4) VL | DIQMTQSPSTLSASVGDRVTITCRASQSISSWL |
| | | AWYQQKPGKAPKLLIYDASSLESGVPSRFSGSG |
| | | SGTEFTLTISSLQPDDFATYYCQQYISYSMLTF |
| | | GQGTKVEIK |
| 46 | OX40(20B7) VH | QVQLVQSGAEVKKPGSSVKVSCKASGGTFSSYA |
| | | ISWVRQAPGQGLEWMGGIIPIFGTANYAQKFQG |
| | | RVTITADKSTSTAYMELSSLRSEDTAVYYCARV |
| | | NYPYSYWGDFDYWGQGTTVTVSS |
| 47 | OX40(20B7) VL | DIQMTQSPSTLSASVGDRVTITCRASQSISSWL |
| | | AWYQQKPGKAPKLLIYDASSLESGVPSRFSGSG |
| | | SGTEFTLTISSLQPDDFATYYCQQYQAFSLTFG |
| | | QGTKVEIK |
| 48 | OX40(CLC-563) VH | EVQLLESGGGLVQPGGSLRLSCAASGFTFSSYA |
| | | MSWVRQAPGKGLEWVSAISGSGGSTYYADSVKG |
| | | RFTISRDNSKNTLYLQMNSLRAEDTAVYYCALD |
| | | VGAFDYWGQGALVTVSS |
| 49 | OX40(CLC-563) VL | EIVLTQSPGTLSLSPGERATLSCRASQSVSSSY |
| | | LAWYQQKPGQAPRLLIYGASSRATGIPDRFSGS |
| | | GSGTDFTLTISRLEPEDFAVYYCQQYGSSPLTF |
| | | GQGTKVEIK |
| 50 | OX40(CLC-564) VH | EVQLLESGGGLVQPGGSLRLSCAASGFTFSSYA |
| | | MSWVRQAPGKGLEWVSAISGSGGSTYYADSVKG |
| | | RFTISRDNSKNTLYLQMNSLRAEDTAVYYCAFD |
| | | VGPFDYWGQGTLVTVSS |
| 51 | OX40(CLC-564) VL | EIVLTQSPGTLSLSPGERATLSCRASQSVSSSY |
| | | LAWYQQKPGQAPRLLIYGASSRATGIPDRFSGS |
| | | GSGTDFTLTISRLEPEDFAVYYCQQYGSSPLTF |
| | | GQGTKVEIK |
| 52 | OX40(17A9) VH | EVQLLESGGGLVQPGGSLRLSCAASGFTFSSYA |
| | | MSWVRQAPGKGLEWVSAISGSGGSTYYADSVKG |
| | | RFTISRDNSKNTLYLQMNSLRAEDTAVYYCARV |
| | | FYRGGVSMDYWGQGTLVTVSS |
| 53 | OX40(17A9) VL | SSELTQDPAVSVALGQTVRITCQGDSLRSYYAS |
| | | WYQQKPGQAPVLVIYGKNNRPSGIPDRFSGSSS |
| | | GNTASLTITGAQAEDEADYYCNSRVMPHNRVFG |
| | | GGTKLTV |
| 54 | HC 1 | QVQLVQSGAEVKKPGSSVKVSCKASGGTFSSYA |
| | (49B4) VHCH1_VHCH1 | ISWVRQAPGQGLEWMGGIIPIFGTANYAQKFQG |
| | Fc knob VH (4B9) | RVTITADKSTSTAYMELSSLRSEDTAVYYCARE |
| | | YYRGPYDYWGQGTTVTVSSASTKGPSVFPLAPS |
| | | SKSTSGGTAALGCLVKDYFPEPVTVSWNSGALT |
| | | SGVHTFPAVLQSSGLYSLSSVVTVPSSSLGTQT |
| | | YICNVNHKPSNTKVDKKVEPKSCDGGGGSGGGG |
| | | SQVQLVQSGAEVKKPGSSVKVSCKASGGTFSSY |
| | | AISWVRQAPGQGLEWMGGIIPIFGTANYAQKFQ |
| | | GRVTITADKSTSTAYMELSSLRSEDTAVYYCAR |
| | | EYYRGPYDYWGQGTTVTVSSASTKGPSVFPLAP |
| | | SSKSTSGGTAALGCLVKDYFPEPVTVSWNSGAL |
| | | TSGVHTFPAVLQSSGLYSLSSVVTVPSSSLGTQ |
| | | TYICNVNHKPSNTKVDKKVEPKSCDKTHTCPPC |
| | | PAPEAAGGPSVFLFPPKPKDTLMISRTPEVTCV |
| | | VVDVSHEDPEVKFNWYVDGVEVHNAKTKPREEQ |
| | | YNSTYRVVSVLTVLHQDWLNGKEYKCKVSNKAL |
| | | GAPIEKTISKAKGQPREPQVYTLPPCRDELTKN |
| | | QVSLWCLVKGFYPSDIAVEWESNGQPENNYKTT |
| | | PPVLDSDGSFFLYSKLTVDKSRWQQGNVFSCSV |
| | | MHEALHNHYTQKSLSLSPGGGGGSGGGGSGGGG |
| | | SGGGGSEVQLLESGGGLVQPGGSLRLSCAASGF |
| | | TFSSYAMSWVRQAPGKGLEWVSAIIGSGASTYY |
| | | ADSVKGRFTISRDNSKNTLYLQMNSLRAEDTAV |
| | | YYCAKGWFGGFNYWGQGTLVTVSS |
| 55 | HC 2 | QVQLVQSGAEVKKPGSSVKVSCKASGGTFSSYA |
| | (49B4) VHCH1_VHCH1 | ISWVRQAPGQGLEWMGGIIPIFGTANYAQKFQG |
| | Fc hole VL (4B9) | RVTITADKSTSTAYMELSSLRSEDTAVYYCARE |
| | | YYRGPYDYWGQGTTVTVSSASTKGPSVFPLAPS |
| | | SKSTSGGTAALGCLVKDYFPEPVTVSWNSGALT |
| | | SGVHTFPAVLQSSGLYSLSSVVTVPSSSLGTQT |
| | | YICNVNHKPSNTKVDKKVEPKSCDGGGGSGGGG |
| | | SQVQLVQSGAEVKKPGSSVKVSCKASGGTFSSY |
| | | AISWVRQAPGQGLEWMGGIIPIFGTANYAQKFQ |
| | | GRVTITADKSTSTAYMELSSLRSEDTAVYYCAR |
| | | EYYRGPYDYWGQGTTVTVSSASTKGPSVFPLAP |
| | | SSKSTSGGTAALGCLVKDYFPEPVTVSWNSGAL |
| | | TSGVHTFPAVLQSSGLYSLSSVVTVPSSSLGTQ |
| | | TYICNVNHKPSNTKVDKKVEPKSCDKTHTCPPC |
| | | PAPEAAGGPSVFLFPPKPKDTLMISRTPEVTCV |
| | | VVDVSHEDPEVKFNWYVDGVEVHNAKTKPREEQ |
| | | YNSTYRVVSVLTVLHQDWLNGKEYKCKVSNKAL |
| | | GAPIEKTISKAKGQPREPQVCTLPPSRDELTKN |
| | | QVSLSCAVKGFYPSDIAVEWESNGQPENNYKTT |
| | | PPVLDSDGSFFLVSKLTVDKSRWQQGNVFSCSV |
| | | MHEALHNHYTQKSLSLSPGGGGGSGGGGSGGGG |
| | | SGGGGSEIVLTQSPGTLSLSPGERATLSCRASQ |
| | | SVTSSYLAWYQQKPGQAPRLLINVGSRRATGIP |
| | | DRFSGSGSGTDFTLTISRLEPEDFAVYYCQQGI |
| | | MLPPTFGQGTKVEIK |
| 56 | LC (49B4) | DIQMTQSPSTLSASVGDRVTITCRASQSISSWL |
| | | AWYQQKPGKAPKLLIYDASSLESGVPSRFSGSG |
| | | SGTEFTLTISSLQPDDFATYYCQQYSSQPYTFG |
| | | QGTKVEIKRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTH |
| | | QGLSSPVTKSFNRGEC |
| 57 | HC 1 | QVQLVQSGAEVKKPGSSVKVSCKASGGTFSSYA |
| | (49B4) VHCH1_VHCH1 | ISWVRQAPGQGLEWMGGIIPIFGTANYAQKFQG |
| | Fc knob VH (28H1) | RVTITADKSTSTAYMELSSLRSEDTAVYYCARE |
| | | YYRGPYDYWGQGTTVTVSSASTKGPSVFPLAPS |
| | | SKSTSGGTAALGCLVKDYFPEPVTVSWNSGALT |
| | | SGVHTFPAVLQSSGLYSLSSVVTVPSSSLGTQT |
| | | YICNVNHKPSNTKVDKKVEPKSCDGGGGSGGGG |
| | | SQVQLVQSGAEVKKPGSSVKVSCKASGGTFSSY |
| | | AISWVRQAPGQGLEWMGGIIPIFGTANYAQKFQ |
| | | GRVTITADKSTSTAYMELSSLRSEDTAVYYCAR |
| | | EYYRGPYDYWGQGTTVTVSSASTKGPSVFPLAP |
| | | SSKSTSGGTAALGCLVKDYFPEPVTVSWNSGAL |
| | | TSGVHTFPAVLQSSGLYSLSSVVTVPSSSLGTQ |
| | | TYICNVNHKPSNTKVDKKVEPKSCDKTHTCPPC |
| | | PAPEAAGGPSVFLFPPKPKDTLMISRTPEVTCV |
| | | VVDVSHEDPEVKFNWYVDGVEVHNAKTKPREEQ |
| | | YNSTYRVVSVLTVLHQDWLNGKEYKCKVSNKAL |
| | | GAPIEKTISKAKGQPREPQVYTLPPCRDELTKN |
| | | QVSLWCLVKGFYPSDIAVEWESNGQPENNYKTT |
| | | PPVLDSDGSFFLYSKLTVDKSRWQQGNVFSCSV |
| | | MHEALHNHYTQKSLSLSPGGGGGSGGGGSGGGG |
| | | SGGGGSEVQLLESGGGLVQPGGSLRLSCAASGF |
| | | TFSSHAMSWVRQAPGKGLEWVSAIWASGEQYYA |
| | | DSVKGRFTISRDNSKNTLYLQMNSLRAEDTAVY |
| | | YCAKGWLGNFDYWGQGTLVTVSS |
| 58 | HC 2 | QVQLVQSGAEVKKPGSSVKVSCKASGGTFSSYA |
| | (49B4) VHCH1_VHCH1 | ISWVRQAPGQGLEWMGGIIPIFGTANYAQKFQG |
| | Fc hole VL (28H1) | RVTITADKSTSTAYMELSSLRSEDTAVYYCARE |
| | | YYRGPYDYWGQGTTVTVSSASTKGPSVFPLAPS |
| | | SKSTSGGTAALGCLVKDYFPEPVTVSWNSGALT |
| | | SGVHTFPAVLQSSGLYSLSSVVTVPSSSLGTQT |
| | | YICNVNHKPSNTKVDKKVEPKSCDGGGGSGGGG |
| | | SQVQLVQSGAEVKKPGSSVKVSCKASGGTFSSY |
| | | AISWVRQAPGQGLEWMGGIIPIFGTANYAQKFQ |
| | | GRVTITADKSTSTAYMELSSLRSEDTAVYYCAR |
| | | EYYRGPYDYWGQGTTVTVSSASTKGPSVFPLAP |
| | | SSKSTSGGTAALGCLVKDYFPEPVTVSWNSGAL |
| | | TSGVHTFPAVLQSSGLYSLSSVVTVPSSSLGTQ |
| | | TYICNVNHKPSNTKVDKKVEPKSCDKTHTCPPC |
| | | PAPEAAGGPSVFLFPPKPKDTLMISRTPEVTCV |
| | | VVDVSHEDPEVKFNWYVDGVEVHNAKTKPREEQ |
| | | YNSTYRVVSVLTVLHQDWLNGKEYKCKVSNKAL |
| | | GAPIEKTISKAKGQPREPQVCTLPPSRDELTKN |
| | | QVSLSCAVKGFYPSDIAVEWESNGQPENNYKTT |
| | | PPVLDSDGSFFLVSKLTVDKSRWQQGNVFSCSV |
| | | MHEALHNHYTQKSLSLSPGGGGGSGGGGSGGGG |
| | | SGGGGSEIVLTQSPGTLSLSPGERATLSCRASQ |
| | | SVSRSYLAWYQQKPGQAPRLLIIGASTRATGIP |
| | | DRFSGSGSGTDFTLTISRLEPEDFAVYYCQQGQ |
| | | VIPPTFGQGTKVEIK |
| 59 | HC 1 | QVQLVQSGAEVKKPGSSVKVSCKASGGTFSSYA |
| | (49B4) VHCH1_VHCH1 | ISWVRQAPGQGLEWMGGIIPIFGTANYAQKFQG |
| | Fc knob VL (4B9) | RVTITADKSTSTAYMELSSLRSEDTAVYYCARE |
| | | YYRGPYDYWGQGTTVTVSSASTKGPSVFPLAPS |
| | | SKSTSGGTAALGCLVKDYFPEPVTVSWNSGALT |
| | | SGVHTFPAVLQSSGLYSLSSVVTVPSSSLGTQT |
| | | YICNVNHKPSNTKVDKKVEPKSCDGGGGSGGGG |
| | | SQVQLVQSGAEVKKPGSSVKVSCKASGGTFSSY |
| | | AISWVRQAPGQGLEWMGGIIPIFGTANYAQKFQ |
| | | GRVTITADKSTSTAYMELSSLRSEDTAVYYCAR |
| | | EYYRGPYDYWGQGTTVTVSSASTKGPSVFPLAP |
| | | SSKSTSGGTAALGCLVKDYFPEPVTVSWNSGAL |
| | | TSGVHTFPAVLQSSGLYSLSSVVTVPSSSLGTQ |
| | | TYICNVNHKPSNTKVDKKVEPKSCDKTHTCPPC |
| | | PAPEAAGGPSVFLFPPKPKDTLMISRTPEVTCV |
| | | VVDVSHEDPEVKFNWYVDGVEVHNAKTKPREEQ |
| | | YNSTYRVVSVLTVLHQDWLNGKEYKCKVSNKAL |
| | | GAPIEKTISKAKGQPREPQVYTLPPCRDELTKN |
| | | QVSLWCLVKGFYPSDIAVEWESNGQPENNYKTT |
| | | PPVLDSDGSFFLYSKLTVDKSRWQQGNVFSCSV |
| | | MHEALHNHYTQKSLSLSPGGGGGSGGGGSGGGG |
| | | SGGGGSEIVLTQSPGTLSLSPGERATLSCRASQ |
| | | SVTSSYLAWYQQKPGQAPRLLINVGSRRATGIP |
| | | DRFSGSGSGTDFTLTISRLEPEDFAVYYCQQGI |
| | | MLPPTFGQGTKVEIK |
| 60 | HC 2 | QVQLVQSGAEVKKPGSSVKVSCKASGGTFSSYA |
| | (49B4) VHCH1_VHCH1 | ISWVRQAPGQGLEWMGGIIPIFGTANYAQKFQG |
| | Fc hole VH (4B9) | RVTITADKSTSTAYMELSSLRSEDTAVYYCARE |
| | | YYRGPYDYWGQGTTVTVSSASTKGPSVFPLAPS |
| | | SKSTSGGTAALGCLVKDYFPEPVTVSWNSGALT |
| | | SGVHTFPAVLQSSGLYSLSSVVTVPSSSLGTQT |
| | | YICNVNHKPSNTKVDKKVEPKSCDGGGGSGGGG |
| | | SQVQLVQSGAEVKKPGSSVKVSCKASGGTFSSY |
| | | AISWVRQAPGQGLEWMGGIIPIFGTANYAQKFQ |
| | | GRVTITADKSTSTAYMELSSLRSEDTAVYYCAR |
| | | EYYRGPYDYWGQGTTVTVSSASTKGPSVFPLAP |
| | | SSKSTSGGTAALGCLVKDYFPEPVTVSWNSGAL |
| | | TSGVHTFPAVLQSSGLYSLSSVVTVPSSSLGTQ |
| | | TYICNVNHKPSNTKVDKKVEPKSCDKTHTCPPC |
| | | PAPEAAGGPSVFLFPPKPKDTLMISRTPEVTCV |
| | | VVDVSHEDPEVKFNWYVDGVEVHNAKTKPREEQ |
| | | YNSTYRVVSVLTVLHQDWLNGKEYKCKVSNKAL |
| | | GAPIEKTISKAKGQPREPQVCTLPPSRDELTKN |
| | | QVSLSCAVKGFYPSDIAVEWESNGQPENNYKTT |
| | | PPVLDSDGSFFLVSKLTVDKSRWQQGNVFSCSV |
| | | MHEALHNHYTQKSLSLSPGGGGGSGGGGSGGGG |
| | | SGGGGSEVQLLESGGGLVQPGGSLRLSCAASGF |
| | | TFSSYAMSWVRQAPGKGLEWVSAIIGSGASTYY |
| | | ADSVKGRFTISRDNSKNTLYLQMNSLRAEDTAV |
| | | YYCAKGWFGGFNYWGQGTLVTVSS |
| 61 | HC 1 | QVQLVQSGAEVKKPGSSVKVSCKASGGTFSSYA |
| | (49B4) VHCH1_VHCH1 | ISWVRQAPGQGLEWMGGIIPIFGTANYAQKFQG |
| | Fc wt knob VH (4B9) | RVTITADKSTSTAYMELSSLRSEDTAVYYCARE |
| | | YYRGPYDYWGQGTTVTVSSASTKGPSVFPLAPS |
| | | SKSTSGGTAALGCLVKDYFPEPVTVSWNSGALT |
| | | SGVHTFPAVLQSSGLYSLSSVVTVPSSSLGTQT |
| | | YICNVNHKPSNTKVDKKVEPKSCDGGGGSGGGG |
| | | SQVQLVQSGAEVKKPGSSVKVSCKASGGTFSSY |
| | | AISWVRQAPGQGLEWMGGIIPIFGTANYAQKFQ |
| | | GRVTITADKSTSTAYMELSSLRSEDTAVYYCAR |
| | | EYYRGPYDYWGQGTTVTVSSASTKGPSVFPLAP |
| | | SSKSTSGGTAALGCLVKDYFPEPVTVSWNSGAL |
| | | TSGVHTFPAVLQSSGLYSLSSVVTVPSSSLGTQ |
| | | TYICNVNHKPSNTKVDKKVEPKSCDKTHTCPPC |
| | | PAPELLGGPSVFLFPPKPKDTLMISRTPEVTCV |
| | | VVDVSHEDPEVKFNWYVDGVEVHNAKTKPREEQ |
| | | YNSTYRVVSVLTVLHQDWLNGKEYKCKVSNKAL |
| | | PAPIEKTISKAKGQPREPQVYTLPPCRDELTKN |
| | | QVSLWCLVKGFYPSDIAVEWESNGQPENNYKTT |
| | | PPVLDSDGSFFLYSKLTVDKSRWQQGNVFSCSV |
| | | MHEALHNHYTQKSLSLSPGGGGGSGGGGSGGGG |
| | | SGGGGSEVQLLESGGGLVQPGGSLRLSCAASGF |
| | | TFSSYAMSWVRQAPGKGLEWVSAIIGSGASTYY |
| | | ADSVKGRFTISRDNSKNTLYLQMNSLRAEDTAV |
| | | YYCAKGWFGGFNYWGQGTLVTVSS |
| 62 | HC 2 | QVQLVQSGAEVKKPGSSVKVSCKASGGTFSSYA |
| | (49B4) VHCH1_VHCH1 | ISWVRQAPGQGLEWMGGIIPIFGTANYAQKFQG |
| | Fc wt hole VL (4B9) | RVTITADKSTSTAYMELSSLRSEDTAVYYCARE |
| | | YYRGPYDYWGQGTTVTVSSASTKGPSVFPLAPS |
| | | SKSTSGGTAALGCLVKDYFPEPVTVSWNSGALT |
| | | SGVHTFPAVLQSSGLYSLSSVVTVPSSSLGTQT |
| | | YICNVNHKPSNTKVDKKVEPKSCDGGGGSGGGG |
| | | SQVQLVQSGAEVKKPGSSVKVSCKASGGTFSSY |
| | | AISWVRQAPGQGLEWMGGIIPIFGTANYAQKFQ |
| | | GRVTITADKSTSTAYMELSSLRSEDTAVYYCAR |
| | | EYYRGPYDYWGQGTTVTVSSASTKGPSVFPLAP |
| | | SSKSTSGGTAALGCLVKDYFPEPVTVSWNSGAL |
| | | TSGVHTFPAVLQSSGLYSLSSVVTVPSSSLGTQ |
| | | TYICNVNHKPSNTKVDKKVEPKSCDKTHTCPPC |
| | | PAPELLGGPSVFLFPPKPKDTLMISRTPEVTCV |
| | | VVDVSHEDPEVKFNWYVDGVEVHNAKTKPREEQ |
| | | YNSTYRVVSVLTVLHQDWLNGKEYKCKVSNKAL |
| | | PAPIEKTISKAKGQPREPQVCTLPPSRDELTKN |
| | | QVSLSCAVKGFYPSDIAVEWESNGQPENNYKTT |
| | | PPVLDSDGSFFLVSKLTVDKSRWQQGNVFSCSV |
| | | MHEALHNHYTQKSLSLSPGGGGGSGGGGSGGGG |
| | | SGGGGSEIVLTQSPGTLSLSPGERATLSCRASQ |
| | | SVTSSYLAWYQQKPGQAPRLLINVGSRRATGIP |
| | | DRFSGSGSGTDFTLTISRLEPEDFAVYYCQQGI |
| | | MLPPTFGQGTKVEIK |
| 63 | CD3-HCDR1 | TYAMN |
| 64 | CD3-HCDR2 | RIRSKYNNYATYYADSVKG |
| 65 | CD3-HCDR3 | HGNFGNSYVSWFAY |
| 66 | CD3-LCDR1 | GSSTGAVTTSNYAN |
| 67 | CD3-LCDR2 | GTNKRAP |
| 68 | CD3-LCDR3 | ALWYSNLWV |
| 69 | CD3 VH | EVQLLESGGGLVQPGGSLRLSCAASGFTFSTYA |
| | | MNWVRQAPGKGLEWVSRIRSKYNNYATYYADSV |
| | | KGRFTISRDDSKNTLYLQMNSLRAEDTAVYYCV |
| | | RHGNFGNSYVSWFAYWGQGTLVTVSS |
| 70 | CD3 VL | QAVVTQEPSLTVSPGGTVTLTCGSSTGAVTTSN |
| | | YANWVQEKPGQAFRGLIGGTNKRAPGTPARFSG |
| | | SLLGGKAALTLSGAQPEDEAEYYCALWYSNLWV |
| | | FGGGTKLTVL |
| 71 | CEA-HCDR1 | EFGMN |
| 72 | CEA-HCDR2 | WINTKTGEATYVEEFKG |
| 73 | CEA-HCDR3 | WDFAYYVEAMDY |
| 74 | CEA-LCDR1 | KASAAVGTYVA |
| 75 | CEA-LCDR2 | SASYRKR |
| 76 | CEA-LCDR3 | HQYYTYPLFT |
| 77 | CEA VH | QVQLVQSGAEVKKPGASVKVSCKASGYTFTEFG |
| | | MNWVRQAPGQGLEWMGWINTKTGEATYVEEFKG |
| | | RVTFTTDTSTSTAYMELRSLRSDDTAVYYCARW |
| | | DFAYYVEAMDYWGQGTTVTVSS |
| 78 | CEA VL | DIQMTQSPSSLSASVGDRVTITCKASAAVGTYV |
| | | AWYQQKPGKAPKLLIYSASYRKRGVPSRFSGSG |
| | | SGTDFTLTISSLQPEDFATYYCHQYYTYPLFTF |
| | | GQGTKLEIK |
| 79 | CEA-HCDR1 | DTYMH |
| | (CEACAM5) | |
| 80 | CEA-HCDR2 | RIDPANGNSKYVPKFQG |
| | (CEACAM5) | |
| 81 | CEA-HCDR3 | FGYYVSDYAMAY |
| | (CEACAM5) | |
| 82 | CEA-LCDR1 | RAGESVDIFGVGFLH |
| | (CEACAM5) | |
| 83 | CEA-LCDR2 | RASNRAT |
| | (CEACAM5) | |
| 84 | CEA-LCDR3 | QQTNEDPYT |
| | (CEACAM5) | |
| 85 | CEA VH (CEACAM5) | QVQLVQSGAEVKKPGSSVKVSCKASGFNIKDTY |
| | | MHWVRQAPGQGLEWMGRIDPANGNSKYVPKFQG |
| | | RVTITADTSTSTAYMELSSLRSEDTAVYYCAPF |
| | | GYYVSDYAMAYWGQGTLVTVSS |
| 86 | CEA VL (CEACAM5) | EIVLTQSPATLSLSPGERATLSCRAGESVDIFG |
| | | VGFLHWYQQKPGQAPRLLIYRASNRATGIPARF |
| | | SGSGSGTDFTLTISSLEPEDFAVYYCQQTNEDP |
| | | YTFGQGTKLEIK |
| 87 | Light chain | DIQMTQSPSSLSASVGDRVTITCKASAAVGTYV |
| | _{”}CEA_{2F1}” | AWYQQKPGKAPKLLIYSASYRKRGVPSRFSGSG |
| | (CEA TCB) | SGTDFTLTISSLQPEDFATYYCHQYYTYPLFTF |
| | | GQGTKLEIKRTVAAPSVFIFPPSDEQLKSGTAS |
| | | VVCLLNNFYPREAKVQWKVDNALQSGNSQESVT |
| | | EQDSKDSTYSLSSTLTLSKADYEKHKVYACEVT |
| | | HQGLSSPVTKSFNRGEC |
| 88 | Light Chain humanized | QAVVTQEPSLTVSPGGTVTLTCGSSTGAVTTSN |
| | CD3_{CH2527} (Crossfab, | YANWVQEKPGQAFRGLIGGTNKRAPGTPARFSG |
| | VL-CH1) | SLLGGKAALTLSGAQPEDEAEYYCALWYSNLWV |
| | (CEA TCB) | FGGGTKLTVLSSASTKGPSVFPLAPSSKSTSGG |
| | | TAALGCLVKDYFPEPVTVSWNSGALTSGVHTFP |
| | | AVLQSSGLYSLSSVVTVPSSSLGTQTYICNVNH |
| | | KPSNTKVDKKVEPKSC |
| 89 | CEA_{CH1A1A98/99} - | QVQLVQSGAEVKKPGASVKVSCKASGYTFTEFG |
| | humanized CD3_{CH2527} | MNWVRQAPGQGLEWMGWINTKTGEATYVEEFKG |
| | (Crossfab VH-Ck) - | RVTFTTDTSTSTAYMELRSLRSDDTAVYYCARW |
| | Fc(knob) P329GLALA | DFAYYVEAMDYWGQGTTVTVSSASTKGPSVFPL |
| | (CEA TCB) | APSSKSTSGGTAALGCLVKDYFPEPVTVSWNSG |
| | | ALTSGVHTFPAVLQSSGLYSLSSVVTVPSSSLG |
| | | TQTYICNVNHKPSNTKVDKKVEPKSCDGGGGSG |
| | | GGGSEVQLLESGGGLVQPGGSLRLSCAASGFTF |
| | | STYAMNWVRQAPGKGLEWVSRIRSKYNNYATYY |
| | | ADSVKGRFTISRDDSKNTLYLQMNSLRAEDTAV |
| | | YYCVRHGNFGNSYVSWFAYWGQGTLVTVSSASV |
| | | AAPSVFIFPPSDEQLKSGTASVVCLLNNFYPRE |
| | | AKVQWKVDNALQSGNSQESVTEQDSKDSTYSLS |
| | | STLTLSKADYEKHKVYACEVTHQGLSSPVTKSF |
| | | NRGECDKTHTCPPCPAPEAAGGPSVFLFPPKPK |
| | | DTLMISRTPEVTCVVVDVSHEDPEVKFNWYVDG |
| | | VEVHNAKTKPREEQYNSTYRVVSVLTVLHQDWL |
| | | NGKEYKCKVSNKALGAPIEKTISKAKGQPRPQE |
| | | VYTLPPCRDELTKNQVSLWCLVKGFYPSDIAVE |
| | | WESNGQPENNYKTTPPVLDSDGSFFLYSKLTVD |
| | | KSRWQQGNVFSCSVMHEALHNHYTQKSLSLSPG |
| | | K |
| 90 | CEA_{CH1A1A98/99} | QVQLVQSGAEVKKPGASVKVSCKASGYTFTEFG |
| | (VH-CH1)- | MNWVRQAPGQGLEWMGWINTKTGEATYVEEFKG |
| | Fc(hole) P329GLALA | RVTFTTDTSTSTAYMELRSLRSDDTAVYYCARW |
| | (CEA TCB) | DFAYYVEAMDYWGQGTTVTVSSASTKGPSVFPL |
| | | APSSKSTSGGTAALGCLVKDYFPEPVTVSWNSG |
| | | ALTSGVHTFPAVLQSSGLYSLSSVVTVPSSSLG |
| | | TQTYICNVNHKPSNTKVDKKVEPKSCDKTHTCP |
| | | PCPAPEAAGGPSVFLFPPKPKDTLMISRTPEVT |
| | | CVVVDVSHEDPEVKFNWYVDGVEVHNAKTKPRE |
| | | EQYNSTYRVVSVLTVLHQDWLNGKEYKCKVSNK |
| | | ALGAPIEKTISKAKGQPREPQVCTLPPSRDELT |
| | | KNQVSLSCAVKGFYPSDIAVEWESNGQPENNYK |
| | | TTPPVLDSDGSFFLVSKLTVDKSRWQQGNVFSC |
| | | SVMHEALHNHYTQKSLSLSPGK |
| 91 | CD3 VH-CL (CEACAM5 | EVQLLESGGGLVQPGGSLRLSCAASGFTFSTYA |
| | TCB) | MNWVRQAPGKGLEWVSRIRSKYNNYATYYADSV |
| | | KGRFTISRDDSKNTLYLQMNSLRAEDTAVYYCV |
| | | RHGNFGNSYVSWFAYWGQGTLVTVSSASVAAPS |
| | | VFIFPPSDEQLKSGTASVVCLLNNFYPREAKVQ |
| | | WKVDNALQSGNSQESVTEQDSKDSTYSLSSTLT |
| | | LSKADYEKHKVYACEVTHQGLSSPVTKSFNRGE |
| | | C |
| 92 | humanized CEA VH- | QVQLVQSGAEVKKPGSSVKVSCKASGFNIKDTY |
| | CH1(EE)-Fc (hole, | MHWVRQAPGQGLEWMGRIDPANGNSKYVPKFQG |
| | P329G LALA) | RVTITADTSTSTAYMELSSLRSEDTAVYYCAPF |
| | (CEACAM5 TCB) | GYYVSDYAMAYWGQGTLVTVSSASTKGPSVFPL |
| | | APSSKSTSGGTAALGCLVEDYFPEPVTVSWNSG |
| | | ALTSGVHTFPAVLQSSGLYSLSSVVTVPSSSLG |
| | | TQTYICNVNHKPSNTKVDEKVEPKSCDKTHTCP |
| | | PCPAPEAAGGPSVFLFPPKPKDTLMISRTPEVT |
| | | CVVVDVSHEDPEVKFNWYVDGVEVHNAKTKPRE |
| | | EQYNSTYRVVSVLTVLHQDWLNGKEYKCKVSNK |
| | | ALGAPIEKTISKAKGQPREPQVCTLPPSRDELT |
| | | KNQVSLSCAVKGFYPSDIAVEWESNGQPENNYK |
| | | TTPPVLDSDGSFFLVSKLTVDKSRWQQGNVFSC |
| | | SVMHEALHNHYTQKSLSLSP |
| 93 | humanized CEA VH- | QVQLVQSGAEVKKPGSSVKVSCKASGFNIKDTY |
| | CH1(EE)-CD3 | MHWVRQAPGQGLEWMGRIDPANGNSKYVPKFQG |
| | VL-CH1-Fc | RVTITADTSTSTAYMELSSLRSEDTAVYYCAPF |
| | (knob, P329G LALA) | GYYVSDYAMAYWGQGTLVTVSSASTKGPSVFPL |
| | (CEACAM5 TCB) | APSSKSTSGGTAALGCLVEDYFPEPVTVSWNSG |
| | | ALTSGVHTFPAVLQSSGLYSLSSVVTVPSSSLG |
| | | TQTYICNVNHKPSNTKVDEKVEPKSCDGGGGSG |
| | | GGGSQAVVTQEPSLTVSPGGTVTLTCGSSTGAV |
| | | TTSNYANWVQEKPGQAFRGLIGGTNKRAPGTPA |
| | | RFSGSLLGGKAALTLSGAQPEDEAEYYCALWYS |
| | | NLWVFGGGTKLTVLSSASTKGPSVFPLAPSSKS |
| | | TSGGTAALGCLVKDYFPEPVTVSWNSGALTSGV |
| | | HTFPAVLQSSGLYSLSSVVTVPSSSLGTQTYIC |
| | | NVNHKPSNTKVDKKVEPKSCDKTHTCPPCPAPE |
| | | AAGGPSVFLFPPKPKDTLMISRTPEVTCVVVDV |
| | | SHEDPEVKFNWYVDGVEVHNAKTKPREEQYNST |
| | | YRVVSVLTVLHQDWLNGKEYKCKVSNKALGAPI |
| | | EKTISKAKGQPREPQVYTLPPCRDELTKNQVSL |
| | | WCLVKGFYPSDIAVEWESNGQPENNYKTTPPVL |
| | | DSDGSFFLYSKLTVDKSRWQQGNVFSCSVMHEA |
| | | LHNHYTQKSLSLSP |
| 94 | humanized | EIVLTQSPATLSLSPGERATLSCRAGESVDIFG |
| | CEA VL-CL(RK) | VGFLHWYQQKPGQAPRLLIYRASNRATGIPARF |
| | (CEACAM5 TCB) | SGSGSGTDFTLTISSLEPEDFAVYYCQQTNEDP |
| | | YTFGQGTKLEIKRTVAAPSVFIFPPSDRKLKSG |
| | | TASVVCLLNNFYPREAKVQWKVDNALQSGNSQE |
| | | SVTEQDSKDSTYSLSSTLTLSKADYEKHKVYAC |
| | | EVTHQGLSSPVTKSFNRGEC |
| 95 | (CH2527) CD3-HCDR1 | TYAMN |
| 96 | (CH2527) CD3-HCDR2 | RIRSKYNNYATYYADSVKG |
| 97 | (CH2527) CD3-HCDR3 | HGNFGNSYVSWFAY |
| 98 | (16D5) Fo1R1-HCDR1 | NAWMS |
| 99 | (16D5) Fo1R1-HCDR2 | RIKSKTDGGTTDYAAPVKG |
| 100 | (16D5) Fo1R1-HCDR3 | PWEWSWYDY |
| 101 | (CH2527-VL7-46-13)- | GSSTGAVTTSNYAN |
| | LCDR1 | |
| 102 | (CH2527-VL7-46-13)- | GTNKRAP |
| | LCDR2 | |
| 103 | (CH2527-VL7-46-13)- | ALWYSNLWV |
| | LCDR3 | |
| 104 | (CH2527) CD3 VH | EVQLLESGGGLVQPGGSLRLSCAASGFTFSTYA |
| | | MNWVRQAPGKGLEWVSRIRSKYNNYATYYADSV |
| | | KGRFTISRDDSKNTLYLQMNSLRAEDTAVYYCV |
| | | RHGNFGNSYVSWFAYWGQGTLVTVSS |
| 105 | (16D5) Fo1R1 VH | EVQLVESGGGLVKPGGSLRLSCAASGFTFSNAW |
| | | MSWVRQAPGKGLEWVGRIKSKTDGGTTDYAAPV |
| | | KGRFTISRDDSKNTLYLQMNSLKTEDTAVYYCT |
| | | TPWEWSWYDYWGQGTLVTVSS |
| 106 | (CH2527-VL7-46-13) | QAVVTQEPSLTVSPGGTVTLTCGSSTGAVTTSN |
| | VL | YANWVQEKPGQAFRGLIGGTNKRAPGTPARFSG |
| | | SLLGGKAALTLSGAQPEDEAEYYCALWYSNLWV |
| | | FGGGTKLTVL |
| 107 | (16D5)VH-CH1- | EVQLVESGGGLVKPGGSLRLSCAASGFTFSNAW |
| | (CH2527)VH-CH1 Fc | MSWVRQAPGKGLEWVGRIKSKTDGGTTDYAAPV |
| | knob PGLALA | KGRFTISRDDSKNTLYLQMNSLKTEDTAVYYCT |
| | | TPWEWSWYDYWGQGTLVTVSSASTKGPSVFPLA |
| | | PSSKSTSGGTAALGCLVKDYFPEPVTVSWNSGA |
| | | LTSGVHTFPAVLQSSGLYSLSSVVTVPSSSLGT |
| | | QTYICNVNHKPSNTKVDKKVEPKSCDGGGGSGG |
| | | GGSEVQLLESGGGLVQPGGSLRLSCAASGFTFS |
| | | TYAMNWVRQAPGKGLEWVSRIRSKYNNYATYYA |
| | | DSVKGRFTISRDDSKNTLYLQMNSLRAEDTAVY |
| | | YCVRHGNFGNSYVSWFAYWGQGTLVTVSSASTK |
| | | GPSVFPLAPSSKSTSGGTAALGCLVKDYFPEPV |
| | | TVSWNSGALTSGVHTFPAVLQSSGLYSLSSVVT |
| | | VPSSSLGTQTYICNVNHKPSNTKVDKKVEPKSC |
| | | DKTHTCPPCPAPEAAGGPSVFLFPPKPKDTLMI |
| | | SRTPEVTCVVVDVSHEDPEVKFNWYVDGVEVHN |
| | | AKTKPREEQYNSTYRVVSVLTVLHQDWLNGKEY |
| | | KCKVSNKALGAPIEKTISKAKGQPREPQVYTLP |
| | | PCRDELTKNQVSLWCLVKGFYPSDIAVEWESNG |
| | | QPENNYKTTPPVLDSDGSFFLYSKLTVDKSRWQ |
| | | QGNVFSCSVMHEALHNHYTQKSLSLSPGK |
| 108 | (16D5)VH-CH1-Fc | EVQLVESGGGLVKPGGSLRLSCAASGFTFSNAW |
| | hole PGLALA | MSWVRQAPGKGLEWVGRIKSKTDGGTTDYAAPV |
| | H435R-Y436F | KGRFTISRDDSKNTLYLQMNSLKTEDTAVYYCT |
| | | TPWEWSWYDYWGQGTLVTVSSASTKGPSVFPLA |
| | | PSSKSTSGGTAALGCLVKDYFPEPVTVSWNSGA |
| | | LTSGVHTFPAVLQSSGLYSLSSVVTVPSSSLGT |
| | | QTYICNVNHKPSNTKVDKKVEPKSCDKTHTCPP |
| | | CPAPEAAGGPSVFLFPPKPKDTLMISRTPEVTC |
| | | VVVDVSHEDPEVKFNWYVDGVEVHNAKTKPREE |
| | | QYNSTYRVVSVLTVLHQDWLNGKEYKCKVSNKA |
| | | LGAPIEKTISKAKGQPREPQVCTLPPSRDELTK |
| | | NQVSLSCAVKGFYPSDIAVEWESNGQPENNYKT |
| | | TPPVLDSDGSFFLVSKLTVDKSRWQQGNVFSCS |
| | | VMHEALHNRFTQKSLSLSPGK |
| 109 | (CH2527-VL7-46-13) | QAVVTQEPSLTVSPGGTVTLTCGSSTGAVTTSN |
| | VL-CL | YANWVQEKPGQAFRGLIGGTNKRAPGTPARFSG |
| | (common light | SLLGGKAALTLSGAQPEDEAEYYCALWYSNLWV |
| | chain) | FGGGTKLTVLGQPKAAPSVTLFPPSSEELQANK |
| | | ATLVCLISDFYPGAVTVAWKADSSPVKAGVETT |
| | | TPSKQSNNKYAASSYLSLTPEQWKSHRSYSCQV |
| | | THEGSTVEKTVAPTECS |
| 110 | human PD-Ll | MRIFAVFIFMTYWHLLNAFTVTVPKDLYVVEYG |
| | (Uniprot Q9NZQ7) | SNMTIECKFPVEKQLDLAALIVYWEMEDKNIIQ |
| | | FVHGEEDLKVQHSSYRQRARLLKDQLSLGNAAL |
| | | QITDVKLQDAGVYRCMISYGGADYKRITVKVNA |
| | | PYNKINQRILVVDPVTSEHELTCQAEGYPKAEV |
| | | IWTSSDHQVLSGKTTTTNSKREEKLFNVTSTLR |
| | | INTTTNEIFYCTFRRLDPEENHTAELVIPELPL |
| | | AHPPNERTHLVILGAILLCLGVALTFIFRLRKG |
| | | RMMDVKKCGIQDTNSKKQSDTHLEET |
| 111 | human PD-1 (Uniprot | MQIPQAPWPVVWAVLQLGWRPGWFLDSPDRPWN |
| | Q15116) | PPTFSPALLVVTEGDNATFTCSFSNTSESFVLN |
| | | WYRMSPSNQTDKLAAFPEDRSQPGQDCRFRVTQ |
| | | LPNGRDFHMSVVRARRNDSGTYLCGAISLAPKA |
| | | QIKESLRAELRVTERRAEVPTAHPSPSPRPAGQ |
| | | FQTLVVGVVGGLLGSLVLLVWVLAVICSRAARG |
| | | TIGARRTGQPLKEDPSAVPVFSVDYGELDFQWR |
| | | EKTPEPPVPCVPEQTEYATIVFPSGMGTSSPAR |
| | | RGSADGPRSAQPLRPEDGHCSWPL |
| 112 | VH (PD-L1) | EVQLVESGGGLVQPGGSLRLSCAASGFTFSDSW |
| | | IHWVRQAPGKGLEWVAWISPYGGSTYYADSVKG |
| | | RFTISADTSKNTAYLQMNSLRAEDTAVYYCARR |
| | | HWPGGFDYWGQGTLVTVSS |
| 113 | VL (PD-L1) | DIQMTQSPSSLSASVGDRVTITCRASQDVSTAV |
| | | AWYQQKPGKAPKLLIYSASFLYSGVPSRFSGSG |
| | | SGTDFTLTISSLQPEDFATYYCQQYLYHPATFG |
| | | QGTKVEIK |
| 114 | VH (PD-L1) | EVQLVESGGGLVQPGGSLRLSCAASGFTFSRYW |
| | | MSWVRQAPGKGLEWVANIKQDGSEKYYVDSVKG |
| | | RFTISRDNAKNSLYLQMNSLRAEDTAVYYCARE |
| | | GGWFGELAFDYWGQGTLVTVSS |
| 115 | VL (PD-L1) | EIVLTQSPGTLSLSPGERATLSCRASQRVSSSY |
| | | LAWYQQKPGQAPRLLIYDASSRATGIPDRFSGS |
| | | GSGTDFTLTISRLEPEDFAVYYCQQYGSLPWTF |
| | | GQGTKVEIK |
| 116 | VH (PD-1) | QVQLVQSGVEVKKPGASVKVSCKASGYTFTNYY |
| | | MYWVRQAPGQGLEWMGGINPSNGGTNFNEKFKN |
| | | RVTLTTDSSTTTAYMELKSLQFDDTAVYYCARR |
| | | DYRFDMGFDYWGQGTTVTVSS |
| 117 | VL (PD-1) | EIVLTQSPATLSLSPGERATLSCRASKGVSTSG |
| | | YSYLHWYQQKPGQAPRLLIYLASYLESGVPARF |
| | | SGSGSGTDFTLTISSLEPEDFAVYYCQHSRDLP |
| | | LTFGGGTKVEIK |
| 118 | VH (PD-1) | QVQLVESGGGVVQPGRSLRLDCKASGITFSNSG |
| | | MHWVRQAPGKGLEWVAVIWYDGSKRYYADSVKG |
| | | RFTISRDNSKNTLFLQMNSLRAEDTAVYYCATN |
| | | DDYWGQGTLVTVSS |
| 119 | VL (PD-1) | EIVLTQSPATLSLSPGERATLSCRASQSVSSYL |
| | | AWYQQKPGQAPRLLIYDASNRATGIPARFSGSG |
| | | SGTDFTLTISSLEPEDFAVYYCQQSSNWPRTFG |
| | | QGTKVEIK |
| 120 | Human (hu) FAP | UniProt no. Q12884 |
| 121 | hu FAP ectodomain + | RPSRVHNSEENTMRALTLKDILNGTFSYKTFFP |
| | poly-lys-tag + | NWISGQEYLHQSADNNIVLYNIETGQSYTILSN |
| | his₆-tag | RTMKSVNASNYGLSPDRQFVYLESDYSKLWRYS |
| | | YTATYYIYDLSNGEFVRGNELPRPIQYLCWSPV |
| | | GSKLAYVYQNNIYLKQRPGDPPFQITFNGRENK |
| | | IFNGIPDWVYEEEMLATKYALWWSPNGKFLAYA |
| | | EFNDTDIPVIAYSYYGDEQYPRTINIPYPKAGA |
| | | KNPVVRIFIIDTTYPAYVGPQEVPVPAMIASSD |
| | | YYFSWLTWVTDERVCLQWLKRVQNVSVLSICDF |
| | | REDWQTWDCPKTQEHIEESRTGWAGGFFVSTPV |
| | | FSYDAISYYKIFSDKDGYKHIHYIKDTVENAIQ |
| | | ITSGKWEAINIFRVTQDSLFYSSNEFEEYPGRR |
| | | NIYRISIGSYPPSKKCVTCHLRKERCQYYTASF |
| | | SDYAKYYALVCYGPGIPISTLHDGRTDQEIKIL |
| | | EENKELENALKNIQLPKEEIKKLEVDEITLWYK |
| | | MILPPQFDRSKKYPLLIQVYGGPCSQSVRSVFA |
| | | VNWISYLASKEGMVIALVDGRGTAFQGDKLLYA |
| | | VYRKLGVYEVEDQITAVRKFIEMGFIDEKRIAI |
| | | WGWSYGGYVSSLALASGTGLFKCGIAVAPVSSW |
| | | EYYASVYTERFMGLPTKDDNLEHYKNSTVMARA |
| | | EYFRNVDYLLIHGTADDNVHFQNSAQIAKALVN |
| | | AQVDFQAMWYSDQNHGLSGLSTNHLYTHMTHFL |
| | | KQCFSLSDGKKKKKKGHHHHHH |
| 122 | mouse FAP | UniProt no. P97321 |
| 123 | Murine FAP | RPSRVYKPEGNTKRALTLKDILNGTFSYKTYFP |
| | ectodomain + | NWISEQEYLHQSEDDNIVFYNIETRESYIILSN |
| | poly-lys-tag + | STMKSVNATDYGLSPDRQFVYLESDYSKLWRYS |
| | his₆-tag | YTATYYIYDLQNGEFVRGYELPRPIQYLCWSPV |
| | | GSKLAYVYQNNIYLKQRPGDPPFQITYTGRENR |
| | | IFNGIPDWVYEEEMLATKYALWWSPDGKFLAYV |
| | | EFNDSDIPIIAYSYYGDGQYPRTINIPYPKAGA |
| | | KNPVVRVFIVDTTYPHHVGPMEVPVPEMIASSD |
| | | YYFSWLTWVSSERVCLQWLKRVQNVSVLSICDF |
| | | REDWHAWECPKNQEHVEESRTGWAGGFFVSTPA |
| | | FSQDATSYYKIFSDKDGYKHIHYIKDTVENAIQ |
| | | ITSGKWEAIYIFRVTQDSLFYSSNEFEGYPGRR |
| | | NIYRISIGNSPPSKKCVTCHLRKERCQYYTASF |
| | | SYKAKYYALVCYGPGLPISTLHDGRTDQEIQVL |
| | | EENKELENSLRNIQLPKVEIKKLKDGGLTFWYK |
| | | MILPPQFDRSKKYPLLIQVYGGPCSQSVKSVFA |
| | | VNWITYLASKEGIVIALVDGRGTAFQGDKFLHA |
| | | VYRKLGVYEVEDQLTAVRKFIEMGFIDEERIAI |
| | | WGWSYGGYVSSLALASGTGLFKCGIAVAPVSSW |
| | | EYYASIYSERFMGLPTKDDNLEHYKNSTVMARA |
| | | EYFRNVDYLLIHGTADDNVHFQNSAQIAKALVN |
| | | AQVDFQAMWYSDQNHGILSGRSQNHLYTHMTHF |
| | | LKQCFSLSDGKKKKKKGHHHHHH |
| 124 | Cynomolgus FAP | RPPRVHNSEENTMRALTLKDILNGTFSYKTFFP |
| | ectodomain + | NWISGQEYLHQSADNNIVLYNIETGQSYTILSN |
| | poly-lys-tag + | RTMKSVNASNYGLSPDRQFVYLESDYSKLWRYS |
| | his₆-tag | YTATYYIYDLSNGEFVRGNELPRPIQYLCWSPV |
| | | GSKLAYVYQNNIYLKQRPGDPPFQITFNGRENK |
| | | IFNGIPDWVYEEEMLATKYALWWSPNGKFLAYA |
| | | EFNDTDIPVIAYSYYGDEQYPRTINIPYPKAGA |
| | | KNPFVRIFIIDTTYPAYVGPQEVPVPAMIASSD |
| | | YYFSWLTWVTDERVCLQWLKRVQNVSVLSICDF |
| | | REDWQTWDCPKTQEHIEESRTGWAGGFFVSTPV |
| | | FSYDAISYYKIFSDKDGYKHIHYIKDTVENAIQ |
| | | ITSGKWEAINIFRVTQDSLFYSSNEFEDYPGRR |
| | | NIYRISIGSYPPSKKCVTCHLRKERCQYYTASF |
| | | SDYAKYYALVCYGPGIPISTLHDGRTDQEIKIL |
| | | EENKELENALKNIQLPKEEIKKLEVDEITLWYK |
| | | MILPPQFDRSKKYPLLIQVYGGPCSQSVRSVFA |
| | | VNWISYLASKEGMVIALVDGRGTAFQGDKLLYA |
| | | VYRKLGVYEVEDQITAVRKFIEMGFIDEKRIAI |
| | | WGWSYGGYVSSLALASGTGLFKCGIAVAPVSSW |
| | | EYYASVYTERFMGLPTKDDNLEHYKNSTVMARA |
| | | EYFRNVDYLLIHGTADDNVHFQNSAQIAKALVN |
| | | AQVDFQAMWYSDQNHGLSGLSTNHLYTHMTHFL |
| | | KQCFSLSDGKKKKKKGHHHHHH |
| 125 | human CEA | UniProt no. P06731 |
| 126 | Human FolR1 | UniProt no. P15328 |
| 127 | Murine FolR1 | UniProt no. P35846 |
| 128 | Cynomolgus FolR1 | UniProt no. G7PR14 |
| 129 | human MCSP | UniProt no. Q6UVK1 |
| 130 | human CD3E | UniProt no. P07766 |
| 131 | cynomolgus CD3E | NCBI GenBank no. BAB71849.1 |
| | | Uniprot Q05115 |
| 132 | G4S peptide | GGGGS |
| | linker | |
| 133 | (G4S)2 | GGGGSGGGGS |
| 134 | (SG4)2 | SGGGGSGGGG |
| 135 | peptide linker | GGGGSGGGGSGGGG |
| 136 | peptide linker | GSPGSSSSGS |
| 137 | (G4S)₃ | GGGGSGGGGSGGGGS₃ |
| | peptide linker | |
| 138 | (G4S)₄ | GGGGSGGGGSGGGGSGGGGS |
| | peptide linker | |
| 139 | peptide linker | GSGSGSGS |
| 140 | peptide linker | GSGSGNGS |
| 141 | peptide linker | GGSGSGSG |
| 142 | peptide linker | GGSGSG |
| 143 | peptide linker | GGSG |
| 144 | peptide linker | GGSGNGSG |
| 145 | peptide linker | GGNGSGSG |
| 146 | peptide linker | GGNGSG |

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991). Amino acids of antibody chains are numbered and referred to according to the numbering systems according to Kabat (Kabat, E. A., et al., Sequences of Proteins of Immunological Interest, 5th ed., Public Health Service, National Institutes of Health, Bethesda, Md. (1991)) as defined above.

### EXAMPLES

The following are examples of methods and compositions of the invention. It is understood that various other embodiments may be practiced, given the general description provided above.

Recombinant DNA Techniques

Standard methods were used to manipulate DNA as described in Sambrook et al., Molecular cloning: A laboratory manual; Cold Spring Harbor Laboratory Press, Cold Spring Harbor, N.Y., 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

DNA Sequencing

DNA sequences were determined by double strand sequencing.

Gene Synthesis

Desired gene segments were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

Cell Culture Techniques

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

Protein Purification

Proteins were purified from filtered cell culture supernatants referring to standard protocols. In brief, antibodies were applied to a Protein A Sepharose column (GE healthcare) and washed with PBS. Elution of antibodies was achieved at pH 2.8 followed by immediate neutralization of the sample. Aggregated protein was separated from monomeric antibodies by size exclusion chromatography (Superdex 200, GE Healthcare) in PBS or in 20 mM Histidine, 150 mM NaCl pH 6.0. Monomeric antibody fractions were pooled, concentrated (if required) using e.g., a MILLIPORE Amicon Ultra (30 MWCO) centrifugal concentrator, frozen and stored at −20° C. or −80° C. Part of the samples were provided for subsequent protein analytics and analytical characterization e.g. by SDS-PAGE, size exclusion chromatography (SEC) or mass spectrometry.

SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGE® Antioxidant running buffer additive) or MOPS (non-reduced gels) running buffer was used.

Analytical Size Exclusion Chromatography

Size exclusion chromatography (SEC) for the determination of the aggregation and oligomeric state of antibodies was performed by HPLC chromatography. Briefly, Protein A purified antibodies were applied to a Tosoh TSKgel G3000SW column in 300 mM NaCl, 50 mM KH₂PO₄/K₂HPO₄, pH 7.5 on an Agilent HPLC 1100 system or to a Superdex 200 column (GE Healthcare) in 2×PBS on a Dionex HPLC-System. The eluted protein was quantified by UV absorbance and integration of peak areas. BioRad Gel Filtration Standard 151-1901 served as a standard.

Mass Spectrometry

This section describes the characterization of the multispecific antibodies with VH/VL exchange (VH/VL CrossMabs) with emphasis on their correct assembly. The expected primary structures were analyzed by electrospray ionization mass spectrometry (ESI-MS) of the deglycosylated intact CrossMabs and deglycosylated/plasmin digested or alternatively deglycosylated/limited LysC digested CrossMabs.

The VH/VL CrossMabs were deglycosylated with N-Glycosidase F in a phosphate or Tris buffer at 37° C. for up to 17 h at a protein concentration of 1 mg/ml. The plasmin or limited LysC (Roche) digestions were performed with 100 μg deglycosylated VH/VL CrossMabs in a Tris buffer pH 8 at room temperature for 120 hours and at 37° C. for 40 min, respectively. Prior to mass spectrometry the samples were desalted via HPLC on a Sephadex G25 column (GE Healthcare). The total mass was determined via ESI-MS on a maXis 4G UHR-QTOF MS system (Bruker Daltonik) equipped with a TriVersa NanoMate source (Advion).

Determination of Binding and Binding Affinity of Multispecific Antibodies to the Respective Antigens Using Surface Plasmon Resonance (SPR) (BIACORE)

Binding of the generated antibodies to the respective antigens is investigated by surface plasmon resonance using a BIACORE instrument (GE Healthcare Biosciences AB, Uppsala, Sweden). Briefly, for affinity measurements Goat-Anti-Human IgG, JIR 109-005-098 antibodies are immobilized on a CMS chip via amine coupling for presentation of the antibodies against the respective antigen. Binding is measured in HBS buffer (HBS-P (10 mM HEPES, 150 mM NaCl, 0.005% Tween 20, ph 7.4), 25° C. (or alternatively at 37° C.). Antigen (R&D Systems or in house purified) was added in various concentrations in solution. Association was measured by an antigen injection of 80 seconds to 3 minutes; dissociation was measured by washing the chip surface with HBS buffer for 3-10 minutes and a KD value was estimated using a 1:1 Langmuir binding model. Negative control data (e.g. buffer curves) are subtracted from sample curves for correction of system intrinsic baseline drift and for noise signal reduction. The respective Biacore Evaluation Software is used for analysis of sensorgrams and for calculation of affinity data.

### Example 1

### Preparation, Purification and Characterization of Anti-FAP/Anti-OX40 Bispecific Antibodies

Anti-FAP/anti-OX40 bispecific antibodies were prepared as described in International Patent Appl. Publ. No. WO 2017/055398 A2 or WO 2017/060144 A1.

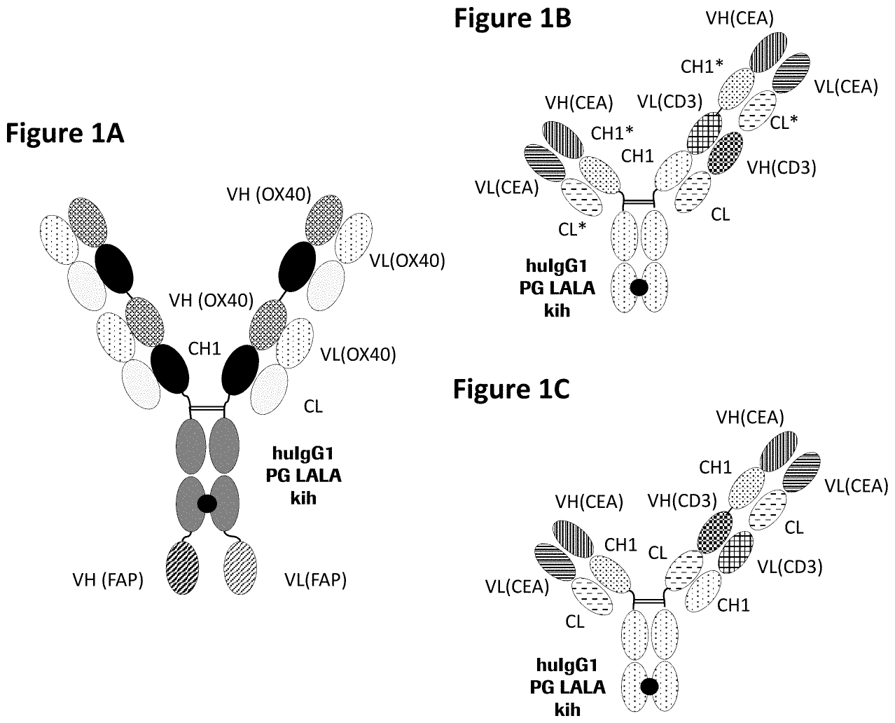

In particular, the molecules according to Example 4.4 of WO 2017/060144 A1 were made, that possess tetravalent binding to OX40 and monovalent binding to FAP. The knob-into-hole technology was applied to allow the assembling of two different heavy chains. A schematic scheme of the bispecific antibodies in 4+1 format is shown in FIG. 1A.

In Molecule A, the first heavy chain (HC 1) was comprised of two Fab units (VHCH1_VHCH1) of the anti-OX40 binder 49B4 followed by Fc knob chain fused by a (G₄S) linker to a VH domain of the anti-FAP binder 4B9. The second heavy chain (HC 2) of the construct was comprised of two Fab units (VHCH1_VHCH1) of the anti-OX40 binder 49B4 followed Fc hole chain fused by a (G₄S) linker to a VL domain of the anti-FAP binder 4B9. Molecule A (FAP OX40 iMAB) thus comprises a first heavy chain comprising the amino acid sequence of SEQ ID NO:54, a second heavy chain comprising the amino acid sequence of SEQ ID NO:55 and four times a light chain of SEQ ID NO: 56.

Molecule B was prepared in analogy to Molecule A, however the FAP binder 4B9 was replaced by FAP binder 28H1. Molecule B comprises a first heavy chain comprising the amino acid sequence of SEQ ID NO:57, a second heavy chain comprising the amino acid sequence of SEQ ID NO:58 and four times a light chain of SEQ ID NO: 56.

In Molecule C, the first heavy chain (HC 1) was comprised of two Fab units (VHCH1_VHCH1) of the anti-OX40 binder 49B4 followed by Fc knob chain fused by a (G4S) linker to the VL domain of the anti-FAP binder 4B9. The second heavy chain (HC 2) of the construct was comprised of two Fab units (VHCH1_VHCH1) of the anti-OX40 binder 49B4 followed Fc hole chain fused by a (G₄S) linker to the VH domain of the anti-FAP binder 4B9. Molecule C comprises a first heavy chain comprising the amino acid sequence of SEQ ID NO:59, a second heavy chain comprising the amino acid sequence of SEQ ID NO:60 and four times a light chain of SEQ ID NO: 56.

In all these molecules the Pro329Gly, Leu234Ala and Leu235Ala mutations were introduced in the constant region of the knob and hole heavy chains to abrogate binding to Fc gamma receptors according to the method described in WO 2012/130831, whereas in Molecule D, the wildtype human IgG1 Fc domain with knob into hole mutations was used. Molecule D comprises a first heavy chain comprising the amino acid sequence of SEQ ID NO:61, a second heavy chain comprising the amino acid sequence of SEQ ID NO:62 and four times a light chain of SEQ ID NO: 56.

The production and characterization of the molecules is described in detail in WO 2017/060144 A1.

### Example 2

### Preparation, Purification and Characterization of T-Cell Bispecific (TCB) Antibodies

TCB molecules have been prepared according to the methods described in WO 2014/131712 A1 or WO 2016/079076 A1.

The preparation of the anti-CEA/anti-CD3 bispecific antibody (CEA CD3 TCB or CEA TCB) used in the experiments is described in Example 3 of WO 2014/131712 A1. CEA CD3 TCB is a “2+1 IgG CrossFab” antibody and is comprised of two different heavy chains and two different light chains (one of them is two times present in the molecule). Point mutations in the CH3 domain (“knobs into holes”) were introduced to promote the assembly of the two different heavy chains. Exchange of the VH and VL domains in the CD3 binding Fab were made in order to promote the correct assembly of the two different light chains. 2+1 means that the molecule has two antigen binding domains specific for CEA and one antigen binding domain specific for CD3. CEACAM5 CD3 TCB has a similar format, but comprises another CEA binder and comprises point mutations in the CH and CL domains of the CD3 binder in order to support correct pairing of the light chains.

CEA CD3 TCB comprises two times a light chain of the amino acid sequence of SEQ ID NO:87, a heavy chain comprising the amino acid sequence of SEQ ID NO:88, a heavy chain compring the amino acid sequence of SEQ ID NO:89 and a light chain compring the amino acid sequence of SEQ ID NO:90. A schematic scheme of the bispecific antibody in 2+1 format is shown in FIG. 1C. CEACAM5 CD TCB comprises two times a light chain of the amino acid sequence of SEQ ID NO:91, a heavy chain comprising the amino acid sequence of SEQ ID NO:92, a heavy chain comprising the amino acid sequence of SEQ ID NO:93 and a light chain comprising the amino acid sequence of SEQ ID NO:94. A schematic scheme of the bispecific antibody in 2+1 format is shown in FIG. 1B.

The preparation of the anti-FolR1/anti-CD3 bispecific antibody (FolR1 CD3 TCB or FolR1 TCB) used in the experiments is described in WO 2016/079076 A1. FolR1 CD3 TCB is shown as “FolR1 TCB 2+1 classical (common light chain)” in FIG. 1D of WO 2016/079076 and is comprised of two different heavy chains and three times the same VLCL light chain (common light chain). Point mutations in the CH3 domain (“knobs into holes”) were introduced to promote the assembly of the two different heavy chains. 2+1 means that the molecule has two antigen binding domains specific for FolR1 and one antigen binding domain specific for CD3. The CD3 binder is fused at the C-terminus of the Fab heavy chain to the N-terminus of of the first subunit of the Fc domain comprising the knob mutation.

FolR1 CD3 TCB comprises a first heavy chain comprising the amino acid sequence of SEQ ID NO:107, a second heavy chain comprising the amino acid sequence of SEQ ID NO:108 and three times a common light chain of SEQ ID NO: 109.

### Example 3

### In Vitro Co-Culture Assays with Human Immune Effector Cells

The immune functions of T cells were tested in in vitro co-culture assays with human immune effector cells (resting PBMC, CD4 or CD8 T cells), target antigen positive tumor cells and FAP positive fibroblasts in the presence of TCBs (CEA CD3 TCB, CEACAM5 CD3 TCB and FolR1 CD3 TCB) and FAP OX40 iMab. Evaluated tumor cell lines were the gastric cancer cell line MKN-45, the ovarian adenocarcinoma cell line SK-OV-3 and the cervical cancer cell line HeLa. The mouse embryonic fibroblast cell line NIH/3T3 transduced to express human FAP was used as FAP positive fibroblast. Effector cells were resting human PBMC and isolated resting CD4 or CD8 T cells. In some assays, TNF-α sensor cells were added to monitor TNF-α induction. Tumor cell lysis (kinetic high content life imaging, endpoint flow cytometry), expression of cell surface activation and maturation markers (end-point flow cytometry) and cytokine secretion (kinetic high content life imaging, endpoint cytometric bead array) was used to monitor the extent of T cell function induced by TCBs and modulated by FAP OX40 iMAB (Molecule A).

### a) Target Cell Lines and Fibroblasts

The SK-OV-3 cells (ATCC, Ca. No. HTP-77) naturally express the folate receptor. HeLa NLR cells (EssenBioscience, Ca. No. 4489) naturally express the folate receptor and MKN45 NLR cells naturally express CEA. Both cell lines harbour the Essen CellPlayer NucLight Red Lentivirus (Essenbioscience, Cat. No. 4476; EF1α, puromycin) to stable express the NucLight Red fluorescent protein restricted to the nucleus. This enables easy separation from non-fluorescent effector T cells or fibroblasts. As the red fluorescence measured per well is directly proportional to the number of red nuclei, and thus healthy tumor cells, real-time assessment of tumor cell lysis or proliferation by high through put life fluorescence microscopy is possible.

HeLa NucLight Red (NLR) cells were cultured in DMEM (GIBCO, Cat. No. 42430-082) containing 10% Fetal Bovine Serum (FBS, Gibco by Life Technology, Cat. No. 16000-044, Lot 941273, gamma-irradiated, mycoplasma-free and heat inactivated at 56° C. for 35 min), 1% (v/v) GlutaMAX I (GIBCO by Life Technologies, Cat. No. 35050 038) and 1 mM Sodium-Pyruvate (SIGMA, Cat. No. S8636).

The MKN45 NucLight Red (NLR) cells naturally express CEA. MKN45 NucLight Red cells were cultured in DMEM (GIBCO, Cat. No 42430-082) containing 10% Fetal Bovine Serum (FBS, Gibco by Life Technology, Cat. No. 16000-044, gamma-irradiated, mycoplasma-free and heat inactivated at 56° C. for 35 min), 1% (v/v) GlutaMAX I (GIBCO by Life Technologies, Cat. No. 35050 038), 1 mM sodium pyruvate (SIGMA, Cat. No. 58636) and 0.5 μg/mL Puromycin (Sigma-Aldrich, Cat. No. ant-pr-1). MKN-45 (DSMZ; ACC409) were transduced with the Essen CellPlayer NucLight Red Lentivirus Reagent (Essenbioscience, Cat. No. 4476; EF1α, puromycin) at an MOI of 5 (TU/cell) in the presence of 8 μg/mL polybrene following the manufacturer's instructions to stable express a nuclear-restricted NucLight Red fluorescent protein. This enables easy separation from non-fluorescent effector T cells or fibroblasts and monitoring of the tumor cell growth by high through put life fluorescence microscopy. Quantification per well over time allows thus real-time assessment of tumor cell lysis or proliferation.

The crosslinking of FAP-binding antibodies by cell surface FAP was provided by human fibroblast activating protein (huFAP) expressing NIH/3T3-huFAP clone 19. This cell line was generated by the transfection of the mouse embryonic fibroblast NIH/3T3 cell line (ATCC CRL-1658) with the expression vector pETR4921 to express huFAP. Cells were cultured in DMEM (GIBCO, Cat. No. 42430-082) containing 10% calf serum (Sigma-Aldrich, Cat. No. C8056-500 ml, gamma-irradiated, mycoplasma-free and heat inactivated at 56° C. for 35 min) and 1.5 μg/mL Puromycin (Sigma-Aldrich, Cat. No. ant-pr-1).

### b) Preparation of Effector Cells

Buffy coats were obtained from the Zurich blood donation center. To isolate fresh peripheral blood mononuclear cells (PBMCs) the buffy coat was diluted with the same volume of DPBS (Gibco by Life Technologies, Cat. No. 14190 326). 50 mL polypropylene centrifuge tubes (TPP, Cat.-No. 91050) were supplied with 15 mL Histopaque 1077 (SIGMA Life Science, Cat.-No. 10771, polysucrose and sodium diatrizoate, adjusted to a density of 1.077 g/mL) and the buffy coat solution was layered above the Histopaque 1077. The tubes were centrifuged for 30 min at 400×g, room temperature and with low acceleration and no break. Afterwards the PBMCs were collected from the interface, washed three times with DPBS and resuspended in T cell medium consisting of RPMI 1640 medium (Gibco by Life Technology, Cat. No. 42401-042) supplied with 10% Fetal Bovine Serum (FBS, Gibco by Life Technology, Cat. No. 16000-044, Lot 941273, gamma-irradiated, mycoplasma-free and heat inactivated at 56° C. for 35 min), 1% (v/v) GlutaMAX I (GIBCO by Life Technologies, Cat. No. 35050 038), 1 mM Sodium Pyruvate (SIGMA, Cat. No. S8636), 1% (v/v) MEM non-essential amino acids (SIGMA, Cat.-No. M7145) and 50 μM β-Mercaptoethanol (SIGMA, M3148). In some cases, RPMI1640 was replaced by FluoroBrite DMEM media (GIBCO, Invitrogen, Cat No A18967-01) for improved high content live microscopy with reduced background fluorescence.

PBMCs were used as effector cells directly after isolation (resting human PBMCs) or certain subfractions, as resting CD4 T cells or CD8 T cells, were isolated using the untouched human CD4+ T cell isolation kit (Miltenyi, Ca. No. 130-096-533) and untouched human CD8+ T cell isolation kit (Miltenyi, Ca. No. 130-096-495) according to manufacturers instructions, respectively. Briefly, human PBMC were centrifuged for 8 min at 400×g, 4° C. and were washed once with MACS buffer (PBS+BSA (0.5% v/w, Sigma-Aldrich, Cat. No. A9418)+EDTA ([2 nM], Ambion, AM9261)). The pellet was resuspended with the respective provided streptavidin labeled negative antibody cocktail and incubated for 5 minutes at 4° C. (per 1*107 cells 40 μL MACS buffer and 10 μL antibody mix) followed by a subsequent incubation with biotinylated magnetic capture beads (per 1*107 cells 30 μL MACS buffer and 20 μL bead mix) for 10 min at 4° C. Labeled non-CD4 or non-C8 T cells were removed by magnetic separation using an LS column (Miltenyi, Ca. No. 130-042-401) according to manufacturer's instructions. The column flow through, containing unlabeled resting CD4 and CD8 T cells, respectively, was centrifuged and washed once with MACS buffer as described above. Cells were adjusted to 2 mio cells/mL in RPMI1640 or Fuorobright DMEM based T cell media.

### c) TNF-α Sensor Cells

TNF-α sensor cells were HEK 293T cells (ATCC, Cat. No. xxx) transduced with the reporter plasmid pETR14327 encoding for green fluorescent protein (GFP) under the control of an NFκB sensitive promotor element. HEK 293T cells express naturally the TNF receptor to which TNF-α secreted by activated T cells can bind. This leads to dose dependent activation of NFκB and translocation to the nucleus, which in turn switches on dose dependent GFP production. The GFP fluorescence can be quantified by high through put life fluorescence microscopy over time and allows thus real-time assessment of TNF-α secretion.

TNF-α sensor cell line was generated by lentiviral transduction of HEK293T cells (ATCC; CRL-3216). Lentivirus-based viral vectors were produced by co-transfection of HEK293T cells with lentiviral packaging plasmids and a lentiviral expression vector (pETR14372) coding for green fluorescent protein (GFP) coupled with the minimal cytomegalovirus (mCMV) promoter in conjunction with the NFκB consensus transcriptional response elements. Plasmid transfections into HEK293T cells were performed with Lipofectamine LTX (Life Technologies) according the manufacturer's instructions. Transfections were done in 6-well plates seeded with 6×10⁵ cells/well the day before transfection and 2.5 μg of plasmid DNA. The lentiviral vector-containing supernatant was collected after 48 h and filtered through a 0.45 μm pore-sized polyethersulfone membrane. To generate stable expressing cell lines, HEK293T cells were seeded at 1.0×10⁶ cells/well in 6-well plates and overlaid with 1 mL of viral vector-containing supernatant. Transductions were carried out by spinoculation at 800×g and at 32° C. for 30 min in an Eppendorf centrifuge 5810 table-top centrifuge (Eppendorf). A TNF-α inducible cell clone was obtained by FACS sorting (FACS ARIA, Becton, Dickinson and Company).

### d) Cytotoxicity and T Cell Activation Assay

Mouse embryonic fibroblast NIH/3T3-huFAP cells, TNF-α sensor cells and MKN45 NLR cells were harvested using cell dissociation buffer (Invitrogen, Cat.-No. 13151-014) for 10 minutes at 37° C. Cells were washed once with DPBS. TNF-α sensor cells or fibroblasts were irradiated in an xRay irradiator using a dose of 4500 RAD to prevent later overgrowth of effector or tumor cell lines. Target cell lines, NIH/3T3-huFAP and in some assays TNF-α sensor cells were cultured at a density of 0.1*10⁵ cells per well in T cell media in a sterile 96-well flat bottom adhesion tissue culture plate (TPP, Cat. No. 92097) overnight at 37° C. and in 5% CO₂ in an incubator (Hera Cell 150).

Resting human PBMC, human CD4 T cells, human CD8 T cells or NLV-specific T cells were prepared as described above and were added at a density of 0.5*105 cells per well. A serial dilution row of TCBs (CEA CD3 TCB or CEA CD3 TCB (2)) and a fixed concentration of FAP OX40 iMab (2 nM) was added to a total volume of 200 uL per well. Cells were cocultured for up to 72 hours at 37° C. and 5% CO₂ in an incubator (Hera Cell 150).

In some assays, plates were monitored by fluorescence microscopy high content life imaging using the Incucyte Zoom System (Essenbioscience, HD phase-contrast, green fluorescence and red fluorescence, 10× objective) in a 3 hours interval for up to 72 hours at 37° C. and 5% CO₂. The integrated red fluorescence of healthy tumor cells (RCUxum2/image), which is proportional to the amount of NLR⁺ cells per well, was quantified using the IncucyteZoom Software to monitor tumor cell growth vs lysis by T cells. Values were plotted for the respective time point and conditions against the used TCB concentration to analyse effects on the cytolytic potential of T cells.

In some assays where TNF-α sensor cells were present, the integrated green RCUxum2/image was quantified using the IncucyteZoom Software to monitor TNF-α induced production of GFP by the TNF-α sensor cells. Values were plotted for the respective time point and conditions against the used TCB concentration to analyze effects on TNF-α secretion by T cells.

After 72 hrs, the supernatant was collected for subsequent analysis of selected cytokine using the cytometric bead array according to manufacturer's instructions. Evaluated cytokines were IL-2 (Human IL-2 CBA Flex-set (Bead A4), BD Bioscience, Ca. No. 558270), IL-17A (Human IL-17A CBA Flex-set (Bead B5), BD Bioscience, Ca. No. 560383), TNF-α (Human TNF-α CBA Flex-set (Bead C4), BD Bioscience, Ca. No. 560112), IFN-γ (IFN-γ CBA Flex-set (Bead E7), BD Bioscience, Ca. No. 558269), IL-4 (Human IL-4 CBA Flex-set (Bead A5), BD Bioscience, Ca. No. 558272), IL-10 (Human IL-10 CBA Flex-set (Bead B7), BD Bioscience, Ca. No. 558274) and IL-9 (Human IL-9 CBA Flex-set (Bead B6), BD Bioscience, Ca. No. 558333).

Thereafter, all cells were detached from the wells by incubation with cell dissociation buffer for 10 minutes at 37° C. followed by centrifugation at 400×g at 4° C. Pellets were washed with ice cold FACS buffer (DPBS (Gibco by Life Technologies, Cat. No. 14190 326) w/BSA (0.1% v/w, Sigma-Aldrich, Cat. No. A9418). Cells were surface-stained with fluorescent dye-conjugated antibodies anti-human CD4 (clone RPA-T4, BioLegend, Cat.-No. 300532), CD8 (clone RPa-T8, BioLegend, Cat.-No. 3010441), CD62L (clone DREG-56, BioLegend, Cat.-No. 304834), CD127 (clone 019D5, BioLegend, Cat.-No. A019D5), CD134 (clone Ber-ACT35, BioLegend, Cat.-No. 350008), CD137 (clone 4B4-1, BioLegend, Cat.-No. 309814), GITR (clone 621, BioLegend, Cat.-No. 3311608) and CD25 (clone M-A251, BioLegend, Cat.-No. 356112) for 20 min at 4° C. in FACS buffer. Then, they were washed once with FACS buffer before being resuspended in 85 μL/well FACS buffer containing 0.2 μg/mL DAPI (Santa Cruz Biotec, Cat. No. Sc-3598) before they were acquired the same day using 5-laser LSR-Fortessa (BD Bioscience with DIVA software). Living CD4 and CD8 T cells were gated (DAPI−, NucLight RED-, CD4 or CD8+) and counts, the mean fluorescence intensity (MFI) of activation marker (CD134, CD137, GITR, CD25) or maturation marker (CD127, CD62L) or percentage of positive cells were plotted for the respective conditions against the used TCB concentration to analyze effects on T activation.

### Results

### 3.1 T Cell Bispecific Antibodies Induce a Dose Dependent Upregulation of OX40 on CD8 and CD4 T Cells

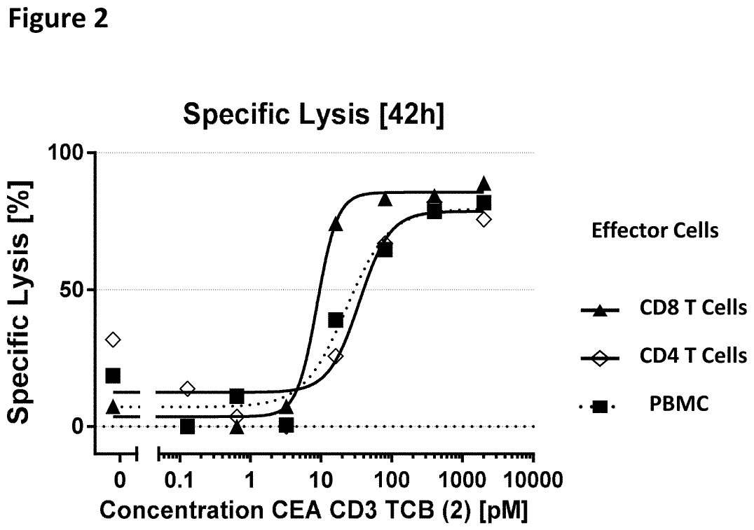

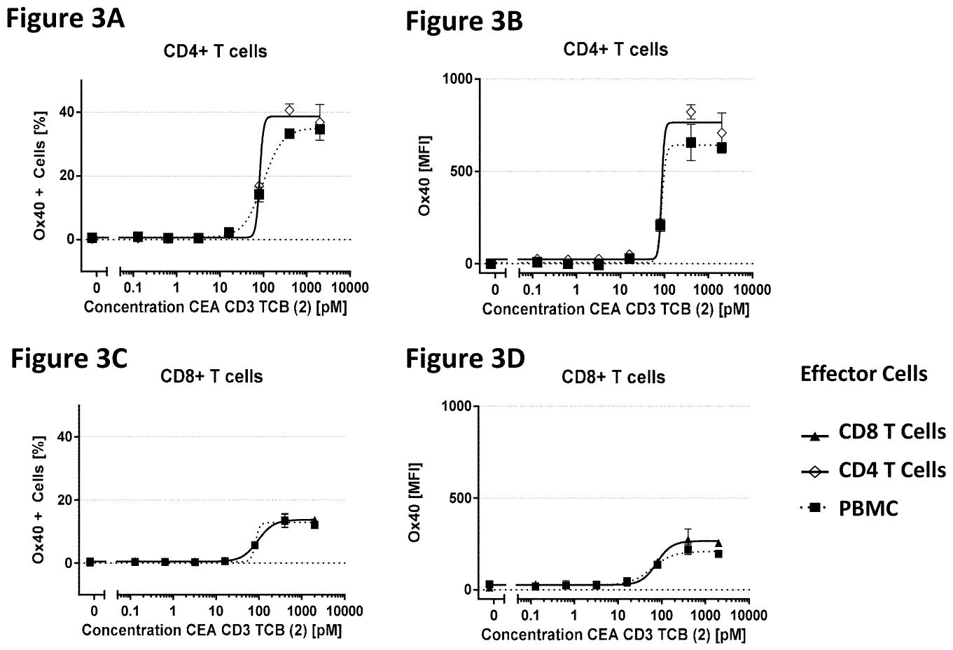

Different human immune effector cell preparations (resting PBMC, CD4 or CD8 T cells, NLV specific CD8 T effector memory cells) were cocultured with MKN-45 NucLight Red cells and irradiated NIH/3T3 huFAP in the presence of a serial dilution row of CEACAM5 CD3 TCB for 48 hrs. The amount of living tumor cells was quantified by fluorescence microscopy high content life imaging using the Incucyte Zoom System and the integrated red fluorescence of healthy tumor cells was used to calculate the specific lysis (FIG. 2). The expression of OX40 was evaluated by flow cytometry on CD4 and CD8 positive T cells (FIGS. 3A-3D).

CEACAM5 CD3 TCB was able to induce lysis of MKN45 NucLight red cells in all used immune effector cell preparations, as shown in FIG. 2 for the 42 hours time point. The EC₅₀ values and the magnitude of lysis differed slightly between the different effector cell preparations and were highest for isolated CD8 T cells. Concomitant to tumor cell lysis, T cells increased surface expression of activation markers including OX40 (FIGS. 3A-3D). Surface expression of OX40 was highest on CD4 positive T cells, but was also detected to a lower extent on CD8 positive T cells. The extent of OX40 expression was not depending on the presence of helper cells (no difference of expression levels in PBMC vs isolated populations for CD4 or CD8 T cells).

3.2 the Presence of FAP-Targeted OX40 Agonists does not Influence the Cytolytic Potential of T Cells

Next we evaluated the influence of OX40 costimulation on TCB mediated tumor cell lysis. As described in 3.1, T cells were cocultured for 48 hours with MKN-45 NucLight Red cells and irradiated NIH/3T3 huFAP in the presence of a serial dilution row of CEACAM5 CD3 TCB with or without a fixed concentration of FAP OX40 iMab, respectively.

The amount of living tumor cells was quantified by fluorescence microscopy high content life imaging using the Incucyte Zoom System in 3 hour intervals and the integrated red fluorescence of healthy tumor cells was used to calculate the specific lysis.

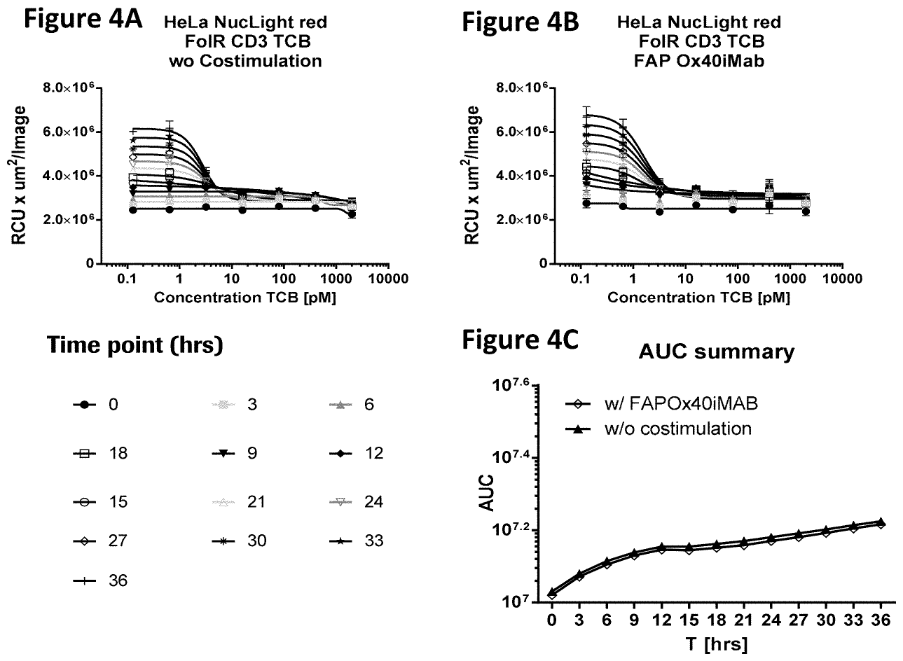

No influence of FAP OX40 iMAB costimulation on the extent of tumor cell lysis was observed for FolR1 CD3 TCB at all evaluated time points (FIGS. 4A-4C). For an easier comparison over time the area under curve (AUC) was calculated for each time point with and w/o FAP OX40 iMAB costimulation and was plotted against time. The AUC was increasing over time as tumor cells were proliferating in the absence of TCB but clearly no costimulation dependent difference in the AUC was detected.

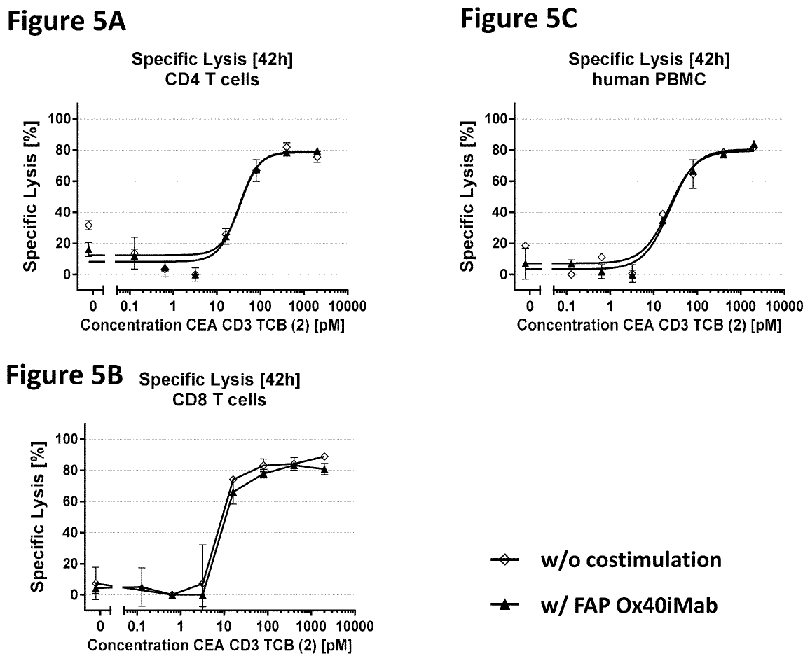

The presence of OX40 costimulation did also neither speed up tumor cell lysis nor increase the magnitude of tumor cell lysis by CEACAM5 CD3 TCB nor decrease the TCB concentration necessary to achieve lysis of a certain percentage of tumor cells (e.g. shift in EC₅₀ values). This was true for all evaluated effector cell preparations and is shown exemplary for the 42 hrs time point in FIGS. 5A-5C.

Similar findings were obtained using CEA CD3 TCB (data not shown).

3.3 the Presence of FAP Targeted OX40 Agonists does Influence the Secretion of Cytokines

In some assays, TNF-α sensor cells were cultured additionally to the above described setting. TNF-α sensor cells naturally express the TNF-α receptor and were genetically modified with GFP under the control of an NFκB sensitive promotor element. Binding of TNF-α secreted by activated T cells leads to dose dependent activation of NFκB and subsequently to expression of GFP. The GFP fluorescence can be quantified by high through put life fluorescence microscopy over time and allows thus real-time assessment of TNF-α secretion. As described in 3.1 above, CD4 T cells were cocultured for 48 hours with MKN-45 NucLight Red cells as target cells and irradiated NIH/3T3 huFAP in the presence of fixed concentration of FAP OX40 iMAB and a serial dilution row of CEACAM5 CD3 TCB, FolR1 CD3 TCB and CEA CD3 TCB, respectively.

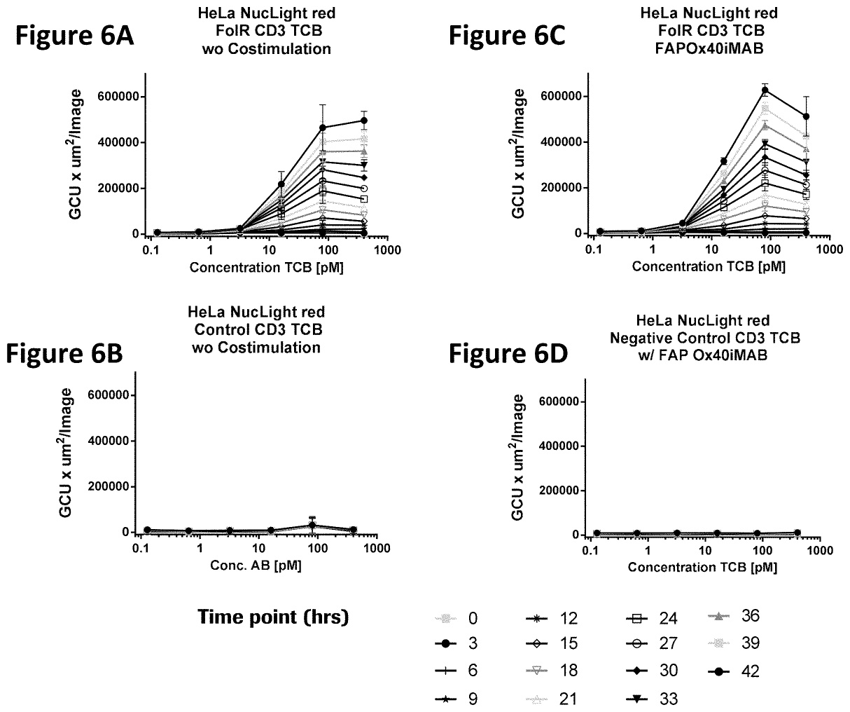

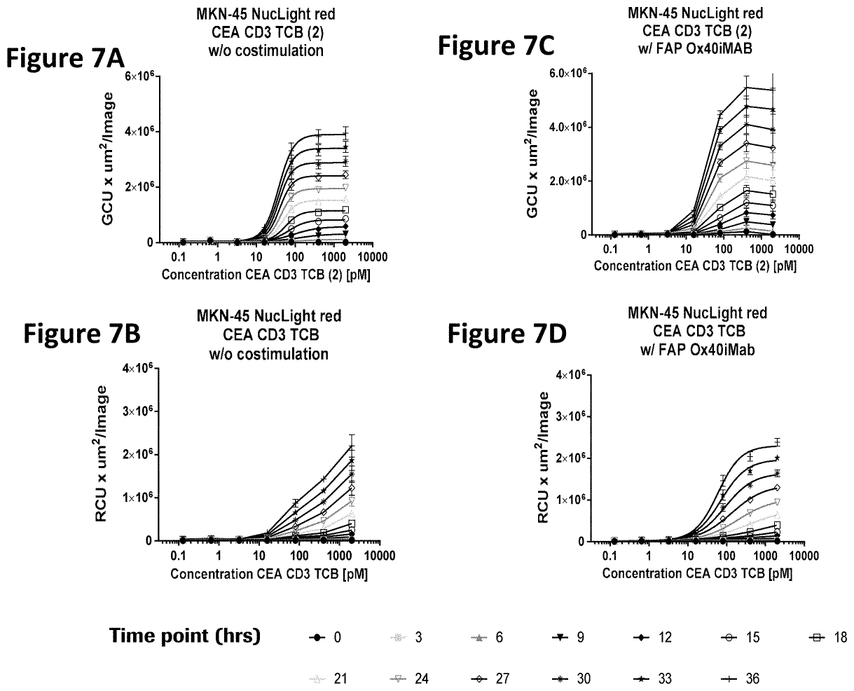

Activation of T cells by the present TCB led to dose dependent release of TNF-α, which led to a dose dependent increase of GFP fluorescence over time in the TNF-α sensor cells. Additional costimulation with FAP OX40 iMab further increased the GFP fluorescence and thus the TNF-α secretion by activated T cells (FIGS. 6A-6D and 7A-7D). This effect was mostly on the extent of TCB mediated TNF-α secretion but did not lower the TCB concentration at which TNF-α secretion was induced (shift in EC₅₀ values). Also, agonistic TCR stimulation was needed to observe this positive impact on cytokine secretion and no unspecific TNF-α secretion was detected in control TCB treated samples.

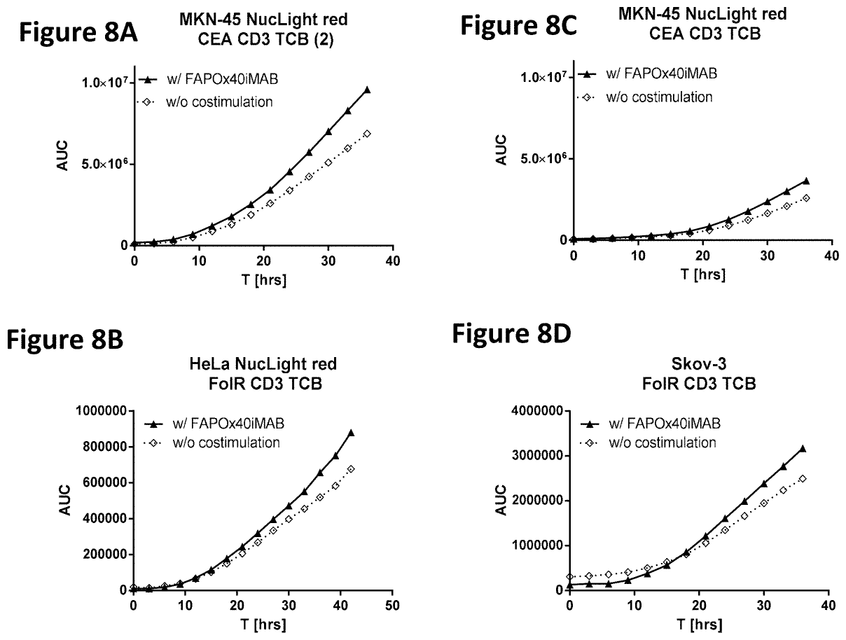

For an easier comparison over time the area under curve (AUC) was calculated for each time point with and w/o OX40 costimulation and was plotted against time (FIGS. 8A-8D). An increased AUC was observed for all tested TCBs (CEA CD3 TCB, FolR1 TCB and CEACAM5 CD3 TCB) and in the presence of different tumor cell lines (MKN45 NLR, HeLa NLR red, Skov-3).

The supernatants of all samples were evaluated at the end point (48 hours) using the cytometric bead array system (BD Bioscience) to quantify the effect on secretion of several cytokines beyond TNF-α. Evaluated cytokines were IL-2 and TNF-α as marker for general T cell activation, IFN-γ (Th1 cytokine), IL-4 (Th2 cytokine), IL-9 (Th9 cytokine) and IL-17A (Th17 cytokine) to monitor a differentiation towards a certain Th subclass, and IL-10 as immunesupressive cytokine.

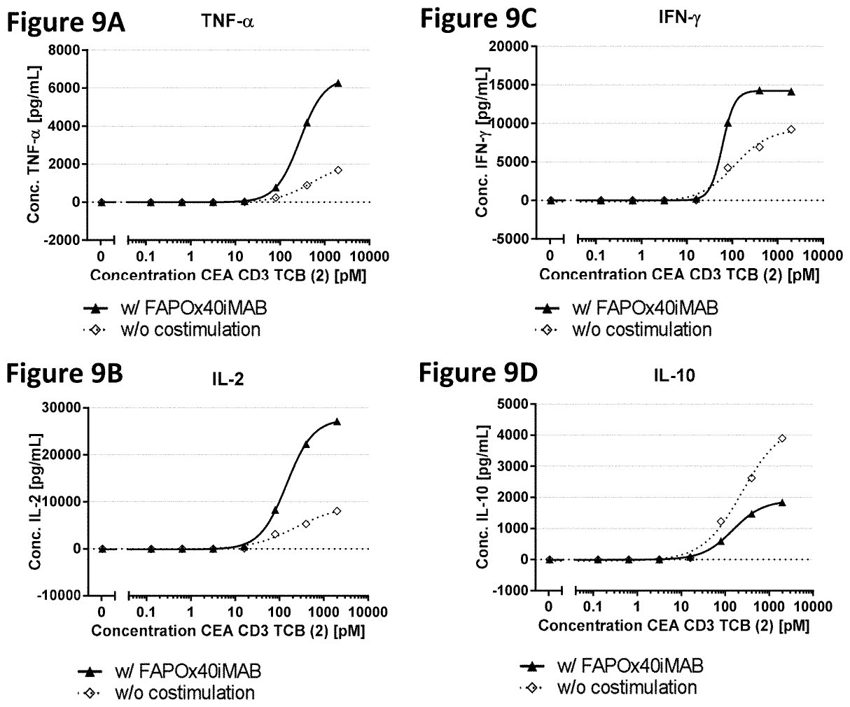

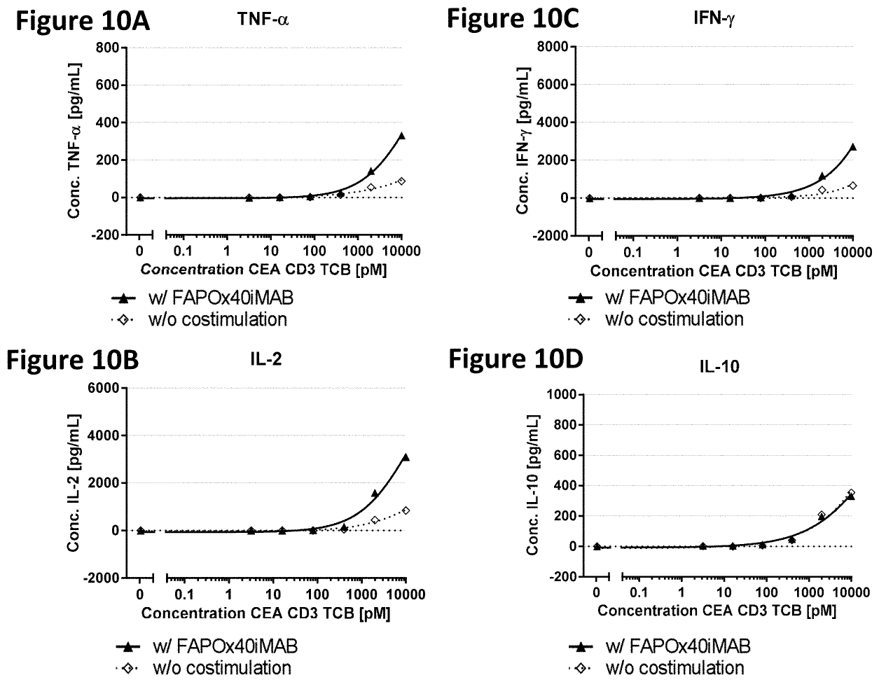

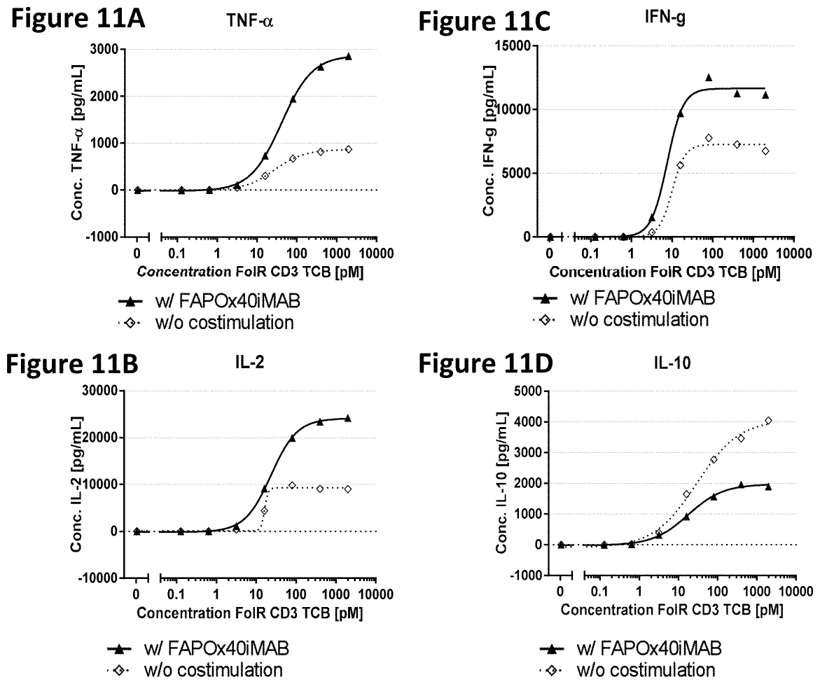

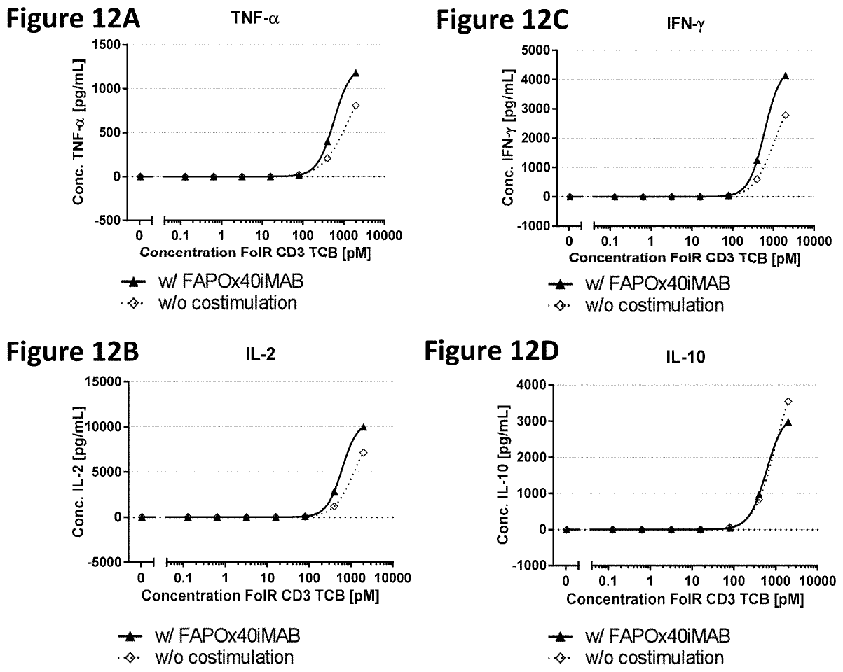

Activation of T cells by the present TCB led, next to TNF-α, to a dose dependent release of all evaluated cytokines, namely IL-2, IL-4, IFN-γ, IL-17a and IL-10 (FIGS. 9A-9D, FIGS. 10A-10D, FIGS. 11A-11D and FIGS. 12A-12D). The extent of this cytokine release differed for the TCBs, when the same target cell line was used. This can be seen from a comparison of FIGS. 9A-9D (CEACAM5 CD3 TCB) and FIGS. 10A-10D (CEA CD3 TCB). But also when the same TCB (FolR CD3 TCB) was used a difference could be observed when different target cell lines were used. FIGS. 11A-11D show the cytokine release with HeLa NLR cells whereas Skov-3 cells were used in FIGS. 12A-12D.

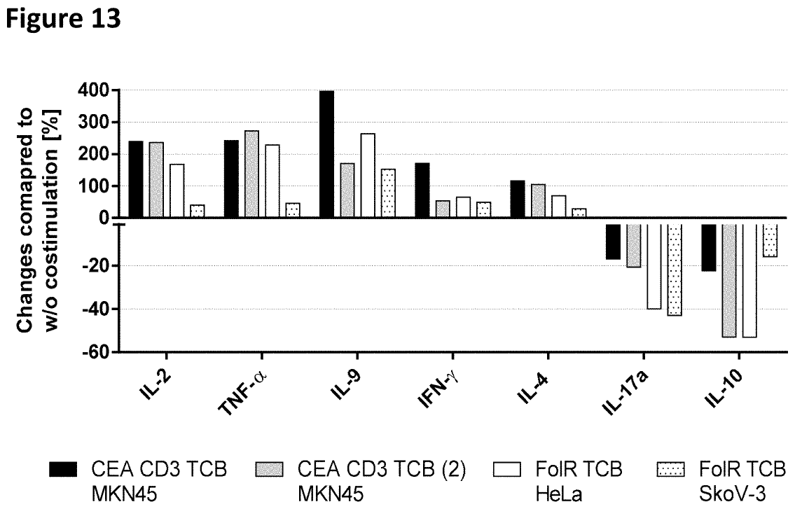

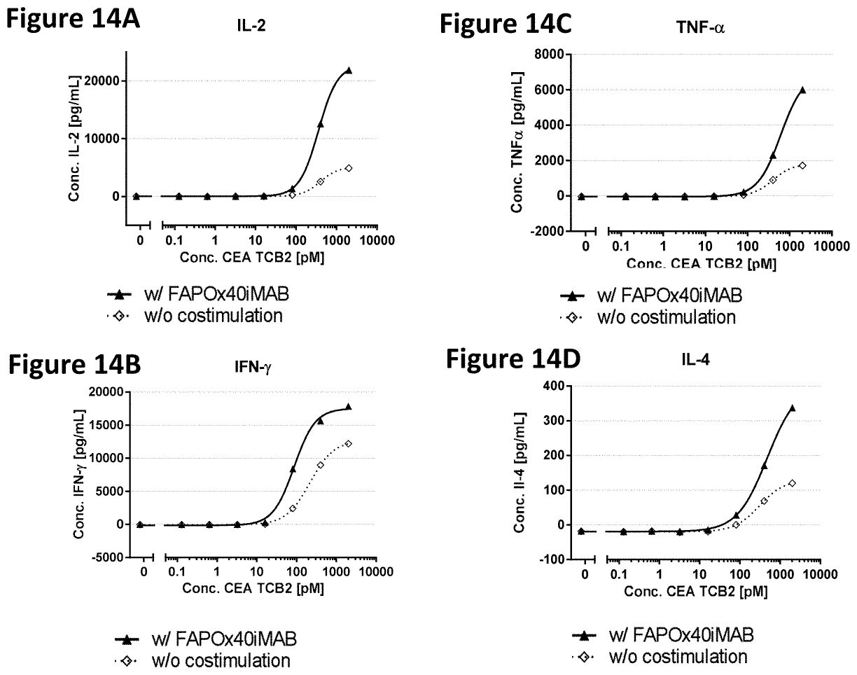

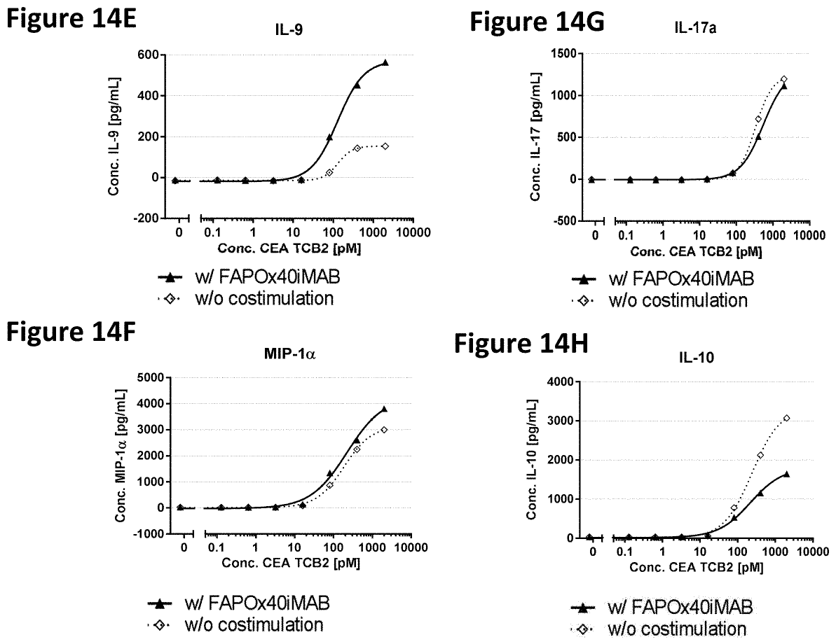

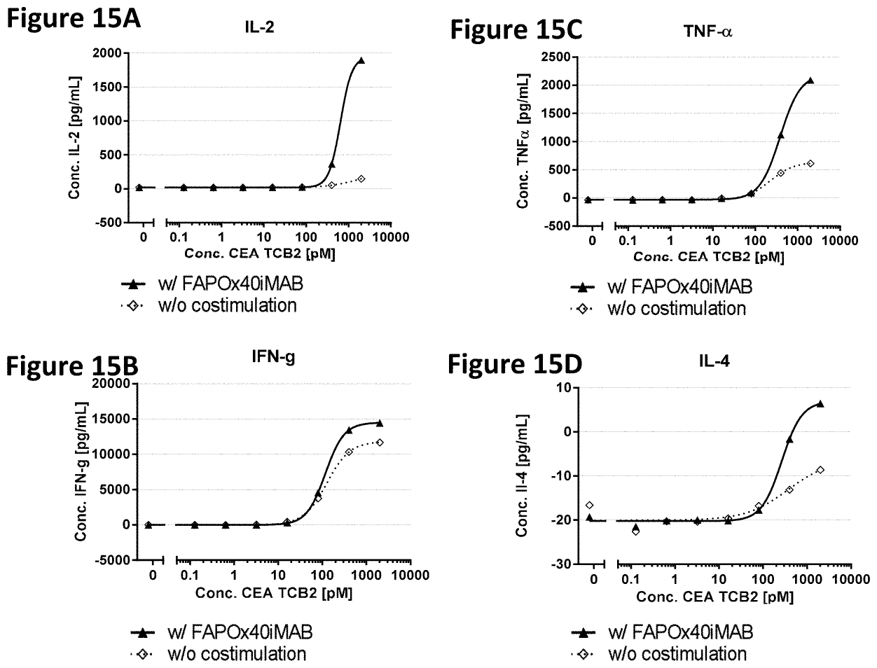

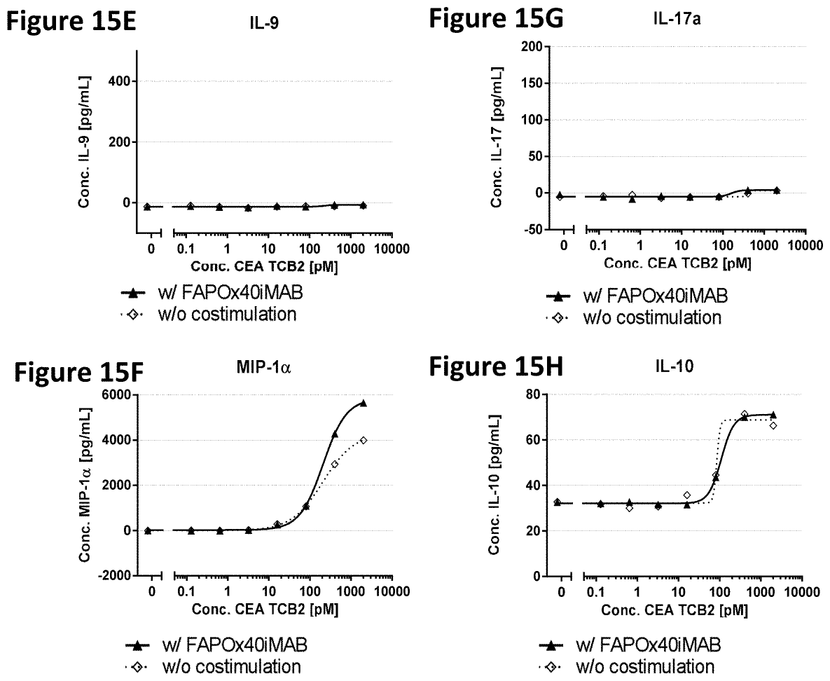

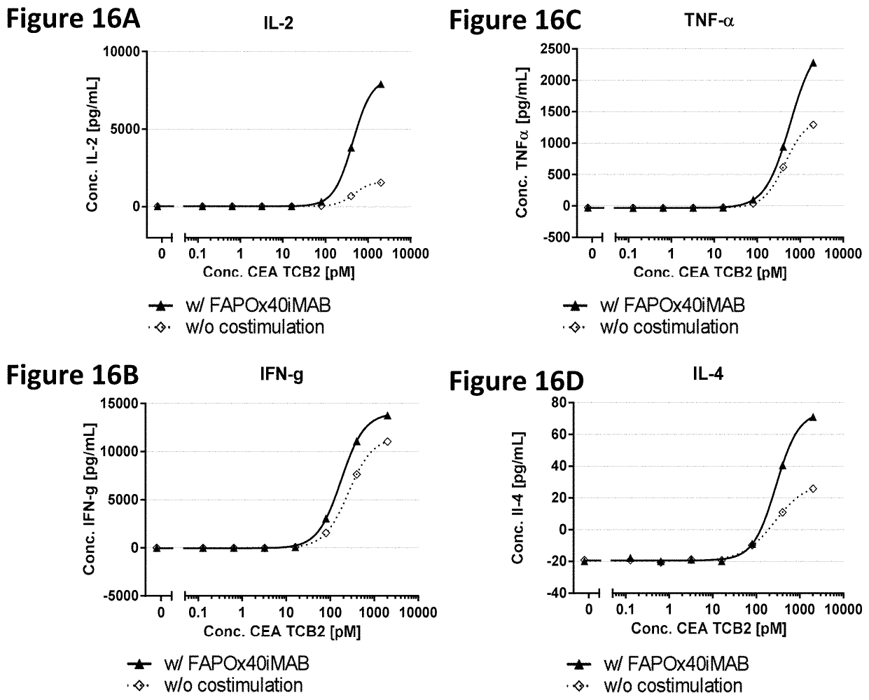

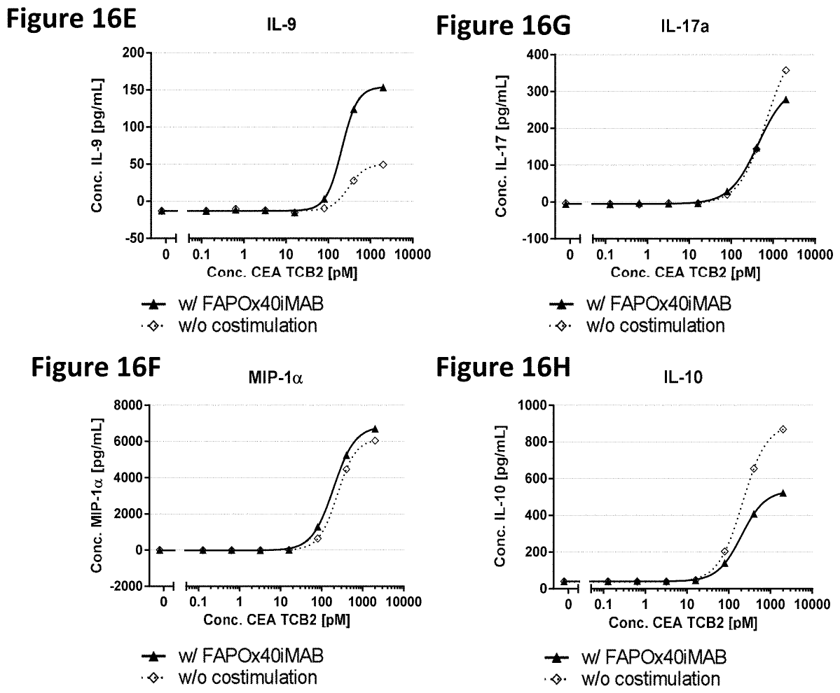

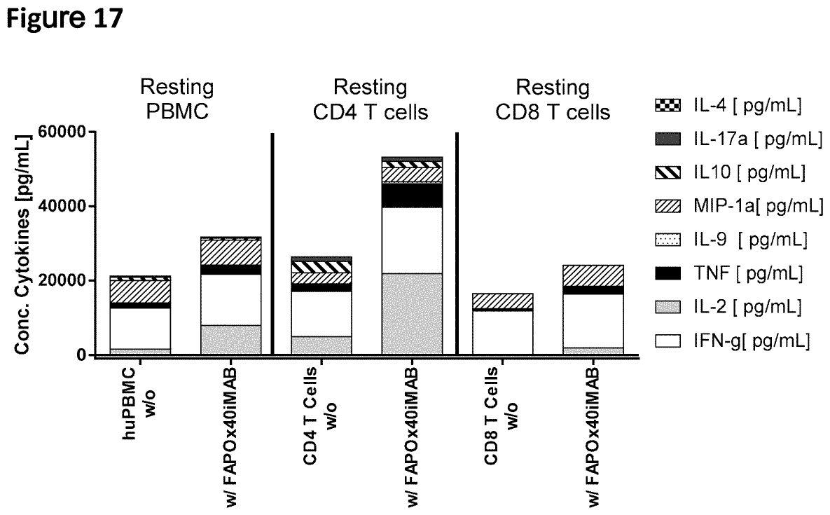

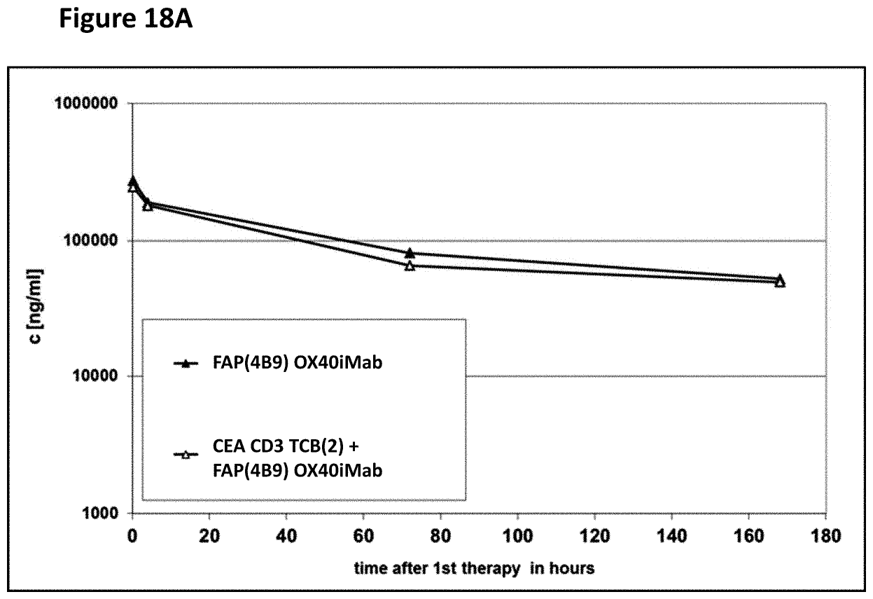

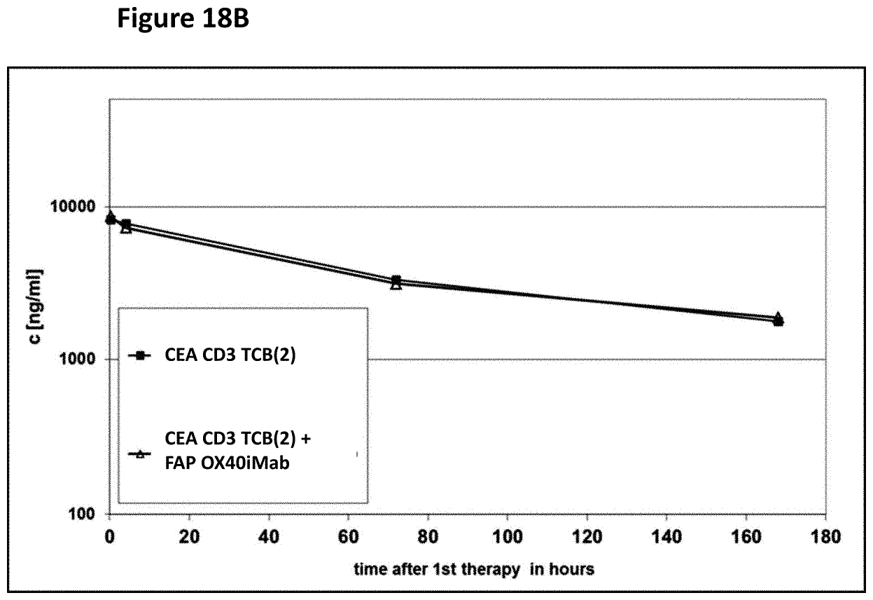

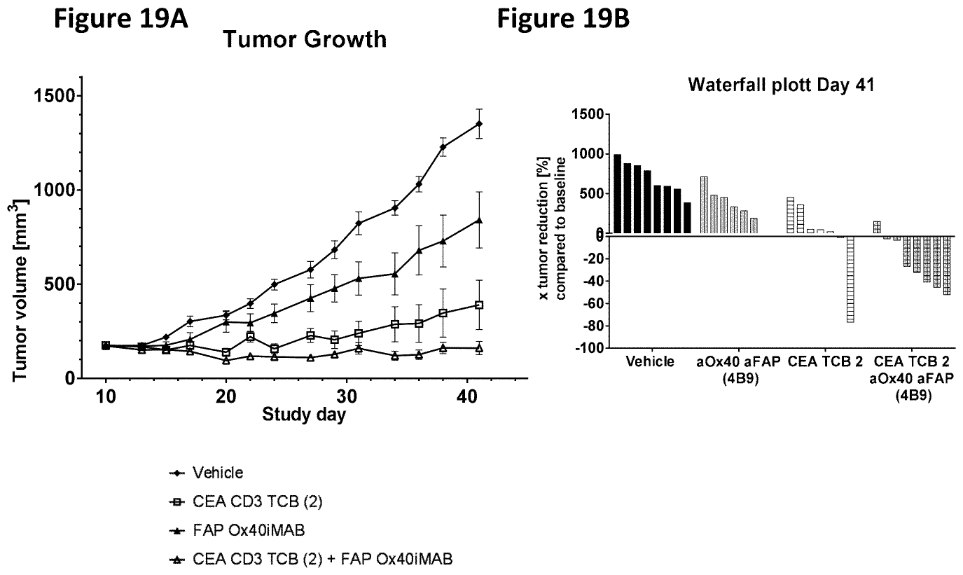

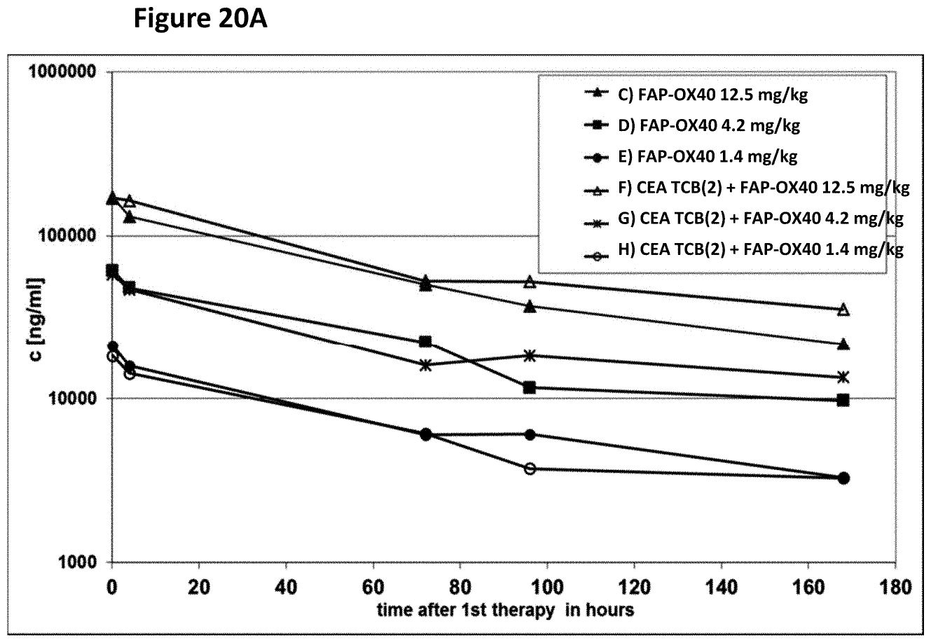

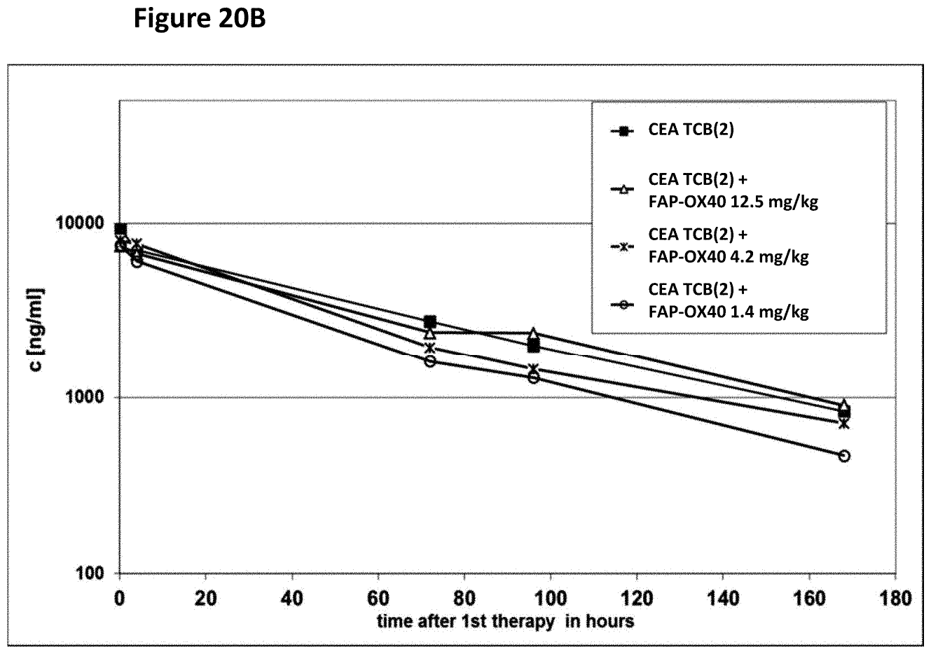

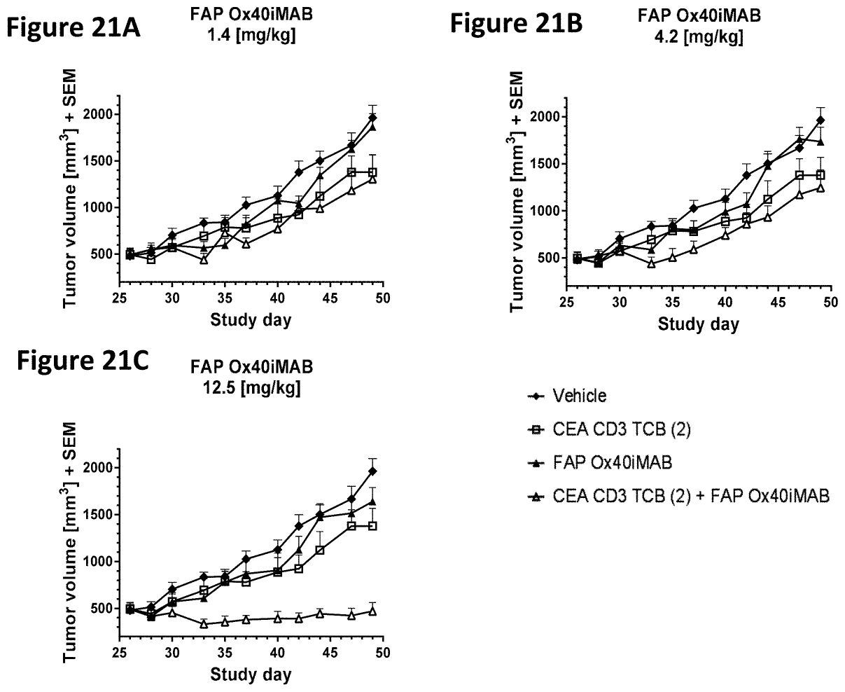

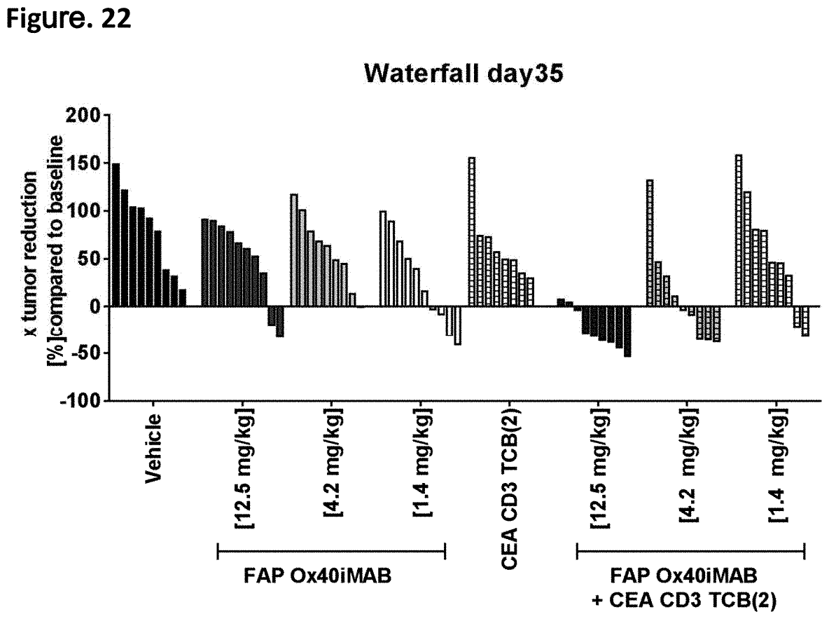

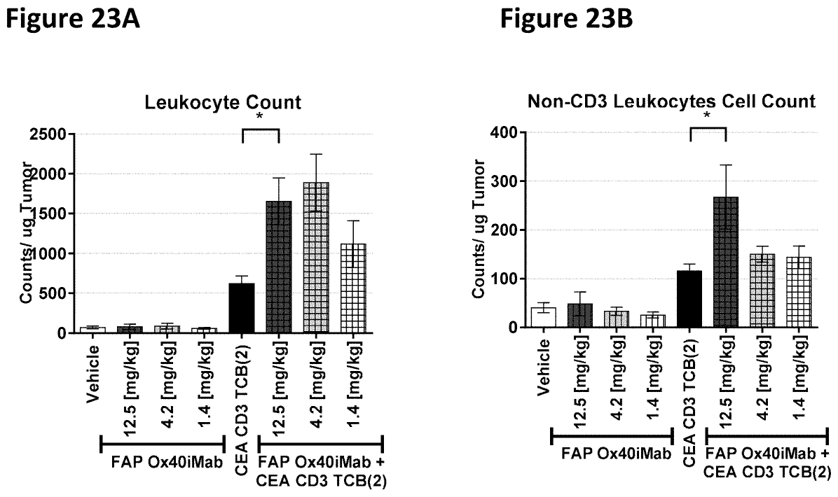

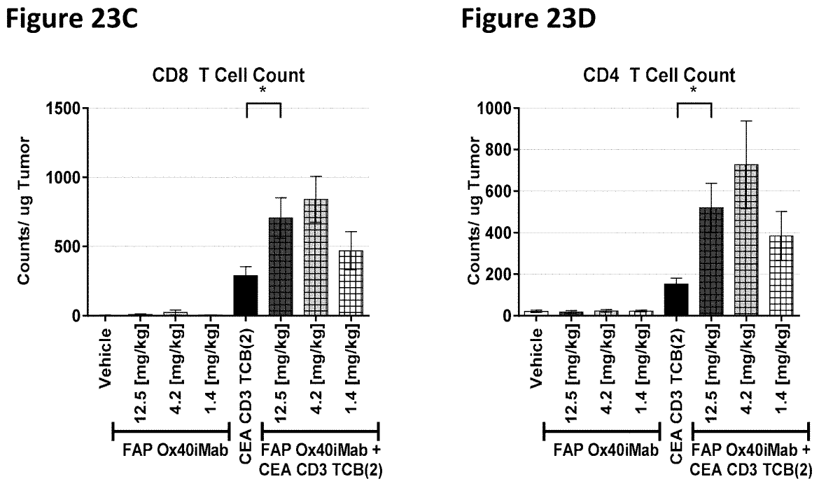

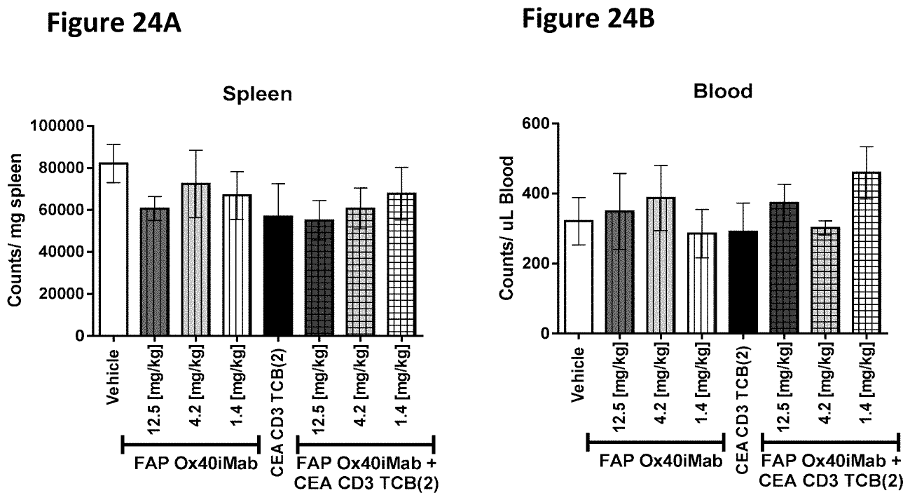

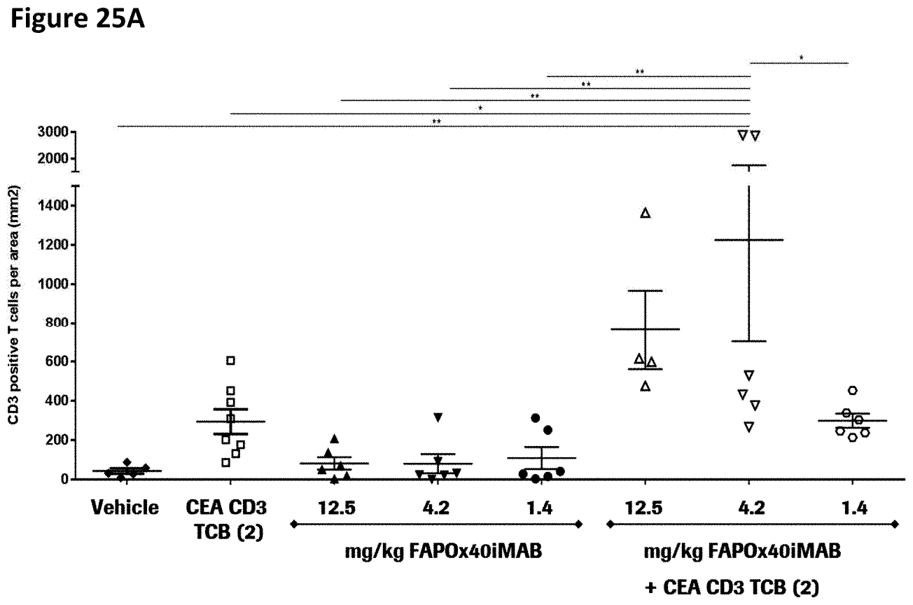

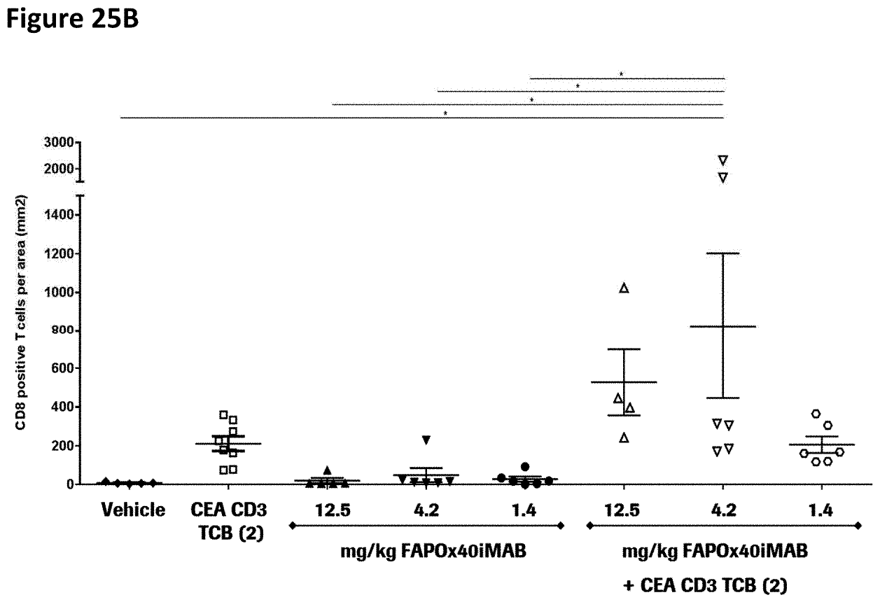

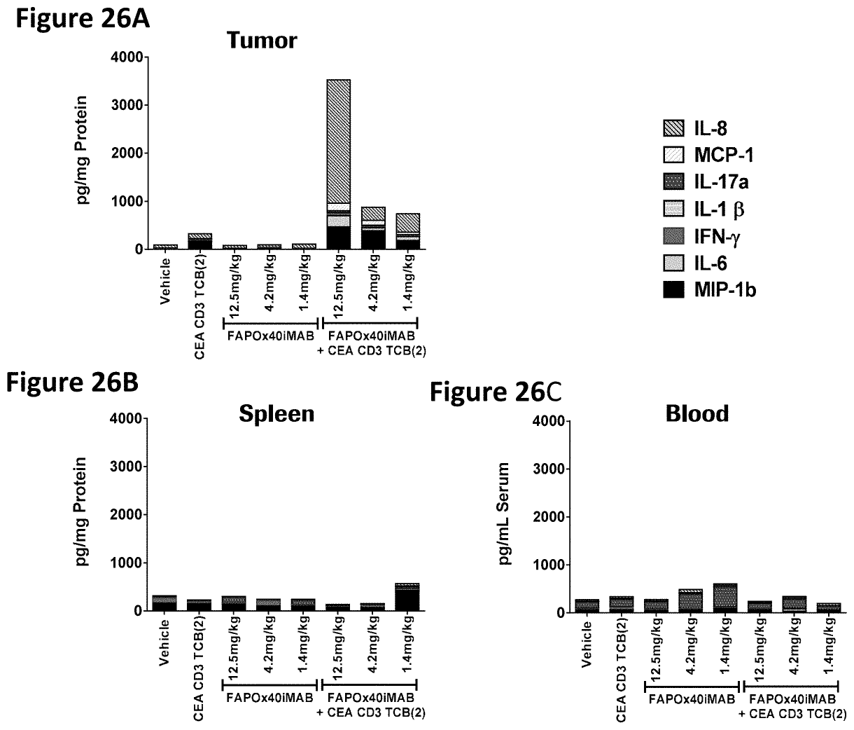

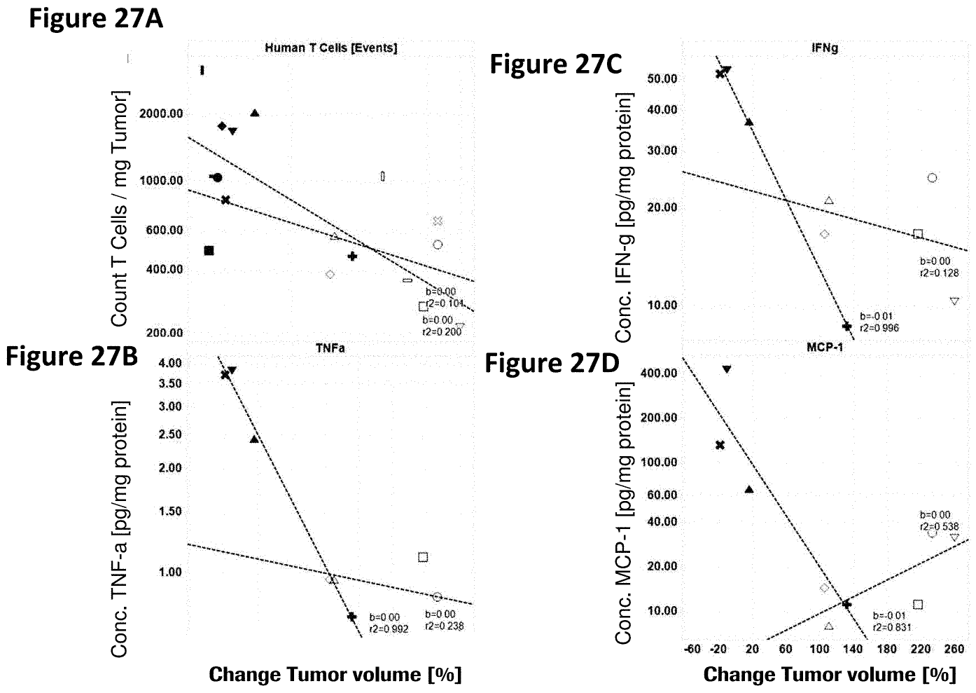

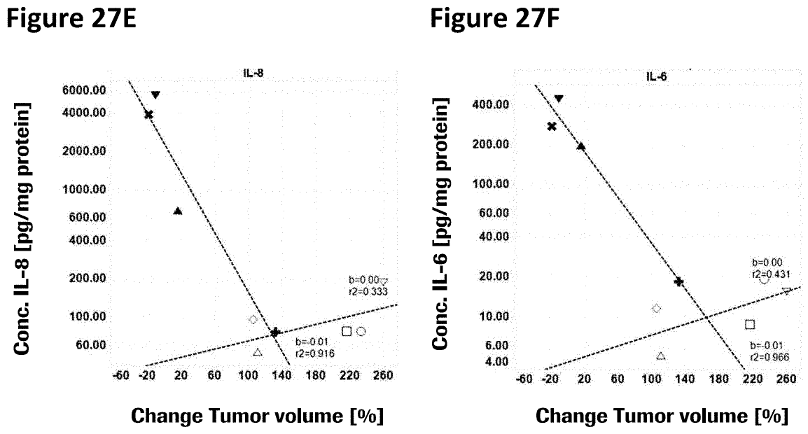

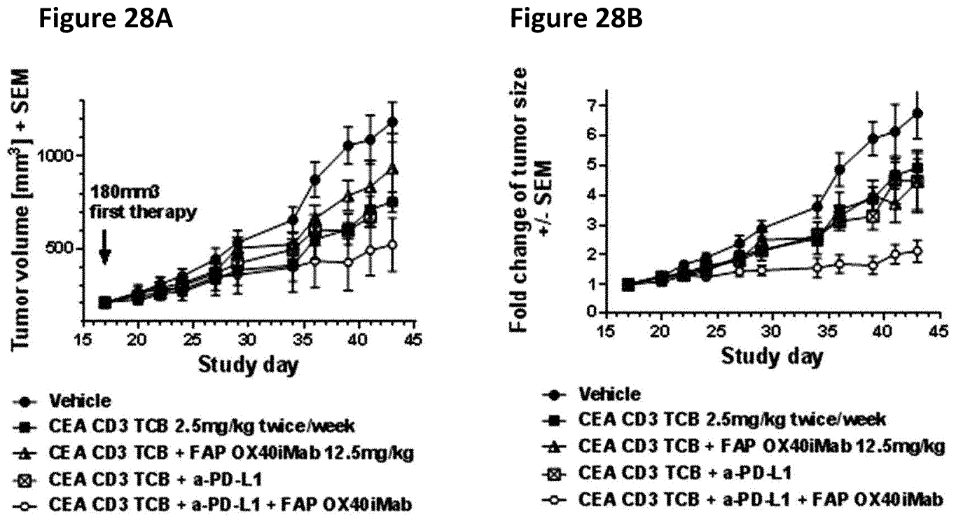

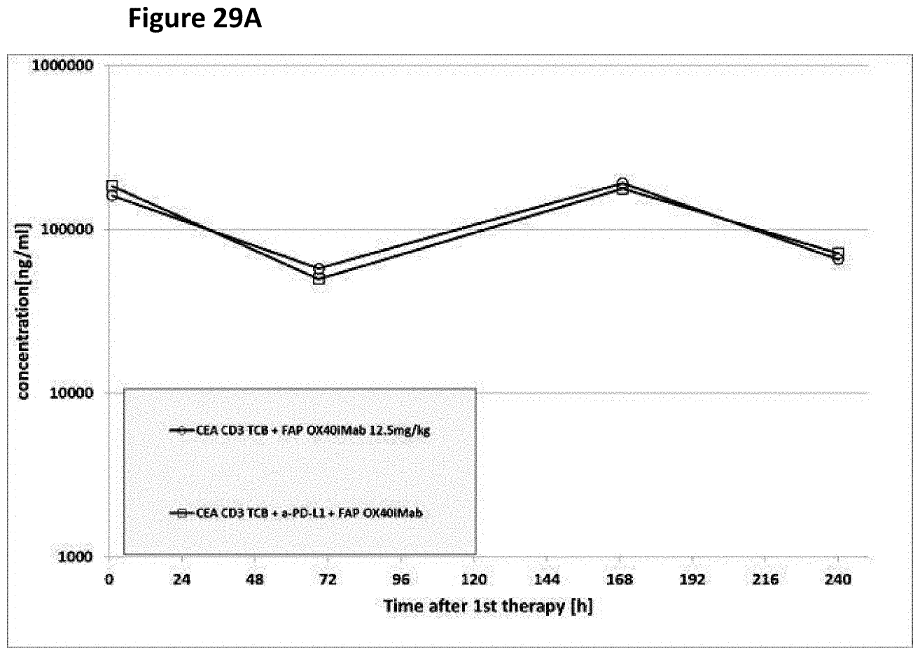

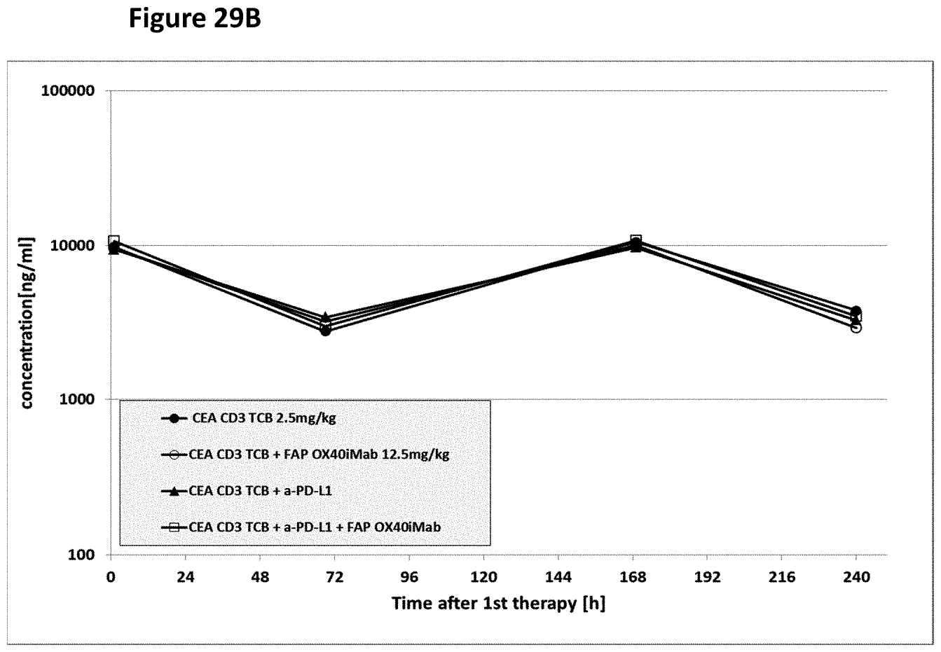

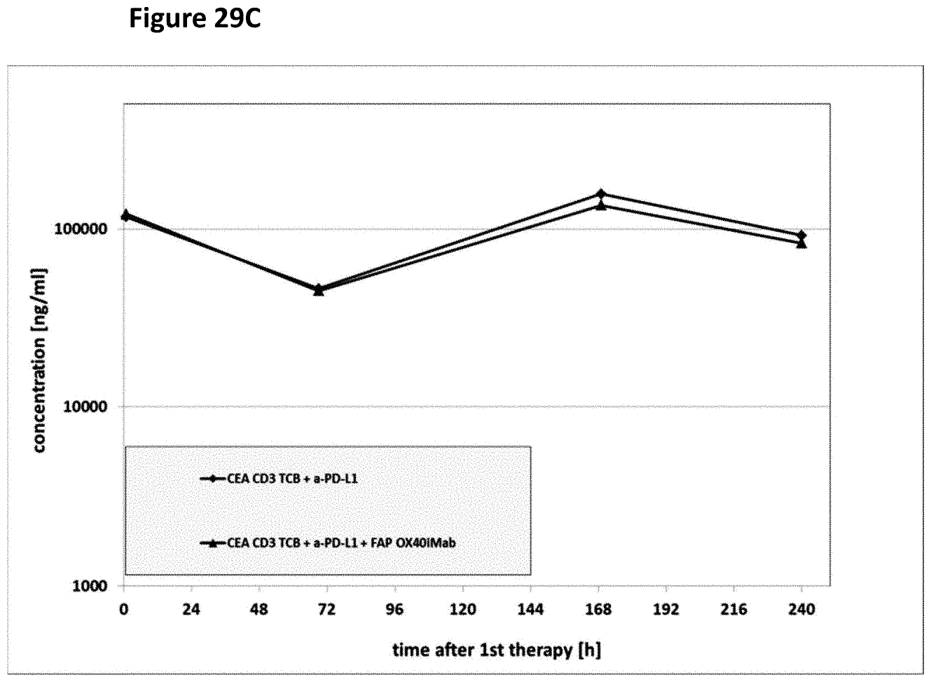

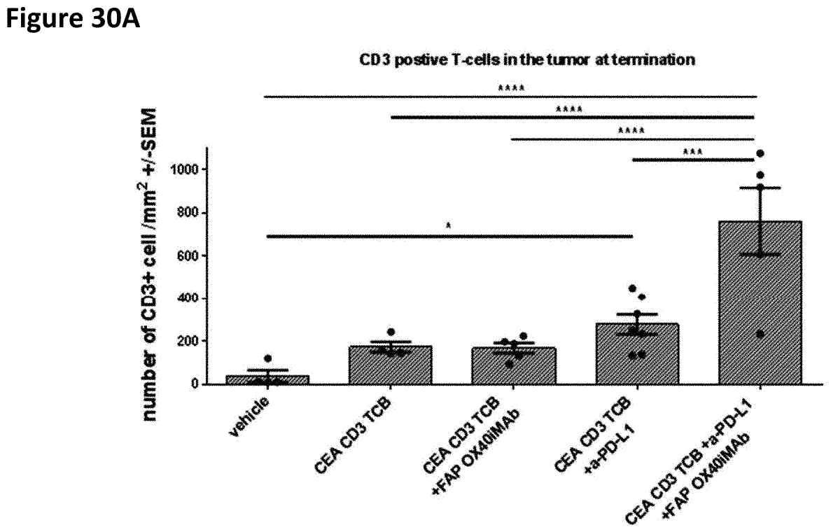

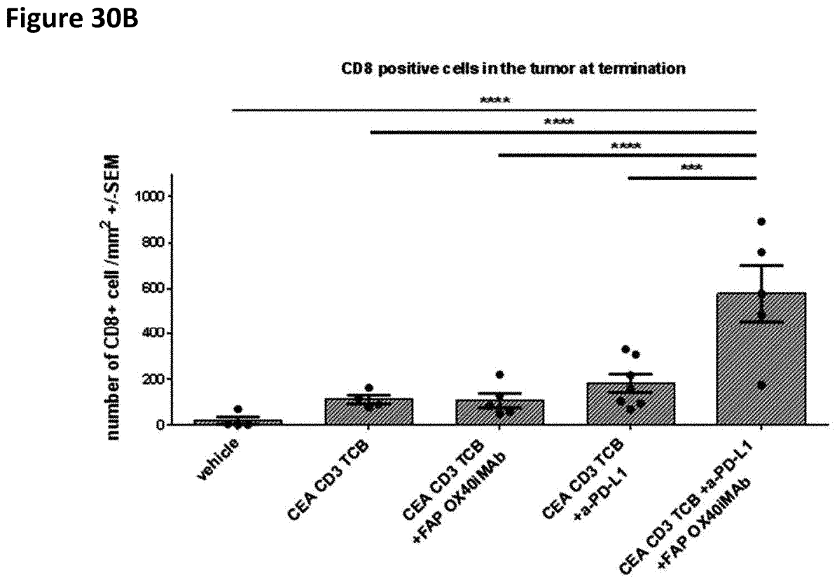

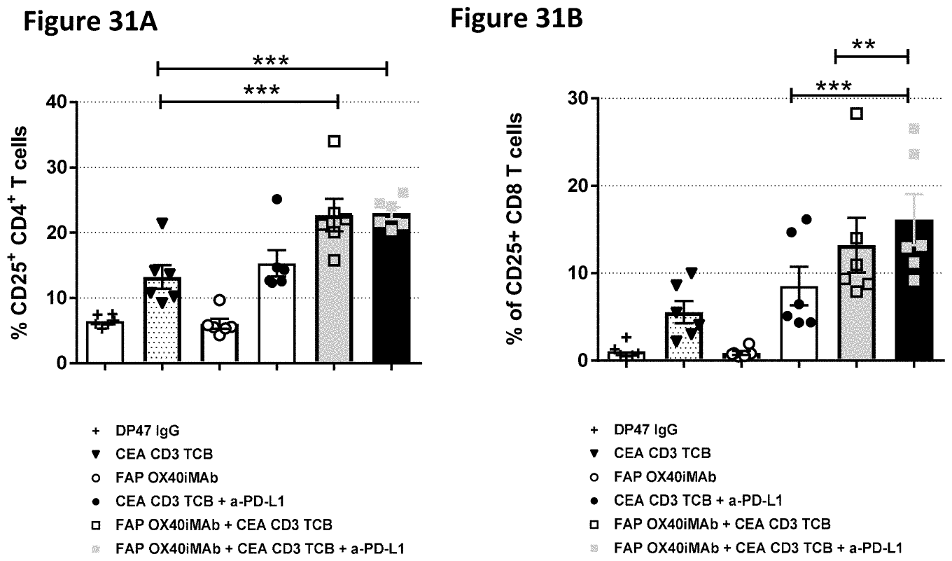

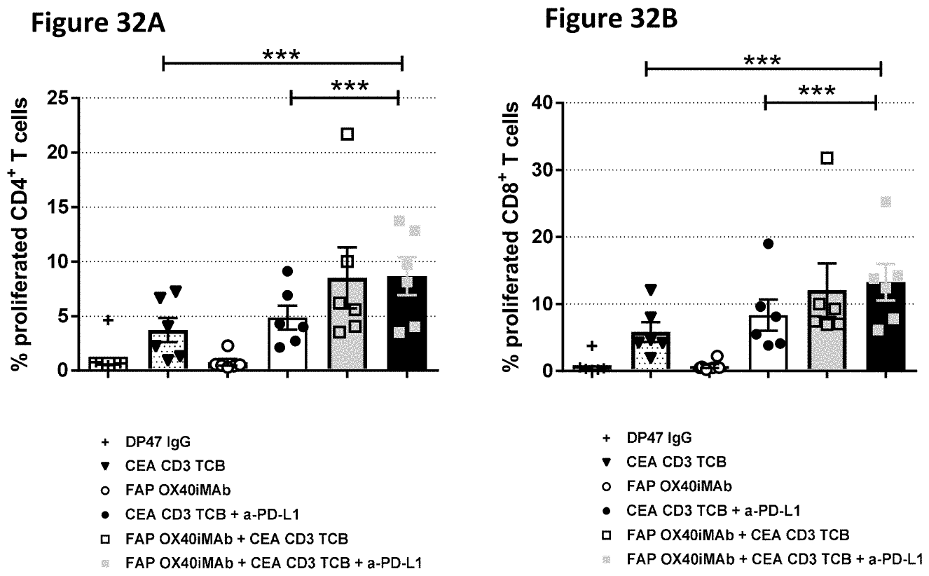

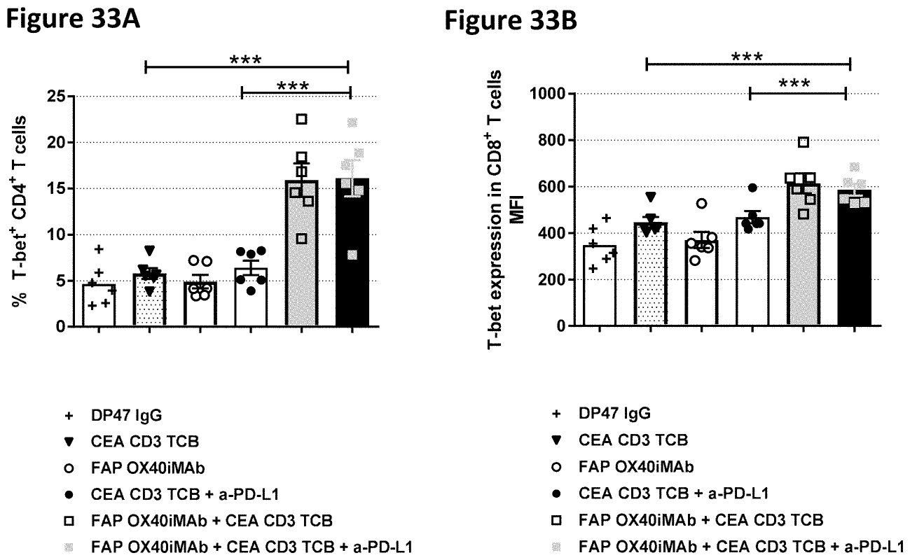

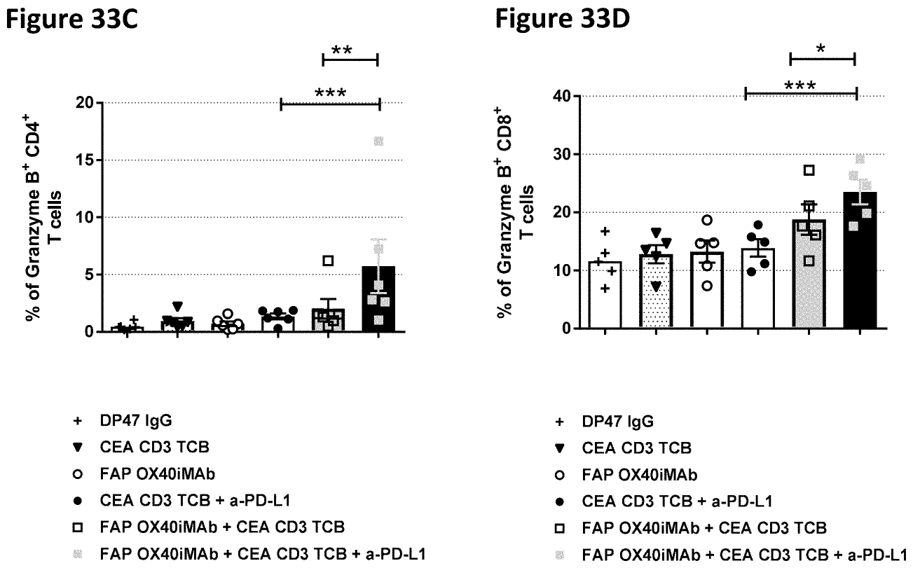

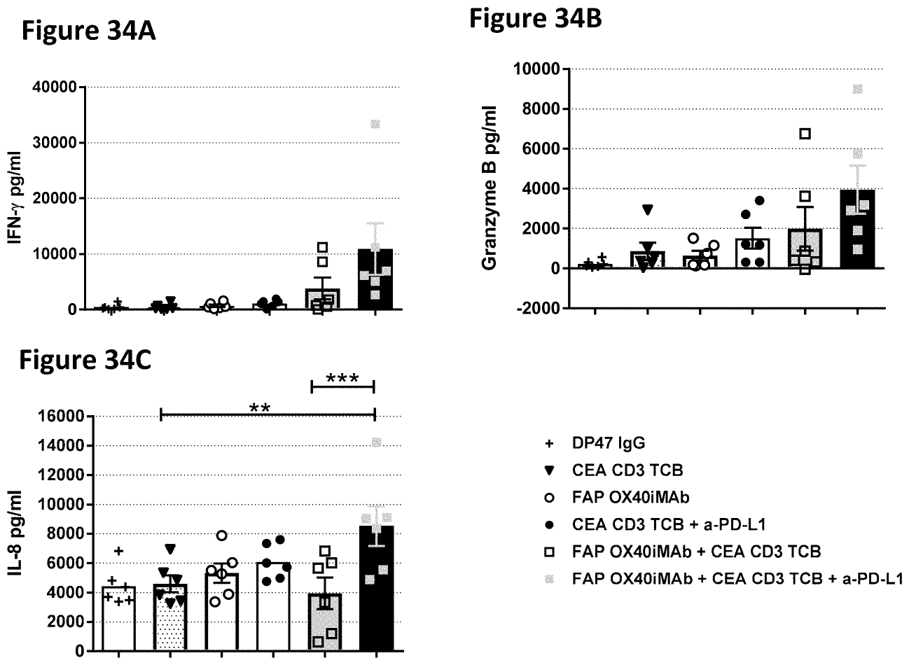

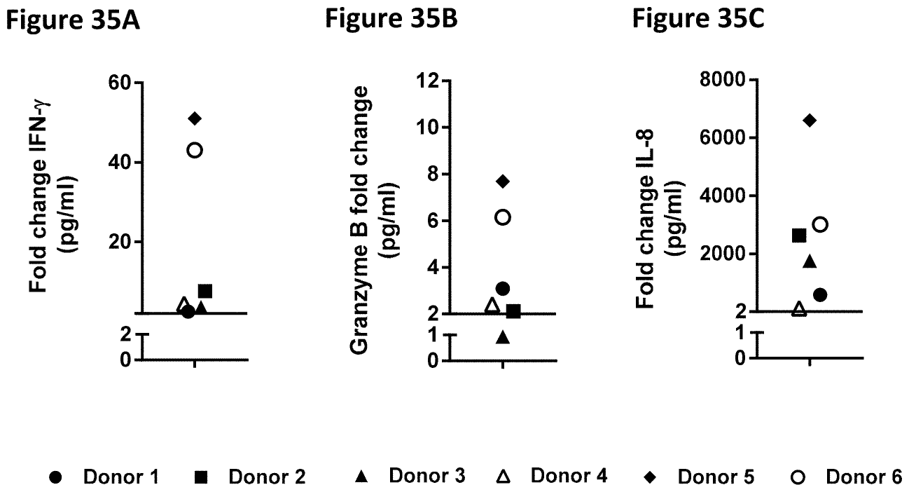

Additional co-stimulation with FAP OX40 iMab modulated the extent of dose-dependent cytokine secretion, but did not lower the TCB threshold concentration needed for cytokine secretion. Thereby, an increase of pro-inflammatory IL-2, TNF-α and IFN-γ secretion was observed, whereby the concentration of immunesuppressive IL-10 was lowered. For an easier comparison, the changes in cytokine concentration in samples with OX40 costimulation were calculated relative to those without costimulation for the TCB plateau concentration (FIG. 13).

Thereby, an increase of pro-inflammatory IL-2, TNF-α and IFN-γ secretion was evident, whereby the concentration of immunesupressive IL-10 was lowered only in some target cell/TCB combinations. There was a trend visible that a more forcefull T cell activation with strong dose-dependent cytokine secretion was also stronger modulated by OX40 costimulation. Especially, the decrease in immunesupressive IL-10 release was coupled to a strong T cell activation.

We also tested the ability of OX40 costimulation to modulate the cytokine secretion of resting CD4 and CD8 T cells and of resting human PBMC. As described in 3.1, resting human PBMC, isolated CD4 or CD8 T cells were co-cultured for 72 hrs with MKN-45 NucLight Red cells and irradiated NIH/3T3 huFAP in the presence of a serial dilution row of CEACAM5 CD3 TCB with or without a fixed concentration of FAP OX40 iMAB. The supernatant was evaluated at 72 hrs using the cytometric bead array (CBA) as described above.

OX40 costimulation supported the secretion of pro-inflammatory cytokines in resting human PBMC and to a lower extent also on CD8 T cells (dose dependency, see FIGS. 14A-14H for resting CD4 T cells, FIGS. 15A-15H for resting CD8 T cells and FIGS. 16A-16H for resting PBMCs). A comparison for top TCB concentration is shown in FIG. 17. Remarkable was especially the impact on IL-2 and TNF-α production by resting CD8 T cells.

Thus, costimulation via OX40 does not increase directly the cytolytic potential of T cells in a 48-72 hour in vitro cytotoxicity assay, but it increased the ability to secrete cytokines and modulated the cytokine microenvironment. A more proinflammatory cytokine mileau in the tumor can shift the tumor microenvironment towards a more immune-activating and less immune-supressive state, e.g. lower level of IL-10 and increased concentrations of IFN-γ can allow myeloid cells in the tumor to mature to Th1 and cytotoxic T cell supporting antigen presenting cells. A shift to a supportive cytokine network will restore a successfully and sustained tumor cell elimination where before the tumor achieved to escape immune control. In line with the preferential expression of OX40 on CD4 T cells, a stronger modulation was observed for cytokine secretion on CD4 T cells vs that of CD8 T cells. However, both cell types were influenced.

### Example 4

### Combination Therapy of FAP OX40 iMab and CEACAM5 TCB In Vivo

### 4.1 Methods

In the following examples we tested if the combination of TCBs and FAP Ox40 iMAb leads to a superior anti-tumor efficacy in vivo compared to the respective monotherapies.

Human monovalent FAP targeted, tetravalent OX40 bispecific antibodies (FAP OX40 iMab) were tested as single agent and in combination with the human CEACAM5 CD3 TCB (CEA CD3 TCB (2)) against vehicle and CEACAM5 CD3 TCB only treated animals treated with CEACAM5 CD3 TCB only. Human gastric MKN45 cancer cells were cografted sub cutaneously with a mouse fibroblast cell line (3T3) in NOG humaniced mice.

### 4.2 Cell Lines and Tumor Model

Human MKN45 cells (human gastric carcinoma) were originally obtained from ATCC and after expansion deposited in the Glycart internal cell bank. Cells were cultured in DMEM containing 10% FCS at 37° C. in a water-saturated atmosphere at 5% CO₂. In vitro passage 7 was used for subcutaneous injection at a viability of 98%. Human fibroblasts NIH-3T3 were originally obtained from ATCC, engineered at Roche Nutley to express human FAP and cultured in DMEM containing 10% Calf serum, 1× Sodium Pyruvate and 1.5 μg/ml Puromycin. Clone 39 was used at an in vitro passage number 9 (Experiment 1, Table 1) and 7 (Experiment 2, Table 2), respectively, at a viability of 98.8% and 98.4%, respectively.

50 microliters cell suspension (1×10⁶ MKN45 cells+1×10⁶ 3 T3-huFAP) mixed with 50 microliters Matrigel were injected subcutaneously in the flank of anaesthetized mice with a 22G to 30G needle.

### 4.3 Mouse Model

NOG female mice were delivered by Taconic and in house transferred with human stem cells. Mice were maintained under specific-pathogen-free condition with daily cycles of 12 h light/12 h darkness according to committed guidelines (GV-Solas; Felasa; TierschG). Experimental study protocol was reviewed and approved by local government (P ZH193/2014). After arrival animals were maintained for one week to get accustomed to new environment and for observation. Continuous health monitoring was carried out on regular basis.

### 4.4 Treatment and Experimental Handling of Experiment 1

The human monovalent FAP-targeted OX40 bispecific antibody with tetravalent binding to OX40 (FAP OX40 iMab, Molecule A as described in Example 1) was tested as single agent and in combination with the human CEACAM5 CD3 TCB. The FAP binder used in the FAP OX40 iMab construct was 4B9. Human gastric MKN45 cancer cells were cografted subcutaneously with a mouse fibroblast cell line (3T3) in NOG humanized mice.

7 days before cell injection mice were bled and screened for the amount of human T-cells in the blood. Mice were injected subcutaneously on study day 0 with 1×10⁶ MKN45 cells mixed with 1×10⁶ 3 T3 fibroblasts. Tumors were measured 2 to 3 times per week during the whole experiment by Caliper. On day 10 mice were randomized for tumor size and human T-cell count with an average T-cell count/μl blood of 140 and an average tumor size of 170 mm³. On the day of randomization mice were injected i.v. with Vehicle, CEACAM5 CD3 TCB, FAP(4B9) OX40 iMab or the combination of the FAP(4B9) OX40 iMab with CEACAM5 CD3 TCB for 5 weeks.

All mice were injected i.v. with 200 μl of the appropriate solution. The mice in the vehicle group were injected with Histidine Buffer and the treatment groups with OX40 agonizing construct, the CEACAM5 CD3 TCB or the combination. To obtain the proper amount of compound per 200 the stock solutions were diluted with Histidine Buffer when necessary. The dose and schedule used for CEACAM5 TCB was 0.5 mg/kg, once/week whereas the FAP OX40iMab was given at a dose of 12.5 mg/kg, once/week.

2 mice/group were bled 10 min, 4 h, 72 h and 168 h after the first therapy to determine the exposure of compounds during the first week. FAP OX40 iMab was measured by sandwich ELISA, binding of the construct to human OX40 and detection of huCH1-domain. CEACAM5 CD3 TCB was detected by sandwich ELISA, binding of the TCB to an anti CD3-CDR specific antibody and detection of human Fc (see FIGS. 18A and 18B).

The experiment was terminated at study day 44. Tumors, blood and spleen were harvested in PBS, single cell suspensions were generated and stained for different immune cell markers and analysed by FACS. Erythrolysis of whole blood samples were performed for 3 minutes at room temperature using the BD Pharm Lyse buffer (BD, Ca. No. 555899) according to manufacturers instructions. Splenocytes were isolated by homogenization of the spleen through a cell strainers (nylon filter 70 um, BD Falcon) followed by erythrolysis as described above. Tumor single cell suspensions were prepared by using the gentleMACS Dissociator (Miltenyi) and digest the homogenate for 30 minutes at 37° C. with DNAse I ([0.025 mG/mL], RocheDiagnostics, Ca. No. 11284932001) and Collagenase D ([1 mG/mL], RocheDiagnostics, Ca. No. 11088882001). Afterwards cell suspensions were filtered through cell strainers (nylon filter 70 um, BD Falcon) to remove debris. All preparations were washed with excess ice cold FACS buffer. Cells were surface-stained with fluorescent dye-conjugated antibodies anti-mouse CD4 (clone GK 1.5, BioLegend, Cat.-No. 100422), CD8 (clone 53-6.7, BioLegend, Cat.-No. 100730), CD45 (clone 30-F11, BioLegend, Cat.-No. 103116), and CD3 (clone 145-2C11, BioLegend, Cat.-100351) in the presence of purified Rat anti-mouse CD16/CD32 (clone 2.4G₂, BD, Ca. No. 553142) for 30 min at 4° C., dark, in FACS buffer. Samples were resuspendend in FACS buffer containing 0.2 μg/mL DAPI (Santa Cruz Biotec, Cat. No. Sc-3598) before they were acquired the same day using 5-laser LSR-Fortessa (BD Bioscience with DIVA software). Living CD4 and CD8 T cells were gated (DAPI−, CD45+, CD3+, CD4 or CD8+), normalized counts (per uL blood, mg spleen or mg tumor) calculated and values plotted for the respective treatment groups.

TABLE 1
Compositions used in the in vivo experiment

| | Dose | | Concentration |
|---|---|---|---|
| Compound | (mg/kg) | Formulation buffer | (mg/mL) |
| FAP(4B9) OX40 iMab | 12.5 | 20 mM Histidine, 140 | 4.41 |
| (Molecule A of | | mM NaCl, pH 6.0, | (=stock |
| Example 1) | | 0.01% Tween-20 | concentration |
| CEACAM5 CD3 TCB | 0.5 | 20 mM Histidine, 140 | 1.72 |
| (Example 2) | | mM NaCl, pH 6.0, | (=stock |
| | | 0.01% Tween20 | concentration |

### 4.5 Treatment and Experimental Handling of Experiment 2

The human monovalent anti-FAP(4B9)/anti-OX40 bispecific antibody (FAP OX40 iMab) was tested in 3 different doses as single agent and in combination with the human CEACAM5 CD3 TCB. Human gastric MKN45 cancer cells were cografted subcutaneously with a mouse fibroblast cell line (3T3) in NOG humanized mice with human stem cells as described above.

7 days before cell injection mice were bled and screened for the amount of human T-cells in the blood. Mice were injected subcutaneously on study day 0 with 1×10⁶ MKN45 cells mixed with 1×10⁶ 3 T3 fibroblasts. Tumors were measured 2 to 3 times per week during the whole experiment by Caliper. On day 26, mice were randomized for tumor size and human T-cell count with an average T-cell count/μl blood of 115 and an average tumor size of 490 mm³. One day after randomization mice were injected i.v. with Vehicle, CEACAM5 CD3 TCB, FAP OX40 iMab or the combinations of FAP OX40 iMab with CEACAM5 CD3 TCB for 4 weeks.

All mice were injected i.v. with 200 μl of the appropriate solution. The mice in the vehicle group were injected with Histidine Buffer and the treatment groups with the OX40 agonizing constructs, the CEACAM5 CD3 TCB or the combination. To obtain the proper amount of compound per 200 the stock solutions were diluted with Histidine Buffer when necessary. The dose and schedule used for CEACAM5 CD3 TCB was 0.5 mg/kg, once/week whereas FAP OX40 iMab was given at a dose of 12.5 mg/kg, 4.2 mg/kg or 1.4 mg/kg, once/week.

The experiment was terminated at study day 50. Tumors, blood and spleen were harvested in PBS, single cell suspensions were generated and stained for different immune cell markers and analysed by FACS.

Spleen and tumor from all remaining mice per group were analysed by flow cytometry at termination. Single cell suspensions were stained for CD45, CD3, CD4 and CD8 and the amount of cells was analysed. Parts of tumors at termination and from animals during the experiment were formalin fixed and afterwards embedded in Paraffin. Samples were cut and stained for CD3 and CD8. Plasma as well as part of spleen and tumor was frozen for Cytokine analysis via Multiplex. Parts of tumors at termination were formalin fixed and afterwards embedded in Paraffin. Samples were cut and stained for CD3 and CD8.

TABLE 2
Compositions used in the in vivo experiment

| | Dose | | Concentration |
|---|---|---|---|
| Compound | (mg/kg) | Formulation buffer | (mg/mL) |
| FAP(4B9) OX40 iMab | 12.5 or | 20 mM Histidine, 140 | 3.2 |
| (Molecule A of | 4.2 or | mM NaCl, pH 6.0, | (=stock |
| Example 1) | 1.4 | 0.01% Tween-20 | concentration |
| CEACAM5 CD3 TCB | 0.5 | 20 mM Histidine, 140 | 3.1 |
| (Example 2) | | mM NaCl, pH 6.0, | (=stock |
| | | 0.01% Tween20 | concentration |

In order to determine the pharmacokinetic profiles of the injected compounds during the first week, 2 mice per Group were bled 10 min, 4 h, 72 h and 7d after the first therapy and injected compounds were analysed by ELISA. OX40 iMAbs were detected via OX40 binding (A) whereas CEACAM5 CD3 TCB was detected via binding to an anti-CD3 CDR antibody (B).

(A) Biotinylated human OX40, test sample, Digoxigenin labelled anti-huCH1 antibody and anti-Digoxigenin detection antibody (POD) were added stepwise to a 96-well streptavidin-coated microtiter plate and incubated after every step for 1 h at room temperature. The plate was washed three times after each step to remove unbound substances. Finally, the peroxidase-bound complex was visualized by adding ABTS substrate solution to form a colored reaction product. The reaction product intensity was photometrically determined at 405 nm (with reference wavelength at 490 nm) and is proportional to the analyte concentration in the serum sample.

(B) Biotinylated anti-huCD3—CDR antibody, test sample, Digoxigenin labelled anti-huFc antibody and anti-Digoxigenin detection antibody (POD) were added stepwise to a 96-well streptavidin-coated microtiter plate and incubated after every step for 1 h at room temperature. The plate was washed three times after each step to remove unbound substances. Finally, the peroxidase-bound complex was visualized by adding ABTS substrate solution to form a colored reaction product. The reaction product intensity was photometrically determined at 405 nm (with reference wavelength at 490 nm) and is proportional to the analyte concentration in the serum sample.

### 4.6 Cytokine Analysis of Tumor, Spleen and Serum Samples

Serum was collected, and subcutaneous tumors and spleen were harvested from animals at termination (day 50), 2 days after last Ab administration. 20-30 mg of snap-frozen spleen and tumor tissues were processed for whole protein isolation at study termination. Briefly, tissue samples were meshed by using the Tissue Lyser system and stainless steel beads in a total volume of 150 μl of lysis buffer. Meshed samples were cleared by centrifugation and whole protein content was analysis by BCA protein assay kit (Fischer Thermo Scientific) in the supernatant according to manufacturer's instructions. At total of 200 μg of whole protein of tumor and spleen lysates as well as a 1:10 dilution of serum samples was used for the analysis of different cytokines/chemokines by the Bio-Plex system following instructions of manufacturer (Bio-Plex Pro™ Human Cytokine 17-plex Assay, BioRad).

### 4.7 Immunhistochemistry

Immunohistochemical analysis was performed of human MKN45 gastric subcutaneous tumors cografted with 3T3 murine fibroblasts derived from the indicated treatment groups in humanized NOG mice. Subcutaneous tumors were harvested from animals at termination day, 2 days after last Ab administration, were fixed in formalin 10% (Sigma, Germany) and later processed for FFPET (Leica 1020, Germany). 4 μm paraffin sections were subsequently cut in a microtome (Leica RM2235, Germany). HuCD8 and HuCD3 immunohistochemistry was performed using anti-human CD8 (Cell Marque Corporation, California) and anti-human CD3 (ThermoFischer Scientific, USA) in the Leica autostainer (Leica ST5010, Germany) following the manufacture's protocols. Quantification of huCD3 and huCD8 positive T cells was performed with Definiens software (Definiens, Germany). Statistics were analyzed by one way ANOVA with multiple comparison tests.

### 4.8 Results of Experiment 1

It could already be shown in in vitro experiments that FAP OX40 iMAb can change T cell activation status and cytokine release. It was also confirmed that the influence of OX40 seems to be stronger on CD4 positive T cells than on CD8 positive T cells.

To test if FAP OX40 iMAb could also in vivo change the immune status to a more beneficial outcome we used a humanized mouse model transferring human stem cells into immunodeficient mice and therefore generating a partially human immune system consisting mainly of T and B cells. We coinjected MKN45, a CEA expressing human gastric cancer cell line, and 3T3 fibroblasts which improve the stroma component and FAP expression in the tumor. CEA is targeted by the CEACAM5 CD3 TCB, crosslinking T cells with tumor cells and inducing T cell mediated killing of tumors cells and T cell activation. Upon T cell activation OX40 is upregulated. FAP OX40 iMAb crosslinks FAP expressing fibroblasts and OX40 expressing T cells and is therefore inducing OX40 signaling. This leads to improved T cell survival and cytokine release.

We could prove in this study that combination therapy of FAP OX40 iMAb and CEACAM5 CD3 TCB leads to improved efficacy compared to monotherapies. Also FAP OX40 iMAb monotherapy showed significant improved efficacy compared to vehicle.

We evaluated the serum concentration of CEACAM5 CD3 TCB as well as FAPOx40iMAB upon the 1rst treatment in the respective monotherapies and in the combination group to rule out differences in exposure as cause of differences in efficacy. As shown in FIGS. 18A and 18B the exposure for all constructs was comparable in mono and combination therapy.

As shown in FIGS. 19A and 19B, FAP OX40 iMAb monotherapy treated animals showed a slightly delayed progression of the tumor, CEACAM5 CD3 TCB a more pronounced one. However, only in the combination therapy a regression of the subcutaneous tumor was achieved (see Table 3).

TABLE 3
Tumor growth inhibition (TGI) at study day 41 and 43

| Group | TGI day 41 [%] | TGI day 43 [%] |
|---|---|---|
| CEACAM5 CD3 TCB | 93.6 | 92.6 |
| FAP OX40 iMab | 55.2 | 35.9 |
| CEACAM5 CD3 TCB + | 103.8 | 103.4 |
| FAP OX40 iMab | | |

### 4.9 Results of Experiment 2

In a second study we tested different doses of FAPDX40iMAB as monotherapy and in combination with CEACAM5 CD3 TCB (CEA CD3 TCB (2)). Here, we also delayed the start of treatment until we reached a median tumor size of 490 mm³ compared to 170 mm³ in the first study.

All groups injected with compounds showed comparable maximum concentrations of the molecules between the different groups, either OX40 targeted compounds or TCB. In FIGS. 20A and 20B the pharmacokinetic profile of the injected compounds during the first week is shown.

As plotted in FIGS. 21A-21C, we could again confirm the superior anti-tumor efficacy of the combination versus the monotherapies. Neither FAP OX40 iMAb in any of the tested doses nor CEACAM5 CD3 TCB as monotherapy was able to slow down progression of the tumor growth, which was most likely due to the considerable tumor burden already at the beginning of treatment. Only the combination treatment significantly prevented the progression of tumor growth over the whole study time (Table 4). Strong prolonged efficacy was observed at doses of 12.5 mg/kg of FAP OX40 iMAB, however, lower doses (4.2 and 1.4 mg/kg) were only temporally able to reduce progression compared to CEACAM5 CD3 TCB monotherapy (FIG. 22). A clear dose dependency was observed. As shown in FIGS. 20A and 20B exposure for all constructs was comparable in mono and combination therapy.

Tumor growth inhibition based on medians was calculated at study day 40 and 49. The values can be found in Table 4 below.

TABLE 4
Tumor growth inhibition (TGI) at study day 40 and 49

| Group | TGI day 40 [%] | TGI day 49 [%] |
|---|---|---|
| CEACAM5 CD3 TCB | 67.3 | 36.7 |
| FAP OX40 iMab | 11.2 | −6.3 |
| 1.4 mg/kg | | |
| FAP OX40 iMab | 16.2 | 20.5 |
| 4.2 mg/kg | | |
| FAP OX40 iMab | 38.1 | 23.9 |
| 12.5 mg/kg | | |
| CEACAM5 CD3 TCB + | 55.2 | 26.7 |
| FAP OX40 iMab 1.4 mg/kg | | |
| CEACAM5 CD3 TCB + | 55.0 | 61.9 |
| FAP OX40 iMab 4.2 mg/kg | | |
| CEACAM5 CD3 TCB + | 108.5 | 102.3 |
| FAP OX40 iMab 12.5 mg/kg | | |

To test for significant differences in group means for multiple comparisons, the standard analysis of variance (ANOVA) is automatically produced, using the Dunnett's method. Dunnett's method tests whether means are different from the mean of a control group.

TABLE 5
p-values: Comparison with a control using Dunnett's
method (AUC = area under the curve)

| | | p-value | p-value | p-value |
|---|---|---|---|---|
| | p-value | AUC until | day 49 vs | AUC until day |
| | day 49 vs | day 49 vs | CEA CD3 | 49 vs CEA |
| Group | vehicle | vehicle | TCB (2) | CD3 TCB (2) |
| Vehicle | 1 | 1 | 0.1051 | 0.2158 |
| CEACAM5 CD3 | 0.1088 | 0.2158 | 1 | 1 |
| TCB | | | | |
| FAP OX40iMab | 0.5990 | 0.7234 | 0.7956 | 0.9171 |
| 12.5 mg/kg | | | | |
| FAP OX40iMab | 0.8848 | 0.9178 | 0.5394 | 0.7588 |
| 4.2 mg/kg | | | | |
| FAP OX40iMab | 0.9986 | 0.7666 | 0.2130 | 0.8886 |
| 1.4 mg/kg | | | | |
| CEACAM5 CD3 | <0.0001* | <0.0001* | 0.0032* | 0.0099* |
| TCB + FAP OX40 | | | | |
| iMab 12.5 mg/kg | | | | |
| CEACAM5 CD3 | 0.0234* | 0.0151* | 0.9924 | 0.8131 |
| TCB + FAP OX40 | | | | |
| iMab 4.2 mg/kg | | | | |
| CEACAM5 CD3 | 0.0449* | 0.0803 | 0.9998 | 0.9970 |
| TCB + FAP OX40 | | | | |
| iMab 1.4 mg/kg | | | | |

Flow cytometric (FIGS. 23A-23D) and histopathological (FIGS. 25A and 25B) evaluation showed an increased infiltration of the tumor mass with human leukocytes. This was already observed for CEACAM5 CD3 TCB monotherapy, but strongly enhanced in the combination of CEACAM5 CD3 TCB with 4.2 or 12.5 mg/kg FAP OX40 iMAB. FAP OX40 iMAB monotherapy per se increased intratumoral leukocyte counts only minimally. Cell types detected were human CD4 as well as CD8 T cells, but also non-T cells (e.g. B cells or myeloid derived cells). Interestingly, the fold increase in the combination therapy compared to CEACAM5 CD3 TCB monotherapy was more pronounced for CD4 T cells than for CD8 T cell counts, which is in line with the biology of OX40, being primarily expressed on CD4 T cells. In the periphery no significant alterations in cell numbers were detected, emphasizing the tumor targeted nature of both compounds (FIGS. 24A and 24B).

We also evaluated the concentrations of spleen, blood and intratumoral cytokines (Bio-Plex Pro™ Human Cytokine 17-plex Assay, BioRad). The group with the highest anti-tumor efficacy showed also the biggest overall increase in intratumoral cytokines (e.g. IL-6, IL-8, IFN-γ, TNF-α, MCP-1, MIP-1β (FIGS. 26A-26C) and was the combination of FAP Ox40 iMAB (12.5 mg/kg) and CEACAM5 CD3 TCB. No significant changes were observed in the periphery (spleen or blood). Thus, the immunological changes triggered by FAP OX40 iMAB and CEACAM5 CD3 TCB treatment were tumor-specific indicating that the cross-linking and activation of human T-cells occurs exclusively in CEA expressing tumors and not in other areas that are negative for CEA like blood and spleen.

We further found a direct negative correlation between tumor progression and the amount of intratumoral cytokine concentration, but not between the intratumoral leukocyte count for combination treated animals (FIGS. 27A-27F). The amount of cytokines present did also not strictly correlate with the number of infiltrating leukocytes for all animals. Especially, when CEACAM5 CD3 TCB monotherapy treated animals were compared with combination treated animals we observed that similar leukocyte counts did not necessarily mean the same anti-tumor efficacy or cytokine content present. This leads to the assumption, that beyond the mere increased number of intratumoral T cells, a higher per cell functionality and potential to secrete cytokines of intratumoral T cells are causative for the enhanced anti-tumor activity of the combination of FAP OX40 iMAB and CEACAM5 CD3 TCB.

An improved cytokine milieu plays a major role in mediating anti tumor efficacy. It can recruit more lymphocytes to the tumor, support proliferation and increase the survival of those T cells and prevents the establishment of suppression and exhaustion. We could show that FAP OX40 iMAb was able to modulate in vitro the TCB mediated secretion of cytokines for different tumor cell lines, effector populations and tumor targets towards a more inflammatory and less suppressive one. Furthermore, we could also show that this translated into improved anti-tumor efficacy in a humanized mouse model simulating the human immune system.

### Example 5

### Combination Therapy of FAP OX40 iMab, CEA TCB and PD-L1 Antibody In Vivo

### 5.1 Experimental Procedure

In the following example the human FAP targeted OX40 agonist FAP OX40 iMAb (FAP binder 4B9) was tested in a concentration of 12.5 mg/kg in combination with the human CEA CD3 TCB and an anti-PD-L1 antibody (a-PD-L1) in a human gastric MKN45 cancer model. MKN45 cells were cografted sub cutaneously with a mouse fibroblast cell line (3T3) in NSG humanized mice.

Human MKN45 cells (human gastric carcinoma) were originally obtained from DSMZ and after expansion deposited in the Glycart internal cell bank. Cells were cultured in DMEM containing 10% FCS at 37° C. in a water-saturated atmosphere at 5% CO2. In vitro passage 13 was used for subcutaneous injection at a viability of 99.1%. Human fibroblasts NIH-3T3 were originally obtained from ATCC, engineered at Hoffmann-La Roche Inc. to express human FAP and cultured in DMEM containing 10% Calf serum, 1× Sodium Pyruvate and 1.5 μg/ml Puromycin. Clone 39 was used at in vitro passage number 8 and at a viability of 97.6%.

50 microliters cell suspension (1×10⁶ MKN45 cells+1×10⁶ 3 T3-huFAP) mixed with 50 microliters Matrigel were injected subcutaneously in the flank of anaesthetized mice with a 22G to 30G needle. NSG female mice (purchased from Charles River), age 5 weeks at start of the experiment, were maintained under specific-pathogen-free condition with daily cycles of 12 h light/12 h darkness according to committed guidelines (GV-Solas; Felasa; TierschG). Experimental study protocol was reviewed and approved by local government authorities. After arrival, animals were maintained for one week to get accustomed to the new environment and for observation. Continuous health monitoring was carried out on a daily basis.

For humanization, mice were injected with Busulfan (20 mg/kg) followed 24 hours later by injection of 100,000 human HSC (purchased from StemCell Technologies).

7-14 days before cell injection mice were bled and screened for the amount of human T cells in the blood. Mice were randomized for human T cells with an average T cell count/ul blood of 131. Mice were injected sub cutaneously on study day 0 with 1×106 MKN45 cells mixed with 1×10⁶ 3 T3 fibroblasts. Tumors were measured 2 to 3 times per week during the whole experiment by Caliper. On day 17, mice were randomized for tumor size with an average tumor size of 205 mm³. On day of randomization mice were injected weekly i.v. with Vehicle, CEA CD3 TCB, CEA CD3 TCB plus a-PD-L1, CEA CD3 TCB plus FAP OX40 iMAb or the triple combination of CEA CD3 TCB, a-PD-L1 and FAP OX40 iMAb for up to 4 weeks. All mice were injected i.v. with 200 μl of the appropriate solution. The mice in the vehicle group were injected with Histidine Buffer and the treatment groups with the CEA CD3 TCB and the combinations of CEA CD3 TCB and/or FAP OX40 iMAb. To obtain the proper amount of compound per 200 μl, the stock solutions were diluted with Histidine Buffer when necessary. The dose and schedule used for CEA CD3 TCB was 2.5 mg/kg twice/week whereas FAP OX40 iMAb was given at a dose of 12.5 mg/kg and a-PD-L1 at a dose of 10 mg/kg once/week (Table 7). The experiment was terminated at study day 44. Some mice had to be sacrificed due to bad health status during the experiment.

TABLE 6
Mice alive on day 44

| | | CEA | CEA CD3 | CEA CD3 | CEA CD3 TCB + |
|---|---|---|---|---|---|
| | | CD3 | TCB + FAP | TCB + | FAP OX40 iMAb + |
| Group | Vehicle | TCB | OX40 iMAb | a-PD-L1 | a-PD-L1 |
| mice | 7/9 | 5/9 | 5/10 | 3/9 | 6/10 |
| alive | | | | | |
| day 44 | | | | | |

Tumors and blood were harvested in PBS, single cell suspensions were generated and stained for different immune cell markers and analysed by FACS. Plasma as well as part of tumor was frozen for Cytokine analysis via Multiplex. Parts of tumors at termination were formalin fixed and afterwards embedded in Paraffin. Samples were cut and stained for CD3 and CD8.

TABLE 7
Compositions used in the in vivo experiment

| | Dose | Concentration | |
|---|---|---|---|
| Compound | (mg/kg) | (mg/mL) | Formulation buffer |
| a-PD-L1 | 10 | 2.54 | 20 mM Histidine, 140 mM |
| (iTME-0005) | | (=stock | NaCl, pH 6.0 |
| | | concentration) | |
| CEA CD3 TCB | 2.5 | 4.82 | 20 mM Histidine, 140 mM |
| | | (=stock | NaCl, 0.01% Tween20, pH |
| | | concentration) | 6.0 |
| FAP OX40 iMAb | 3.2 | 4.82 | 20 mM Histidine, 140 mM |
| | | (=stock | NaCl, pH 6.0 |
| | | concentration) | |

### 5.2 Results

In this study we wanted to prove for the first time that FAP OX40 iMAb can improve efficacy mediated by the combination of CEA CD3 TCB and a-PD-L1. a-PD-L1 is an immune checkpoint inhibitor and is well established in the field of cancer immunotherapy. The a-PD-L1 binder is crossreactive to mouse PD-L1 and was produced in a murine IgG format. CEA CD3 TCB is targeting CEA expressed on cancer cells and FAP OX40 iMAb binds to FAP expressing fibroblasts in the tumor stroma. FAP OX40 iMAb was given weekly at a dose of 12.5 mg/kg and a-PD-L1 at a dose of 10 mg/kg whereas the CEA CD3 TCB was given at the dose of 2.5 mg/kg twice per week.

To test our human constructs human immune cells and specifically T cells have to be present in the mouse system. For this reason, we used humanized mice meaning mice transferred with human stem cells. These mice develop over time a partially human immune system consisting mainly of T and B cells.

We coinjected MKN45, a CEA expressing human gastric cancer cell line, and 3T3 fibroblasts which improve the stroma component in the tumor. CEA is targeted by the CEA CD3 TCB, crosslinking T cells with tumor cells and inducing T cell mediated killing of tumor cells and T cell activation. Upon T cell activation OX40 is upregulated as well as PD-1. FAP OX40 iMAb crosslinks FAP expressing stroma cells and OX40 expressing T cells and is therefore inducing OX40 signaling. This leads to improved cytokine secretion, survival and proliferation of the T cells. PD-L1 is mainly expressed by tumor cells, blocking of PD-L1 prevents crosslinking with PD-1 expressing T cells and therefore prevents PD-1 dependent inactivation of T cells.

We could show in this study that CEA CD3 TCB in combination with a-PD-L1 and FAP OX40 iMAb mediates improved efficacy in terms of tumor growth inhibition compared to the vehicle group (FIGS. 28A and 28B). Tumor growth inhibition based on medians was calculated at study day 36, 38, 41 and 43. The Group treated with CEA CD3 TCB+a-PD-L1+FAP OX40iMAb shows the strongest inhibition of tumor growth.

TABLE 8
Tumor growth inhibition (TGI) on day 36, 38, 41 and 43

| Group | Day 36 | Day 38 | Day 41 | Day 43 |
|---|---|---|---|---|
| CEA CD3 TCB | 62.77 | 55.13 | 47.30 | 32.73 |
| CEA CD3 TCB + FAP | 36.76 | 40.63 | 32.97 | 19.48 |
| OX40 iMab | | | | |
| 12.5 mg/kg | | | | |
| CEA CD3 TCB + | 45.91 | 55.32 | 41.35 | 37.60 |
| a-PD-L1 | | | | |
| CEA CD3 TCB + | 81.28 | 82.63 | 71.61 | 59.21 |
| a-PD-L1 + | | | | |
| FAP OX40iMab | | | | |

Considering the area under the curve (AUC) until day day 43 only the combination of CEA CD3 TCB+a-PD-L1+FAP OX40iMAb is significant different from vehicle monotherapy.

TABLE 9
One Way Analysis of tumor volumes until
day 43, AUC, comparison with vehicle

| Means Comparisons with a control | |
|---|---|
| using Dunnett's Method (sAUC) | p-Value |
| Control Group = vehicle | — |
| CEA CD3 TCB | 0.0563 |
| CEA CD3 TCB + FAP | 0.6395 |
| OX40iMAb | |
| CEA CD3 TCB + | 0.1318 |
| a-PD-L1 | |
| CEA CD3 TCB + | 0.0079* |
| a-PD-L1 + | |
| FAP OX40iMAb | |

TABLE 10
One Way Analysis of tumor volumes on
day 43, AUC, comparison with vehicle

| Comparisons with a control using | |
|---|---|
| Dunnett's Method (day 43) | p-Value |
| Control Group = vehicle | |
| CEA CD3 TCB | 0.1311 |
| CEA CD3 TCB + FAP OX40iMAb | 0.7221 |
| CEA CD3 TCB + a-PD-L1 | 0.1186 |
| CEA CD3 TCB + a-PD-L1 + FAP OX40iMAb | 0.0024* |

All other groups (monotherapies as well as double therapies) could not significantly improve efficacy compared to vehicle.

The pharmacokinetic profile of the injected compounds during the first week was studied as described in Example 4. In addition, for detecting a-PD-L1 biotinylated anti human Fc, PD-L1-huFc, test sample and polyclonal anti murine IgG (HRP) are added stepwise to a 96-well streptavidin-coated microtiter plate and incubated after every step for 1 h at room temperature. The plate was washed three times after each step to remove unbound substances. Finally, the peroxidase-bound complex is visualized by adding ABTS substrate solution to form a colored reaction product. The reaction product intensity, which is photometrically determined at 405 nm (with reference wavelength at 490 nm), is proportional to the analyte concentration in the serum sample. 2 mice per Group were bled 1 h and 72h after 1^{st} and 3^{rd} therapy and the injected compounds were analysed by ELISA. All groups injected with compounds show comparable exposure of the molecules between the different groups, either FAP OX40 iMAb, CEA CD3 TCB or a-PD-L1 (see FIGS. 29A, 29B and 29C).

T-cell infiltration in the tumor at termination by IHC (Immune histochemistry) on day 44 is significantly increased in the triple combination group compared to all other groups (see FIGS. 30A and 30B).

### Example 6

### Combination Therapy of FAP OX40 iMab, CEA TCB and PD-L1 Antibody In Vitro

### 6.1 Experimental Procedure

In this assay FAP OX40 iMAb was tested for its potential to activate human PBMCs (isolated from buffy coat, frozen and stored in liquid nitrogen) in the presence or absence of CEA CD3 TCB and atezolizumab (Tecentriq, anti-human PD-L1-specific humanized human IgG1κ antibody) similar as described in Example 5. To mimic the tumor environment PBMCs of six different donors were incubated with FAP-expression NIH/3T3-huFAP fibroblast cell line and with CEA-expressing MKN45-FolR1-PDL1 gastric cancer cell line for four days in the presence of absence of 2 nM FAP OX40 iMab and/or 100 nM CEA CD3 TCB and/or 80 nM atezolizumab. For determining PBMC activation CD4 and CD8 T cells were analyzed by flow cytometry for proliferation (CFSE-dilution), CD25 (IL-2Rα), 4-1BB (CD137), OX-40 (CD134), T-bet (T-box transcription factor), Eomes (Eomesodermin), Granzyme B, and PD-1 expression. Supernatant was analyzed by Multiplex for IFNγ, TNFα, GM-CSF, Granzyme B, IL-2, IL-8 and IL-10.

### a) Preparation of PBMCs

Buffy coats were obtained from the Zurich blood donation center. To isolate fresh peripheral blood mononuclear cells (PBMCs) the buffy coat was diluted with the same volume of DPBS (Gibco by Life Technologies, Cat. No. 14190326). 50 mL Falcon centrifuge tubes (TPP, Cat.-No. 91050) were supplied with 15 mL Histopaque 1077 (SIGMA Life Science, Cat.-No. 10771, polysucrose and sodium diatrizoate, adjusted to a density of 1.077 g/mL) and the buffy coat solution was over-layered on 15 mL Histopaque 1077. The tubes were centrifuged for 30 min at 400×g, room temperature and with low acceleration and no break. Afterwards the PBMCs were collected from the interface, washed three times with DPBS and resuspended in T cell freezing medium consisting of 90% (v/v) Fetal Bovine Serum (FBS, Gibco by Life Technology, Cat. No. 16000-044, Lot 941273, gamma-irradiated, mycoplasma-free and heat inactivated at 56° C. for 35 min) and 10% Dimethyl sulfoxide (Sigma, Cat.-No. D2650) 10% (v/v). 1 mL were transferred quickly to sterile Cryovials, transferred to Cryoboxes and stored for 24 h at −80° C. Afterwards vials were transferred to liquid nitrogen containers or Vapor phase containers.

Vials from 6 donors were thawed in the water bath at 37° C. and washed in assay medium consisting of RPMI 1640 medium supplied with 10% (v/v) Fetal Bovine Serum (FBS), 1% (v/v) GlutaMAX I, 1 mM Sodium pyruvate (SIGMA, Cat. No. S8636), 1% (v/v) MEM non-essential amino acids (SIGMA, Cat.-No. M7145) and 50 μM β-Mercaptoethanol (SIGMA, M3148). After thawing the cells were rested for 2 hours at 37° C. and 5% CO₂ in cell incubator. Cells were counted, washed with DPBS and resuspended in 37° C. DPBS to 1×10⁶ cells/mL. CFDA-SE was added to a final concentration of 200 nM and incubated for 10 min at 37° C. Afterwards FBS was added, cells were washed and set in assay medium to 2×10⁶ cells/mL).

### b) Target Cell Lines

T150 flasks containing NIH/3T3-huFAP clone 19 were washed with DPBS and incubated with enzyme-free PBS-based dissociation buffer for 8 min at 37° C. Cells were collected, washed, resuspended in assay medium and irradiated with 50 Gy using X-Ray Irradiator RS 2000. Cells were set in assay medium to 1×10⁶ cells/mL.

T150 flasks containing MKN45-FolR1-PDL1 gastric cancer cell line were washed with DPBS and incubated with enzyme-free PBS-based dissociation buffer for 8 min at 37° C. Cells were collected, washed with DPBS and resuspended in C diluent (at least 250 μL, 8×10⁷ cells/mL or lower). The same amount of C diluent was supplied with 4 μL/mL PKH-26 dye and mixed well. This dye solution was added to the cells and mixed well and immediately. Cells were incubated for 5 min at room temperature. Afterwards FBS was added, cells were washed in assay, resuspended in assay medium and irradiated with 50 Gy with the X-Ray Irradiator RS 2000 (Rad source). Cells were set in assay medium to 1×10⁶ cells/mL.

### c) Assay Setup

For the test compounds master solutions were prepared of each component in assay medium as follows 16 nM FAP OX40 iMAB, 800 nM CEA CD3 TCB and 640 nM Atezolizumab. Cells and components were combined in 96-well round bottom tissue culture plates (TTP, Cat.-No. 92097) in amounts of 50 μL of PKH-26 red labeled MKN45-FolR1-PD-L1 (10,000 cells/well), 50 μL of NIH/3T3-huFAP clone 19 (10,000 cells/well), 25 μL of PBMC of one donor (50,000 cells/well), 25 μL of 16 nM FAP OX40 iMAB solution or assay medium (final concentration 2 nM), 25 μL of 800 nM CEA CD3 TCB solution or assay medium (final concentration 100 nM), and 25 μL of 640 nM Atezolizumab solution or assay medium (final concentration 80 nM). Plates were then incubated for four days at 37° C. and 5% CO₂ in a humidified cell incubator.

After four days 50 μL supernatant was removed and stored at −80° C. to be later analyzed for cytokine content (see below). To perform a flow cytometry analysis of T-cell proliferation and surface expression of T cell activation markers, plates were centrifuged and washed once with cold DPBS. Samples were divided in equal volumes in two 96-welled plate for 2 individual staining panels. For staining panel 1, cells were stained for 15 min at room temperature (RT) in 50 μL/well DPBS supplied with 1:800 diluted LIVE/DEAD Fixable Aqua Dead Cell Stain. Cells were washed once with 200 μL/well FACS buffer (centrifugation 350×g 4 min at 4° C., flick off). After, they were resuspended in 25p. L/well staining solution composed of FACS-buffer containing antibodies anti-human CD4 (clone A161A1, Biolegend, Cat. No.-357410), CD8 (clone RPA-T8, Biolegend, Cat.-No. 301040), CD25 (clone BC96, Biolegend, Cat.-No. 302636), PD-1 (clone EH12.2H7, Biolegend, Cat.-No. 329920), CD134 (clone Ber-ACT35, Biolegend, Cat. No.-350008), CD137 (clone 4B4-1, Biolegend, Cat. No.-309814) and incubated for 20 min at 4° C. Cells were washed once with 200 μL/well FACS-buffer (centrifugation 350×g 4 min 4° C., flick off) and resuspended in 120 μL/well FACS buffer) before they were acquired the same day using 4-laser LSRII (BD Bioscience with DIVA software).

For Staining Panel 2, cells were stained for 15 min at room temperature (RT) in 50 μL/well DPBS supplied with 1:800 diluted LIVE/DEAD Fixable Aqua Dead Cell Stain and were washed once with 200 4/well FACS-buffer (centrifugation 350×g 4 min 4° C., flick off). Cells were resuspended in 25 4/well staining solution composed of FACS-buffer containing antibodies anti-human CD4 (clone RPA-T4, Biolegend, Cat.-No. 300558), CD8 (SK-1, Biolegend, Cat.-No. 344710), CCR7 (clone G043H7, Biolegend, Cat.-No. 353204), CD45RO (clone BC96, Biolegend, Cat.-No. 304236) and incubated for 20 min at 4° C. Cells were washed once with 200 μL/well FACS-buffer (centrifugation 350×g 4 min 4° C., flick off) and resuspended in 100 μL/well of Foxp3 Fixation/Permeabilization working solution by mixing 1 part of Foxp3 Fixation/Permeabilization Concentrate with 3 parts of Foxp3 Fixation/Permeabilization Diluent (FoxP3/Transcription Factor Staining Buffer Set, eBiosciences, Cat. No.-005523-00) for 60 min at RT. Cells were then washed once with Permeabilization Buffer working solution by mixing 1 part Permeabilization buffer with 9 parts of water (FoxP3/Transcription Factor Staining Buffer Set, eBiosciences, Cat. No.-005523-00) and were resuspended in 504/well staining solution composed of Permeabilization buffer working solution containing antibodies anti-human EOMES (clone Danl lmag, eBiosciences, Cat. No.-25-4857-80), T-bet (clone 4B10, Biolegend, Cat. No.-644815) and Granzyme B (clone GB11, Biolegend, Cat. No. 515406) for 40 mins at RT. Cells were then washed twice with 2004/well Permeabilization Buffer working solution and resuspended in 120 μL/well FACS buffer) before they were acquired the same day using 4-laser LSRII (BD Bioscience with DIVA software). Data was analyzed using FlowJo v10.3 for PC (FlowJo LLC), Microsoft Excel (professional Plus 2010) and GraphPad Prism v6.07 (GraphPad Software, Inc). Living CD4 and CD8 T cells were gated (Zombie Aqua-, CD4 or CD8+) and counts, the mean fluorescence intensity (MFI) of activation marker (CD134, CD137, CD25, PD-1) or maturation marker (CCR7, CD45RO) or Transcription factors (T-bet, Eomes) or cytokine (Granzyme B) and percentage of positive cells or mean fluorescent intensity (MFI) were plotted for each condition.

To analyze the released cytokines in the supernatant, the previous frozen samples were taken and analyzed for IFNγ, GM-CSF, TNFα, IL-2, Granzyme B, IL-8 and IL-10 using the cytometric bead array according to manufacturer's instructions. Evaluated cytokines were IL-2 (Human IL-2 CBA Flex-set (Bead A4), BD Bioscience, Ca. No. 558270), TNF-α (Human TNF-α CBA Flex-set (Bead C4), BD Bioscience, Ca. No. 560112), IFN-γ (IFN-γ CBA Flex-set (Bead E7), BD Bioscience, Ca. No. 558269), IL-10 (Human IL-10 CBA Flex-set (Bead B7), BD Bioscience, Ca. No. 558274), TNF (Human TNF CBA Flex-set (Bead C4), BD Bioscience, Ca. No. 560112), IL-8 (Human IL-8 CBA Flex-set (Bead A9), BD Bioscience, Ca. No. 558277), Granzyme B (Human Granzyme B CBA Flex-set (Bead D7), BD Bioscience, Ca. No. 560304).

### 6.2 Results

FIGS. 31 to 35 relate to the results of an in vitro assay testing the efficacy of the combination of CEA CD3 TCB and FAP OX40iMAb as well as the triple combination of CEA CD3 TCB and FAP-4-1BBL with anti-PD-L1 antibody (atezolizumab). PBMCs were incubated for four days in the presence of MKN45-PD-L1 and NIH/3T3-huFAP cells and different combinations of T cell activator CEA CD3 TCB, checkpoint inhibitor a-PD-L1 (atezolizumab) and immunomodulator FAP OX40 iMAb. At day 4, the endpoint of the experiment, cells were stained for surface or intracellular markers and supernatant was stored for cytokine analysis. Each symbol indicates an individual donor (each group was tested in triplicate), each color/pattern indicates a specific treatment combination, the bar indicates the mean with SEM. The effect of the combinations compared to the single components and combinations thereof on surface expression of CD25 on CD4 (FIG. 31A) and CD8 T cells (FIG. 31B), proliferation on CD4 (FIG. 32A) and CD8 T cells (FIG. 32B) and intracellular expression of T-bet on CD4 (FIG. 33A) and CD8 T cells (FIG. 33B), and Granzyme B on CD4 (FIG. 33C) and CD8 T cells (FIG. 33D), respectively, is shown for 6 different donors. Statistical significance between different treatment groups was calculated using 2-way ANOVA (Tukey's multiple comparisons test), wherein the average of 6 donors with experimental triplicates per group was calculated. Stars (*) shown in the graphs indicate p-value, * indicates p value <0.05, ** indicates p value <0.01, *** indicates p values <0.001.

6.2.1 Combination of CEA CD3 TCB and FAP OX40 iMAb was Superior to Combination with a-PD-L1

As shown in FIGS. 31A and 31B, the addition of 100 nM CEA CD3 TCB (dotted filled bars, filled triangles) but not 2 nM FAP OX40 iMAb alone (open bars, open circles) could increase the expression of activation markers CD25, and proliferation of CD4 and CD8 T cells. Combination of FAP OX40 iMAb with CEA CD3 TCB (grey bars, open squares) and/or aPD-L1 (black bars, grey filled squares) led to highest activation and proliferation of CD4 and CD8 T cells as compared to combination treatment with CEA CD3 TCB and aPD-L1 (open bars, filled black circles) as shown in FIGS. 31A and 31B. Additionally, FAP OX40 iMAB and CEA CD3 TCB combination treatment led to higher percentages of T cell transcription factor (T-bet) on CD4 T cells and higher expression of T-bet on CD8 T cells (FIGS. 32A and 32B). T-bet expression regulates T helper 1 cell lineage commitment and these results show FAP OX40 iMAb treatment results in driving a Th1 T cell response. Further as shown in FIGS. 33A to 33D, combination of FAP OX40 iMAB and CEA CD3 TCB treatment leads to higher percentages of Granzyme B expressing CD4 and CD4 T cells, suggesting higher cytotoxic potential of T cells. Analysis of cytokine in the supernatant at the endpoint of the experiment showed higher amounts of pro-inflammatory cytokines IFN-γ and Granzyme B in CEA CD3 TCB and FAP OX40 iMAb as compared to CEA CD3 TCB and aPD-L1 treatment, however due to high donor to donor variability the differences were not statistically significant. Taken together, combination of CEA CD3 TCB with FAP OX40 iMAb resulted in superior activation, proliferation and Th1 differentiation of both CD4 and CD8 T cells.

6.2.2 Triple Combination of CEA CD3 TCB, FAP OX40 iMAb and PD-L1 Leads to Highest Cytokine Secretion

As shown in FIGS. 34A to 34C, triple combination of CEA CD3 TCB, FAP OX40 iMAb and PD-L1 (black bars, grey filled squares) was the most effective in release of immune cell-activating proinflammatory cytokines such as IFN-γ, Granzyme B and IL-8 as compared to all other treatment groups. As shown in FIG. 34C, triple combination treatment also led to highest intracellular expression of cytolytic enzyme Granzyme B on both CD4 and CD8 T cells, in concordance with our results measuring secreted cytokines. Fold increase of cytokines comparing the triple combination with the combination of CEA CD3 TCB and aPD-L1 is shown in FIGS. 35A to 35C. Despite the strong differences in level of cytokine secretion between different donors, triple combination led to higher than 2-fold difference in majority of the tested donors. Highest fold changes were observed for IL-8 and IFNγ. As shown in FIGS. 31A and 31B, triple combination (black bars, grey filled circles) did not lead to changes in proliferation and activation of CD4 and CD8 T cells as compared to CEA CD3 TCB and FAP OX40 iMAb combination treatment. Taken together, FAP OX40 iMAB co-stimulation when combined with CEA CD3 TCB leads to strong effects in stimulating T cell activation, proliferation and intracellular expression of Th1 lineage promoting, transcription factor T-bet and Granzyme B expression. Addition of PD-L1 to this combination further enhances the cytotoxic potential of both CD4 and CD8 T cells as seen, by increased expression of intracellular and secreted granzyme B and pro-inflammatory cytokine IFN-γ.

1. A method for treating or delaying progression of cancer in a patient, comprising administering to the patient a bispecific OX40 antibody comprising at least one antigen binding domain capable of specific binding to a tumor-associated antigen in combination with a T-cell activating anti-CD3 bispecific antibody specific for a tumor-associated antigen.

2. The method of claim 1, wherein the T-cell activating anti-CD3 bispecific antibody is an anti-CEA/anti-CD3 bispecific antibody or an anti-FolR1/anti-CD3 bispecific antibody.

3. The method of claim 1, wherein the bispecific OX40 antibody comprising at least one antigen binding domain capable of specific binding to a tumor-associated antigen and the T-cell activating anti-CD3 bispecific antibody are administered together in a single composition or administered separately in two or more different compositions.

4. The method of claim 1, wherein the bispecific OX40 antibody comprising at least one antigen binding domain capable of specific binding to a tumor-associated antigen acts synergistically with the T-cell activating anti-CD3 bispecific antibody.

5. The method of claim 1, wherein the bispecific OX40 antibody comprising at least one antigen binding domain capable of specific binding to a tumor-associated antigen is an anti-Fibroblast activation protein (FAP)/anti-OX40 bispecific antibody.

6. The method of claim 5, wherein the bispecific OX40 antibody comprises at least one antigen binding domain capable of specific binding to FAP comprising
(a) a heavy chain variable region (V_{H}FAP) comprising (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3, and a light chain variable region (V_{L}FAP) comprising (iv) CDR-L1 comprising the amino acid sequence of SEQ ID NO:4, (v) CDR-L2 comprising the amino acid sequence of SEQ ID NO:5, and (vi) CDR-L3 comprising the amino acid sequence of SEQ ID NO:6, or
(b) a heavy chain variable region (VHFAP) comprising (i) CDR-H1 comprising the amino acid sequence of SEQ ID NO:9, (ii) CDR-H2 comprising the amino acid sequence of SEQ ID NO:10, and (iii) CDR-H3 comprising the amino acid sequence of SEQ ID NO:11, and a light chain variable region (VL FAP) comprising (iv) CDR-L1 comprising the amino acid sequence of SEQ ID NO:12, (v) CDR-L2 comprising the amino acid sequence of SEQ ID NO:13, and (vi) CDR-L3 comprising the amino acid sequence of SEQ ID NO:14.

7. The method of claim 5, wherein the bispecific OX40 antibody comprises at least one antigen binding domain capable of specific binding to FAP comprising a heavy chain variable region (V_{H}FAP) comprising an amino acid sequence of SEQ ID NO:7 and a light chain variable region (V_{L}FAP) comprising an amino acid sequence of SEQ ID NO:8; or a heavy chain variable region (V_{H}FAP) comprising an amino acid sequence of SEQ ID NO:15 and a light chain variable region (V_{L}FAP) comprising an amino acid sequence of SEQ ID NO:16.

8. The method of claim 1, wherein the bispecific OX40 antibody comprises at least one antigen binding domain capable of specific binding to OX40 comprising
(a) a heavy chain variable region (V_{H}OX40) comprising (i) CDR-H1 comprising the amino acid sequence of SEQ ID NO:17, (ii) CDR-H2 comprising the amino acid sequence of SEQ ID NO:19, and (iii) CDR-H3 comprising the amino acid sequence of SEQ ID NO:22, and a light chain variable region (V_{L}OX40) comprising (iv) CDR-L1 comprising the amino acid sequence of SEQ ID NO:28, (v) CDR-L2 comprising the amino acid sequence of SEQ ID NO:31, and (vi) CDR-L3 comprising the amino acid sequence of SEQ ID NO:35, or
(b) a heavy chain variable region (V_{H}OX40) comprising (i) CDR-H1 comprising the amino acid sequence of SEQ ID NO:17, (ii) CDR-H2 comprising the amino acid sequence of SEQ ID NO:19, and (iii) CDR-H3 comprising the amino acid sequence of SEQ ID NO:21, and a light chain variable region (V_{L}OX40) comprising (iv) CDR-L1 comprising the amino acid sequence of SEQ ID NO:28, (v) CDR-L2 comprising the amino acid sequence of SEQ ID NO:31, and (vi) CDR-L3 comprising the amino acid sequence of SEQ ID NO:34, or
(c) a heavy chain variable region (V_{H}OX40) comprising (i) CDR-H1 comprising the amino acid sequence of SEQ ID NO:17, (ii) CDR-H2 comprising the amino acid sequence of SEQ ID NO:19, and (iii) CDR-H3 comprising the amino acid sequence of SEQ ID NO:23, and a light chain variable region (V_{L}OX40) comprising (iv) CDR-L1 comprising the amino acid sequence of SEQ ID NO:28, (v) CDR-L2 comprising the amino acid sequence of SEQ ID NO:31, and (vi) CDR-L3 comprising the amino acid sequence of SEQ ID NO:36, or
(d) a heavy chain variable region (V_{H}OX40) comprising (i) CDR-H1 comprising the amino acid sequence of SEQ ID NO:17, (ii) CDR-H2 comprising the amino acid sequence of SEQ ID NO:19, and (iii) CDR-H3 comprising the amino acid sequence of SEQ ID NO:24, and a light chain variable region (V_{L}OX40) comprising (iv) CDR-L1 comprising the amino acid sequence of SEQ ID NO:28, (v) CDR-L2 comprising the amino acid sequence of SEQ ID NO:31, and (vi) CDR-L3 comprising the amino acid sequence of SEQ ID NO:37, or
(e) a heavy chain variable region (V_{H}OX40) comprising (i) CDR-H1 comprising the amino acid sequence of SEQ ID NO:18, (ii) CDR-H2 comprising the amino acid sequence of SEQ ID NO:20, and (iii) CDR-H3 comprising the amino acid sequence of SEQ ID NO:25, and a light chain variable region (V_{L}OX40) comprising (iv) CDR-L1 comprising the amino acid sequence of SEQ ID NO:29, (v) CDR-L2 comprising the amino acid sequence of SEQ ID NO:32, and (vi) CDR-L3 comprising the amino acid sequence of SEQ ID NO:38, or
(f) a heavy chain variable region (V_{H}OX40) comprising (i) CDR-H1 comprising the amino acid sequence of SEQ ID NO:18, (ii) CDR-H2 comprising the amino acid sequence of SEQ ID NO:20, and (iii) CDR-H3 comprising the amino acid sequence of SEQ ID NO:26, and a light chain variable region (V_{L}OX40) comprising (iv) CDR-L1 comprising the amino acid sequence of SEQ ID NO:29, (v) CDR-L2 comprising the amino acid sequence of SEQ ID NO:32, and (vi) CDR-L3 comprising the amino acid sequence of SEQ ID NO:38, or
(g) a heavy chain variable region (V_{H}OX40) comprising (i) CDR-H1 comprising the amino acid sequence of SEQ ID NO:18, (ii) CDR-H2 comprising the amino acid sequence of SEQ ID NO:20, and (iii) CDR-H3 comprising the amino acid sequence of SEQ ID NO:27, and a light chain variable region (V_{L}OX40) comprising (iv) CDR-L1 comprising the amino acid sequence of SEQ ID NO:30, (v) CDR-L2 comprising the amino acid sequence of SEQ ID NO:33, and (vi) CDR-L3 comprising the amino acid sequence of SEQ ID NO:39.

9. The method of claim 1, wherein the bispecific OX40 antibody comprises at least one antigen binding domain capable of specific binding to OX40 comprising
(a) a heavy chain variable region (V_{H}OX40) comprising an amino acid sequence of SEQ ID NO:40 and a light chain variable region (V_{L}OX40) comprising an amino acid sequence of SEQ ID NO:41, or
(b) a heavy chain variable region (V_{H}OX40) comprising an amino acid sequence of SEQ ID NO:42 and a light chain variable region (V_{L}OX40) comprising an amino acid sequence of SEQ ID NO:43, or
(c) a heavy chain variable region (V_{H}OX40) comprising an amino acid sequence of SEQ ID NO:44 and a light chain variable region (V_{L}OX40) comprising an amino acid sequence of SEQ ID NO:45, or
(d) a heavy chain variable region (V_{H}OX40) comprising an amino acid sequence of SEQ ID NO:46 and a light chain variable region (V_{L}OX40) comprising an amino acid sequence of SEQ ID NO:47, or
(a) a heavy chain variable region (V_{H}OX40) comprising an amino acid sequence of SEQ ID NO:48 and a light chain variable region (V_{L}OX40) comprising an amino acid sequence of SEQ ID NO:49, or
(a) a heavy chain variable region (V_{H}OX40) comprising an amino acid sequence of SEQ ID NO:50 and a light chain variable region (V_{L}OX40) comprising an amino acid sequence of SEQ ID NO:51, or
(a) a heavy chain variable region (V_{H}OX40) comprising an amino acid sequence of SEQ ID NO:52 and a light chain variable region (V_{L}OX40) comprising an amino acid sequence of SEQ ID NO:53.

10. The method of claim 1, wherein the bispecific OX40 antibody comprises an IgG Fc domain, specifically an IgG1 Fc domain or an IgG4 Fc domain.

11. The method of claim 10, wherein the bispecific OX40 antibody comprises a Fc domain that comprises one or more amino acid substitution that reduces binding to an Fc receptor and/or effector function.

12. The method of claim 1, wherein the bispecific OX40 antibody comprises monovalent binding to a tumor associated target and tetravalent binding to OX40.

13. The method of claim 12, wherein the bispecific OX40 antibody comprises a first Fab fragment capable of specific binding to OX40 fused at the C-terminus of the CH1 domain to the VH domain of a second Fab fragment capable of specific binding to OX40 and a third Fab fragment capable of specific binding to OX40 fused at the C-terminus of the CH1 domain to the VH domain of a fourth Fab fragment capable of specific binding to OX40.

14. The method of claim 1, wherein the bispecific OX40 antibody comprises
(i) a first heavy chain comprising an amino acid sequence of SEQ ID NO:54, a second heavy chain comprising an amino acid sequence of SEQ ID NO:55, and four light chains comprising an amino acid sequence of SEQ ID NO:56, or
(ii) a first heavy chain comprising an amino acid sequence of SEQ ID NO:57, a second heavy chain comprising an amino acid sequence of SEQ ID NO:58, and four light chains comprising an amino acid sequence of SEQ ID NO:56, or
(i) a first heavy chain comprising an amino acid sequence of SEQ ID NO:59, a second heavy chain comprising an amino acid sequence of SEQ ID NO:60, and four light chains comprising an amino acid sequence of SEQ ID NO:56, or
(ii) a first heavy chain comprising an amino acid sequence of SEQ ID NO:61, a second heavy chain comprising an amino acid sequence of SEQ ID NO:62, and four light chains comprising an amino acid sequence of SEQ ID NO:56.

15. The method of claim 1, wherein the T-cell activating anti-CD3 bispecific antibody is an anti-CEA/anti-CD3 bispecific antibody.

16. The method of claim 15, wherein the T-cell activating anti-CD3 bispecific antibody comprises a first antigen binding domain comprising a heavy chain variable region (V_{H}CD3) and a light chain variable region (V_{L}CD3), and a second antigen binding domain comprising a heavy chain variable region (V_{H}CEA) and a light chain variable region (V_{L}CEA).

17. The method of claim 16, wherein the T-cell activating anti-CD3 bispecific antibody comprises a first antigen binding domain comprising a heavy chain variable region (V_{H}CD3) comprising CDR-H1 sequence of SEQ ID NO:63, CDR-H2 sequence of SEQ ID NO:64, and CDR-H3 sequence of SEQ ID NO:65; and a light chain variable region (V_{L}CD3) comprising CDR-L1 sequence of SEQ ID NO:66, CDR-L2 sequence of SEQ ID NO:67, and CDR-L3 sequence of SEQ ID NO:68.

18. The method of claim 16, wherein the T-cell activating anti-CD3 bispecific antibody comprises a first antigen binding domain comprising a heavy chain variable region (V_{H}CD3) comprising the amino acid sequence of SEQ ID NO:69 and a light chain variable region (V_{L}CD3) comprising the amino acid sequence of SEQ ID NO:70.

19. The method of claim 16, wherein the second antigen binding domain comprising
(a) a heavy chain variable region (V_{H}CEA) comprising CDR-H1 sequence of SEQ ID NO:71, CDR-H2 sequence of SEQ ID NO:72, and CDR-H3 sequence of SEQ ID NO:73, and a light chain variable region (V_{L}CEA) comprising CDR-L1 sequence of SEQ ID NO:74, CDR-L2 sequence of SEQ ID NO:75, and CDR-L3 sequence of SEQ ID NO:76, or
(b) a heavy chain variable region (V_{H}CEA) comprising CDR-H1 sequence of SEQ ID NO:79, CDR-H2 sequence of SEQ ID NO:80, and CDR-H3 sequence of SEQ ID NO:81, and a light chain variable region (V_{L}CEA) comprising CDR-L1 sequence of SEQ ID NO:82, CDR-L2 sequence of SEQ ID NO:83, and CDR-L3 sequence of SEQ ID NO:84.

20. The method of claim 16, wherein the second antigen binding domain comprising a heavy chain variable region (V_{H}CEA) comprising the amino acid sequence of SEQ ID NO:77 and a light chain variable region (V_{L}CEA) comprising the amino acid sequence of SEQ ID NO:78; or a heavy chain variable region (V_{H}CEA) comprising the amino acid sequence of SEQ ID NO:85 and a light chain variable region (V_{L}CEA) comprising the amino acid sequence of SEQ ID NO:86.

21. The method of claim 15, wherein the anti-CEA/anti-CD3 bispecific antibody comprises a third antigen binding domain that binds to CEA.

22. The method of claim 1, wherein the T-cell activating anti-CD3 bispecific antibody comprises an Fc domain comprising one or more amino acid substitutions that reduce binding to an Fc receptor and/or effector function.

23. The method of claim 1, wherein the T-cell activating anti-CD3 bispecific antibody is an anti-FolR1/anti-CD3 bispecific antibody.

24. The method of claim 23, wherein the T-cell activating anti-CD3 bispecific antibody comprises a first antigen binding domain comprising a heavy chain variable region (V_{H}CD3), a second antigen binding domain comprising a heavy chain variable region (V_{H}FolR1) and a common light chain variable region.

25. The method of claim 24, wherein the T-cell activating anti-CD3 bispecific antibody comprises a first antigen binding domain comprising a heavy chain variable region (V_{H}CD3) comprising CDR-H1 sequence of SEQ ID NO:95, CDR-H2 sequence of SEQ ID NO:96, and CDR-H3 sequence of SEQ ID NO:97; the second antigen binding domain comprising a heavy chain variable region (V_{H}FolR1) comprising CDR-H1 sequence of SEQ ID NO:98, CDR-H2 sequence of SEQ ID NO:99, and CDR-H3 sequence of SEQ ID NO:100; and a common light chain comprising a CDR-L1 sequence of SEQ ID NO:101, CDR-L2 sequence of SEQ ID NO:102, and CDR-L3 sequence of SEQ ID NO:103.

26. The method of claim 24, wherein the T-cell activating anti-CD3 bispecific antibody comprises a first antigen binding domain comprising a heavy chain variable region (V_{H}CD3) comprising the sequence of SEQ ID NO:104; a second antigen binding domain comprises a heavy chain variable region (V_{H}FolR1) comprising the sequence of SEQ ID NO:105; and a common light chain comprising the sequence of SEQ ID NO:106.

27. The method of claim 23, wherein the anti-FolR1/anti-CD3 bispecific antibody comprises a third antigen binding domain that binds to FolR1.

28. The method of claim 24, wherein the anti-FolR1/anti-CD3 bispecific antibody comprises a first heavy chain comprising the amino acid sequence of SEQ ID NO:107, a second heavy chain comprising the amino acid sequence of SEQ ID NO:108 and a common light chain of SEQ ID NO: 109.

29. The method of claim 1, further comprising administering to the patient an agent blocking PD-L1/PD-1 interaction.

30. The method of claim 29, wherein the agent blocking PD-L1/PD-1 interaction is an anti-PD-L1 antibody or an anti-PD1 antibody.

31. The method of claim 30, wherein the anti-PD-L1 antibody is atezolizumab.

32. A pharmaceutical composition comprising an anti-FAP/anti-OX40 bispecific antibody, an anti-CEA/anti-CD3 bispecific antibody or anti-FolR1/anti-CD3 bispecific antibody, and a pharmaceutically acceptable excipient.

33. The pharmaceutical composition of claim 32, further comprising an agent blocking PD-L1/PD-1 interaction.

34. The pharmaceutical composition of claim 33, wherein the agent blocking PD-L1/PD-1 interaction is an anti-PD-L1 antibody or an anti-PD1 antibody.

35. (canceled)

36. (canceled)

37. (canceled)

38. The pharmaceutical composition of claim 34, wherein the agent blocking PD-L1/PD-1 interaction is atezolizumab.
